SEQUENCE LISTING (570 sequences)

SEQ ID NO: 1 - protein, 16 aa, synthetic construct
FIADYFETSSQCSKPS

SEQ ID NO: 2 - protein, 16 aa, synthetic construct
FSRCCFSFAEQEIPLR

SEQ ID NO: 3 - protein, 16 aa, synthetic construct
FVVDYYETSSLCSQPA

SEQ ID NO: 4 - protein, 16 aa, synthetic construct
IFKTIVAKEICADPKQ

SEQ ID NO: 5 - protein, 16 aa, synthetic construct
IVRNFHYLLIKDGSRV

SEQ ID NO: 6 - protein, 16 aa, synthetic construct
LLESYFETNSECSKPG

SEQ ID NO: 7 - protein, 16 aa, synthetic construct
LRCVCLQTTQGVHPKM

SEQ ID NO: 8 - protein, 16 aa, synthetic construct
PEWVNTPSTCCLKYYE

SEQ ID NO: 9 - protein, 16 aa, synthetic construct
RITNIQCPKEAVIFKT

SEQ ID NO: 10 - protein, 16 aa, synthetic construct
CADPSESWVQEYVYDL

SEQ ID NO: 11 - protein, 16 aa, synthetic construct
VCADPSESWVQEYVYD

SEQ ID NO: 12 - protein, 16 aa, synthetic construct
ITLFASDFVLMWAVFF

SEQ ID NO: 13 - protein, 16 aa, synthetic construct
ALAFIYVLMFLANAIG

SEQ ID NO: 14 - protein, 16 aa, synthetic construct
ALAFIYVLMFLCNAIG

SEQ ID NO: 15 - protein, 16 aa, synthetic construct
ALAFIYVLMFLSNAIG

SEQ ID NO: 16 - protein, 16 aa, synthetic construct
IVTIIITFFLSWSPYH

SEQ ID NO: 17 - protein, 16 aa, synthetic construct
IYLLNLAISDLFFLLT

SEQ ID NO: 18 - protein, 16 aa, synthetic construct
IYLLNLAISNLLFLVT

SEQ ID NO: 19 - protein, 16 aa, synthetic construct
KIIFSYVVVFLVAWLP

SEQ ID NO: 20 - protein, 16 aa, synthetic construct
ALFSIFWWIFAVIIAI

SEQ ID NO: 21 - protein, 16 aa, synthetic construct
KLIFAIVVAYFLSWGP

SEQ ID NO: 22 - protein, 16 aa, synthetic construct
KQAALFSIFWWIFAVI

SEQ ID NO: 23 - protein, 16 aa, synthetic construct
KQACLFSIFWWIFAVI

SEQ ID NO: 24 - protein, 16 aa, synthetic construct
KQASLFSIFWWIFAVI

SEQ ID NO: 25 - protein, 16 aa, synthetic construct
ALIQMLEICIGFVVPF

SEQ ID NO: 26 - protein, 16 aa, synthetic construct
KVLWALQYIGLGFFAA

SEQ ID NO: 27 - protein, 16 aa, synthetic construct
LAFIYVLMFLANAIGN

SEQ ID NO: 28 - protein, 16 aa, synthetic construct
LAKLEAFFLNLSIYWS

SEQ ID NO: 29 - protein, 16 aa, synthetic construct
LALSISLLAEVLMFLF

SEQ ID NO: 30 - protein, 16 aa, synthetic construct
LATFKILIAIVWGVMP

SEQ ID NO: 31 - protein, 16 aa, synthetic construct
LATFKILICIVWGVMP

SEQ ID NO: 32 - protein, 16 aa, synthetic construct
LAVKIVFSFIWGVLPT

SEQ ID NO: 33 - protein, 16 aa, synthetic construct
LDFILALVGNTLALWL

SEQ ID NO: 34 - protein, 16 aa, synthetic construct
LENMLFASFYLLDFIL

SEQ ID NO: 35 - protein, 16 aa, synthetic construct
LFGSIGNFLVIFTITW

SEQ ID NO: 36 - protein, 16 aa, synthetic construct
LFIGIKIVYAFLWGIF

SEQ ID NO: 37 - protein, 16 aa, synthetic construct
LFIGIKIVYCFLWGIF

SEQ ID NO: 38 - protein, 16 aa, synthetic construct
LFMFVLSVLGNLTVVA

SEQ ID NO: 39 - protein, 16 aa, synthetic construct
LFMFVLSVLGNLTVVC

SEQ ID NO: 40 - protein, 16 aa, synthetic construct
LFMFVLSVLGNLTVVS

SEQ ID NO: 41 - protein, 16 aa, synthetic construct
LFSFNRFFCQLVYFIY

SEQ ID NO: 42 - protein, 16 aa, synthetic construct
LFSIFWWIFAVIIAIP

SEQ ID NO: 43 - protein, 16 aa, synthetic construct
LFTLYVSTLDFCIVFI

SEQ ID NO: 44 - protein, 16 aa, synthetic construct
LFVMLFSYGFTLRTLF

SEQ ID NO: 45 - protein, 16 aa, synthetic construct
LGIILFSYVEILRTLF

SEQ ID NO: 46 - protein, 16 aa, synthetic construct
LGIMLFAYSMIIRTLQ

SEQ ID NO: 47 - protein, 16 aa, synthetic construct
LGVWLMAIVGTFLNVL

SEQ ID NO: 48 - protein, 16 aa, synthetic construct
ALSISLLAEVLMFLFP

SEQ ID NO: 49 - protein, 16 aa, synthetic construct
LIWVISLLVSLPQIIY

SEQ ID NO: 50 - protein, 16 aa, synthetic construct
LIYLFLLLWEDTQGWG

SEQ ID NO: 51 - protein, 16 aa, synthetic construct
LLFFVFSFPYHVLNLL

SEQ ID NO: 52 - protein, 16 aa, synthetic construct
LLIMLFSYGFTLRTLF

SEQ ID NO: 53 - protein, 16 aa, synthetic construct
ALYSLLFLLGLLGNGA

SEQ ID NO: 54 - protein, 16 aa, synthetic construct
LLMLGLELLFGFFIPL

SEQ ID NO: 55 - protein, 16 aa, synthetic construct
LLQALYSAVVPLIYSC

SEQ ID NO: 56 - protein, 16 aa, synthetic construct
LLSVLFTLYVSTLDFS

SEQ ID NO: 57 - protein, 16 aa, synthetic construct
LLTWLAGLIFSVPAAV

SEQ ID NO: 58 - protein, 16 aa, synthetic construct
LMFMIFAYTFIVKTLV

SEQ ID NO: 59 - protein, 16 aa, synthetic construct
LMLVFSFVSMLASLAH

SEQ ID NO: 60 - protein, 16 aa, synthetic construct
LPFWGISVAWHWVFGS

SEQ ID NO: 61 - protein, 16 aa, synthetic construct
LPTMVFSFFYVIFCKA

SEQ ID NO: 62 - protein, 16 aa, synthetic construct
LPTMVFSFFYVIFSKA

SEQ ID NO: 63 - protein, 16 aa, synthetic construct
LQTVITFALYVGQFLA

SEQ ID NO: 64 - protein, 16 aa, synthetic construct
LSISLLAEVLMFLFPN

SEQ ID NO: 65 - protein, 16 aa, synthetic construct
LSLSISLLAEVLMFLF

SEQ ID NO: 66 - protein, 16 aa, synthetic construct
LSWKVLLTLVWGAAPV

SEQ ID NO: 67 - protein, 16 aa, synthetic construct
LTGFLFYLNMYASIYF

SEQ ID NO: 68 - protein, 16 aa, synthetic construct
LTLKVILGFFLPFVVM

SEQ ID NO: 69 - protein, 16 aa, synthetic construct
ANVTFTGLIAAFSWFM

SEQ ID NO: 70 - protein, 16 aa, synthetic construct
LVFIFGFVGNMLVVLI

SEQ ID NO: 71 - protein, 16 aa, synthetic construct
LVFIIFWLPYHLTLFV

SEQ ID NO: 72 - protein, 16 aa, synthetic construct
LVFLLSLVGNSLVLWV

SEQ ID NO: 73 - protein, 16 aa, synthetic construct
LVFVIGLVGNILVVLV

SEQ ID NO: 74 - protein, 16 aa, synthetic construct
LVLGLLLNSLALWVFC

SEQ ID NO: 75 - protein, 16 aa, synthetic construct
LVLGLLLNSLALWVFS

SEQ ID NO: 76 - protein, 16 aa, synthetic construct
LVTLPFWGISVAWHWV

SEQ ID NO: 77 - protein, 16 aa, synthetic construct
LVTMAVWVASILSSIL

SEQ ID NO: 78 - protein, 16 aa, synthetic construct
LWVVVFQFQHIMVGLI

SEQ ID NO: 79 - protein, 16 aa, synthetic construct
LYAHLAAAFLWVVVAV

SEQ ID NO: 80 - protein, 16 aa, synthetic construct
LYAHLASAFLWVVVAV

SEQ ID NO: 81 - protein, 16 aa, synthetic construct
LYGVVFLFGSIGNFLV

SEQ ID NO: 82 - protein, 16 aa, synthetic construct
LYSLLFLLGLLGNGAV

SEQ ID NO: 83 - protein, 16 aa, synthetic construct
MFLLNLAIADLLFLVT

SEQ ID NO: 84 - protein, 16 aa, synthetic construct
MIFTWNLVLSQFFSIL

SEQ ID NO: 85 - protein, 16 aa, synthetic construct
MLFASFYLLDFILALV

SEQ ID NO: 86 - protein, 16 aa, synthetic construct
MMTWFYAFFYSTVQRA

SEQ ID NO: 87 - protein, 16 aa, synthetic construct
MSFFSGMLLLLCISID

SEQ ID NO: 88 - protein, 16 aa, synthetic construct
MVFSFFYVIFAKALHD

SEQ ID NO: 89 - protein, 16 aa, synthetic construct
MVFSFFYVIFSKALHD

SEQ ID NO: 90 - protein, 16 aa, synthetic construct
MVIWVLAFFLSSPSLV

SEQ ID NO: 91 - protein, 16 aa, synthetic construct
MVWANLLVFVVSFLPL

SEQ ID NO: 92 - protein, 16 aa, synthetic construct
NVTFTGLIAAFSWFML

SEQ ID NO: 93 - protein, 16 aa, synthetic construct
PCGIITSVLAWVTAIL

SEQ ID NO: 94 - protein, 16 aa, synthetic construct
PGPWMNILWAWFIFWW

SEQ ID NO: 95 - protein, 16 aa, synthetic construct
PSWIISFSVWMAAILL

SEQ ID NO: 96 - protein, 16 aa, synthetic construct
ASLFSIFWWIFAVIIA

SEQ ID NO: 97 - protein, 16 aa, synthetic construct
PVKQASLFSIFWWIFA

SEQ ID NO: 98 - protein, 16 aa, synthetic construct
PVTLFLYGVVFLFGSI

SEQ ID NO: 99 - protein, 16 aa, synthetic construct
PYHWGWVLGDFLAKLL

SEQ ID NO: 100 - protein, 16 aa, synthetic construct
QAALFSIFWWIFAVII

SEQ ID NO: 101 - protein, 16 aa, synthetic construct
QACLFSIFWWIFAVII

SEQ ID NO: 102 - protein, 16 aa, synthetic construct
QASLFSIFWWIFAVII

SEQ ID NO: 103 - protein, 16 aa, synthetic construct
ASMILESLFAALVTLI

SEQ ID NO: 104 - protein, 16 aa, synthetic construct
QHNLFFLLSLGIILFC

SEQ ID NO: 105 - protein, 16 aa, synthetic construct
QYLKAFLAAVLGWHLA

SEQ ID NO: 106 - protein, 16 aa, synthetic construct
RFGVWLMSIVGTFLNM

SEQ ID NO: 107 - protein, 16 aa, synthetic construct
RFRQYLKAFLAAVLGW

SEQ ID NO: 108 - protein, 16 aa, synthetic construct
RLGVWLMSIVGTFLNV

SEQ ID NO: 109 - protein, 16 aa, synthetic construct
ATFKILISIVWGVMPV

SEQ ID NO: 110 - protein, 16 aa, synthetic construct
RPVKQACLFSIFWWIF

SEQ ID NO: 111 - protein, 16 aa, synthetic construct
RPVKQASLFSIFWWIF

SEQ ID NO: 112 - protein, 16 aa, synthetic construct
RVTLTALAVWGLALLF

SEQ ID NO: 113 - protein, 16 aa, synthetic construct
RVTLTSLAVWGLSLLF

SEQ ID NO: 114 - protein, 16 aa, synthetic construct
SAVLTLKVILGFFLPF

SEQ ID NO: 115 - protein, 16 aa, synthetic construct
SESALAFIYVLMFLCN

SEQ ID NO: 116 - protein, 16 aa, synthetic construct
ATSWVVIFWILAALMG

SEQ ID NO: 117 - protein, 16 aa, synthetic construct
SFYISLFAGVSFLINL

SEQ ID NO: 118 - protein, 16 aa, synthetic construct
SFYVAMFASLSFITEI

SEQ ID NO: 119 - protein, 16 aa, synthetic construct
SGIFFIILLTIDRYLA

SEQ ID NO: 120 - protein, 16 aa, synthetic construct
SGIITSVLAWVTAILA

SEQ ID NO: 121 - protein, 16 aa, synthetic construct
ATYMLLVLYYAMLPLM

SEQ ID NO: 122 - protein, 16 aa, synthetic construct
SGYDMWLMLVYSVMIP

SEQ ID NO: 123 - protein, 16 aa, synthetic construct
SIFWWIFAVIIAIPHF

SEQ ID NO: 124 - protein, 16 aa, synthetic construct
SIILMFLSDFSFIFSS

SEQ ID NO: 125 - protein, 16 aa, synthetic construct
SIYLLNLAISDLLFLF

SEQ ID NO: 126 - protein, 16 aa, synthetic construct
SLCLSISLLAEVLMFL

SEQ ID NO: 127 - protein, 16 aa, synthetic construct
SLFAALVTLITSLFGS

SEQ ID NO: 128 - protein, 16 aa, synthetic construct
SLFCALVTLITSLFGS

SEQ ID NO: 129 - protein, 16 aa, synthetic construct
SLFSALVTLITSLFGS

SEQ ID NO: 130 - protein, 16 aa, synthetic construct
SLFSIFWWIFAVIIAI

SEQ ID NO: 131 - protein, 16 aa, synthetic construct
SLSISLLAEVLMFLFP

SEQ ID NO: 132 - protein, 16 aa, synthetic construct
SLVFIFGFVGNMLVIL

SEQ ID NO: 133 - protein, 16 aa, synthetic construct
SMILESLFAALVTLIT

SEQ ID NO: 134 - protein, 16 aa, synthetic construct
SMILESLFSALVTLIT

SEQ ID NO: 135 - protein, 16 aa, synthetic construct
SMVIWVLAFFLSSPSL

SEQ ID NO: 136 - protein, 16 aa, synthetic construct
AVFYALLFVFSLLGNS

SEQ ID NO: 137 - protein, 16 aa, synthetic construct
SNVTFTGLISAFSWFM

SEQ ID NO: 138 - protein, 16 aa, synthetic construct
AVKIVFSFIWGVLPTM

SEQ ID NO: 139 - protein, 16 aa, synthetic construct
SVSLVNLLTIIGCLWV

SEQ ID NO: 140 - protein, 16 aa, synthetic construct
SWIISFSVWMAAILLS

SEQ ID NO: 141 - protein, 16 aa, synthetic construct
SWKVLLTLVWGAAPVI

SEQ ID NO: 142 - protein, 16 aa, synthetic construct
SYRSTYIILLLTWFAG

SEQ ID NO: 143 - protein, 16 aa, synthetic construct
TASFYVAMFASLSFIT

SEQ ID NO: 144 - protein, 16 aa, synthetic construct
TEFDYDEDATPCVFTD

SEQ ID NO: 145 - protein, 16 aa, synthetic construct
TENFEYDDLAEACYIG

SEQ ID NO: 146 - protein, 16 aa, synthetic construct
TFGVVTSVITWLVAVF

SEQ ID NO: 147 - protein, 16 aa, synthetic construct
TFGVVTSVITWVVAVF

SEQ ID NO: 148 - protein, 16 aa, synthetic construct
TFKILICIVWGVMPVV

SEQ ID NO: 149 - protein, 16 aa, synthetic construct
TFKILISIVWGVMPVV

SEQ ID NO: 150 - protein, 16 aa, synthetic construct
AVWGLSLLFALPDFIF

SEQ ID NO: 151 - protein, 16 aa, synthetic construct
TFTGLIAAFSWFMLSL

SEQ ID NO: 152 - protein, 16 aa, synthetic construct
ACFYVAMFASLCFITE

SEQ ID NO: 153 - protein, 16 aa, synthetic construct
AWFLPIMYSIICFVGL

SEQ ID NO: 154 - protein, 16 aa, synthetic construct
AWFLPIMYSIISFVGL

SEQ ID NO: 155 - protein, 16 aa, synthetic construct
TIWLVGFLLALPEILF

SEQ ID NO: 156 - protein, 16 aa, synthetic construct
AWLPYYIGISIDSFIL

SEQ ID NO: 157 - protein, 16 aa, synthetic construct
TLFASDFVLMWAVFFM

SEQ ID NO: 158 - protein, 16 aa, synthetic construct
TLFASDFVLMWCVFFM

SEQ ID NO: 159 - protein, 16 aa, synthetic construct
TLPFWGISVAWHWVFG

SEQ ID NO: 160 - protein, 16 aa, synthetic construct
TNFDFMILNLSFADLF

SEQ ID NO: 161 - protein, 16 aa, synthetic construct
AYDLFIGIKIVYAFLW

SEQ ID NO: 162 - protein, 16 aa, synthetic construct
AYDLFIGIKIVYCFLW

SEQ ID NO: 163 - protein, 16 aa, synthetic construct
AYDLFIGIKIVYSFLW

SEQ ID NO: 164 - protein, 16 aa, synthetic construct
TNWKLLAEIYEYLFFF

SEQ ID NO: 165 - protein, 16 aa, synthetic construct
TRFGVWLMSIVGTFLN

SEQ ID NO: 166 - protein, 16 aa, synthetic construct
TRLGVWLMAIVGTFLN

SEQ ID NO: 167 - protein, 16 aa, synthetic construct
TSVITWLVAVFASVPG

SEQ ID NO: 168 - protein, 16 aa, synthetic construct
TTEFDYDEDATPCVFT

SEQ ID NO: 169 - protein, 16 aa, synthetic construct
TTVILILAFFASWLPY

SEQ ID NO: 170 - protein, 16 aa, synthetic construct
TVFLSIFYSVIFAIGL

SEQ ID NO: 171 - protein, 16 aa, synthetic construct
TVITFALYVGQFLAYV

SEQ ID NO: 172 - protein, 16 aa, synthetic construct
TVITFSLYVGQFLAYV

SEQ ID NO: 173 - protein, 16 aa, synthetic construct
TVKLIFAIVVAYFLSW

SEQ ID NO: 174 - protein, 16 aa, synthetic construct
TVLATFKILIAIVWGV

SEQ ID NO: 175 - protein, 16 aa, synthetic construct
TVLATFKILICIVWGV

SEQ ID NO: 176 - protein, 16 aa, synthetic construct
TVLATFKILISIVWGV

SEQ ID NO: 177 - protein, 16 aa, synthetic construct
TVLLSWKVLLTLVWGA

SEQ ID NO: 178 - protein, 16 aa, synthetic construct
TVLLSWKVLLTMVWGA

SEQ ID NO: 179 - protein, 16 aa, synthetic construct
ACLFSIFWWIFAVIIA

SEQ ID NO: 180 - protein, 16 aa, synthetic construct
TWNLVLSQFFSILATM

SEQ ID NO: 181 - protein, 16 aa, synthetic construct
TYIILLLTWFAGLIFS

SEQ ID NO: 182 - protein, 16 aa, synthetic construct
VAYSLIFLLGVIGNVL

SEQ ID NO: 183 - protein, 16 aa, synthetic construct
VCILVWLLAFCVSLPD

SEQ ID NO: 184 - protein, 16 aa, synthetic construct
CALVTLITSLFGSIFK

SEQ ID NO: 185 - protein, 16 aa, synthetic construct
VFGTVFLSIFYSVIFA

SEQ ID NO: 186 - protein, 16 aa, synthetic construct
VFLFGSIGNFLVIFTI

SEQ ID NO: 187 - protein, 16 aa, synthetic construct
VFLLSLLGNSLVMLVI

SEQ ID NO: 188 - protein, 16 aa, synthetic construct
VFLLSLVGNSLVLWVL

SEQ ID NO: 189 - protein, 16 aa, synthetic construct
VFLSIFYSVIFAIGLV

SEQ ID NO: 190 - protein, 16 aa, synthetic construct
VFYSLLFVFSLLGNSL

SEQ ID NO: 191 - protein, 16 aa, synthetic construct
VGALGNSLVILVYWYC

SEQ ID NO: 192 - protein, 16 aa, synthetic construct
VIGFLVPLLAMSFCYL

SEQ ID NO: 193 - protein, 16 aa, synthetic construct
VIMMTWFYAFFYSTVQ

SEQ ID NO: 194 - protein, 16 aa, synthetic construct
VITFCLYVGQFLAYVR

SEQ ID NO: 195 - protein, 16 aa, synthetic construct
VITWLVAVFASVPGII

SEQ ID NO: 196 - protein, 16 aa, synthetic construct
VKQAALFSIFWWIFAV

SEQ ID NO: 197 - protein, 16 aa, synthetic construct
VKQACLFSIFWWIFAV

SEQ ID NO: 198 - protein, 16 aa, synthetic construct
VKQASLFSIFWWIFAV

SEQ ID NO: 199 - protein, 16 aa, synthetic construct
VLGDFLCKLLNMIFSI

SEQ ID NO: 200 - protein, 16 aa, synthetic construct
VLGLLLNSLALWVFAA

SEQ ID NO: 201 - protein, 16 aa, synthetic construct
VLGLLLNSLALWVFSS

SEQ ID NO: 202 - protein, 16 aa, synthetic construct
CFLWGIFPTVIFSYFY

SEQ ID NO: 203 - protein, 16 aa, synthetic construct
VLLSWKVLLTMVWGAA

SEQ ID NO: 204 - protein, 16 aa, synthetic construct
VLTLKVILGFFLPFVV

SEQ ID NO: 205 - protein, 16 aa, synthetic construct
VLVIGSLVARWLLGIQ

SEQ ID NO: 206 - protein, 16 aa, synthetic construct
VLYFIADEVYTVLLSW

SEQ ID NO: 207 - protein, 16 aa, synthetic construct
CGIITSVLAWVTAILA

SEQ ID NO: 208 - protein, 16 aa, synthetic construct
VSILVWLLAFSVSLPD

SEQ ID NO: 209 - protein, 16 aa, synthetic construct
CILVWLLAFCVSLPDT

SEQ ID NO: 210 - protein, 16 aa, synthetic construct
VTLFLYGVVFLFGSIG

SEQ ID NO: 211 - protein, 16 aa, synthetic construct
VTLTSLAVWGLSLLFA

SEQ ID NO: 212 - protein, 16 aa, synthetic construct
VTSLLIWVISLLVSLP

SEQ ID NO: 213 - protein, 16 aa, synthetic construct
VTSVITWLVAVFASVP

SEQ ID NO: 214 - protein, 16 aa, synthetic construct
VVCILVWLLAFCVSLP

SEQ ID NO: 215 - protein, 16 aa, synthetic construct
VVFLFGSIGNFLVIFT

SEQ ID NO: 216 - protein, 16 aa, synthetic construct
VVLIFLLSWLPYNLVL

SEQ ID NO: 217 - protein, 16 aa, synthetic construct
VVTSVITWLVAVFASV

SEQ ID NO: 218 - protein, 16 aa, synthetic construct
VVTSVITWVVAVFASL

SEQ ID NO: 219 - protein, 16 aa, synthetic construct
VWLMAIVGTFLNVLVI

SEQ ID NO: 220 - protein, 16 aa, synthetic construct
VWLMCIVGTFLNVLVI

SEQ ID NO: 221 - protein, 16 aa, synthetic construct
VWLMSIVGTFLNMLVI

SEQ ID NO: 222 - protein, 16 aa, synthetic construct
VWLMSIVGTFLNVLVI

SEQ ID NO: 223 - protein, 16 aa, synthetic construct
VWVASILSSILDTIFH

SEQ ID NO: 224 - protein, 16 aa, synthetic construct
VYTVLLSWKVLLTLVW

SEQ ID NO: 225 - protein, 16 aa, synthetic construct
WFLPIMYSIICFVGLL

SEQ ID NO: 226 - protein, 16 aa, synthetic construct
WGAAPVIMMTWFYAFF

SEQ ID NO: 227 - protein, 16 aa, synthetic construct
WGLALLFALPDFIFLS

SEQ ID NO: 228 - protein, 16 aa, synthetic construct
WGWVLGDFLCKLLNMI

SEQ ID NO: 229 - protein, 16 aa, synthetic construct
WHWVFGSFLAKMVSTL

SEQ ID NO: 230 - protein, 16 aa, synthetic construct
WIFAVIIAIPHFMVVT

SEQ ID NO: 231 - protein, 16 aa, synthetic construct
WKLLAEIYEYLFFFSF

SEQ ID NO: 232 - protein, 16 aa, synthetic construct
WLMCIVGTFLNVLVIT

SEQ ID NO: 233 - protein, 16 aa, synthetic construct
WLMSIVGTFLNMLVIT

SEQ ID NO: 234 - protein, 16 aa, synthetic construct
WMNILWAWFIFWWPHG

SEQ ID NO: 235 - protein, 16 aa, synthetic construct
WNNFHTIMRNILGLVL

SEQ ID NO: 236 - protein, 16 aa, synthetic construct
WSLVFMAIERINYFAY

SEQ ID NO: 237 - protein, 16 aa, synthetic construct
WVASILSSILDTIFHK

SEQ ID NO: 238 - protein, 16 aa, synthetic construct
WVFIFSSIQLATVVFL

SEQ ID NO: 239 - protein, 16 aa, synthetic construct
WVFIFSSIQLCTVVFL

SEQ ID NO: 240 - protein, 16 aa, synthetic construct
WVLGDFLCKLLNMIFS

SEQ ID NO: 241 - protein, 16 aa, synthetic construct
WVLVIGSLVARWLLGI

SEQ ID NO: 242 - protein, 16 aa, synthetic construct
WVVVFQFQHIMVGLIL

SEQ ID NO: 243 - protein, 16 aa, synthetic construct
WWIFAVIIAIPHFMVV

SEQ ID NO: 244 - protein, 16 aa, synthetic construct
YDLFIGIKIVYAFLWG

SEQ ID NO: 245 - protein, 16 aa, synthetic construct
YDLFIGIKIVYCFLWG

SEQ ID NO: 246 - protein, 16 aa, synthetic construct
YDLFIGIKIVYSFLWG

SEQ ID NO: 247 - protein, 16 aa, synthetic construct
YDMWLMLVYSVMIPVM

SEQ ID NO: 248 - protein, 16 aa, synthetic construct
YHWGWVLGDFLCKLLN

SEQ ID NO: 249 - protein, 16 aa, synthetic construct
YLKAFLAAVLGWHLAP

SEQ ID NO: 250 - protein, 16 aa, synthetic construct
YLLDFILALVGNTLAL

SEQ ID NO: 251 - protein, 16 aa, synthetic construct
YLLNLAISDLLFVFSL

SEQ ID NO: 252 - protein, 16 aa, synthetic construct
YLYVYLDIFSVVSVSL

SEQ ID NO: 253 - protein, 16 aa, synthetic construct
YSAFLSGMTSLLLVLL

SEQ ID NO: 254 - protein, 16 aa, synthetic construct
YVAMFASLSFITEIAL

SEQ ID NO: 255 - protein, 16 aa, synthetic construct
YVSFISTTILFLIFSV

SEQ ID NO: 256 - protein, 16 aa, synthetic construct
YVSTLDFAIVFILMII

SEQ ID NO: 257 - protein, 16 aa, synthetic construct
DFAAILLYIDDFSAYF

SEQ ID NO: 258 - protein, 16 aa, synthetic construct
DFLCKLLNMIFSISLY

SEQ ID NO: 259 - protein, 16 aa, synthetic construct
DFLSKLLNMIFSISLY

SEQ ID NO: 260 - protein, 16 aa, synthetic construct
DFSAILLYIDDFSAYF

SEQ ID NO: 261 - protein, 16 aa, synthetic construct
DLFIGIKIVYSFLWGI

SEQ ID NO: 262 - protein, 16 aa, synthetic construct
DLTNWKLLAEIYEYLF

SEQ ID NO: 263 - protein, 16 aa, synthetic construct
AEIYEYLFFFSFFFLA

SEQ ID NO: 264 - protein, 16 aa, synthetic construct
DVYLLNLAISDLLFVF

SEQ ID NO: 265 - protein, 16 aa, synthetic construct
DYSIILMFLSDFSFIF

SEQ ID NO: 266 - protein, 16 aa, synthetic construct
EFDYDEDATPCVFTDV

SEQ ID NO: 267 - protein, 16 aa, synthetic construct
EIYEYLFFFSFFFLAL

SEQ ID NO: 268 - protein, 16 aa, synthetic construct
ELFLAVKIVFSFIWGV

SEQ ID NO: 269 - protein, 16 aa, synthetic construct
ESLFAALVTLITSLFG

SEQ ID NO: 270 - protein, 16 aa, synthetic construct
ESLFCALVTLITSLFG

SEQ ID NO: 271 - protein, 16 aa, synthetic construct
ESLFSALVTLITSLFG

SEQ ID NO: 272 - protein, 16 aa, synthetic construct
FAIVVAYFLSWGPYNF

SEQ ID NO: 273 - protein, 16 aa, synthetic construct
FAQLVYFIYHAVASYS

SEQ ID NO: 274 - protein, 16 aa, synthetic construct
FARAIDIIYSLITSAN

SEQ ID NO: 275 - protein, 16 aa, synthetic construct
FASDFVLMWAVFFMTV

SEQ ID NO: 276 - protein, 16 aa, synthetic construct
FASFYLLDFILALVGN

SEQ ID NO: 277 - protein, 16 aa, synthetic construct
FCALVTLITSLFGSIF

SEQ ID NO: 278 - protein, 16 aa, synthetic construct
FDFMILNLSFCDLFIC

SEQ ID NO: 279 - protein, 16 aa, synthetic construct
FDYDEDATPCVFTDVL

SEQ ID NO: 280 - protein, 16 aa, synthetic construct
AFLWGIFPTVIFSYFY

SEQ ID NO: 281 - protein, 16 aa, synthetic construct
FFSGMLLLLSISIDRY

SEQ ID NO: 282 - protein, 16 aa, synthetic construct
FFSQLVYFIYHAVSSY

SEQ ID NO: 283 - protein, 16 aa, synthetic construct
FFYYLYVYLDIFSVVA

SEQ ID NO: 284 - protein, 16 aa, synthetic construct
FFYYLYVYLDIFSVVC

SEQ ID NO: 285 - protein, 16 aa, synthetic construct
FFYYLYVYLDIFSVVS

SEQ ID NO: 286 - protein, 16 aa, synthetic construct
FGFFIPLMFMIFSYTF

SEQ ID NO: 287 - protein, 16 aa, synthetic construct
FGLLGNILVVITFAFY

SEQ ID NO: 288 - protein, 16 aa, synthetic construct
FGVITSIVTWGLAVLA

SEQ ID NO: 289 - protein, 16 aa, synthetic construct
FGVVTSVITWLVAVFA

SEQ ID NO: 290 - protein, 16 aa, synthetic construct
FGVVTSVITWVVAVFA

SEQ ID NO: 291 - protein, 16 aa, synthetic construct
FGVWLMSIVGTFLNML

SEQ ID NO: 292 - protein, 16 aa, synthetic construct
FIFVIGMIANSVVVWV

SEQ ID NO: 293 - protein, 16 aa, synthetic construct
FIGFFGSIFFITVISI

SEQ ID NO: 294 - protein, 16 aa, synthetic construct
FIGFFSGIFFIILLTI

SEQ ID NO: 295 - protein, 16 aa, synthetic construct
FIGIKIVYCFLWGIFP

SEQ ID NO: 296 - protein, 16 aa, synthetic construct
FIGIKIVYSFLWGIFP

SEQ ID NO: 297 - protein, 16 aa, synthetic construct
FILALVGNTLALWLFI

SEQ ID NO: 298 - protein, 16 aa, synthetic construct
FILYQFELFGFTLIFF

SEQ ID NO: 299 - protein, 16 aa, synthetic construct
FIYVLMFLCNAIGNSL

SEQ ID NO: 300 - protein, 16 aa, synthetic construct
FLAFLRSGYDMWLMLV

SEQ ID NO: 301 - protein, 16 aa, synthetic construct
FLFGSIGNFLVIFTIT

SEQ ID NO: 302 - protein, 16 aa, synthetic construct
FLFHLAVADLLLVFIL

SEQ ID NO: 303 - protein, 16 aa, synthetic construct
FLGILGNGLVIIIATF

SEQ ID NO: 304 - protein, 16 aa, synthetic construct
FLGLMASVWTHLGMAL

SEQ ID NO: 305 - protein, 16 aa, synthetic construct
FLGLMCSVWTHLGMAL

SEQ ID NO: 306 - protein, 16 aa, synthetic construct
FLLSLGIILFSYVEIL

SEQ ID NO: 307 - protein, 16 aa, synthetic construct
FLSIFYSVIFAIGLVG

SEQ ID NO: 308 - protein, 16 aa, synthetic construct
FLVVVYSIVSFLGILG

SEQ ID NO: 309 - protein, 16 aa, synthetic construct
FLWGIFPTVIFSYFYV

SEQ ID NO: 310 - protein, 16 aa, synthetic construct
FLYGVVFLFGSIGNFL

SEQ ID NO: 311 - protein, 16 aa, synthetic construct
FMFVLSVLGNLTVVAV

SEQ ID NO: 312 - protein, 16 aa, synthetic construct
FMFVLSVLGNLTVVCV

SEQ ID NO: 313 - protein, 16 aa, synthetic construct
FMIFAYTFIVKTLVQA

SEQ ID NO: 314 - protein, 16 aa, synthetic construct
FMIFCYTFIVKTLVQA

SEQ ID NO: 315 - protein, 16 aa, synthetic construct
FSAILLYIDDFSAYFS

SEQ ID NO: 316 - protein, 16 aa, synthetic construct
FSALVTLITSLFGSIF

SEQ ID NO: 317 - protein, 16 aa, synthetic construct
FSFFYVIFSKALHDVT

SEQ ID NO: 318 - protein, 16 aa, synthetic construct
FSIFWWIFAVIIAIPH

SEQ ID NO: 319 - protein, 16 aa, synthetic construct
FSIIAVFYFLLARAIS

SEQ ID NO: 320 - protein, 16 aa, synthetic construct
FSRAIDIIYSLITSSN

SEQ ID NO: 321 - protein, 16 aa, synthetic construct
FSTFSGMLVYILLLPV

SEQ ID NO: 322 - protein, 16 aa, synthetic construct
FSYVVVFLVSWLPYHV

SEQ ID NO: 323 - protein, 16 aa, synthetic construct
FTGMPPADEDYSPCML

SEQ ID NO: 324 - protein, 16 aa, synthetic construct
FTWNLVLSQFFSILAT

SEQ ID NO: 325 - protein, 16 aa, synthetic construct
FWAVDAVANWYFGNFL

SEQ ID NO: 326 - protein, 16 aa, synthetic construct
FWTPYNIVLFLETLVE

SEQ ID NO: 327 - protein, 16 aa, synthetic construct
FWWIFAVIIAIPHFMV

SEQ ID NO: 328 - protein, 16 aa, synthetic construct
FYISLFAGVSFLINLS

SEQ ID NO: 329 - protein, 16 aa, synthetic construct
FYVAMFASLCFITEIA

SEQ ID NO: 330 - protein, 16 aa, synthetic construct
FYVAMFASLSFITEIA

SEQ ID NO: 331 - protein, 16 aa, synthetic construct
FYVITLFASDFVLMWC

SEQ ID NO: 332 - protein, 16 aa, synthetic construct
FYYIGFYSSMFFITLM

SEQ ID NO: 333 - protein, 16 aa, synthetic construct
FYYLYVYLDIFSVVAV

SEQ ID NO: 334 - protein, 16 aa, synthetic construct
GDFLAKLLNMIFSISL

SEQ ID NO: 335 - protein, 16 aa, synthetic construct
GFFIPLMFMIFSYTFI

SEQ ID NO: 336 - protein, 16 aa, synthetic construct
GIFFIILLTIDRYLAV

SEQ ID NO: 337 - protein, 16 aa, synthetic construct
GIITSVLAWVTAILAT

SEQ ID NO: 338 - protein, 16 aa, synthetic construct
GMGPGPWMNILWAWFI

SEQ ID NO: 339 - protein, 16 aa, synthetic construct
GMPPADEDYSPCMLET

SEQ ID NO: 340 - protein, 16 aa, synthetic construct
GPGPWMNILWAWFIFW

SEQ ID NO: 341 - protein, 16 aa, synthetic construct
GSMLLSVLFTLYVSTL

SEQ ID NO: 342 - protein, 16 aa, synthetic construct
GSVSLVNLLTIIGCLW

SEQ ID NO: 343 - protein, 16 aa, synthetic construct
GTVFLSIFYSVIFAIG

SEQ ID NO: 344 - protein, 16 aa, synthetic construct
GVVTSVITWLVAVFAS

SEQ ID NO: 345 - protein, 16 aa, synthetic construct
GVVTSVITWVVAVFAS

SEQ ID NO: 346 - protein, 16 aa, synthetic construct
GVWLMAIVGTFLNMLV

SEQ ID NO: 347 - protein, 16 aa, synthetic construct
GVWLMAIVGTFLNVLV

SEQ ID NO: 348 - protein, 16 aa, synthetic construct
GVWLMSIVGTFLNVLV

SEQ ID NO: 349 - protein, 16 aa, synthetic construct
GWVFIFSSIQLATVVF

SEQ ID NO: 350 - protein, 16 aa, synthetic construct
GWVFIFSSIQLCTVVF

SEQ ID NO: 351 - protein, 16 aa, synthetic construct
GWVLGDFLCKLLNMIF

SEQ ID NO: 352 - protein, 16 aa, synthetic construct
HIGYFGGIFFIILLTI

SEQ ID NO: 353 - protein, 16 aa, synthetic construct
HNKTLSFISLLILSFL

SEQ ID NO: 354 - protein, 16 aa, synthetic construct
HRTVKLIFAIVVAYFL

SEQ ID NO: 355 - protein, 16 aa, synthetic construct
HTLRMTIFCLVLPLLV

SEQ ID NO: 356 - protein, 16 aa, synthetic construct
HTVLLSWKVLLTMVWG

SEQ ID NO: 357 - protein, 16 aa, synthetic construct
HWGWVLGDFLCKLLNM

SEQ ID NO: 358 - protein, 16 aa, synthetic construct
IAAFSWFMLSLPLLFL

SEQ ID NO: 359 - protein, 16 aa, synthetic construct
IAVFGLLGNILVVITF

SEQ ID NO: 360 - protein, 16 aa, synthetic construct
IAYSLIAVFGLLGNIL

SEQ ID NO: 361 - protein, 16 aa, synthetic construct
IAYSLISVFGLLGNIL

SEQ ID NO: 362 - protein, 16 aa, synthetic construct
IFAIVVAYFLSWGPYN

SEQ ID NO: 363 - protein, 16 aa, synthetic construct
IFFIILLTIDRYLAIV

SEQ ID NO: 364 - protein, 16 aa, synthetic construct
IFFIILLTIDRYLAVV

SEQ ID NO: 365 - protein, 16 aa, synthetic construct
IFWWIFAVIIAIPHFM

SEQ ID NO: 366 - protein, 16 aa, synthetic construct
IIALVVWGLSVIISSS

SEQ ID NO: 367 - protein, 16 aa, synthetic construct
IIFSYVVVFLVAWLPY

SEQ ID NO: 368 - protein, 16 aa, synthetic construct
IIFSYVVVFLVSWLPY

SEQ ID NO: 369 - protein, 16 aa, synthetic construct
IIIWALAILASMPGLY

SEQ ID NO: 370 - protein, 16 aa, synthetic construct
IISLVVWGLSVIISSS

SEQ ID NO: 371 - protein, 16 aa, synthetic construct
IITSVLAWVTAILATL

SEQ ID NO: 372 - protein, 16 aa, synthetic construct
ILAFFAAWLPYYIGIS

SEQ ID NO: 373 - protein, 16 aa, synthetic construct
ILAFFASWLPYYIGIS

SEQ ID NO: 374 - protein, 16 aa, synthetic construct
ILALVGNTLALWLFIR

SEQ ID NO: 375 - protein, 16 aa, synthetic construct
ILMFLSDFSFIFSSWA

SEQ ID NO: 376 - protein, 16 aa, synthetic construct
ILYQFELFGFTLIFFK

SEQ ID NO: 377 - protein, 16 aa, synthetic construct
IMYSIIAFVGLLGNGL

SEQ ID NO: 378 - protein, 16 aa, synthetic construct
AALFSIFWWIFAVIIA

SEQ ID NO: 379 - protein, 16 aa, synthetic construct
ISLFAGVSFLINLSMD

SEQ ID NO: 380 - protein, 16 aa, synthetic construct
ISLLAEVLMFLFPNII

SEQ ID NO: 381 - protein, 16 aa, synthetic construct
ISVAWHWVFGSFLSKM

SEQ ID NO: 382 - protein, 16 aa, synthetic construct
GNSTEGRCIELIGDFD

SEQ ID NO: 383 - protein, 16 aa, synthetic construct
HEDGKKVGLCLSTEYT

SEQ ID NO: 384 - protein, 16 aa, synthetic construct
KSDEYELFTVEYCGTN

SEQ ID NO: 385 - protein, 16 aa, synthetic construct
KSDVYELFTVEYCGTN

SEQ ID NO: 386 - protein, 16 aa, synthetic construct
NDKKVGLCLSTEYTDF

SEQ ID NO: 387 - protein, 16 aa, synthetic construct
SACSGDCVCVHRDNEP

SEQ ID NO: 388 - protein, 16 aa, synthetic construct
SGLCLSTTYIDFSEYG

SEQ ID NO: 389 - protein, 16 aa, synthetic construct
SGNCTCYHEDGKKVGL

SEQ ID NO: 390 - protein, 16 aa, synthetic construct
VGLCLSTEYTDFSEYP

SEQ ID NO: 391 - protein, 16 aa, synthetic construct
VGLCLSTEYTDFTKFP

SEQ ID NO: 392 - protein, 16 aa, synthetic construct
CVEITYFGDFGDPSQD

SEQ ID NO: 393 - protein, 16 aa, synthetic construct
DKKVGLCLSTEYTDFS

SEQ ID NO: 394 - protein, 16 aa, synthetic construct
EKSDEYELFTVEYCGT

SEQ ID NO: 395 - protein, 16 aa, synthetic construct
ENDKKVGLCLSTEYTD

SEQ ID NO: 396 - protein, 16 aa, synthetic construct
EEDDYTAYAPLTCYFT

SEQ ID NO: 397 - protein, 16 aa, synthetic construct
LEEEDDYTAYAPLTCY

SEQ ID NO: 398 - protein, 16 aa, synthetic construct
LTDGTACYVVERKVWD

SEQ ID NO: 399 - protein, 16 aa, synthetic construct
TDGTACYVVERKVWDR

SEQ ID NO: 400 - protein, 16 aa, synthetic construct
DEDYEDFFKPVTCYFA

SEQ ID NO: 401 - protein, 16 aa, synthetic construct
EEEDDYTAYAPLTCYF

SEQ ID NO: 402 - protein, 16 aa, synthetic construct
EDDYTAYAPLTCYFTN

SEQ ID NO: 403 - protein, 16 aa, synthetic construct
EDEDYEDFFKPVTCYF

SEQ ID NO: 404 - protein, 16 aa, synthetic construct
TYLLFALSLVLSAALS

SEQ ID NO: 405 - protein, 16 aa, synthetic construct
VNASIAWFFDFGACRM

SEQ ID NO: 406 - protein, 16 aa, synthetic construct
YLLFALSLVLSAALSA

SEQ ID NO: 407 - protein, 16 aa, synthetic construct
YLYILFLSAIIINGRD

SEQ ID NO: 408 - protein, 16 aa, synthetic construct
YLYILFLSCIIINGRD

SEQ ID NO: 409 - protein, 16 aa, synthetic construct
YSLVFVILMAIPFSFQ

SEQ ID NO: 410 - protein, 16 aa, synthetic construct
YSLVFVILMCIPFSFQ

SEQ ID NO: 411 - protein, 16 aa, synthetic construct
YSLVFVILMSIPFSFQ

SEQ ID NO: 412 - protein, 16 aa, synthetic construct
DLAGLDSLTSPVYVMA

SEQ ID NO: 413 - protein, 16 aa, synthetic construct
DMAYYRGDCLEVVYVT

SEQ ID NO: 414 - protein, 16 aa, synthetic construct
DSHMPGRADTHKLITN

SEQ ID NO: 415 - protein, 16 aa, synthetic construct
AIILLVYVYATFATKG

SEQ ID NO: 416 - protein, 16 aa, synthetic construct
GSMMLSLLIAGMSLML

SEQ ID NO: 417 - protein, 16 aa, synthetic construct
IILLVYVYATFATKGI

SEQ ID NO: 418 - protein, 16 aa, synthetic construct
IKAESKGSELILLDPP

SEQ ID NO: 419 - protein, 16 aa, synthetic construct
INTILIFASLFVASFA

SEQ ID NO: 420 - protein, 16 aa, synthetic construct
IYGLIASLIFVTSSTA

SEQ ID NO: 421 - protein, 16 aa, synthetic construct
AMMLSLLIAGMSLMLG

SEQ ID NO: 422 - protein, 16 aa, synthetic construct
LIKAAILLLAGGLAES

SEQ ID NO: 423 - protein, 16 aa, synthetic construct
LLFALSLVLSAALSAP

SEQ ID NO: 424 - protein, 16 aa, synthetic construct
LLLALLGAFTNAAPLL

SEQ ID NO: 425 - protein, 16 aa, synthetic construct
LSIILLVYVYATFATK

SEQ ID NO: 426 - protein, 16 aa, synthetic construct
LSLLIAGMSLMLGHAG

SEQ ID NO: 427 - protein, 16 aa, synthetic construct
LYLYILFLSSIIINGR

SEQ ID NO: 428 - protein, 16 aa, synthetic construct
MIYGLIAALIFVTSST

SEQ ID NO: 429 - protein, 16 aa, synthetic construct
MIYGLIASLIFVTSST

SEQ ID NO: 430 - protein, 16 aa, synthetic construct
MKSVLYLYILFLSAII

SEQ ID NO: 431 - protein, 16 aa, synthetic construct
MKSVLYLYILFLSCII

SEQ ID NO: 432 - protein, 16 aa, synthetic construct
MKSVLYLYILFLSSII

SEQ ID NO: 433 - protein, 16 aa, synthetic construct
MKSYILLLLLSAIIII

SEQ ID NO: 434 - protein, 16 aa, synthetic construct
MLSLLIAGMSLMLGHA

SEQ ID NO: 435 - protein, 16 aa, synthetic construct
MLSLLIAGMSLMLGHS

SEQ ID NO: 436 - protein, 16 aa, synthetic construct
MMIYGLIAALIFVTSS

SEQ ID NO: 437 - protein, 16 aa, synthetic construct
MMIYGLIASLIFVTSS

SEQ ID NO: 438 - protein, 16 aa, synthetic construct
MMLSLLIAGMSLMLGH

SEQ ID NO: 439 - protein, 16 aa, synthetic construct
MTPSYILLVYMFVVVS

SEQ ID NO: 440 - protein, 16 aa, synthetic construct
MVAVALYAYGLAFRLM

SEQ ID NO: 441 - protein, 16 aa, synthetic construct
MYSLVFVILMAIPFSF

SEQ ID NO: 442 - protein, 16 aa, synthetic construct
MYSLVFVILMCIPFSF

SEQ ID NO: 443 - protein, 16 aa, synthetic construct
RDTNPVSMNRTEALAL

SEQ ID NO: 444 - protein, 16 aa, synthetic construct
RLEELYLTVASMFERL

SEQ ID NO: 445 - protein, 16 aa, synthetic construct
AVLLLALLGAFTNAAP

SEQ ID NO: 446 - protein, 16 aa, synthetic construct
SMMLSLLIAGMSLMLG

SEQ ID NO: 447 - protein, 16 aa, synthetic construct
STYLLFALSLVLSAAL

SEQ ID NO: 448 - protein, 16 aa, synthetic construct
SVLYLYILFLSSIIIN

SEQ ID NO: 449 - protein, 16 aa, synthetic construct
SYILLLLLSSIIIINS

SEQ ID NO: 450 - protein, 16 aa, synthetic construct
TILIFASLFVASFAND

SEQ ID NO: 451 - protein, 16 aa, synthetic construct
CIILLVYVYATFATKG

SEQ ID NO: 452 - protein, 16 aa, synthetic construct
EEEDYTAYAPLTCYFT

SEQ ID NO: 453 - protein, 16 aa, synthetic construct
EEDDWTAYAPLTCYFT

SEQ ID NO: 454 - protein, 16 aa, synthetic construct
EEDDYDAYAPLTCYFT

SEQ ID NO: 455 - protein, 16 aa, synthetic construct
EEDDYEAYAPLTCYFT

SEQ ID NO: 456 - protein, 16 aa, synthetic construct
EEDDYSAYAPLTCYFT

SEQ ID NO: 457 - protein, 16 aa, synthetic construct
EEDDYWAYAPLTCYFT

SEQ ID NO: 458 - protein, 16 aa, synthetic construct
EEDDYTDYAPLTCYFT

SEQ ID NO: 459 - protein, 16 aa, synthetic construct
EEDDYTEYAPLTCYFT

SEQ ID NO: 460 - protein, 16 aa, synthetic construct
EEDDYTAYADLTCYFT

SEQ ID NO: 461 - protein, 16 aa, synthetic construct
EEDDYTAYAPFTCYFT

SEQ ID NO: 462 - protein, 16 aa, synthetic construct
EEDDYTAYAPITCYFT

SEQ ID NO: 463 - protein, 16 aa, synthetic construct
EEDDYTAYAPQTCYFT

SEQ ID NO: 464 - protein, 16 aa, synthetic construct
EEDDYTAYAPTTCYFT

SEQ ID NO: 465 - protein, 16 aa, synthetic construct
EEDDYTAYAPVTCYFT

SEQ ID NO: 466 - protein, 16 aa, synthetic construct
EEDDYTAYAPYTCYFT

SEQ ID NO: 467 - protein, 16 aa, synthetic construct
EEDDYTAYAPLHCYFT

SEQ ID NO: 468 - protein, 16 aa, synthetic construct
EEDDYTAYAPLICYFT

SEQ ID NO: 469 - protein, 16 aa, synthetic construct
EEDDYTAYAPLVCYFT

SEQ ID NO: 470 - protein, 16 aa, synthetic construct
EEDDYTAYAPLWCYFT

SEQ ID NO: 471 - protein, 16 aa, synthetic construct
EEDDYTAYAPLTCYFC

SEQ ID NO: 472 - protein, 16 aa, synthetic construct
EEDDYTAYAPLTCYFD

SEQ ID NO: 473 - protein, 16 aa, synthetic construct
ELNCWHDMPPPVSTDT

SEQ ID NO: 474 - protein, 16 aa, synthetic construct
KVANRMQGNVTYTCPV

SEQ ID NO: 475 - protein, 16 aa, synthetic construct
YDYTEGCPFVVLGNGT

SEQ ID NO: 476 - protein, 16 aa, synthetic construct
VLKLLGCHYFCNGTLC

SEQ ID NO: 477 - protein, 16 aa, synthetic construct
DEDYEDFFKPVTCYFA

SEQ ID NO: 478 - protein, 16 aa, synthetic construct
DYEDFFKPVTCYFANS

SEQ ID NO: 479 - protein, 16 aa, synthetic construct
EDEDYEDFFKPVTCYF

SEQ ID NO: 480 - protein, 16 aa, synthetic construct
EDYEDFFKPVTCYFAN

SEQ ID NO: 481 - protein, 16 aa, synthetic construct
ERKVWDRMTPMLWYES

SEQ ID NO: 482 - protein, 16 aa, synthetic construct
LTDGTAAYVVERKVWD

SEQ ID NO: 483 - protein, 16 aa, synthetic construct
LTDGTACYVVERKVWD

SEQ ID NO: 484 - protein, 16 aa, synthetic construct
LTDGTASYVVERKVWD

SEQ ID NO: 485 - protein, 16 aa, synthetic construct
TDGTACYVVERKVWDR

SEQ ID NO: 486 - protein, 16 aa, synthetic construct
TDGTASYVVERKVWDR

SEQ ID NO: 487 - protein, 16 aa, synthetic construct
YPCYNLTEHQAKNLTT

SEQ ID NO: 488 - protein, 16 aa, synthetic construct
EEDDYTAYAPLTCYFT

SEQ ID NO: 489 - protein, 16 aa, synthetic construct
LEEEDDYTAYAPLTAY

SEQ ID NO: 490 - protein, 16 aa, synthetic construct
LEEEDDYTAYAPLTCY

SEQ ID NO: 491 - protein, 16 aa, synthetic construct
LEEEDDYTAYAPLTSY

SEQ ID NO: 492 - protein, 16 aa, synthetic construct
DDYTAYAPLTCYFTNS

SEQ ID NO: 493 - protein, 16 aa, synthetic construct
DLEEEDDYTAYAPLTS

SEQ ID NO: 494 - protein, 16 aa, synthetic construct
EEEDDYTAYAPLTCYF

SEQ ID NO: 495 - protein, 16 aa, synthetic construct
EDDYTAYAPLTCYFTN

SEQ ID NO: 496 - protein, 16 aa, synthetic construct
EEDDYTAYAPLTAYFT

SEQ ID NO: 497 - protein, 16 aa, synthetic construct
EEDDYTAYAPLTSYFT

SEQ ID NO: 498 - protein, 16 aa, synthetic construct
EEEDDYTAYAPLTAYF

SEQ ID NO: 499 - protein, 16 aa, synthetic construct
EEEDDYTAYAPLTSYF

SEQ ID NO: 500 - protein, 16 aa, synthetic construct
GAPVVGIGGLDNKTWH

SEQ ID NO: 501 - protein, 16 aa, synthetic construct
SCVAKVDSLSKEEATV

SEQ ID NO: 502 - protein, 16 aa, synthetic construct
YNIVITKNKTLVVNCT

SEQ ID NO: 503 - protein, 16 aa, synthetic construct
EEDDYDAYAPLTCYFT

SEQ ID NO: 504 - protein, 16 aa, synthetic construct
EEDDYEAYAPLTCYFT

SEQ ID NO: 505 - protein, 16 aa, synthetic construct
EEDDWTAYAPLTCYFT

SEQ ID NO: 506 - protein, 16 aa, synthetic construct
EEDDYTDYAPLTCYFT

SEQ ID NO: 507 - protein, 16 aa, synthetic construct
EEDDYTAYAPITCYFT

SEQ ID NO: 508 - protein, 16 aa, synthetic construct
EEDDYWAYAPLTCYFT

SEQ ID NO: 509 - protein, 16 aa, synthetic construct
EEDDYTWYAPLTCYFT

SEQ ID NO: 510 - protein, 16 aa, synthetic construct
EEDDYTAYWPLTCYFT

SEQ ID NO: 511 - protein, 16 aa, synthetic construct
EEDDYTAYAPLWCYFT

SEQ ID NO: 512 - protein, 16 aa, synthetic construct
EEDDYTAYAPLTCYFC

SEQ ID NO: 513 - protein, 16 aa, synthetic construct
DEDDYTAYAPLTCYFT

SEQ ID NO: 514 - protein, 16 aa, synthetic construct
EDDDYTAYAPLTCYFT

SEQ ID NO: 515 - protein, 16 aa, synthetic construct
EEDDYTAYAPLTCYFD

SEQ ID NO: 516 - protein, 16 aa, synthetic construct
EEDDWDAYAPLTCYFT

SEQ ID NO: 517 - protein, 16 aa, synthetic construct
EEDDWTAYAPITCYFT

SEQ ID NO: 518 - protein, 16 aa, synthetic construct
EEDDYDAYAPITCYFT

SEQ ID NO: 519 - protein, 16 aa, synthetic construct
EEDDWDAYAPITCYFT

SEQ ID NO: 520 - protein, 16 aa, synthetic construct
DEDDWDDYAPITCYFT

SEQ ID NO: 521 - protein, 16 aa, synthetic construct
DDDDWDAYAPITCYFD

SEQ ID NO: 522 - protein, 16 aa, synthetic construct
DDDDWDDYAPITCYFD

SEQ ID NO: 523 - protein, 16 aa, synthetic construct
EDDDWDDYAPITCYFD

SEQ ID NO: 524 - protein, 16 aa, synthetic construct
EEDDWDDYAPITCYFD

SEQ ID NO: 525 - protein, 16 aa, synthetic construct
DDDDYDAYAPITCYFD

SEQ ID NO: 526 - protein, 16 aa, synthetic construct
DEDDWDAYAPITCYFD

SEQ ID NO: 527 - protein, 16 aa, synthetic construct
DDDDWDAYAPLTCYFD

SEQ ID NO: 528 - protein, 16 aa, synthetic construct
EEDDYDAYAPITCYFD

SEQ ID NO: 529 - protein, 16 aa, synthetic construct
DEDDWDAYAPLTCYFD

SEQ ID NO: 530 - protein, 16 aa, synthetic construct
DEDDWDAYAPIWCYFT

SEQ ID NO: 531 - protein, 16 aa, synthetic construct
EEDDWDAYAPIWCYFT

SEQ ID NO: 532 - protein, 16 aa, synthetic construct
DDDDWDAYAPIWCYFT

SEQ ID NO: 533 - protein, 16 aa, synthetic construct
DEDDYDAYAPIWCYFT

SEQ ID NO: 534 - protein, 16 aa, synthetic construct
EEDDYDAYAPIWCYFD

SEQ ID NO: 535 - protein, 16 aa, synthetic construct
DDDDWWDYAPLTCYFD

SEQ ID NO: 536 - protein, 16 aa, synthetic construct
EWDDWTDYAPLVCYFD

SEQ ID NO: 537 - protein, 16 aa, synthetic construct
DDDDYWDYAPLTCYFD

SEQ ID NO: 538 - protein, 16 aa, synthetic construct
EWDDYWDYAPITCYFD

SEQ ID NO: 539 - protein, 16 aa, synthetic construct
EEDDWTDYWPITCYFD

SEQ ID NO: 540 - protein, 16 aa, synthetic construct
EDEDYWDFFKPVTCYF

SEQ ID NO: 541 - protein, 16 aa, synthetic construct
EDEDYFDFFKPVTCYF

SEQ ID NO: 542 - protein, 16 aa, synthetic construct
EDEDYEDFFAPVTCYF

SEQ ID NO: 543 - protein, 16 aa, synthetic construct
EDEDYEDFFWPVTCYF

SEQ ID NO: 544 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 545 - protein, 272 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGEEDDYTAYAPLTCYFTGGGGGGGGEEDDYTAYAPLTCYFT

SEQ ID NO: 546 - protein, 5 aa, synthetic construct
GGGGS

SEQ ID NO: 547 - protein, 4 aa, synthetic construct
GGGG

SEQ ID NO: 548 - protein, 10 aa, synthetic construct
GGGGSGGGGS

SEQ ID NO: 549 - protein, 5 aa, synthetic construct
GSGGS

SEQ ID NO: 550 - protein, 5 aa, synthetic construct
GSSGS

SEQ ID NO: 551 - protein, 6 aa, synthetic construct
GGSGGS

SEQ ID NO: 552 - protein, 5 aa, synthetic construct
GGGGG

SEQ ID NO: 553 - protein, 8 aa, synthetic construct
GGGGGGGG

SEQ ID NO: 554 - protein, 5 aa, synthetic construct
EEEDY

SEQ ID NO: 555 - protein, 5 aa, synthetic construct
EEDDY

SEQ ID NO: 556 - protein, 5 aa, synthetic construct
EDEDY

SEQ ID NO: 557 - protein, 5 aa, synthetic construct
EDDDY

SEQ ID NO: 558 - protein, 6 aa, synthetic construct
PLTCYF

SEQ ID NO: 559 - protein, 6 aa, synthetic construct
PVTCYF

SEQ ID NO: 560 - DNA, 18 bp, synthetic construct
  source 1..18: mol_type = other DNA
gcagcctcttcatctggc

SEQ ID NO: 561 - DNA, 15 bp, synthetic construct
  source 1..15: mol_type = other DNA
ggtggaggatccgga

SEQ ID NO: 562 - DNA, 18 bp, synthetic construct
  source 1..18: mol_type = other DNA
ggaggcgccgagggtgac

SEQ ID NO: 563 - DNA, 42 bp, synthetic construct
  source 1..42: mol_type = other DNA
ataggcatttgtagcaatagaaaaaacgaacatagatgcaag

SEQ ID NO: 564 - DNA, 38 bp, synthetic construct
  source 1..38: mol_type = other DNA
ctattgctacaaatgcctatgcagcctcttcatctggc

SEQ ID NO: 565 - DNA, 35 bp, synthetic construct
  source 1..35: mol_type = other DNA
tcgtcaccctcggcgcctcctccggatcctccacc

SEQ ID NO: 566 - DNA, 61 bp, synthetic construct
  source 1..61: mol_type = other DNA
acactctttccctacacgacgctcttccgatctctagcgctatgcctatgcagcctcttca

SEQ ID NO: 567 - DNA, 60 bp, synthetic construct
  source 1..60: mol_type = other DNA
gactggagttcagacgtgtgctcttccgatctcgtctgcgatgacaacaaccatcgccca

SEQ ID NO: 568 - DNA, 26 bp, synthetic construct
  source 1..26: mol_type = other DNA
atgcctatgcagcctcttcatctggc

SEQ ID NO: 569 - DNA, 116 bp, synthetic construct
  source 1..116: mol_type = other DNA
ggtggaggatccggaggaggcgccgagggtgacgatcccgcaaaagcggcctttaactccctgcaagcctcagcgaccgaatatatcggttatgcgtgggcgatggttgttgtcat

SEQ ID NO: 570 - protein, 16 aa, synthetic construct
TLETDTFYECPDAYAY

(19) United States
(12) Patent Application Publication
(10) Pub. No.: US 2026/0234188 A1
(43) Pub. Date: Aug. 13, 2026

(54) CHEMOKINE-BINDING PEPTIDES

(71) Applicant: Oxford University Innovation Limited, Oxford (GB)

(72) Inventor: Shoumo Bhattacharya, Oxford (GB)

(73) Assignee: Oxford University Innovation Limited, Oxford (GB)

(21) Appl. No.: 18/879,791

(22) PCT Filed: Jun. 29, 2023
(86) PCT No.: PCT/GB2023/051699

§ 371 Date: Dec. 29, 2024

(30) Foreign Application Priority Data
Jun. 29, 2022 (GB) 2209520.2

(51) Int. Cl. C07K 7/08; A61K 38/00

(52) U.S. Cl. CPC C07K 7/08; A61K 38/00

(57) ABSTRACT

The invention relates to chemokine-binding peptides and combinations thereof, and methods and uses thereof.

### FIELD OF THE INVENTION

The invention relates to chemokine-binding peptides and combinations thereof, and methods and uses thereof.

### BACKGROUND OF THE INVENTION

Chemokines are a structurally-related class of signalling proteins that are expressed in the vascular endothelium in response to injury/infection. The chemokine network plays a crucial role in the inflammatory response. CC and CXC-chemokines are the primary drivers of chemotaxis in inflammation. Chemokines have been observed to heterodimerise with multiple other chemokines in order to play a role in the inflammatory response. Some viruses and organisms (such as ticks) have evolved to express proteins that bind to and inhibit downstream signalling of chemokines, thereby inhibiting the inflammatory response and the consequent immune response.

The 46 human chemokines are grouped into CC, CXC, CX3C and XC classes based on the spacing of their N-terminal cysteine residues. CC-chemokines constitute the largest class, with 26 members in humans, while CXC chemokines have 17 members. The binding of chemokines to a family of 18 G-protein coupled receptors (GPCRs) activates signalling, leading to chemotaxis or directed migration of leucocytes to the site of chemokine expression.

However, the chemokine network is highly redundant, with multiple chemokine types expressed at the site of disease, multiple receptor types present on leucocytes and multiple connections between receptor subtypes and chemokine subtypes. Removal or inhibition of a single chemokine type (e.g. with a monoclonal antibody) or a single receptor type (e.g. with a drug) has minimal impact on the network. This has led to a failure of drugs that target single elements of the network in clinical trials, and as of 2021 no anti-inflammatory therapeutic that targets the chemokine system is approved. Chemokine-binding proteins from ticks and viruses target multiple chemokine types, thereby overcoming redundancy, are effective in several disease models (see (Bonvin, 2016), (Yaron, 2020), WO 2019/034883 and WO 2017/149311). Other tick chemokine binding proteins have been identified. Viral and tick chemokine-binding proteins have not been clinically translated or entered clinical trials, possibly due to high cost of manufacture, immunogenicity of large foreign proteins, lack of oral delivery routes, or combinations thereof.

There is a need to provide additional chemokine binding entities for use in inhibition and detection of chemokines.

### SUMMARY OF THE INVENTION

The inventors have identified the existence of a large number of short chemokine-binding peptides in a range of tick proteins, viral proteins, human chemokines, and human/viral chemokine receptors and additional chemokine binding proteins. The inventors have further conceived that a combinatorial peptide entity, comprising short chemokine-binding peptides, would allow for enhanced chemokine binding compared to individual chemokine binding proteins. The modular nature of such combinatorial peptide entities allows for various configurations of chemokine-binding peptides and means that they may, for example, be tailored to provide chemokine-binding peptides to match the chemokine profile of, for example, an inflammatory disease tissue. Neutralisation of chemokines by administration of such a combinatorial peptide entity may be used to reduce chemokine activity and inflammation, and hence have therapeutic impact, and off-target effects on chemokines not involved in the disease profile could be minimised.

The combinatorial peptide entities conceived by the inventors are advantageous compared to naturally occurring chemokine-binding proteins such as tick evasions and viral proteins as they are, for example, easier to manufacture and are expected to elicit a weaker immunogenic response when administered to an individual.

The invention therefore provides a combinatorial peptide entity comprising a plurality of independently disposed heterologous chemokine-binding peptides.

### The Invention Also Provides a Chemokine-Binding Peptide Comprising:

(a) the amino acid sequence of SEQ ID NO: 396, or a variant thereof that retains the ability to one or more CC-class chemokines and one or more CXC-class chemokines;
(b) the amino acid sequence of any one of SEQ ID NO: 396, 403 and 392, or a variant thereof that retains the ability to bind to one or more CC-class chemokines and one or more CXC-class chemokines; or
(c) the amino acid sequence of any one of SEQ ID NOs: 1 to 543, or a variant thereof that retains the ability to bind to at least one.

The invention further provides a chemokine-binding peptide capable of binding to one or more CC-class chemokines and one or more CXC- and/or XC-class chemokines, the chemokine binding peptide comprising an amino acid sequence of the formula:

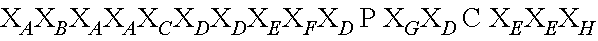

wherein X_{A} is D or E, X_{B} is D, E or W, X_{C} is Y or W, X_{D} is any amino acid, X_{E} is an aromatic amino acid, preferably Y or F, X_{F} is an aromatic amino acid or absent, preferably Y, F or absent, X_{G} is a hydrophobic amino acid, preferably V, L or I, and X_{H} is T, C, D or absent.

The invention additionally provides a combinatorial peptide entity comprising a plurality of chemokine-binding peptides according to the invention.

The invention further provides is a polynucleotide that encodes a chemokine-binding peptide of the invention, a vector that encodes the polynucleotide, or a host cell comprising the polynucleotide or the vector.

The invention also provides a pharmaceutical composition comprising the chemokine-binding peptide of the invention, a combinatorial peptide entity of the invention, or a vector, a polynucleotide or a host cell of the invention; and (b) a pharmaceutically acceptable carrier or diluent.

The invention also provides a library comprising a plurality of chemokine-binding peptides of the invention.

Additionally, the invention provides a method of producing a combinatorial peptide entity, comprising (a) identifying the chemokines associated with a disease, (b) identifying a combination of two or more chemokine binding peptides that bind to the chemokines associated with the disease, and (c) producing a combinatorial peptide entity from said two or more peptides.

The invention further provides a method of treating a disease associated with aberrant chemokine expression in a subject, comprising administering a pharmaceutical composition of the invention.

Also provided is a method of binding a plurality of chemokines, comprising contacting said chemokines with a combinatorial peptide entity of the invention.

Also provided is a method of detecting one or more chemokines in a tissue, comprising contacting the tissue with a detectably-labelled combinatorial peptide entity according to any one of claims 1 to 9 and 14 to 17, or a detectably labelled chemokine binding peptide according to any one of claims 10 to 13, and detecting the binding of the combinatorial peptide entity or the chemokine binding peptide to one or more chemokines in the tissue.

The invention also provides a method of identifying a chemokine-binding peptide, comprising (a) constructing a phage-display library encoding a bacteriophage coat protein fused to 10-mer to 20-mer peptides of a chemokine-binding protein at single amino acid resolution to thereby produce a phage-display library of overlapping peptides; (b) contacting the phage-display library with one or more chemokines to thereby produce an enriched library comprising peptides capable of binding the one or more chemokines; and (c) sequencing the enriched library to thereby identify a chemokine-binding peptide.

The invention additionally provides a method of enhancing and/or expanding chemokine binding activity of a chemokine-binding peptide, the method comprising (a) substituting one or more codons encoding a chemokine-binding peptide in a phage-display system with the sequence NNK, to thereby produce a mutant phage display library encoding mutated chemokine-binding peptide; (b) contacting the mutated phage-display library with one or more chemokines to thereby produce an enriched library comprising peptides capable of binding the one or more chemokines; (c) sequencing the enriched library to thereby identify a mutated chemokine-binding peptide with enhanced or expanded chemokine binding activity. The method may further comprise (d) constructing a combinatorial mutant phage display library comprising combinations of the substitutions in the one of more mutated chemokine-binding peptides identified in step (c); (e) contacting the combinatorial mutant phage display library with one or more chemokines to thereby produce an enriched combinatorial mutant library comprising peptides capable of binding the one or more chemokines; and (f) sequencing the enriched combinatorial mutant library to thereby identify combinatorially mutated chemokine-binding peptides with further enhanced or expanded chemokine binding activity.

### DESCRIPTION OF THE FIGURES

FIG. 1. Exemplar results from phage display screens. Phage-display libraries were constructed and analysed as described in (McLaughlin, 2013) (Tonikian, 2007). Screening was performed using chemokines displayed on streptavidin beads. FIG. 1A, CC chemokines against evasin A library. FIG. 1B, CXC chemokines against the evasin B library. FIG. 1C, CXC and CX3C chemokines against the viral chemokine binding protein library. FIG. 1D, CXC chemokines against the chemokine library. Each subpanel shows hexadecapeptides (as black tiles), identified by screening an individual hexadecapeptide phage-display library. Identified hexadecapeptides were mapped to the position in the originating protein (x-axis) and to fold enrichment (log 2 E, left y-axis). Regions of interest (ROI) were defined where the cumulative residue log 2 E, (clog 2 E:r, right y-axis) exceeds the 95% upper confidence interval of the median clog 2 E:r for the entire protein (indicated as a horizontal dotted line in each panel). Proteins are referred to by their UniProt ID.

FIG. 2. Activity of HD2 (EEDDYTAYAPLTCYFT), and EB429 (CVEITYFGDFGDPSQD) peptides, described in Table 5A, in inhibiting chemotaxis. FIG. 2A-FIG. 2E, Inhibition of human chemokine induced THP-1 cell migration in a transwell assay (Darlot, 2020)) by BK1.2, HD2, and HD2SCR (scrambled version of HD2, TLETDTFYECPDAYAY, as negative control) peptides, each at 10 μM. BK1.2 is described in (Darlot, 2020). FIG. 2F-FIG. 2H, Inhibition of human chemokine induced activated T-cell migration in a transwell assay as described (Lee, 2019)). X-axis in each panel shows constituents of the experiment. Chemokines used are indicated by the name, followed in parenthesis by the supplier code (P in supplier code indicates Peprotech). Y axis in each panel shows normalized count of migrated cells. All experiments were performed at EC₈₀ doses of indicated chemokine. Data are shown as box-whisker plots of three technical and three biological replicates. Statistically significant differences (compared to control, indicated by #), using Dunnett's test with correction for multiple comparisons, are indicated by asterisks: ****=P≤0.0001, ***=P≤0.001, **=P≤0.01, *=P≤0.05.

FIG. 3. Alanine scanning mutants of HD2 (EEDDYTAYAPLTCYFT) were generated and tested for ability to inhibit chemotaxis in response to CCL5 (FIG. 3A), to CCL7 (FIG. 3B), and to CCL8 (FIG. 3C) in order to identify important functional residues to guide further improvements of the peptide. Inhibition of human chemokine induced THP-1 cell migration in a transwell assay (performed as described (Darlot, 2020)) by HD2 (control), alanine-scanning mutants of HD2, and HD2SCR (scrambled version of HD2, TLETDTFYECPDAYAY) peptides, each at 10 μM. The position of the alanine mutants of HD2 are indicated as HD2_A1 to HD2_A16. X-axis in each panel shows constituents of the experiment. Chemokines used are indicated by the name, followed in parenthesis by the supplier code (P in supplier code indicates Peprotech). Y axis in each panel shows normalized count of migrated THP-1 cells. Cell counts were normalized by setting the median value obtained for cells with chemokine alone to 10000. All experiments were performed at EC₈₀ doses of indicated chemokine. Data are shown as box-whisker plots of three biological replicates, shown as individual data points. Statistically significant differences (compared to control, indicated by #), using Dunnett's test with correction for multiple comparisons, are indicated by asterisks: ****=P≤0.0001, ***=P≤ 0.001, **=P≤0.01, *=P≤0.05.

FIG. 4. FIG. 4A-FIG. 4C, Representative dose-response curves showing inhibition of human chemokine induced THP-1 cell migration by peptide HD2. The y axis shows the percentage of migration of THP-1 cells normalized to chemokine alone, which was set at 100%. Technical replicates are shown as shown as individual data points. The x axis shows inhibitor concentration (molar). The response curve (solid line) and its 95% confidence interval (dotted lines) were calculated using a four-parameter log-logistic plot to estimate IC50. The agonist and estimated IC50 (vertical dashed line) are indicated in each plot. FIG. 4D, summary IC50 values for inhibition of CCL5, CCL7 and CCL8-induced THP-1 cell migration by HD2, and EVA4_RHISA protein. The y axis shows IC50 (molar). The data are shown as box-whisker plots of three biological replicates, shown as individual data points.

FIG. 5. A-FIG. 5C, Disease chemokine coverage by single peptides (FIG. 5A), and by combinatorial peptide entities (CPEs, FIG. 5B, FIG. 5C). CPEs were chosen from all possible two-peptide and three-peptide combinations of the peptides identified by binding in phage-display experiments provided in Table 5A to maximise number of disease chemokines bound and minimise the number of off-target chemokines bound. Y-axis shows the organ, disease, and CPE. X-axis shows the number of chemokines expressed in diseased tissue, stacked by those bound by the CPE (i.e targeted, black), and those not bound (i.e. missed, white) by the CPE. Combinations of peptides are predicted to increase binding avidity by providing multiple binding sites and can increase the numbers of disease chemokines bound by the combination. Preferred combinations may be similarly designed using chemokine inhibition data rather than chemokine binding data.

FIG. 6. Exemplar peptide structures and combinatorial entities that can be created using established methods. FIG. 6A, HD2. FIG. 6B, VP6130. FIG. 6C, HD2 dimer created by disulfide bond linkage at Cys residues. FIG. 6D, cyclic HD2 created e.g. using head-to-tail cyclisation (Hayes, 2021). FIG. 6E, cyclic VP6130 e.g. created using head-to-tail cyclisation (Hayes, 2021). FIG. 6F, Bicyclic VP6130, e.g. created using a TBMB (2,4,6-Tri (bromomethyl)benzene linker (Heinis, 2009; Ahangarzadeh, 2019; Ernst, 2018). FIG. 6G, HD2-HD2, e.g. created using a PEG linker (Hamley, 2014). FIG. 6H, HD2-VP6130, e.g. created using a PEG linker (Hamley, 2014). I, Branched HD2-VP6130, e.g. created using a lysine core scaffold (Brunetti, 2018). Tri-branched or tetra-branched entities may be created using a lysine core scaffold (Brunetti, 2018). Other combinatorial entities may be created using e.g. nanoparticles (Jeong, 2018), or dendrimers (Sapra, 2019) or as multicyclic peptides (Loktev, 2017). Peptides may be modified using a variety of approaches to improve their function including binding to human serum albumin, lipidation, cyclization, D-amino-acid substitution, replacement of labile amino acids, PEGylation, or amide terminated e.g. to reduce proteolytic degradation, or addition of tags to allow detection (Tan, 2018) or addition of moieties that allow the peptide and its bound chemokine to be targeted for degradation (e.g. a LYTACs) (Banik, 2020) (Ahn, 2021).

FIG. 7. Exemplar results from phage display screens using saturation mutagenesis. A phage-display library was constructed using the HD2 sequence, replacing each residue encoding codon with the degenerate sequence NNK. The 16 degenerate sequences (which also encoded the parental HD2 sequence) were pooled and cloned into display phage. Library screening was performed using the indicated chemokines displayed on streptavidin beads. Exemplar peptides were selected such that log 2 E for at least one chemokine was greater than 5, and log 2 E for binding to control (CO5, was less than zero). X-axis shows individual residue changes by location in the peptide, and Y-axis shows the log 2 of fold enrichment (log 2 E) of the mutated sequence following chemokine affinity selection. The fold enrichment of parental HD2 is also indicated. The mutant peptide sequences and chemokines bound are presented in Table 5B.

FIG. 8. Promiscuous peptides. FIG. 8A, Tileplot showing log 2 E of 30 peptides that specifically bind at least 3 chemokines with log 2 E>5. Rows show the selecting chemokine and columns the peptide. Peptides are arranged by total numbers of chemokines bound with log 2 E>5. Scale bar shows log 2 E values. Grey tiles indicates that the peptide was not recovered following the screen. Chemokine types (I=inflammatory, D=dual, H=homeostatic) are indicated. FIG. 8B, Neighbour-joining tree of peptides. Peptide identities are indicated at the tree tips. The ancestral node was defined by midpoint rooting. Peptides derived from EVA4, EV672 and EV974 are indicated in azure, navy, and orange respectively. The heatmap shows the number of CC or XC chemokines bound with log 2 E>5. Mutant (MUT) and wild-type (WT) tip nodes are indicated. FIG. 8C, Box-whisker plot showing the effect of Cys mutation to Ala or Ser (MUT) compared to wild-type (WT) for the subset of 30 peptides. Y-axis indicates the total log 2 E. Individual peptide data are indicated as points. Statistically significant differences (compared to WT, coloured blue), using ANOVA is indicated by asterisk: *=P≤0.05, F=5.782, df=1, n=18 WT and 12 MUT. FIG. 8D, Sequence alignment and logo for wild-type EVA4 and EV672-derived peptides. Amino acid residues are coloured using the Taylor scale.

FIG. 9. Binding of chemokines using biolayer interferometry. FIG. 9A-9H, Representative BLI dose-response sensorgrams showing purified SUMO:HD2 binding to indicated doses of chemokines respectively. Plots display wavelength shift (y axis; nm) versus time (x-axis; seconds). Vertical dotted line indicates the onset of dissociation. Raw data are indicated by solid lines and fitted data (using a 1:1 or 2:1 binding model as indicated) by dotted lines. K_{D} estimates (mean±standard error) are indicated in each plot.

FIG. 10. Effect of exemplar peptide on chemotaxis. FIG. 10A-10I, Box-whisker plots showing the effect of the exemplar peptides HD2 and HD845 on cell migration induced by indicated human chemokines. All experiments were performed as three technical and three biological replicates, and individual data points are indicated. Y-axis in each panel shows cell count normalized to the median value of migrated cells in the presence of chemokine alone, set at 10000 cells. X-axis shows constituents of each experiment as blue-filled dots. Chemokine and cell type names are indicated. HD2SCR is a scrambled version of HD2 used as negative control. All peptides were at 10 μM final concentration and chemokines at EC₈₀ doses. Statistically significant differences (compared to control, coloured blue), using Dunnett's test with correction for multiple comparisons, are indicated by asterisks: ****=P≤0.0001, ***=P≤ 0.001, **=P≤0.01; n=9 (3 technical and 3 biological replicates) in each group.

FIG. 11. Effect of HD2 and EVA4 on chemotaxis response. FIG. 11A-11H, Representative dose-response curves showing effect of human chemokine induced THP1 or activated T cell (ATC) or Jurkat CXCR1 (J:CXCR1) migration by HD2 or EVA4. Y-axis shows percent migrated cells normalized to chemokine alone (set at 100%). Technical replicates are shown as individual data points. X-axis shows inhibitor concentration (molar). The response curve (solid blue line) and its 95% confidence interval (grey ribbon) were calculated using a 4-parameter log-logistic plot. Dotted green lines indicate IC50 and dotted black lines IC20 and IC80. FIG. 11I, 11J, Box-whisker plots showing the effect of HD2, scrambled version HD2SCR, and parental evasin EVA4 on cell migration induced by CXCL10 and CXCL6 respectively. HD2, HD2SCR and EVA4 were at 20 μM final concentration in FIGS. 11I and 10 μM in FIG. 11J. Y-axis shows cell count normalized to the median value of migrated cells in the presence of chemokine alone, set at 10000 cells. X-axis shows constituents of each experiment as blue-filled dots. Statistically significant differences (compared to control, coloured blue), using Dunnett's test with correction for multiple comparisons, are indicated by asterisks: ****=P≤0.0001, ***=P≤0.001, **=P≤0.01; n=9 (3 technical and 3 biological replicates, individual data points indicated) in each group. FIG. 11K, Summary IC50 values for inhibition by HD2 peptide and parental evasin EVA4. Y-axis shows IC50 (molar) and X-axis the constituents of each experiment. Data is shown as a box-whisker plot of biological replicates, shown as individual data points.

FIG. 12. Effect of HD2 mutations on phage binding. Box-whisker plots (FIG. 12A, 12C-12F) showing impact of HD2 residue mutation to alanine, conservative, anionic (glutamic acid, aspartic acid), cationic (lysine, arginine), and hydrophobic (leucine, isoleucine, methionine, and valine) residues respectively (X-axis) upon log 2 E (Y-axis). Statistically significant differences (compared to control, coloured blue), using Dunnett's test with correction for multiple comparisons, are indicated by asterisks: ****=P≤0.0001, ***=P≤0.001, **=P≤0.01, *=P≤0.05, n=48 per group for anionic or cationic mutations, 24 for wild-type, alanine, and conservative mutations, and 96 per group for hydrophobic mutations. Boxes showing a positive value for difference from control (identified from Dunnett's test) are coloured yellow. FIG. 12B, Tile plot of HD2 alanine mutations with tile colour showing Δlog 2 E, which is the difference in log 2 E between the parental wild-type peptide and the mutant variant following phage-display selection. Rows show the selecting chemokine and columns the mutation. Scale bar shows Δlog 2 E values.

FIG. 13. Effect of HD2 mutations on phage binding. Tileplots of HD2 mutations with tile colour showing Δog2 E, which is the difference in log 2 E between the parental wild-type peptide and the mutant variant following phage-display selection. Rows show the selecting chemokine and columns the mutation. Scale bar shows Δlog 2 E values. FIG. 13A, Conservative residue substitutions FIG. 13B, 13C, Anionic residue substitutions. FIG. 13D, 13E, Cationic residue substitutions. FIG. 13F-13I, Hydrophobic residue substitutions.

FIG. 14. Effect of HD2 mutations on phage binding. Box-whisker plots showing impact of HD2 residue anionic (glutamic acid (A), aspartic acid (B)), cationic (lysine (C), arginine (D)), and hydrophobic (leucine (E), isoleucine (I), valine (V) and methionine (M)) residues respectively (X-axis) upon log 2 E (Y-axis). Statistically significant differences (compared to control, coloured blue), using Dunnett's test with correction for multiple comparisons, are indicated by asterisks: ****=P≤0.0001, ***=P≤0.001, **=P≤0.01, *=P≤0.05. Boxes showing a positive value for difference from control (identified from Dunnett's test) are shown as yellow.

FIG. 15. Effect of HD2 alanine mutations on chemotaxis. a-e, Box-whisker plots showing the effect of the indicated alanine mutant peptides on cell migration induced by indicated human chemokines. All experiments were performed as three technical and at least three biological replicates, and individual data points are indicated. Y-axis in each panel shows migrated cell count normalized to the median value of migrated cells in the presence of chemokine alone, set at 10000 cells. X-axis shows constituents of each experiment as blue-filled dots. Chemokine names are indicated. HD2SCR is a scrambled version of HD2. All peptides were at 10 μM final concentration and chemokines at EC₈₀ doses. Statistically significant differences (compared to parental peptide HD2, coloured blue), using Dunnett's test with correction for multiple comparisons, are indicated by asterisks: ****=P≤0.0001, ***=P≤0.001, **=P≤0.01, *=P≤0.05.

FIG. 16. Effect of HD2 alanine mutations on chemotaxis. FIG. 16A, Box-whisker plot showing impact of HD2 residue mutation to alanine (X-axis) upon migrated cell count (Y-axis). All experiments were performed as three technical and three biological replicates, and individual data points are indicated, and coloured by the chemokine used. Statistically significant differences (compared to control, coloured blue), using Dunnett's test with correction for multiple comparisons, are indicated by asterisks: ****=P≤0.0001, ***=P≤0.001, **=P≤0.01, *=P≤0.05, n=45 observations per group (three technical and three biological replicates for each chemokine). FIG. 16B, Faceted plots of change in median normalised cell count between alanine mutant and wild-type HD2 peptide obtained in cell migration assays (Y-axis, Δmedian normalised cell count) versus alanine mutated residue (X-axis). Chemokines used for each experiment are indicated in the strip to the right of each plot. Statistically significant differences compared to parental HD2 are shown as red dots. FIG. 16C, Correlation of phage binding and inhibitory potency for alanine mutant HD2 peptides. Scatterplot of Δmedian normalised cell count (Y-axis, obtained in cell migration assays) versus Δlog 2 E (obtained in phage-display experiments) for peptide:chemokine pairs. Individual data points indicate peptide chemokine pairs for which both cell migration and Δlog 2 E were available. Linear regression plot with 95% confidence interval, Spearman correlation coefficient (R) and statistical significance (p) are shown.

FIG. 17. Predicted binding modes of HD2 and receptors to chemokines. FIG. 17A, Left panels: Ribbon diagrams showing predicted poses for CCL8:HD2 using AlphaFold2-Multimer (top) or AutoDock CrankPep (middle) and for CCL8:CCR1 using AlphaFold2-Multimer (bottom). Chemokines are in gray, and the peptide or receptor in colour. Right panels: Corresponding heatmaps of weighted proximity scores mapped onto ribbon diagrams of CCL8. FIG. 17B, Left panels: Ribbon diagrams showing predicted poses for CXCL10:HD2 using AlphaFold2-Multimer (top) or AutoDock CrankPep (middle) and for CXCL10:CXCR3 using AlphaFold2-Multimer (bottom). Right panels: Corresponding heatmaps of weighted proximity scores mapped onto ribbon diagrams of CXCL3. NT indicates the N-terminus, β1 and β3 indicate β-strands, αindicates the C-terminal α-helix. FIG. 17C, Stacked bar charts of weighted proximity scores (y-axis) for HD2 (top panel) and for receptor (middle panel), plotted for each chemokine residue (x-axis) following multiple sequence alignment (bottom panel). Models of chemokine:HD2 and chemokine:receptor (CCL2:CCR2, CCL3:CCR1, CCL5:CCR5, CCL7:CCR1, CCL8:CCR1, CXCL10:CXCR3, CXCL11:CXCR3 and CXCL6:CXCR1) were generated using AlphaFold2-Multimer. Secondary structural elements for CCL8 are indicated at the bottom of the top and middle panels. Chemokines and secondary structural elements are coloured as indicated in the legend. Amino acid residues are coloured using the Taylor scale. FIG. 17D, Stacked bar chart of chemokine:HD2 interchain interactions identified by Arpeggio from AlphaFold2-Multimer predictions. X-axis shows HD2 peptide residue and Y-axis the average number of interactions per residue. Interaction types are coloured as shown in the legend; “proximal” indicates residues within 5 Å of the chemokine chain.

FIG. 18. Selection of HD2 mutations. FIG. 18A, 18B, Box-whisker plots showing impact of HD2 mutations at each residue location that maximally enhance either mean log 2 E (a) or peak log 2 E (b) for all chemokines studied (top panel, n=22 per group), for CC chemokines (middle panel (n=15 per group), and for XC chemokines (bottom panel, n=7 per group). Statistically significant differences using Dunnett's test with correction for multiple comparisons, are indicated by asterisks: ****=P≤0.0001, ***=P≤0.001, **=P≤0.01, *=P≤0.05, n=22 per group. The control box in each panel is coloured blue, while boxes showing a positive value for difference from control >0.55 (identified from Dunnett's test) shown as yellow.

FIG. 19. Effect of selected HD2 mutations on chemotaxis. Box-whisker plot showing impact of HD2 residue mutation of indicated residue (X-axis) upon migrated cell count (Y-axis). All experiments were performed as at least three technical and three biological replicates, and individual data points are indicated, and coloured by the chemokine used, for all chemokines studied (n=16 chemokines). Statistically significant differences (compared to control, coloured blue), using Dunnett's test with correction for multiple comparisons, are indicated by asterisks: ****=P≤0.0001, ***=P≤0.001, **=P≤0.01, *=P≤0.05.

FIG. 20. Phage display-combinatorial mutagenesis. FIG. 20A, Tile-plot (bottom panel) showing impact of selected HD2 mutations on Δlog 2 E for all chemokines studied. Δlog 2 E is the difference in log 2 E between the parental wild-type peptide and the mutant variant following phage-display selection. See methods for a description of the strategy used for selecting individual mutations. FIG. 20B, All possible combinations of 16 selected single mutations (coloured using the Taylor scale) at 11 residue locations were combined to create a phage-display library with 3585 mutation combinations. The library included the parental HD2 sequence and single mutations as well. Library cloning, screening and analysis was performed as described in the examples.

FIG. 21. Combinatorial HD2 mutations. Combinations shown are those with the largest effect size (FIG. 21A-21C), with greatest binding to inflammatory/dual chemokines (FIG. 21D-21F), and with greatest binding to CC or XC chemokines (FIG. 21G-21I). FIG. 21A, 21D, 21G, Faceted box-whisker plots showing impact of 16 single and combinatorial (CM) mutations selected for the largest effect on median Δlog 2 E. Y-axis shows log 2 E, and X-axis individual single or combinatorial mutants (CM). In panel a, the first five CMs from left are those maximally reducing median Δlog 2 E, the next 5 are those maximally increasing median Δlog 2 E. Panels are labelled as ALL indicating all chemokines, H, indicating homeostatic, I/D indicating inflammatory/dual, CC indicating CC-chemokines and XC indicating XCX/CX3C chemokines. Statistically significant differences using Dunnett's test with correction for multiple comparisons, are indicated by asterisks: ****=P≤0.0001, ***=P≤0.001, **=P≤0.01, *=P≤0.05. The control box (parental HD2) is coloured blue, while boxes showing a positive value for difference from control >0.55 (identified from Dunnett's test) shown as yellow. FIG. 21B, 21E, 21H Tile-plots showing impact of the above mutations on Δlog 2 E. FIG. 21C, 21F, 21I. Sequence alignments and derived logos for the indicated CM peptides. Residues are coloured using the Taylor scale, and the parental HD2 sequence placed below each alignment for comparison.

FIG. 22. Effect of selected combinatorial HD2 mutations on chemotaxis. FIG. 22A. Box-whisker plot showing the effect of the exemplar single and combinatorically mutant (CM) HD2 peptides on activated-T cell (ATC) migration induced by CXCL12. All experiments were performed as at least three technical and three biological replicates, and individual data points are indicated. Y-axis in each panel shows cell count normalized to the median value of migrated cells in the presence of chemokine alone, set at 10000 cells. X-axis shows constituents of each experiment as blue-filled dots. HD2SCR is a scrambled version of HD2. All peptides were at 10 mM final concentration and chemokines at EC₈₀ doses. Statistically significant differences (compared to control, HD2_Y5W, coloured blue), using Dunnett's test with correction for multiple comparisons, are indicated by asterisks: ****=P≤0.0001, ***=P≤0.001, **=P≤0.01, *=P≤0.05. All comparisons were made to HD2_Y5W, as it is present in CM539, CM452, and CM304. FIG. 22B, Representative dose-response curves showing inhibition of human CXCL12 induced activated T cell (ATC) migration by HD2 single and combinatorial mutants. Y-axis shows percent migrated cells normalized to chemokine alone (set at 100%). Technical replicates are shown as individual data points. X-axis shows inhibitor concentration (molar). The response curve (solid blue line) and its 95% confidence interval (grey ribbon) were calculated using a 4-parameter log-logistic plot. The agonist and estimated IC50 (dotted green line) are indicated.

FIG. 23. Tile plots showing impact of HD2 mutations on Δlog 2 E at each residue location that maximally enhance either mean log 2 E (a) or peak log 2 E (b) for all chemokines. Top panels: mutations maximising overall chemokine binding, middle panels: mutations maximising CC binding, bottom panels: mutations maximising mean XC binding.

FIGS. 24 and 25. Effect of exemplar mutant HD2 peptides on chemotaxis. Box-whisker plots showing the effect of the exemplar mutant HD2 peptides on cell migration induced by indicated human chemokines. All experiments were performed as at least three technical and between one to three biological replicates, and individual data points are indicated. Y-axis in each panel shows cell count normalized to the median value of migrated cells in the presence of chemokine alone, set at 10000 cells. X-axis shows constituents of each experiment as blue-filled dots. Chemokine and cell type names are indicated. HD2SCR is a scrambled version of HD2 used as negative control. All peptides were at 10 μM final concentration and chemokines at EC80 doses. Statistically significant differences (compared to control, coloured blue), using Dunnett's test with correction for multiple comparisons, are indicated by asterisks: ****=P≤0.0001, ***=P≤0.001, **=P≤0.01, *=P≤0.05.

FIG. 26. Anti-chemokine peptibody development. FIG. 26A, Design of peptibody. FIG. 26B, Peptibodies were constructed using the design shown in Figure al (IgG1Fc:linker1:peptide 1:linker2:peptide2), where peptide 1 and peptide 2 positions were replaced by HD2 to generate bFc:HD2, and by HD2SCR to generate bFc:HD2SCR. HD2SCR is a scrambled version of HD2 and serves as negative control. Linker1 is 5 glycine residues, linker2 is eight glycine residues. The constructs were cloned into a pET backbone vector, expressed in E. coli BL21DE3 and purified using Protein-A-sepharose affinity matrix and size-fractionation. SDS-PAGE of bFc:HD2 peptibody (lane 2) showing dimeric (D) and monomeric (M) species observed under non-reducing conditions. Molecular weight marker (lane 1) sizes are shown in KDa. FIG. 26C, Biolayer interferometry dose-response sensorgram showing CCL8 (immobilised to streptavidin biosensor) binding to indicated doses of bFc:HD2. Plots display wavelength shift (y axis; nm) versus time (x-axis; seconds). Raw data are indicated by colored lines and fitted data (using a 1:1 binding model) by black lines. K_{D}=8.37 E−09±1 E−08 molar (mean±standard error). FIG. 26D, Box-whisker plots showing the effect of bFc:HD2 peptibody on THP1 cell migration induced by indicated human chemokines. All experiments were performed as three technical replicates, and individual data points are indicated. Y-axis in each panel shows cell count normalized to the median value of migrated cells in the presence of chemokine alone, set at 10000 cells. X-axis shows constituents of each experiment as blue-filled dots. Chemokine and cell type names are indicated. Peptibodies were at 10 μM final concentration and chemokines at EC80 doses. Statistically significant differences (compared to bFc:HD2SCR negative control, coloured blue), using Dunnett's test with correction for multiple comparisons, are indicated by asterisks: ****=P≤0.0001.

FIG. 27. Selection of HD845 mutations. FIG. 27A, 27B, Box-whisker plots showing impact of HD845 mutations at each residue location that maximally enhance either mean log 2 E (FIG. 27A) or peak log 2 E (FIG. 27B) for all chemokines studied (top panel, n=25 per group), for CC chemokines (middle panel (n=15 per group), and for XC chemokines (bottom panel, n=10 per group). Statistically significant differences using Dunnett's test with correction for multiple comparisons, are indicated by asterisks: ****=P≤0.0001, ***=P≤0.001, **=P≤0.01, *=P≤0.05, n=22 per group. The control box in each panel is coloured blue, while boxes showing a positive value for difference from control >0.55 (identified from Dunnett's test) shown as yellow.

FIG. 28. Characteristics of selected mutations. FIG. 28A, Tile-plot showing impact of selected HD845 mutations on Δlog 2 E for all chemokines studied. Δlog 2 E is the difference in log 2 E between the parental wild-type peptide and the mutant variant following phage-display selection. See methods for a description of the strategy used for selecting individual mutations. FIG. 28B, Box-whisker plot showing impact of selected HD2 mutations at each residue location on mean log 2 E for all chemokines studied. Statistically significant differences using Dunnett's test with correction for multiple comparisons, are indicated by asterisks: ****=P≤0.0001, ***=P≤0.001, **=P≤0.01, *=P≤0.05, n=25 per group. The control box in each panel is coloured blue, while boxes showing a positive value for difference from control >0.55 (identified from Dunnett's test) shown as yellow.

FIG. 29. Peptide superconsensus. Sequence alignments and derived sequence logo for the indicated peptides are shown. Residues are coloured using the Taylor scale. Peptides were selected if they showed statistically significant binding to chemokines in phage display or had statistically significant inhibitory effect in chemotaxis assays. Residue codes: X_{A} is D or E, X_{B} is D, E or W, X_{C} is Y or W, X_{D} is any amino acid, X_{E} is an aromatic amino acid, preferably Y or F, X_{F} is an aromatic amino acid or absent, preferably Y, F or absent, or absent, X_{G} is a hydrophobic amino acid, preferably V, L or I, and X_{H} is T, C, D or absent. Using these codes a chemokine binding peptide of the formula X_{A} X_{B} X_{A} X_{A} X_{C} X_{D} X_{D} X_{E} X_{F} X_{D} P X_{G} X_{D} C X_{E} X_{E} X_{H} may be created.

### DESCRIPTION OF THE SEQUENCE LISTING

SEQ ID NOs: 1 to 451 are provided in Table 5A.

SEQ ID NOs: 452 to 472 are provided in Table 5B.

SEQ ID NOs: 473-502 are provided in Table 6.

SEQ ID NOs: 503-539 are provided in Table 7.

SEQ ID NO; 540-543 are provided in Table 8.

SEQ ID NO: 544 is an Fc region:

DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFS
CSVMHEALHNHYTQKSLSLSPGK

SEQ ID NO: 545 is an exemplary chain of a peptibody of the invention. Fc region is shown in bold, linkers are italicised and the chemokine-binding peptide (HD2) is underlined.

DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFS
CSVMHEALHNHYTQKSLSLSPGKGGGGGEEDDYTAYAPLTCYFTGGGGGGG
GEEDDYTAYAPLTCYFT

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a peptide” includes two or more such peptides, or reference to “an entity” includes two or more such entities and the like.

All publications, patents and patent applications cited herein, whether supra or infra, are hereby incorporated by reference in their entirety. The disclosures of PCT/GB2017/050563, PCT/GB2018/052331 and U.S. Ser. No. 16/784,920 (publications numbers WO 2017/149311, WO 2019/034883 and US-2020-0247855-A1, respectively) are specifically incorporated by reference.

As used herein, the term “binds” has the same meaning as, and may be replaced by the term, “capable of binding”.

### Combinatorial Peptide Entities

The invention relates to combinatorial peptide entities, which comprise a plurality of chemokine binding peptides. Such chemokine binding peptides specifically bind to chemokines. The combinatorial peptide entity has an improved ability to bind chemokines than a single chemokine-binding peptide alone.

The combinatorial peptide entity may be a homomer of chemokine binding peptides, e.g. comprise two or more copies of a chemokine binding peptide that comprise the same amino acid sequence. The combinatorial peptide entity may be a heteromer of chemokine binding peptides, e.g. comprise two or more different chemokine binding peptides, which may be heterologous chemokine binding peptides. The combinatorial peptide entity may comprise homomeric chemokine binding peptides and heteromeric chemokine binding peptides, e.g. comprise two or more copies of a chemokine binding peptide that comprise the same amino acid sequence and additionally comprise one or more different chemokine binding peptides.

The chemokine binding peptides in the combinatorial peptide entity may have different chemokine binding profiles. The chemokine binding peptides in the combinatorial peptide entity may alternatively have the same chemokine binding profile (e.g. when homomeric), and enhance chemokine binding activity the combinatorial peptide entity when compared to the chemokine binding peptides individually.

The chemokine binding peptides in the combinatorial peptide entity may be linked to each other by any means. The chemokine binding peptides may be independently disposed within the combinatorial peptide entity. Alternatively, the chemokine binding peptides may be linked in-series, for example directly joined by peptide bonds.

The chemokine binding peptides may be any peptides that specifically bind to chemokines. A chemokine binding peptide may be of synthetic origin, for example identified via a phage display library, or identified by in silico modelling or screening. A chemokine binding peptide may represent a fragment of a naturally occurring protein, such as a naturally occurring chemokine binding protein, or a variant thereof. A chemokine binding peptide may be a variant of a synthetic or naturally occurring chemokine binding peptide, e.g. comprising one or more amino acid substitutions, deletions or additions when compared to the starting peptide, such as two or more, three or more, four or more, five or more, six or more, seven or more, eight of more, nine or more or ten or more amino acid substitutions, deletions or additions when compared to the starting peptide. Preferably, the chemokine binding peptide represents a fragment of a human chemokine, a tick evasin, a viral protein, or a human/viral chemokine receptor or other chemokine binding protein, such as a fragment of a protein set out in Tables 3A-3F.

A combinatorial peptide entity comprises a plurality of, e.g. two or more, chemokine binding peptides. For example, the combinatorial peptide entity may comprise three or more chemokine binding peptides. The combinatorial peptide entity may comprise four or more chemokine binding peptides. The combinatorial peptide entity may comprise five or more, six or more, seven or more, eight or more, nine or more, ten or more, fifteen or more, twenty or more, fifty or more or one hundred or more chemokine binding peptides. The combinatorial peptide entity may comprise between two and twenty chemokine binding peptides, such as between three and ten, or four and eight chemokine binding peptides. The combinatorial peptide entity may comprise two, three, four, five, six, seven, eight, nine, ten or more CKPBs.

A combinatorial peptide entity may comprise two or more different chemokine binding peptides, typically two or more heterologous chemokine binding peptides. Peptides are different when they do not consist of the same amino acid sequence. Peptides are heterologous when they are of different origin, for example representing fragments of different chemokine-binding proteins of origin. For example, a peptide which represents a fragment of CCL1 is heterologous to a peptide which represents a fragment of CCL2. The combinatorial peptide entity may comprise three or more different chemokine binding peptides. The combinatorial peptide entity may comprise four or more different chemokine binding peptides. The combinatorial peptide entity may comprise between two and ten different chemokine binding peptides, such as between three and eight different chemokine binding peptides or between four and six different chemokine binding peptides. The combinatorial peptide entity may comprise three or four different chemokine binding peptides. The combinatorial peptide entity may comprise two, three, four, five, six, seven, eight, nine, ten or more different CKPBs.

A combinatorial peptide entity may comprise two or more copies of the same chemokine binding peptide or two or more variants thereof. A combinatorial peptide entity may thus be a homomer, such as a homomultimer, of a chemokine binding peptide. Inclusion of additional copies of the same chemokine binding peptide (i.e, wherein the combinatorial peptide entity comprises two or more of the same chemokine binding peptide) typically provides for an altered chemokine binding profile. An altered chemokine binding profile due to the presence of two or more copies of the same chemokine binding peptide may be increased avidity, as discussed in more detail below. The combinatorial peptide entity may comprise three or more copies of the same chemokine binding peptide. The combinatorial peptide entity may comprise four or more copies of the same chemokine binding peptide. The combinatorial peptide entity may comprise five or more, six or more, seven or more, eight or more, nine or more, ten or more, fifteen or more, twenty or more, fifty or more or one hundred or more copies of the same chemokine binding peptide. The combinatorial peptide entity may comprise two, three, four, five, six, seven, eight, nine, ten or more copies of the same chemokine binding peptide.

A combinatorial peptide entity may be a homomer of independently disposed chemokine-binding peptides, wherein the combinatorial peptide entity comprises three or more independently disposed chemokine-binding peptides. A combinatorial peptide entity may be a heteromer of independently disposed chemokine binding peptides, wherein the combinatorial peptide entity comprises two or more independent disposed different chemokine binding peptides, typically two or more independently disposed heterologous chemokine binding peptides.

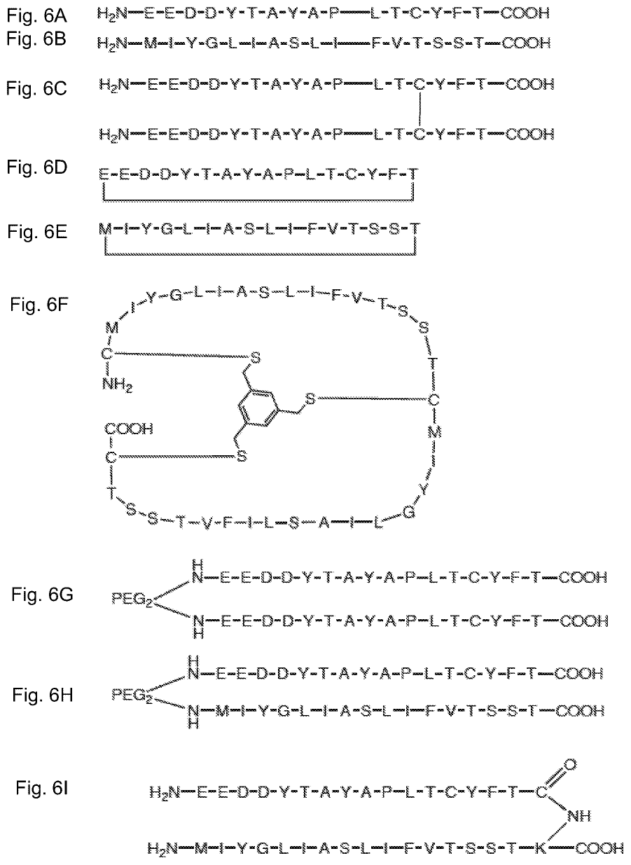

The combinatorial peptide entities described herein may comprise chemokine binding peptides that are independently disposed. A plurality of chemokine binding peptides may thus be presented separately from each other within the entity, for example at different sites or positions within the entity. The chemokine binding peptides may be independently disposed on the surface of the combinatorial peptide entity for example at different sites or positions on the surface of the entity. Typically, the peptides are not directly linked by peptide bonds. The peptides are typically not connected as a linear series of peptides, or as a monocyclic series of peptides. The peptides may form part of different monocyclic peptides within a multicyclic peptide. For example, the peptides may be independently disposed in a bicyclic peptide as shown in FIG. 6F. The chemokine binding peptides in the combinatorial peptide entities described herein may be fused via their terminal amino acids. For instance, the amino terminus of a first chemokine binding peptide may be fused to the amino terminus of a second chemokine binding peptide, and so on. The carboxy terminus of a first chemokine binding peptide may be fused to the carboxy terminus of a second chemokine binding peptide. Combinatorial peptide entities comprising independently disposed peptides allow for advantages over use of larger polypeptide chains that are generally more costly to produce, are less stable, and typically elicit stronger immunogenic responses in a host.

The chemokine binding peptides in the combinatorial peptide entity may alternatively or additionally be attached or linked to each other. The chemokine binding peptides may be attached in any manner, for example, directly or indirectly. The chemokine binding peptides in the combinatorial peptide entity may be covalently bound to each other. The chemokine binding peptides in the combinatorial peptide entity may be indirectly linked to each other. There may be no direct covalent bond between the chemokine binding peptides of the combinatorial peptide entity. The chemokine binding peptides may be linked to each other via other moieties within the combinatorial peptide entity. Alternatively, the chemokine binding peptides in the combinatorial peptide entity may be attached via a direct covalent bond (e.g. a side-chain linkage or a disulphide bond) between the chemokine binding peptides.

The chemokine binding peptides in the combinatorial peptide entity may be linked in series, e.g. as part of a linear polypeptide. This combinatorial peptide entity may comprise a linker between chemokine binding peptides. Typical linkers are usually short amino acid sequences, that may comprise primarily (e.g. >80%) small flexible residues like glycine and/or serine residues. Linkers may be less than 20 amino acids in length, such as less than 15, less than 10, less than 9, less than 8, less than 7, less than 6 or less than 5 amino acids in length. Suitable linkers include GGGGS, GGGS, GGGGSGGGGS, GSGGS, GSSGS, GGSGGS and the like.

The combinatorial peptide entity may be, for example, a peptibody, a branched peptide, a nanoparticle, a multicyclic peptide, a terminally linked peptide, a PEG-linked peptide, a dendrimer, a bacteria displaying the peptides, or a bacteriophage displaying the peptides. The combinatorial peptide entity may be a linear polypeptide.

The combinatorial peptide may be a peptibody. A peptibody of the invention typically comprises chemokine binding peptides grafted onto an Fc domain. The advantages of peptibodies include a long serum half-life, increased avidity via dimerization (see (Shimamoto, 2012)). In the context of the invention, a peptibody may independently present a number of chemokine binding peptides on each polypeptide chain. For example, each chain of a peptibody may comprise one or more chemokine binding peptides, such as two or more, three or more, or four or more chemokine binding peptides. Each chain of a peptibody may comprise one, two, three, four or more chemokine binding proteins. The Fc region may be modified to improve effector functions such as to reduce immunogenicity and/or to prolong plasma half-life. Such modifications are well known in the art, for example

L234A/L235A/G237A/P238S/H268A/A330S/P331S (see Saunders (2019) Frontiers in immunology, 10, 1296, which also describes known modifications to improve half-life). The Fc region may be from any of the types of subject discussed below. The Fc region may be human. The Fc region may be derived from any isotype of antibody, such as IgA, IgD, IgG, IgE or IgM. The Fc region may be an IgG, such as IgG1. The peptibody is typically a dimer.

Accordingly, the invention provides a peptibody comprising a plurality of chemokine-binding peptides as further described herein. The peptibody preferably comprises one or more chemokine-binding peptides selected from the group of HD2, HD845 and EB429, or variants of HD2, HD845 and EB429 as described herein. The peptibody may comprise one or more chemokine-binding peptides selected from SEQ ID NOs: 1-543, or variants thereof as described herein. The peptibody may comprise one or more copies of HD2 or a variant thereof as described herein. The peptibody may comprise one or more copies of HD845 or a variant thereof as described herein. The peptibody may comprise one or more copies of EB429 or a variant thereof as described herein. The peptibody may comprise one or more copies of HD2, or a variant thereof as described herein, and one or more copies of HD845, or a variant thereof as described herein. The peptibody may comprise one or more copies of HD2, or a variant thereof as described herein, and one or more copies of EB429, or a variant thereof as described herein. The peptibody may comprise one or more copies of EB429, or a variant thereof as described herein, and one or more copies of HD845, or a variant thereof as described herein. The peptibody may comprise one or more copies of HD2, or a variant thereof as described herein, one or more copies of HD845, or a variant thereof as described herein, and one or more copies of EB429, or a variant thereof as described herein. The peptibody may comprise a plurality of heterologous chemokine-binding peptides.

The peptibody may comprise one or more chemokine binding peptides capable of binding to one or more CC-class chemokines and one or more CXC- and/or XC-class chemokines.

The peptibody may comprise one or more chemokine-binding peptides on the C-terminus of an Fc region. The peptibody may comprise one or more chemokine-binding peptides on the N-terminus of an Fc region. The peptibody may comprise one or more chemokine-binding peptides on the C-terminus and on the N-terminus of an Fc region. The peptibody may comprise a linker between the Fc region and the one or more chemokine-binding peptides. The peptibody may comprise a linker between two or more chemokine-binding peptides in the same polypeptide chain. Typical linkers are usually short amino acid sequences, that may comprise primarily (e.g. >80%) small flexible residues like glycine and/or serine residues. Linkers may be less than 20 amino acids in length, such as less than 15, less than 10, less than 9, less than 8, less than 7, less than 6 or less than 5 amino acids in length. Suitable linkers include GGGGS, GGGS, GGGGGGGGS, GSGGS, GSSGS, GGSGGS, GGGGG and GGGGGGGG and the like.

Each chain of the peptibody may comprise (from N-terminal to C-terminal) an Fc region, an optional linker and a chemokine-binding peptide. Each chain may comprise an Fc region, an optional linker, a chemokine-binding peptide, and (an optional linker and a chemokine-binding peptide) N, wherein N=0, 1 or more, 2 or more or 3 or more. Each chain of the peptibody may comprise (from N-terminal to C-terminal) a chemokine-binding peptide, an optional linker and an Fc region. Each chain may comprise (a chemokine-binding peptide and an optional linker) N, a chemokine-binding peptide, an optional linker and an Fc region, wherein N=0, 1 or more, 2 or more or 3 or more. The one or more chemokine-binding peptide(s) within a single chain may be the same or different. The one or more chemokine-binding peptide(s) on different chains of the peptibody may be the same or different. The peptibody may comprise two or more chemokine binding peptides, e.g. one on each chain. The peptibody may comprise four or more chemokine binding peptides, e.g. two on each chain. Each chain of a peptibody may comprise one or more chemokine-binding peptides, such as two or more, three or more or four or more chemokine binding peptides.

The Fc region of the peptibody may comprise the amino sequence set out in SEQ ID NO: 544. The Fc region may comprise an amino acid sequence having at least 70% identity to the amino acid sequence set out in SEQ ID NO: 544, such as at least 80%, at least 90%, at least 95%, at least 96%, at least 97%, at least 98%, at least 99% or 100% identity to the amino acid sequence set out in SEQ ID NO: 544.

An exemplary sequence of a single chain of a peptibody of the invention is set out in SEQ ID NO: 545.

A branched peptide of the invention typically comprises chemokine binding peptides attached to each other via the side chains of one or more amino acids. Chemokine binding peptides may be attached to the side chains of a ‘core’ peptide, which may not be a chemokine binding peptide itself. The chemokine binding peptides may be attached to the side chains of other chemokine binding peptides, for example, via the addition of a small number of amino acids at the N- or C-terminus of the peptide that allows for the branching of the peptide. For example, peptides may be branched at the side chains of lysine, aspartic acid, glutamic acid, serine or threonine. Typically, peptides are branched at the side chain of lysine. The chemokine binding peptides may be modified by addition of, or substitution with, a residue to be used as the branching site, such as lysine. Said modification may comprise the addition of an N-terminal or C-terminal residue, such as by addition of lysine at the C-terminus of one or more chemokine binding peptide as shown in FIG. 6I. Tri-branched or tetra-branched combinatorial peptide entities may be created using a lysine core scaffold, for example as taught in (Brunetti, 2018).

Any side chain linkage may be used to attach the chemokine binding peptides. For example, a disulphide bond may be formed between two chemokine binding peptides. The chemokine binding peptides may be modified by addition of, or substitution with, a residue to be used as the side chain linkage site, such as an amino acid substitution with a cysteine residue. An exemplary side chain linkage is shown in FIG. 6C, which displays a homodimer of the chemokine binding peptide HD2, linked by a disulphide bond with the sequence of each peptide. Other multimers, including heteromers may be created using disulphide bonds between the constituent chemokine binding peptides of the combinatorial peptide entity.

The combinatorial peptide entity may be a cyclic peptide, such as a multicyclic peptide. A multicyclic peptide comprises a series of cyclised chemokine binding peptides attached to one another. For example, the chemokine binding peptides may be cyclised using a TBMB (2,4,6-Tri (bromomethyl)benzene linker (Heinis, 2009) (Ahangarzadeh, 2019) (Ernst, 2018). Other methods of cyclisation are well known in the art, see for example (Loktev, 2017). The cyclic peptides may be attached to each other to form a combinatorial peptide entity via covalent linkage. Such methods are known in the art.

The combinatorial peptide entity may be a monocyclic peptide. In this case, the combinatorial peptide entity typically comprises a linear polypeptide comprising the chemokine peptide entities arranged in-series, optionally with linkers as described above. The polypeptide may be cyclised, for example, using head-to-tail cyclisation (Hayes, 2021).

A combinatorial peptide entity may comprise a plurality of chemokine binding peptides attached to a nanoparticle. The chemokine binding peptides may be attached by any means known in the art. For example, the nanoparticle may be a gold nanoparticle decorated by polyethylene glycol (PEG) thiol and/or oligo ethylene glycol (OEG) thiols as self-assembled monolayers. Methods for producing nanoparticles are well known in the art (Jeong, 2018).

The invention thus provides a nanoparticle comprising a plurality of chemokine binding peptides, which may be independently disposed on its surface. The chemokine binding peptides of the nanoparticle may be created as described for combinatorial peptide entities of the invention.

The chemokine binding peptides in the combinatorial peptide entity may be linked at their termini. For example, the chemokine binding peptides may be linked at their N-termini, their C-termini, or at a mix of their N- and C-termini, As shown in FIGS. 6G and 6H, a combinatorial peptide entity may, for example, comprise the chemokine binding peptides joined by a PEG linker, as described in (Hamley, 2014).

The combinatorial peptide entity may be a dendrimer comprising two or more chemokine binding peptides. Suitable dendrimer molecules are discussed in (Sapra, 2019).

A combinatorial peptide entity may be bacteria displaying the peptides, preferably a lactic acid bacteria (LAB) comprising a plurality of chemokine binding peptides displayed on the outer surface of the bacteria, typically independently disposed. Lactic acid bacteria have been proposed as anti-inflammatory therapeutics themselves and can lead to the production of anti-inflammatory cytokines such as IL-10. The chemokine binding peptides may be cloned into a LAB with a secretion signal and a surface anchor, as shown in (Škrlec, 2017). The invention thus provides a bacterium, such as a LAB comprising a plurality of chemokine binding peptides displayed on its outer surface. Chemokine binding peptides of the LAB may be as described for combinatorial peptide entities of the invention.

As illustrated in the examples, bacteriophages may also be used to display chemokine-binding peptides. Accordingly, a combinatorial peptide entity may be a bacteriophage displaying a plurality of chemokine binding peptides, typically independently disposed on its surface. The invention thus provides a bacteriophage comprising a plurality of chemokine binding peptides displayed on its outer surface. The chemokine binding peptides of the bacteriophage may be as described for combinatorial peptide entities of the invention.

The peptides in the combinatorial peptide entities may be modified using a variety of approaches to improve their function including binding to human serum albumin, lipidation, cyclization, D-amino-acid substitution, replacement of labile amino acids, PEGylation, or amide terminated e.g. to reduce proteolytic degradation, or addition of tags to allow detection (Tan, 2018). In some cases, the combinatorial peptide entities are preferably modified to become a lysosome-targeting chimaera (LYTAC). A LYTAC of the invention comprises the combinatorial peptide entity fused to a glycopeptide ligand that targets a lysosomal receptor, to thereby remove the LYTAC and any bound chemokines from the circulation. The glycopeptide ligand may be a first generation LYTAC ligand, i.e. a molecule that targets the cation independent mannose-6-phosphate receptor (CI-M6PR), for example via multiple serine-O-mannose-6-phosphonate (M6Pn) residues as described in (Banik, 2020), which is herein incorporated by reference. The glycopeptide ligand may be a second generation LYTAC ligand, i.e. a molecule that targets the asialoglycoprotein receptor (ASGPR), for example, via a triantennerrary N-acetylgalactosamine (tri-GalNAc) motif as described in (Ahn, 2021), which is herein incorporated by reference. The LYTAC may be generated biosynthetically, for example by fusing sequences containing one or more short N-glycosylation motifs (N—X—S/T) to the peptide and then expressing it, for example, in yeast (Buentzel, 2017). Thus, the combinatorial peptide entity may be modified with a residue that targets chemokines for degradation.

The combinatorial peptide entities of the invention typically have an improved ability to bind chemokines compared to any single chemokine-binding peptide (comprised within the entity) alone. The improved ability to bind a chemokine may be an altered chemokine binding profile when compared to any single chemokine-binding peptide (comprised within the entity) alone.

An altered chemokine binding profile for the combinatorial peptide entity may comprise the ability to bind a different selection of chemokines as compared to one of the chemokine binding peptides comprised within the entity individually. The combinatorial peptide entity may thus be able to bind one or more chemokines not bound by one of the chemokine binding peptides comprised within the entity individually, due to binding activity of another chemokine binding peptide comprised within the entity. The chemokine binding profile of the combinatorial peptide entity may be substantially identical or identical to the cumulative (combined) chemokine binding profile of each of the chemokine binding peptides comprised within the entity.

An improved chemokine binding ability for the combinatorial peptide entity may comprise the ability to bind one or more chemokines more strongly as compared to any one of the chemokine binding peptides comprised within the entity individually. For example, the combinatorial peptide entity may comprise a plurality of copies of the same chemokine binding peptide or variants thereof and thus bind the chemokine more strongly as compared to a single chemokine binding peptide individually. An improved chemokine binding ability for the combinatorial peptide entity may thus comprised increased avidity for one or more chemokines. Many chemokines form heterodimers and homodimers and thus where two chemokine binding peptides are located in a combinatorial peptide entity, the binding of a first chemokine binding peptide to a first chemokine in a dimer may improve the binding of the second chemokine binding peptide to the second chemokine in the dimer, thus increasing the avidity of the combinatorial peptide entity as compared to either chemokine binding peptide individually.

It should be understood that the altered or improved chemokine binding ability of the combinatorial peptide entity is typically by comparison to that of any single chemokine binding peptide that is comprised within the combinatorial peptide entity, taken individually. Thus, the combinatorial peptide entity displays an altered or improved chemokine binding ability compared to any single chemokine binding peptide that it represents.

The combinatorial peptide entity may bind at least one additional chemokine compared to a first chemokine binding peptide comprised within the combinatorial peptide entity. The additional chemokine binding for the combinatorial peptide entity may be provided by the presence of at least one additional (second) chemokine binding peptide that is different from the first chemokine binding peptide. The second chemokine binding peptide thus may bind one or more different chemokines compared to the first chemokine binding peptide. The combinatorial peptide entity may bind at least two, at least three, at least four, at least five, at least six, or at least eight additional chemokines as compared to the first chemokine binding peptide. The combinatorial peptide entity may bind two, three, four, five, six, seven, eight or more additional chemokines as compared to the first chemokine binding protein.

The combinatorial peptide entity may bind in total at least two, at least three, at least four, at least five, at least six, at least eight, at least ten, at least twelve, at least fourteen or more different chemokines. The combinatorial peptide entity may bind in total two, three, four, five, six, seven, eight, nine, ten, eleven, twelve, thirteen, fourteen or more different chemokines. The combinatorial peptide entity may bind up to five, up to ten, up to twelve, up to fifteen or up to twenty different chemokines. The combinatorial peptide entity may bind two to five, two to eight, two to ten, two to twelve, two to fifteen, or two to twenty different chemokines. The combinatorial peptide entity may bind five to ten, five to fifteen, or five to twenty different chemokines.

The combinatorial peptide entity may bind all chemokines bound by the plurality of (for example the two or more) different chemokine binding peptides comprised within the combinatorial peptide entity.

The combinatorial peptide entities of the invention bind to chemokines. The chemokines may be selected from any known chemokines or chemokines newly identified in the future which are bound by chemokine binding peptides. The chemokines are preferably human chemokines. However, chemokines may also be selected from other animals of veterinary importance (e.g. dog, cat, pig, sheep, cow, horse) and scientific importance (e.g. mouse, rat, monkey).

The combinatorial peptide entities may bind at least one CC chemokine and at least one other class of chemokine, such as a CXC, a CX3C and/or a XC class chemokine, e.g. at least one chemokine of the CC class and at least one chemokine of the CXC class. Known human CC, CXC, CX3C and XC class chemokines are indicated in Table 3 E and the combinatorial peptide entities may bind any of the CC, CXC, CX3C and XC class chemokines shown in Table 3 E. The combinatorial peptide entities may bind to (i) one or more CC class chemokines, (ii) one or more CXC class chemokines, (iii) one or more CX3C class chemokines and/or (iv) an XC class chemokine, such as: (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or, (i), (ii), (iii) and (iv).

Binding of different classes of chemokines is of particular utility when matching chemokine expression in a disease where different classes of chemokines are expressed. For example, binding of at least one CC and at least one CXC chemokine is of particular utility in matching to chemokine expression in disease where both CC and CXC chemokines are expressed. A CC chemokine may be selected from any of the disease expressed CC chemokines shown in Table 4. A CXC chemokine may be selected from any of the disease-expressed CXC chemokines shown in Table 4. The combinatorial peptide entity may bind at least two CC chemokines and at least one CXC chemokine, at least three CC chemokines and at least one CXC chemokine, at least five CC chemokines and at least one CC chemokine, at least six CC chemokines and at least one CXC chemokine, at least eight CC chemokines and at least one CXC chemokine, at least ten CC chemokines and at least one CXC chemokine, at least twelve CC chemokines and at least one CXC chemokine, at least fourteen CC chemokines and at least one CXC chemokine, or at least sixteen CC chemokines and at least one CXC chemokine. The combinatorial peptide entity may bind two, three, four, five, six, seven, eight, nine, ten, eleven, twelve, thirteen, fourteen or more CC chemokines and at least one CXC chemokine. The combinatorial peptide entity may bind any of the above minimum numbers of different CC chemokines and at least two different CXC chemokines, at least three CXC chemokines, at least four CXC chemokines, at least five CXC chemokines, or at least six CXC chemokines. The combinatorial peptide entity may bind one CC class chemokine and at least two, at least three, at least four, at least five CXC or at least six CXC chemokines. The combinatorial peptide entity may bind any of the above minimum numbers of different CC chemokines and two, three, four, five, six or more different CXC chemokines,

A combinatorial peptide entity may comprise two or more different chemokine binding peptides, typically two or more heterologous chemokine binding peptides, and bind at least one CC and at least one CXC chemokine. Such combinatorial peptide entity may comprise three or more, or four or more, different chemokines binding peptides. Such combinatorial peptide entity may comprise three, four or more different chemokine binding proteins.

Ideally a combinatorial peptide entity should bind CC and CXC chemokines expressed in the disease, and preferably further associated with the pathophysiology of a particular disease. The combinatorial peptide entity may be designed to bind CX3C and CC chemokines or CX3C and CXC chemokines, or CX3C, CC and CXC chemokines if a CX3C chemokine is expressed in the disease, and preferably further associated with the pathophysiology of the disease. For example, a combinatorial peptide entity may bind to at least 50% of the chemokines associated with a disease, such as at least 60%, at least 70%, at least 80%, at least 90%, at least 95% or all of the chemokines associated with a disease.

A combinatorial peptide entity may thus comprise a plurality of chemokine-binding peptides wherein the combinatorial peptide entity binds to CXC-class chemokines and another class of chemokines. A combinatorial peptide entity may comprise a plurality of chemokine-binding peptides, wherein the combinatorial peptide entity binds to CC-class chemokines and another class of chemokines. A combinatorial peptide entity may comprise a plurality of chemokine-binding peptides, wherein the combinatorial peptide entity binds to CXC-class chemokines and a CC class of chemokines. The combinatorial peptide entity may be a homomer of a chemokine binding peptide, wherein the chemokine binding peptide binds to more than one class of chemokines. The combinatorial peptide entity may be a heteromer of chemokine binding peptides, comprising at least one chemokine binding peptide that is capable of binding a CC class chemokine and at least one chemokine binding peptide that is capable of binding a CXC class chemokine. In all of the above combinatorial peptide entities, the chemokine-binding peptides may be independently disposed.

A combinatorial peptide entity may comprise one or more chemokine binding peptides that are fragments of a class A evasin selected from the group of EVA4, EV672, EV974 and EV546, or variants thereof.

### Chemokine Binding Peptides

The combinatorial peptide entities comprise a plurality of chemokine binding peptides. Chemokine binding peptides are peptides that specifically bind to chemokines. Chemokines are well known in the art. When a chemokine binding peptide binds to a chemokine, it typically blocks downstream inflammatory signalling pathways by said chemokine, for instance by preventing dimerization of a chemokine or preventing interaction of the chemokine with its cognate chemokine receptor. The combinatorial peptide entity provided herein may bind to a range of chemokines to thereby reduce chemokine activity (e.g. dimerization or receptor binding) and reduce inflammation in a subject.

A peptide “specifically binds” to a chemokine when it binds with preferential or high affinity to chemokines but does not substantially bind, does not bind or binds with only low affinity to other non-chemokine polypeptides. For instance, a peptide “specifically binds” to chemokines when it binds with preferential or high affinity to CCL2_HUMAN but does not substantially bind, does not bind or binds with only low affinity to other non-chemokine polypeptides, such as serum albumin.

A chemokine binding peptide typically binds to three or more different chemokines. The chemokine binding peptides may bind to four or more different chemokines, such as five or more different chemokines, or six or more different chemokines. The chemokine binding proteins may bind to three, four, five, six or more chemokines A combinatorial peptide entity of the invention may comprise two or more different chemokine binding peptides, wherein each chemokine binding peptide binds to at least three different chemokines, such as at four or more, five or more, or six or more different chemokines. A combinatorial peptide entity of the invention may comprise two or more different chemokine binding proteins, wherein each chemokine binding protein binds to three, four, five, six or more different chemokines. A combinatorial peptide entity of the invention may comprise three or more different chemokine binding peptides, wherein each chemokine binding peptide binds to at least three different chemokines, such as at four or more, five or more, or six or more different chemokines. A combinatorial peptide entity of the invention may comprise three or more different chemokine binding proteins, wherein each chemokine binding protein binds to three, four, five, six or more different chemokines. A combinatorial peptide entity of the invention may comprise four or more different chemokine binding peptides, wherein each chemokine binding peptide binds to at least three different chemokines, such as at four or more, five or more, or six or more different chemokines. A combinatorial peptide entity of the invention may comprise four or more different chemokine binding proteins, wherein each chemokine binding protein binds to three, four, five, six or more different chemokines.

The chemokines may be selected from any known chemokines or chemokines newly identified in the future which are bound by chemokine binding peptides. The chemokines are preferably human chemokines. However, chemokines may also be selected from mammals and/or other animals of veterinary importance (e.g. dog, cat, pig, sheep, cow, horse) and scientific importance (e.g. mouse, rat, monkey).

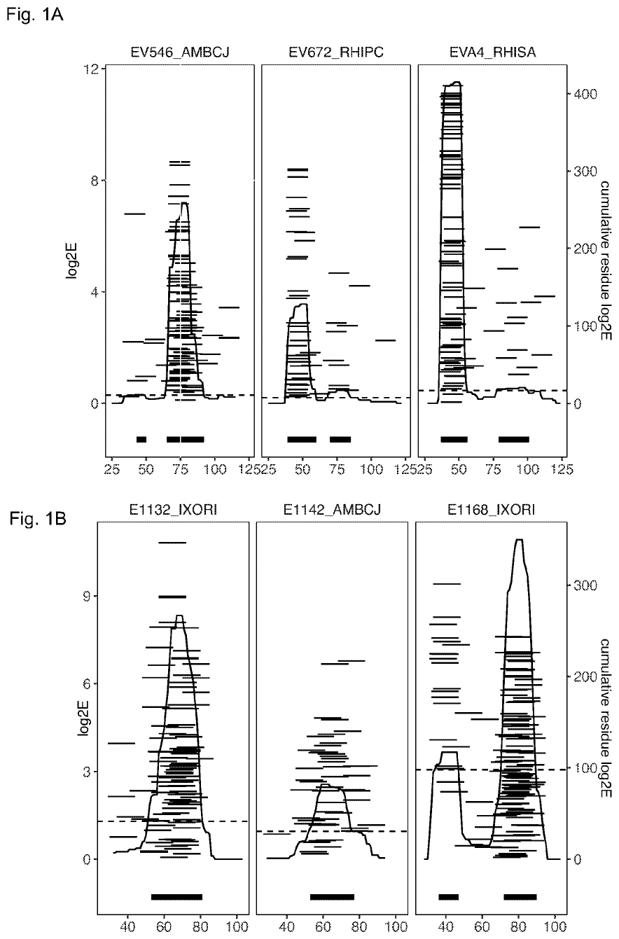

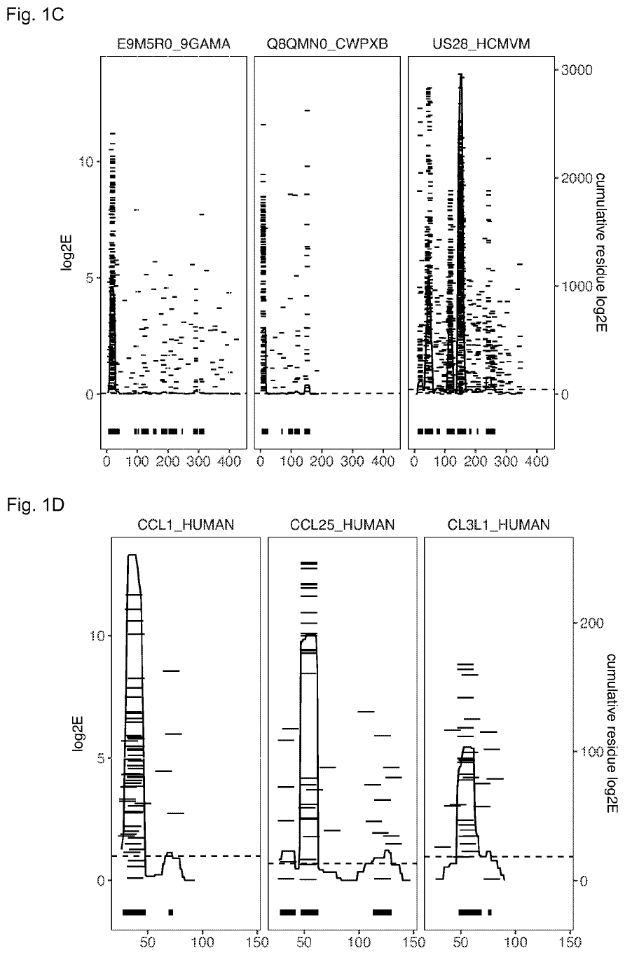

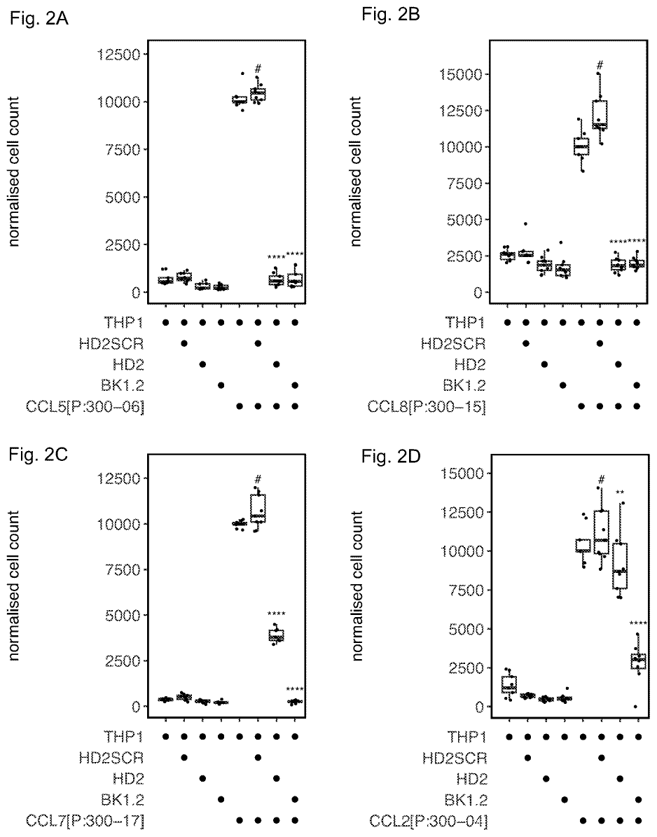

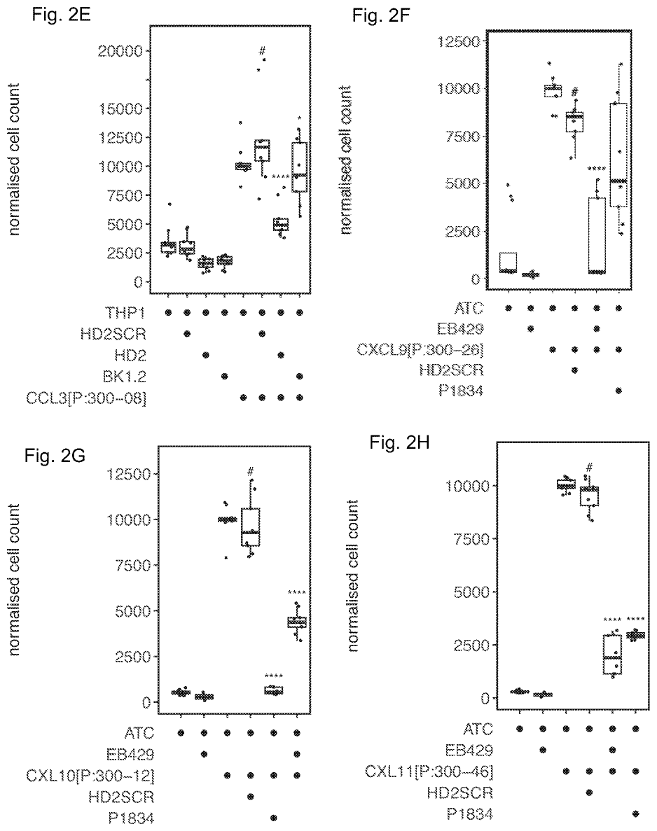

The chemokine binding of a peptide may be determined by any means known in the art. For example, chemokine binding may be determined by phage display and next generation sequencing (see Example 1, FIG. 1), fluorescence spectroscopy of chemokine dimerization, affinity purification (e.g. peptides immobilised on beads), coimmunoprecipitation, X ray crystallography, NMR, isothermal titration calorimetry, circular dichroism, mass spectrometry, microscale thermophoresis, biointerferometry, HDX-MS, mass spectrometry (MS) dimerization, Alphascreen, surface plasmon resonance, fluorescent polarization, FRET based assays, cell migration assays (see Example 2, FIGS. 2-4), and the like.

Any chemokine binding peptide may be used in a combinatorial peptide entity. A chemokine binding peptide may be a chemokine binding peptide having an amino acid sequence of any one of SEQ ID NOs: 1 to 472 or a variant thereof. A chemokine binding peptide may be a chemokine binding peptide having an amino acid sequence of any one of SEQ ID NOs: 1 to 543 or a variant thereof. A chemokine binding peptide may be a chemokine binding peptide having an amino acid sequence of any one of SEQ ID NOs: 473 to 502 or a variant thereof. A chemokine binding peptide may be a chemokine binding peptide having an amino acid sequence of any one of SEQ ID NOs: 503 to 539 or a variant thereof. A chemokine binding peptide may be a chemokine binding peptide having an amino acid sequence of any one of SEQ ID NOs: 540 to 543 or a variant thereof. The chemokine binding peptide may be a chemokine binding peptide identified in European patent no. EP 1 519 945, (McNaughton, 2018), and/or (Abraham, 2017), all of which are hereby incorporated by reference.

The source of the chemokine binding peptide is not particularly limited. For example, the chemokine binding peptide may be of a synthetic origin, such as identified via a (random) phage display library, or identified by in silico modelling or screening. The chemokine binding peptide may be a fragment of a naturally occurring protein, such as a naturally occurring chemokine binding protein. Such proteins are known. For example, the chemokine binding protein may be a fragment of a human chemokine, a tick evasin, a viral protein, or a human or viral chemokine receptor or other chemokine binding protein. Example 1 and Table 5A provides chemokine binding peptides which are fragments of proteins from each of these sources. Example 6 and Table 5B provide chemokine binding peptides which are synthetic variants of the HD2 peptide in Table 5A. Table 6 provides chemokine binding peptides which were identified in the phage-display screening method described in Example 9. Table 7 provides particularly improved variants of the HD2 peptide described in the Examples. Table 8 provides particularly improved variants of the HD845 peptide described in the Examples. In one aspect, a combinatorial peptide entity of the invention may comprise a plurality of chemokine-binding peptides, wherein at least one chemokine-binding peptide is a fragment of a viral chemokine-binding proteins or a variant thereof. In one aspect, a combinatorial peptide entity of the invention may comprise a plurality of chemokine-binding peptides, wherein at least one chemokine-binding peptide is a fragment of a human or viral chemokine receptor, such as a human chemokine GPCR. In another aspect, a combinatorial peptide entity of the invention may comprise a plurality of chemokine-binding peptides, wherein (a) at least one chemokine-binding peptide is a fragment of a viral chemokine-binding protein or a variant thereof, and (b) (i) at least one chemokine-binding peptide is a fragment of a tick evasin or a variant thereof, (ii) at least one chemokine-binding peptide is a fragment of a chemokine or a variant thereof, and/or (iii) at least one chemokine-binding peptide is a fragment of a chemokine receptor or a variant thereof. In another aspect, a combinatorial peptide entity of the invention may comprise a plurality of chemokine-binding peptides, wherein (a) at least one chemokine-binding peptide is a fragment of a chemokine receptor or a variant thereof, and (b) (i) at least one chemokine-binding peptide is a fragment of a tick evasin or a variant thereof, (ii) at least one chemokine-binding peptide is a fragment of a chemokine or a variant thereof, and/or (iii) at least one chemokine-binding peptide is a fragment of a viral chemokine binding protein or a variant thereof. In the above combinatorial peptide entities, the chemokine-binding peptides may be independently disposed.

The chemokine binding peptide may be a fragment of a class A evasin selected from the group of EVA4, EV672, EV974 and EV546, or a variant thereof.

As used herein, the term ‘fragment’ may refer to a contiguous amino acid sequence of a parental polypeptide/protein. The fragment may be 50 amino acids or less in length, such as 40 amino acids or less, 30 amino acids or less, 25 amino acids or less or 20 amino acids or less in length. The fragment may be 3-50 amino acids in length, such as 4-40 amino acids in length, 5-30 amino acids in length, 6-25 amino acids in length, 10-20 amino acids in length, or 14-18 amino acids in length. The fragment may be 16 amino acids in length.

The chemokine-binding peptide may bind to one or more CC-class chemokines and one or more CXC-class chemokines. The inventors have surprisingly found that short peptides derived from full-length proteins that have the ability to bind chemokines, such as tick evasins, may bind to a greater range of chemokines than the parent protein from which the peptide is derived. In particular, the inventors have identified that whilst class A evasins proteins may only bind to CC-class chemokines, peptides derived from these class A evasin proteins may additionally bind to CXC-class chemokines. Accordingly, the chemokine-binding peptide may be a peptide from a class A evasin of any one of Tables 5-7.

The chemokine-binding peptide may comprise or consist of the amino acid sequence of SEQ ID NO: 396, 403 or 392. The chemokine-binding peptide may comprise or consist of an amino acid sequence having at least 40% sequence identity to the amino acid sequence of any one of SEQ ID NOs: 396, 403 or 392, such as at least 50%, at least 60%, at least 70%, at least 80%, or at least 90% sequence identity to the amino acid sequence of any one of SEQ ID NOs: 396, 403 or 392.

The peptides described herein can be prepared by any suitable technique. They may be made in accordance with the invention as discussed in more detail below.

The peptide may be made by solid-phase peptide synthesis (SPPS). This involves formation of the peptide on small solid beads. Using SPPS, the peptide remains covalently attached to a bead during synthesis. The peptide is synthesised using repeated cycles of coupling-washing-deprotection-washing. In particular, the free N-terminal amine of a solid-phase attached peptide is coupled to a single N-protected amino acid unit. This unit is then deprotected, revealing a new N-terminal amine to which a further protected amino acid is attached. These steps are repeated until the peptide is complete. The peptide may be cleaved from the beads using a suitable reagent, if necessary.

Suitable protecting groups, reagents, solvents and reaction conditions for SPPS are well known to those skilled in the art and as such conditions can be determined by one skilled in the art by routine optimization procedures.

The peptide can be purified, where required, by any suitable technique. High pressure liquid chromatography (HPLC) can be used, for example.

The peptides may be modified using a variety of approaches to improve their function including binding to human serum albumin, lipidation, cyclization, D-amino-acid substitution, replacement of labile amino acids, PEGylation, or amide terminated e.g. to reduce proteolytic degradation, or addition of tags to allow detection (Tan, 2018).

In some cases, the chemokine binding peptides are preferably modified to become a lysosome-targeting chimaera (LYTAC). A LYTAC of the invention comprises the chemokine binding peptide fused to a glycopeptide ligand that targets a lysosomal receptor, to thereby remove the LYTAC and any bound chemokines from the circulation. The glycopeptide ligand may be a first generation LYTAC ligand, i.e. a molecule that targets the cation independent mannose-6-phosphate receptor (CI-M6PR), for example via multiple serine-O-mannose-6-phosphonate (M6Pn) residues as described in (Banik, 2020), which is herein incorporated by reference. The glycopeptide ligand may be a second generation LYTAC ligand, i.e. a molecule that targets the asialoglycoprotein receptor (ASGPR), for example, via a triantennerrary N-acetylgalactosamine (tri-GalNAc) motif as described in (Ahn, 2021), which is herein incorporated by reference. The LYTAC may be generated biosynthetically, for example by fusing sequences containing one or more short N-glycosylation motifs (N—X—S/T) to the peptide and then expressing it, for example, in yeast (Buentzel, 2017). Thus, the chemokine binding peptides may be modified with a residue that targets chemokines for degradation.

The term “peptide” includes not only molecules in which amino acid residues are joined by peptide (—CO—NH—) linkages but also molecules in which the peptide bond is reversed. Such retro-inverso peptidomimetics may be made using methods known in the art, for example such as those described in (Mézière, 1997). This approach involves making pseudopeptides containing changes involving the backbone, and not the orientation of side chains.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH₂→—NH(Me) or —N(Me)₂). Other potential modifications include thioether cyclization and intra- and/or inter-peptide disulphide bonds.

Peptides according to the invention may also include peptide variants that increase or decrease the peptide's half-life in vivo. Examples of analogues capable of increasing the half-life of peptides disclosed herein include peptoid analogues of the peptides, D-amino acid derivatives of the peptides, and peptide-peptoid hybrids. A further embodiment of the variant peptides used according to the invention comprises D-amino acid forms of the peptide. The preparation of peptide using D-amino acids rather than L-amino acids greatly decreases any unwanted breakdown of such an agent by normal metabolic processes, decreasing the amounts of agent which needs to be administered, along with the frequency of its administration.

The peptides may also be derived from amino acid mutants, glycosylation variants and other covalent derivatives of the parent peptides. Exemplary derivatives include molecules wherein the peptides are covalently modified by substitution, chemical, enzymatic, or other appropriate means with a moiety other than a naturally occurring amino acid. Further included are naturally occurring variant amino acid sequences of the parent peptides. Such a variant amino acid sequence may be encoded by an allelic variant from a population.

Modifications as described above may be prepared during synthesis of the peptide or by post-production modification, or when the peptide is in recombinant form using the known techniques of site-directed mutagenesis, random mutagenesis, or enzymatic cleavage and/or ligation of nucleic acids.

The peptides described herein may also be modified to improve physicochemical characteristics. Thus, for example, original amino acid sequences may be altered to improve their solubility, and accordingly a peptide having a variant sequence will preferably be more soluble than a peptide having the corresponding original amino acid sequence under equivalent conditions. Methods for evaluating the solubility of peptides are well known in the art.

### Novel Chemokine Binding Peptides

The inventors have identified a large number of novel chemokine-binding peptides from a range of tick proteins, viral proteins, human chemokines and human or viral chemokine receptors or other chemokine binding proteins, and have determined the peptides' chemokine-binding specificity. The identified peptides are hexadecapeptides that bind to at least three different human chemokines.

Accordingly, provided herein is a chemokine binding peptide comprising the amino acid sequence of any one of SEQ ID NOs: 1 to 472, or a variant thereof. Particular chemokine binding activities determined for each of the peptides are shown in Tables 5A and 5B.

Further provided herein is a chemokine binding peptide comprising the amino acid sequence of any one of SEQ ID NOs: 1 to 543, or a variant thereof.

Further provided herein is a chemokine binding peptide comprising the amino acid sequence of any one of SEQ ID NOs: 473 to 502, or a variant thereof. Further provided herein is a chemokine binding peptide comprising the amino acid sequence of any one of SEQ ID NOs: 503 to 539, or a variant thereof. Further provided herein is a chemokine binding peptide comprising the amino acid sequence of any one of SEQ ID NOs: 540 to 543, or a variant thereof.

The chemokine binding peptides typically bind to three or more chemokines. The chemokine binding peptides may bind to four or more peptides, such as five or more peptides, or six or more peptides.

A combinatorial peptide entity may thus comprise at least one chemokine binding peptide comprising the amino acid sequence of any one of SEQ ID NOs: 1 to 472 or a variant thereof that retains the ability to bind to at least one chemokine, and at least one other chemokine binding peptide. The at least one other chemokine binding peptide may be heterologous to the chemokine binding peptide comprising the amino acid sequence of any one of SEQ ID NOs: 1 to 472 or a variant thereof. The at least one other chemokine binding peptide may be the same as the chemokine binding peptide comprising the amino acid sequence of any one of SEQ ID NOs: 1 to 472. The chemokine binding peptides may be independently disposed.

A combinatorial peptide entity may comprise at least three chemokine binding peptides comprising the amino acid sequence of any one of SEQ ID NOs: 1 to 472 or a variant thereof that retains the ability to bind to at least one chemokine. For example, a combinatorial peptide entity may comprise at least three identical chemokine binding peptides that comprise the same amino acid sequence of any one of SEQ ID NOs: 1 to 472, or variants thereof that retain the ability to bind at least one chemokine. The combinatorial peptide entity may comprise at least four identical chemokine binding peptides that comprise the amino acid sequence of any one of SEQ ID NOs: 1 to 472, or variants thereof that retain the ability to bind at least one chemokine; for example, the combinatorial peptide entity may be a homotetramer of a chemokine binding peptide disclosed herein. The chemokine binding peptides may be independently disposed.

The chemokine binding peptides may alternatively be selected from any one of SEQ ID NOs: 1 to 402 and 404-472 or a variant thereof that retains the ability to bind to at least one chemokine. In some embodiments, the combinatorial peptide entity does not comprise a chemokine binding peptide comprising the amino acid sequence of SEQ ID NO: 403 or a variant thereof, or may only comprise a chemokine binding peptide comprising the amino acid sequence of any one of SEQ ID NOs: 403 or a variant thereof in combination with one or more chemokine binding peptides comprising the amino acid sequence of any one of SEQ ID NOs: 1 to 402 and 404-472 or variants thereof. A combinatorial peptide entity may comprise at least two, such as at least four, independently-disposed chemokine binding peptides, wherein the at least two chemokine binding peptides comprise the same or variant amino acid sequences, and wherein the at least two chemokine binding peptides are selected from any one of SEQ ID NOs: 1 to 402 and 404-472 or a variant thereof that retains the ability to bind to at least one chemokine.

A combinatorial peptide entity may thus comprise at least one chemokine binding peptide comprising the amino acid sequence of any one of SEQ ID NOs: 1 to 543 or a variant thereof that retains the ability to bind to at least one chemokine, and at least one other chemokine binding peptide. The chemokine binding peptide comprising the amino acid sequence of any one of SEQ ID NOs: 1 to 543 or a variant thereof may be a chemokine-binding peptide that comprises an unpaired cysteine. The at least one other chemokine binding peptide may be heterologous to the chemokine binding peptide comprising the amino acid sequence of any one of SEQ ID NOs: 1 to 543 or a variant thereof. The at least one other chemokine binding peptide may be the same as the chemokine binding peptide comprising the amino acid sequence of any one of SEQ ID NOs: 1 to 543. The chemokine binding peptides may be independently disposed.

A combinatorial peptide entity may comprise at least three chemokine binding peptides comprising the amino acid sequence of any one of SEQ ID NOs: 1 to 543 or a variant thereof that retains the ability to bind to at least one chemokine. The chemokine binding peptide comprising the amino acid sequence of any one of SEQ ID NOs: 1 to 543 or a variant thereof may be a chemokine-binding peptide that comprises an unpaired cysteine. For example, a combinatorial peptide entity may comprise at least three identical chemokine binding peptides that comprise the same amino acid sequence of any one of SEQ ID NOs: 1 to 543, or variants thereof that retain the ability to bind at least one chemokine. The combinatorial peptide entity may comprise at least four identical chemokine binding peptides that comprise the amino acid sequence of any one of SEQ ID NOs: 1 to 543, or variants thereof that retain the ability to bind at least one chemokine; for example, the combinatorial peptide entity may be a homotetramer of a chemokine binding peptide disclosed herein. The chemokine binding peptides may be independently disposed.

The chemokine binding peptides may alternatively be selected from any one of SEQ ID NOs: 1 to 402 and 404-543 or a variant thereof that retains the ability to bind to at least one chemokine. The chemokine binding peptide comprising the amino acid sequence of any one of SEQ ID NOs: 1 to 402 and 404-543 or a variant thereof may be a chemokine-binding peptide that comprises an unpaired cysteine. In some embodiments, the combinatorial peptide entity does not comprise a chemokine binding peptide comprising the amino acid sequence of SEQ ID NO: 403 or a variant thereof, or may only comprise a chemokine binding peptide comprising the amino acid sequence of any one of SEQ ID NOs: 403 or a variant thereof in combination with one or more chemokine binding peptides comprising the amino acid sequence of any one of SEQ ID NOs: 1 to 402 and 404-543 or variants thereof. A combinatorial peptide entity may comprise at least two, such as at least four, independently-disposed chemokine binding peptides, wherein the at least two chemokine binding peptides comprise the same or variant amino acid sequences, and wherein the at least two chemokine binding peptides are selected from any one of SEQ ID NOs: 1 to 402 and 404-543 or a variant thereof that retains the ability to bind to at least one chemokine.

A chemokine binding peptide may be selected from a chemokine binding peptide selected from any one of SEQ ID NOs: 1 to 472 (see Tables 5A and 5B) that binds to a CXC class chemokine, a CX3C class chemokine and/or a XC class chemokine (see Table 3 E for chemokine class information). A chemokine binding peptide selected from any one of SEQ ID NOs: 1 to 472 may be a chemokine binding peptide that binds to a CC class chemokine and a chemokine selected from a CXC-class, CX3C class or an XC class chemokine. A chemokine binding peptide selected from any one of SEQ ID NOs: 1 to 472 may be a fragment of (i) a class A Evasin, (ii) a class B evasins, (iii) a viral chemokine binding proteins or (iv) a human chemokine or (v) a human or viral chemokine receptor or other chemokine-binding protein. For example, a chemokine binding peptide selected from any one of SEQ ID NOs: 1 to 472 may be selected from: (i); (ii); (iii); (iv); (v); (i) and (ii); (i) and (iii); (i) and (iv); (i) and (iv); (ii) and (iii); (ii) and (iv); (ii) and (v); (iii) and (iv); (iii) and (v); (iv) and (v); (i), (ii) and (iii); (i), (ii) and (iv); (i), (ii) and (v); (i), (iii) and (iv); (i), (iii) and (v); (i), (iv) and (v); (ii), (iii) and (iv); (ii), (iii) and (v); (ii), (iv) and (v); (i), (ii), (iii) and (iv); (i), (ii), (iii) and (v); (i), (ii), (iv) and (v); (i), (iii), (iv) and (v); (ii), (iii), (iv) and (v); or (i), (ii), (iii), (iv) and (v). The class A evasins, class B evasins, viral chemokine binding proteins, human chemokines and human or viral chemokine receptors or other chemokine binding proteins may be selected from those listed in Tables 3A to 3F. A combinatorial peptide entity of the invention may comprise at least one chemokine binding peptide selected from the selections provided above.

A chemokine binding peptide may be selected from a chemokine binding peptide selected from any one of SEQ ID NOs: 1 to 543 (see Tables 5-8) that binds to a CXC class chemokine, a CX3C class chemokine and/or a XC class chemokine (see Table 3 E for chemokine class information). A chemokine binding peptide selected from any one of SEQ ID NOs: 1 to 543 may be a chemokine binding peptide that binds to a CC class chemokine and a chemokine selected from a CXC-class, CX3C class or an XC class chemokine. A chemokine binding peptide selected from any one of SEQ ID NOs: 1 to 543 may be a fragment of (i) a class A Evasin, (ii) a class B evasins, (iii) a viral chemokine binding proteins or (iv) a human chemokine or (v) a human or viral chemokine receptor or other chemokine-binding protein. For example, a chemokine binding peptide selected from any one of SEQ ID NOs: 1 to 543 may be selected from: (i); (ii); (iii); (iv); (v); (i) and (ii); (i) and (iii); (i) and (iv); (i) and (iv); (ii) and (iii); (ii) and (iv); (ii) and (v); (iii) and (iv); (iii) and (v); (iv) and (v); (i), (ii) and (iii); (i), (ii) and (iv); (i), (ii) and (v); (i), (iii) and (iv); (i), (iii) and (v); (i), (iv) and (v); (ii), (iii) and (iv); (ii), (iii) and (v); (ii), (iv) and (v); (i), (ii), (iii) and (iv); (i), (ii), (iii) and (v); (i), (ii), (iv) and (v); (i), (iii), (iv) and (v); (ii), (iii), (iv) and (v); or (i), (ii), (iii), (iv) and (v). The class A evasins, class B evasins, viral chemokine binding proteins, human chemokines and human or viral chemokine receptors or other chemokine binding proteins may be selected from those listed in Tables 3A to 3F. A combinatorial peptide entity of the invention may comprise at least one chemokine binding peptide selected from the selections provided above.

Preferred chemokine binding peptides of the invention are HD2 (SEQ ID NO: 396) and EB429 (SEQ ID NO: 392). Preferably, a combinatorial peptide entity of the invention comprises HD2 and/or EB429, or variants thereof.

Preferred chemokine binding peptides of the invention are HD2 (SEQ ID NO: 396), HD845 (SEQ ID NO: 403) and EB429 (SEQ ID NO: 392). Preferably, a combinatorial peptide entity of the invention comprises HD2, HD845 and/or EB429, or variants thereof.

Chemokine Binding Peptides that Bind to Both CC- and CXC-Class Chemokines

The inventors have surprisingly identified a chemokine binding motif that is predictive of the ability to bind chemokines in the CC-class and the CXC- and/or XC-class, as described in the examples. Said motif comprises an N-terminal acidic region and a C-terminal hydrophobic portion. The inventors have surprisingly identified that the presence of one or more unpaired cysteine residues in the C-terminal portion contributes to the ability of the peptide to bind CXC- and XC-class chemokines.

Accordingly, a chemokine-binding peptide may comprise an N-terminal acidic region and a C-terminal hydrophobic region. The chemokine-binding peptide is capable of binding to one or more CC-class chemokines and one or more CXC- and/or XC-class chemokines. The chemokine-binding peptide typically comprises one or more unpaired cysteine residues.

The chemokine-binding peptide may be 20 or fewer amino acids in length, such as 19 or fewer, 18 or fewer, 17 or fewer or 16 or fewer amino acids in length. The chemokine binding peptide may be 10-20 amino acids in length, such as 12-19, 14-18, 15-17 or about 16 amino acids in length.

The N-terminal acidic region may be 5 to 10 amino acids in length, such as 6-9 or 7-8 amino acids in length. The N-terminal acidic region typically comprises 3 or more acidic amino acids (e.g. D or E), such as 4 or more or 5 acidic amino acids. The N-terminal acidic region typically comprises 1 or more aromatic amino acids, such as 2 or 3 aromatic amino acids. The aromatic amino acids are preferably selected from Y or W, more preferably W.

The C-terminal hydrophobic region may be 5 to 10 amino acids in length, such as 6-9 or 7-8 amino acids in length. The C-terminal regional typically comprises The C-terminal region typically comprises 3 or more hydrophobic residues, such as 4 hydrophobic residues. The hydrophobic residues may be selected from A, V, C, P, L, I, M, W and F. The hydrophobic region typically comprises 2 or more aromatic amino acids, such as 3 aromatic amino acids. The aromatic amino acids are selected from W, Y and F. The hydrophobic region may comprise 1 or more polar amino acids and/or 1 acidic amino acid. The polar amino acids may be selected from S, T or Y. The acidic acid may be D. The acidic amino acid (if present) is typically provided at the C-terminus of the peptide. In some cases, the C-terminal region may not comprise a charged amino acid (i.e. H, K, R, D or E). The C-terminal region may comprise one or more proline residue.

The one or more unpaired cysteine residues may be present in the C-terminal region or the N-terminal region. Typically, the one or more unpaired cysteine residues is present in the C-terminal region. An unpaired cysteine residue may be present in the 3^{rd}, 4^{th} or 5^{th} position from the C-terminus of the peptide. The one or more proline residues may be present in the 6th, 7^{th} or 8^{th} position from the C-terminus of the peptide.

The chemokine binding peptide may comprise an amino acid sequence of the formula:

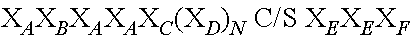

wherein:
X_{A} is D or E
X_{B} is D, E or W
X_{C} is an aromatic amino acid, preferably Y or W
X_{D} is any amino acid. N is 5-10, more preferably 6-9, more preferably 7 or 8
X_{E} is an aromatic amino acid, preferably Y or F
X_{F} is any amino acid or absent.

### The Chemokine Binding Peptide May Comprise an Amino Acid Sequence of the Formula:

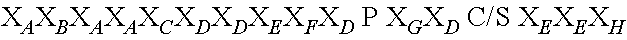

wherein:
X_{A} is D or E
X_{B} is D, E or W
X_{C} is an aromatic amino acid, preferably Y or W
X_{D} is any amino acid.
X_{E} is an aromatic amino acid, preferably Y or F
X_{F} is an aromatic amino acid or absent, preferably Y or F or absent
X_{G} is any amino acid, preferably a hydrophobic amino acid, more preferably V, L or I
X_{H} is any amino acid or absent.

### The Chemokine Binding Peptide May Comprise an Amino Acid Sequence of the Formula:

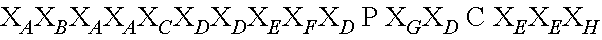

wherein:
X_{A} is D or E or absent
X_{B} is D, E or W
X_{C} is Y or W
X_{D} is any amino acid
X_{E} is an aromatic amino acid, preferably Y or F
X_{F} is an aromatic amino acid or absent, preferably Y, F or absent
X_{G} is a hydrophobic amino acid, preferably V, L or I
X_{H} is T, C, D or absent.

### The Chemokine Binding Peptide May Comprise an Amino Acid Having the Sequence:

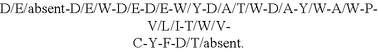

### Variants

A variant of a chemokine binding peptide typically retains the ability to bind at least one chemokine bound by its parent chemokine binding peptide. Typically, a variant binds to three or more chemokines. The term ‘parent’ as used herein is to mean the starting peptide that is modified, as described below, to produce the variant.

The variant may retain the ability to bind at least two of the chemokines bound by the parent chemokine binding peptide, such as at least three, at least four, or at least five of the chemokines as the parent chemokine binding peptide. The variant may retain the ability to bind two, three, four, five or more of the chemokines bound by the parent chemokine binding protein. The variant may retain the ability to bind at least 50% of the chemokines that the parent chemokine binding peptide can bind, such as at least 60%, at least 70%, at least 80% or at least 90% of the chemokines that the parent chemokine binding peptide can bind. The variant typically retains the ability to bind substantially all or all of the chemokines bound by the parent chemokine binding peptide. The term ‘substantially all’ as used herein means that the variant binds to all of the chemokines bound by the parent chemokine binding peptide that can be reliably detected using methods such as those described in relation to determining the chemokine binding of a peptide.

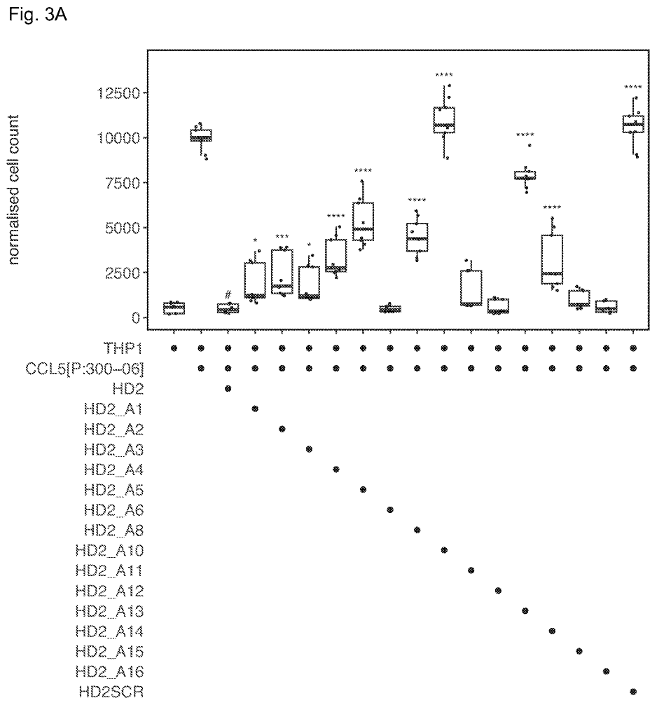

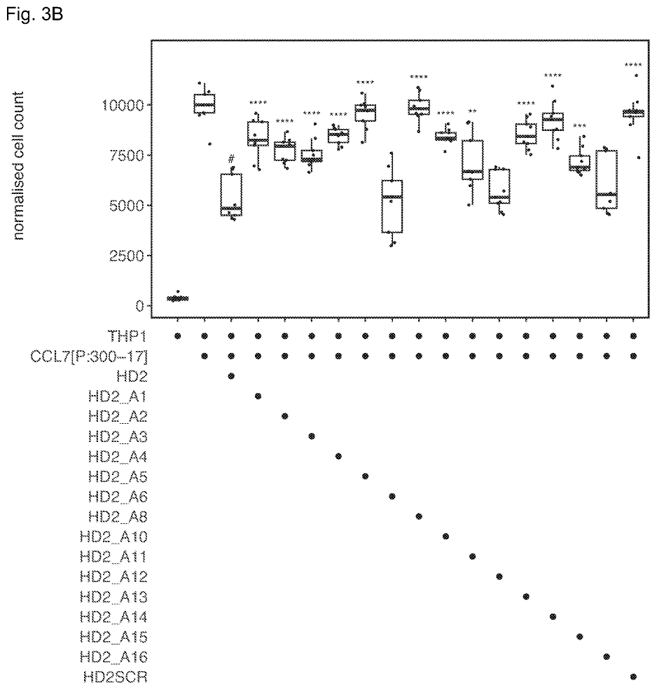

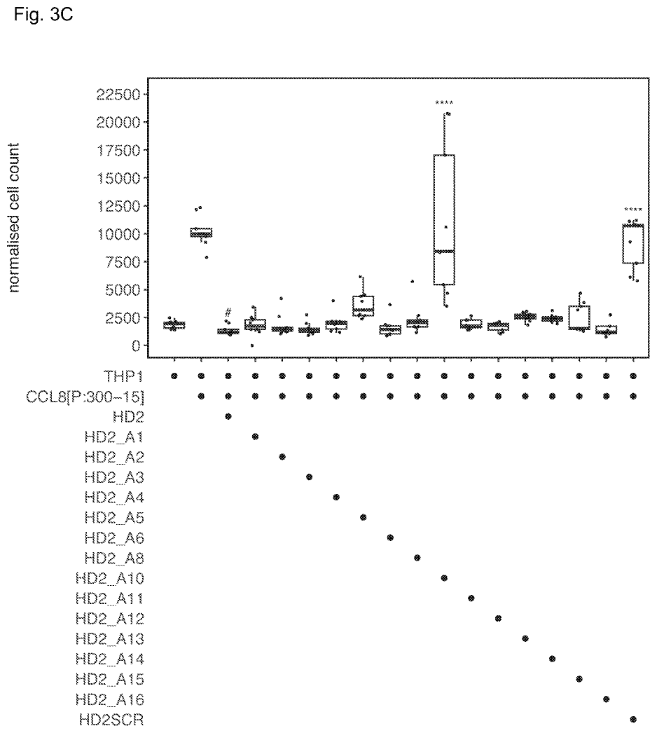

As shown in FIG. 3, alanine-scanning mutagenesis experiments have demonstrated that variants of the hexadecapeptides may retain their ability to bind chemokines. For example, substitution of the chemokine binding peptide ‘HD2’ (SEQ ID NO: 396) at each of positions 1-6, 8 and 11-16 with alanine does not lead to a significant change in the chemokine-inhibitory specificity to the chemokine CCL8 (see FIG. 3C). Thus, the skilled person would be able to identify variants of the chemokine binding peptides of the invention using routine experimentation and standard experimental techniques.

As demonstrated by Tables 5A and 5B, variants of chemokine binding peptides may retain the ability to bind three or more chemokines, and/or retain the ability to bind at least one chemokine that the ‘parent’ peptide binds. For example, SEQ ID NOs: 13 to 15 differ by a single amino acid at position 12, yet all three retain the ability to bind to human CCL19 and CCL20. SEQ ID NOs: 23 and 24 differ by a single amino acid at position 4 and each bind inter alia human CCL1, CCL15, CCL17, CCL19, CCL20, CCL22, X3CL1, CXL10, CXL11, CXL13 and IL8. SEQ ID NOs: 20, 42, 123, 230, 243, 326, 327 and 365 have 9 amino acids in common and bind similar chemokines. SEQ ID NOs: 386 and 390 have 12 amino acids in common and bind similar chemokines. SEQ ID NOs: 408, 430 and 431 have 11 amino acids in common and each bind chemokines CXL10, CXL11 and IL8. These examples are not limiting and demonstrate the principle that a chemokine binding peptide may be modified yet retain chemokine binding activity. Accordingly, the chemokine binding peptides described herein may be varied while retaining the ability to bind chemokines.

Variants of peptides may also be discovered by saturation mutagenesis followed by selection for binding. For instance, codons encoding each residue may be replaced with the sequence NNK or NNS (which encode all 20 amino acids), the mutant peptide pool expressed for instance by phage-display or mRNA-display or yeast-surface display, and selected using the target chemokine. An example of such saturation mutagenesis followed by phage display selection using chemokines has identified variants that have either wider chemokine binding or enhanced chemokine binding in comparison to the parental peptide. Exemplary peptide sequences with wider and/or enhanced affinity derived from SEQ ID NO: 396 (HD2) are provided in Table 5B (SEQ ID NOs: 452 to 472) and FIG. 7. Accordingly, in any of the aspects of the invention discussed herein, the chemokine binding peptide may be a variant of HD2 having enhanced and/or wider chemokine binding activity than HD2, such as those provided in SEQ ID NOs: 452 to 472.

The chemokine-binding peptide may be a variant of HD2, HD845 or EB429, for example, may comprise or consist of an amino acid sequence having at least 40% sequence identity to the amino acid sequence of SEQ ID NO: 396, 403 or 392, such as at least 50%, at least 60%, at least 70%, at least 80%, or at least 90% sequence identity to the amino acid sequence of SEQ ID NO: 396, 403 or 392.

The chemokine-binding peptide may be a variant of HD2, for example, may comprise or consist of an amino acid sequence having at least 40% sequence identity to the amino acid sequence of SEQ ID NO: 396, such as at least 50%, at least 60%, at least 70%, at least 80%, or at least 90% sequence identity to the amino acid sequence of SEQ ID NO: 396. The chemokine-binding peptide may be a variant of HD845, for example, may comprise or consist of an amino acid sequence having at least 40% sequence identity to the amino acid sequence of SEQ ID NO: 396, such as at least 50%, at least 60%, at least 70%, at least 80%, or at least 90% sequence identity to the amino acid sequence of SEQ ID NO: 403. The variant of HD2, HD845 or EB429 typically has enhanced and/or wider chemokine binding activity than HD2, HD845 or EB429 respectively. The variant of HD2 or HD845 typically has enhanced and/or wider chemokine binding activity than HD2 or HD845, respectively, and retains the ability to bind one or more CC-class chemokines, one or more-CXC-class chemokines and/or one or more XC-class chemokines.

The variant of HD2 (SEQ ID NO: 396) may comprise one or more of the following amino acid substitutions: E1D, E2D/W, D3 E, D4 E, Y5W, T6D/E/W, A7W/D, A9W, L11I, T12W and T16C/D. The variant may comprise one or more of Y5W, T6W, A9W and T16C. The amino acid sequence may comprise the amino acid of sequence of any one of SEQ ID NOs: 503-539, or a further variant thereof comprising 5 or fewer conservative amino acid substitutions, such as 4 or fewer, 3 or fewer, 2 or fewer or 1 conservative amino acid substitution(s).

The variant of HD845 (SEQ ID NO: 403) may comprise one or more of the following amino acid substitutions: E6W, E6F, K10A and K10W. The variant may comprise one or more of E6W, E6F and K10A. The amino acid sequence may comprise the amino acid of sequence of any one of SEQ ID NOs: 540-543, or a further variant thereof comprising 5 or fewer conservative amino acid substitutions, such as 4 or fewer, 3 or fewer, 2 or fewer or 1 conservative amino acid substitution(s).

Preferably, the variant of HD2 or HD845 comprises a cysteine residue, for example a cysteine residue corresponding to the unpaired cysteine residue in SEQ ID NO: 396 (HD2) or 403 (HD845). More preferably, the variant of HD2 or HD845 comprises an unpaired cysteine residue, for example an unpaired cysteine residue corresponding to the unpaired cysteine residue in SEQ ID NO: 396 (HD2) or 403 (HD845).

A variant of a ‘parent’ chemokine binding peptide may comprise at least 50% amino acid sequence identity with the ‘parent’ chemokine binding peptide. For example, a variant of a ‘parent’ chemokine binding peptide may comprise at least 60%, at least 70%, at least 80%, at least 90% or at least 95% amino acid sequence identity with the ‘parent’ chemokine binding peptide.

A variant of a parent chemokine binding peptide may comprise an amino acid sequence having one or more amino acid modifications compared to the sequence of a ‘parent’ chemokine binding peptide. The variant may comprise two or more, three or more, four or more, five or more, six of more, seven or more, eight or more, nine or more or ten amino acid modifications compared to a ‘parent’ chemokine binding peptide. The variant may comprise two, three, four, five, six, seven, eight, nine, ten or more amino acid modifications compared to a ‘parent’ chemokine binding protein. The variant may comprise one to ten, such as one to nine, one to eight, one to seven, one to six, one to five, one to four, or one to three modifications compared to a ‘parent’ chemokine binding peptide. Preferably, a variant comprises at least ten amino acids, such as at least ten contiguous amino acids, of a ‘parent’ chemokine binding peptide. For example, a variant may comprise at least 11, at least 12, at least 13, at least 14 or at least 15 amino acids of a ‘parent’ chemokine binding peptide, such as at least 11, at least 12, at least 13, at least 14 or at least 15 contiguous amino acids of a ‘parent’ chemokine binding peptide. A variant may comprise ten, eleven, twelve, thirteen, fourteen, fifteen or more amino acids of a ‘parent’ chemokine binding protein, such as ten, eleven, twelve, thirteen, fourteen, fifteen or more contiguous amino acids of a ‘parent’ chemokine binding protein.

A modification may be the deletion of an amino acid. A modification may be the addition of an amino acid. A modification may be the substitution of an amino acid with another amino acid. A variant polypeptide may comprise one or more deletions when compared to the sequence of a ‘parent’ chemokine binding peptide, such as two or more, three or more, four or more, five or more, six of more, seven or more, eight or more, nine or more or ten amino acid deletions of amino acids when compared to the sequence of a ‘parent’ chemokine binding peptide. A variant polypeptide may comprise one, two, three, four, five, six, seven, eight, nine, ten or more deletions when compared to the sequence of a ‘parent’ chemokine binding protein. Preferably, a variant polypeptide comprises fewer than six deletions of an amino acid when compared to the sequence of a ‘parent’ chemokine binding peptide, such as five or fewer, four or fewer, three or fewer, two or one deletions of amino acids when compared to the sequence of a ‘parent’ chemokine binding peptide. A variant polypeptide may comprise six, five, four, three, two or one deletions of an amino acid when compared to the sequence of a ‘parent’ chemokine binding protein.

Substitutions may be Ala to Cys, Cys to Ala, Ala to Ser, Ser to Ala, Cys to Ser, and/or Ser to Cys. These modifications have been shown to alter the chemokine binding profile of the chemokine binding peptides exemplified herein (see Table 5A, column ‘peptide source’, where ‘CS’ or ‘CA’ following Uniprot accession indicates a Cys to Ser or and Cys to Ala substitution, respectively). Other substitutions are possible and may be used to widen or enhance the chemokine binding activity of a peptide, as shown in Example 6 and Table 5B. The substitution may be a conservative substitution. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A variant polypeptide may comprise one or more substitutions of an amino acid when compared to the sequence of a ‘parent’ chemokine binding peptide, such as two or more, three or more, four or more, five or more, six or more, seven or more, eight or more, nine or more or ten amino acid substitutions of an amino acid when compared to the sequence of a ‘parent’ chemokine binding peptide. A variant polypeptide may comprise one, two, three, four, five, six, seven, eight, nine, ten or more substitutions of an amino acid when compared to the sequence of a ‘parent’ chemokine binding protein. Preferably, a variant polypeptide comprises fewer than four substitutions of an amino acid when compared to the sequence of a ‘parent’ chemokine binding peptide, such as three or fewer, two or one substitutions of an amino acid when compared to the sequence of a ‘parent’ chemokine binding peptide. A variant polypeptide may comprise four, three, two or one substitutions of an amino acid when compared to the sequence of a ‘parent’ chemokine binding protein.

Where the modification comprises an addition of an amino acid, the added amino acid may be inserted at any position in the ‘parent’ chemokine binding peptide. An amino acid may be added at the N-terminus and/or C-terminus of the ‘parent’ chemokine binding peptide. An amino acid may be added at a position internal to the sequence of the ‘parent’ chemokine binding peptide, i.e, wherein the added amino acid is flanked on its N- and C-ends by one or more amino acids of the ‘parent’ chemokine binding peptide. When the modification comprises an addition of an amino acid, an amino acid is typically added at the N-terminus and/or C-terminus of the ‘parent’ chemokine binding peptide and is not added at a position internal to the sequence of the ‘parent’ chemokine binding peptide. The modification may comprise the addition of a tyrosine residue to the N-terminus of the chemokine-binding peptide. The modification may comprise the addition of amino acids (such as a number of amino acid additions specified below) to the N- or C-terminus of the chemokine binding peptide that correspond to the amino acids that natively flank the N- or C-terminus of the peptide in the protein of origin, i.e, wherein the chemokine binding peptide represents a fragment of said protein of origin. A variant polypeptide may comprise one or more additions of an amino acid when compared to the sequence of a ‘parent’ chemokine binding peptide, such as two or more, three or more, four or more, five or more, six or more, seven or more, eight or more, nine or more or ten amino acid additions of an amino acid when compared to the sequence of a ‘parent’ chemokine binding peptide. A variant polypeptide may comprise one, two, three, four, five, six, seven, eight, nine, ten or more additions of an amino acid when compared to the sequence of a ‘parent’ chemokine binding protein. Preferably, a variant polypeptide comprises fewer than six additions of an amino acid when compared to the sequence of a ‘parent’ chemokine binding peptide, such as five or fewer, four or fewer, three or fewer, two or one additions of an amino acid when compared to the sequence of a ‘parent’ chemokine binding peptide. A variant polypeptide may comprise six, five, four, three, two or one additions of an amino acid when compared to the sequence of a ‘parent’ chemokine binding protein.

A variant chemokine binding peptide may comprise any combination of additions, deletions and/or substitutions of amino acids, when compared to the sequence of a ‘parent’ chemokine binding peptide.

The ability of a variant to bind to and preferably inhibit a chemokine can be assayed using any method known in the art. Suitable methods are described in the Examples and Figures, and include yeast surface display and biolayer interferometry (for binding) and chemotaxis assays (for inhibition).

N- or C-terminal truncations may be made to any chemokine binding peptide described herein to provide a “minimal” chemokine-binding peptide (for one or more chemokines of interest). A minimal chemokine binding peptide has N- and/or C-terminal deletions yet retains the ability to bind the same chemokine(s) as the parent peptide. The minimal chemokine binding peptide typically does not include other amino acids of the parent chemokine binding peptide that are not essential for the relevant chemokine-binding activity. Truncation variants of a chemokine binding peptides that comprise minimal chemokine-binding sequences may also be screened for their ability to inhibit or neutralize chemokine activity, for example by performing a chemokine-induced cell migration assay, for example the assay as described in Example 2, or as shown in FIGS. 2 to 4.

Additionally, chemokine binding peptide variants may be identified based on sequence alignment and structural modelling of chemokine binding proteins having a high amino acid sequence identity (typically at least 50%, such as at least 60%, at least 70%, at least 80% or at least 90%) and structural similarity to the chemokine binding proteins from which the chemokine binding peptides herein have been identified (see Example 1 and Tables 3A-3 E for the chemokine binding proteins used to identify to the novel chemokine binding peptides identified herein). For example, conserved cysteine sets present in tick chemokine binding proteins (e.g. evasins) allow for alignment of their sequences. Thus, the position of a chemokine-binding sequence identified in one tick chemokine binding protein can be aligned against other tick chemokine binding proteins of the same sub-family to identify a variant chemokine binding peptide.

Structural modelling may also be used to assist identification of variant chemokine binding peptides. For example, a published structure is available for Evasin-1 (3FPU), in complex with CCL3; structural models for other tick evasins can be generated using this template, thereby predicting peptides in the modelled tick evasin that form an interface with a chemokine, and a location for a chemokine-binding sequence in the primary sequence. Structural modelling of complexes may also be used to help identify residues in a chemokine binding peptide that are not involved in the interaction with a chemokine, and thus may be modified without affecting the binding properties of the peptide to the chemokine(s) of interest. Structural modelling and identification of important residues may be performed in the absence of a template using computational approaches such as Alphafold2 (Jumper J, 2021) to model peptide interaction with chemokine, and by computationally aligning peptides with similar sequences to identify conserved residues. Function of peptides may additionally be computationally predicted using Deep Proteomic approaches e.g. “ProtVec” as described in (Asgari E, 2015).

Experimental approaches to support structural modelling to determine important peptide residues that bind chemokine may use solution NMR spectroscopy, X-ray crystallography, hydrogen-deuterium exchange mass-spectrometry, or systematic mutagenesis of peptide residues followed by studies of binding or inhibition of a chemokine.

### Characteristics

A chemokine binding peptide is 50 or fewer amino acids in length. A chemokine binding peptide may be 40 or fewer, 30 or fewer, or 20 or fewer amino acids in length. Typically, the chemokine binding peptide is at least 4 amino acids in length, such as at least 5, at least 6, at least 7, preferably at least 8, at least 9, and least 10, more preferably at least 12 or at least 15 amino acids in length. The chemokine binding protein may be four, five, six, seven, eight, nine, ten, eleven, twelve, thirteen, fourteen, fifteen, sixteen, seventeen, eighteen or more amino acids in length. A chemokine binding peptide may be 5 to 50 amino acids in length, such as 10 to 40, 10 to 30, 10 to 20, or 15 to 20 amino acids in length.

The peptide typically comprises naturally-occurring amino acid residues. The peptide may contain non-naturally-occurring amino acids. The peptide typically comprises L-amino acids. The peptide may comprise D-amino acids.

The combinatorial peptide entity may be chemically modified to enhance (a) bioactivity and/or (b) absorption, distribution, metabolism and/or excretion characteristics.

### Labels

The combinatorial peptide entity and/or the chemokine binding peptide may be labelled with a detectable label. The combinatorial peptide entity may be labelled at any site, including a site other than a chemokine binding peptide, For example, where the combinatorial peptide entity is a branched peptide, a ‘core’ peptide may be labelled. The detectable label may be any suitable label which allows the peptide to be detected. Suitable labels include, but are not limited to, fluorescent molecules, radioisotopes, e.g. ¹²⁵I, ³⁵S, enzymes, antibodies, antigens, polynucleotides and ligands such as biotin. The label may be a tracer that is suitable for positron emission tomography (PET), such as fluorine (¹⁸F). The label may be a tracer suitable for magnetic resonance imaging (MRI), such as fluorine (¹⁹F). The label may be a Fluorescein isothiocyanate (FITC) moiety.

One or more of, such as all of, the chemokine binding peptides in a combinatorial peptide entity may be labelled with a detectable label. The label may be any of those discussed above. Different chemokine binding peptides in the combination may be labelled with the same detectable label or different detectable labels.

The combinatorial peptide entity and/or the chemokine binding peptide may be labelled with an epitope tag or purification tag or cell-surface display tag or a tag that enables or facilitates systemic peptide delivery or delivery and targeting to a specific organ or to a tumour, or facilitates transfer across a barrier such as skin or gut or blood brain barrier. Suitable tags are known in the art. Suitable tags include, but are not limited to, AviTag, calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, BCCP (Biotin Carboxyl Carrier Protein), Glutathione-S-transferase-tag, Green fluorescent protein-tag, Halo-tag, Maltose binding protein-tag, Nus-tag, Thioredoxin-tag, Strep-tag, Skin permeating and cell entering (SPACE)-tag, TD1-tag, magainin tag, TAT-tag, penetratin-tag, cell penetrating peptide (CPP)-tag, Fc tag. The second peptide or polypeptide may be a signal peptide, such as an IgK signal peptide.

### Polynucleotides, Vectors and Cells

The invention also provides a polynucleotide which encodes a peptide of the invention. The peptide may be any of those discussed above.

The invention also provides a polynucleotide which encodes two or more peptides comprised within a combinatorial peptide entity of the invention. The coding sequences for the two or more peptides may be present in a single polynucleotide of the invention. This is typically the case when the combination is encoded by a single vector of the invention. In this case, the coding sequence of the two or more peptides are typically present in separate open reading frames. The coding sequence of the two or more peptides may be present in a single open reading frame, but the produced polypeptide may comprise cleavage sites for cleavage of the polypeptide into the two or more peptides, e.g. by enzymatic cleavage.

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxy guanosine diphosphate (dGDP), deoxy guanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hydroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

The polynucleotide sequence encodes the relevant polypeptide(s) on the basis of the genetic code, including its degeneracy.

The polynucleotide may be a ribonucleic acid modified to reduce immunogenicity and increase stability for instance by substitution of uridine and cytidine with 1-methylpseudouridine and 5-methylcytidine, and/or placing an Anti-Reverse Cap Analog (ARCA) cap at the 5′ end. Such modified ribonucleic acids can be delivered using nanoparticles and other transfection reagents.

Polynucleotide sequences may be derived and replicated using standard methods in the art, for example using PCR involving specific primers. It is straightforward to generate polynucleotide sequences using such standard techniques. These are discussed in more detail below.

The invention also provides a combination of two or more polynucleotides each of which encodes a peptide of the invention, i.e. each of which encodes a different peptide of the invention. The combination may encode two or more peptides of the invention. The combination may encode all of the peptides comprised within a combinatorial peptide entity.

The combination may comprise any number of different polynucleotides. For instance, the combination may comprise three or more different polynucleotides of the invention such as four or more, five or more, six or more, seven or more, eight or more, or nine or more different polynucleotides of the invention. The combination may comprise three, four, five, six, seven, eight, nine or more different polynucleotides of the invention.

The invention also provides a vector comprising a polynucleotide of the invention or a combination of two or more polynucleotides of the invention.

The vector may be a cloning vector. The amplified sequences may be incorporated into a recombinant replicable vector such as a cloning vector. The vector may be used to replicate the polynucleotide in a compatible host cell. Thus polynucleotide sequences may be made by introducing the polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The vector may be an expression vector. The polynucleotide sequence may be cloned into any suitable expression vector. In an expression vector, the polynucleotide of the invention or the combination of the invention is typically operably linked to a control sequence which is capable of providing for the expression of the polynucleotide or the combination by the host cell. Such expression vectors can be used to express one or more polypeptides of the invention.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The term “control sequence” is intended to include promoters, enhancers, internal ribosomal entry sites (IRES), and other expression control elements (e.g. transcription termination signals, such as polyadenylation signals and poly-U sequences). Control sequences may ensure expression of the polynucleotide in certain types of cells. Such control sequences are known in the art.

Control sequences may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “control sequence” are enhancer elements, such as WPRE; CMV enhancers; the R—U5′ segment in LTR of HTLV-I; SV40 enhancer; and the intron sequence between exons 2 and 3 of rabbit β-globin. It will be appreciated by those skilled in the art that the design of the expression vector can depend on such factors as the choice of the host cell to be transformed, the level of expression desired, etc.

The expression vector may then be introduced into a suitable host cell. Thus, polypeptide of the invention can be produced by inserting a polynucleotide or a combination into an expression vector, introducing the vector into a compatible bacterial host cell, and growing the host cell under conditions which bring about expression of the polynucleotide or combination. The vectors may be for example, plasmid, virus or phage vectors provided with an origin of replication, optionally a promoter for the expression of the said polynucleotide or combination and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The vector may be used to administer a polynucleotide of the invention or a combination of two or more polynucleotides to a subject as discussed in more detail below. Conventional viral and non-viral based gene transfer methods can be used to introduce the polynucleotide or combination into cells. Non-viral vector delivery systems include DNA plasmids, RNA, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides are known. The preparation of lipid: nucleic acid complexes, including targeted liposomes such as immunolipid complexes, is well known to one of skill in the art.

Conventional viral based expression systems could include retroviral, lentivirus, adenoviral, adeno-associated (AAV) and herpes simplex virus (HSV) vectors for gene transfer. Methods for producing and purifying such vectors are known in the art.

Exemplary vector systems for using the invention are a virus, such as rAAV, that comprises or consists essentially of an exogenous polynucleotide encoding the polypeptide, fusion polypeptide or polypeptide combination of the invention, e.g., a cassette comprising or consisting essentially of a promoter, a polynucleotide encoding the polypeptide, fusion polypeptide or polypeptide combination of the invention and a terminator.

Since AAV is a DNA virus, the polynucleotides used in AAV or rAAV are advantageously DNA.

The vector may be delivered using nanoparticle delivery systems. Such delivery systems include, but are not limited to, lipid-based systems, liposomes, micelles, microvesicles, exosomes, and gene gun. Lipid Nanoparticles, Spherical Nucleic Acid (SNA™) constructs, nanoplexes and other nanoparticles (particularly gold nanoparticles) are also contemplated as a means for delivery of a polynucleotide or a polynucleotide of the invention. The invention provides any of these deliver systems comprising a vector of the invention, a polynucleotide of the invention or a polynucleotide combination of the invention.

In some embodiments, the vector may form a component of an inducible system. The inducible nature of the system would allow for spatiotemporal control of expression of a polypeptide of the invention or a combination of such polypeptides using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc), or light inducible systems (Phytochrome, LOV domains, or cryptochrome).

As will be clear from below, the polynucleotide of the invention or a polynucleotide combination of the invention or any expression vector containing these components may be present in a population of cells. The cells may be administered to the subject. Suitable ways of modifying and administering cells are known in the art.

The invention also provides a host cell which comprises a polynucleotide of the invention, a polynucleotide combination of the invention or a vector of the invention. The host cell may be used to replicate the polynucleotide, combination or vector. The host cell may be used to express a peptide of the invention or a combination of peptides of the invention in vitro. The host cell may be used to deliver the polynucleotide, combination or vector to a subject in need thereof as discussed below.

Host cells will be chosen to be compatible with the cloning or expression vector used to transform the cell. Suitable conditions are known in the art.

Suitable cells for use in the invention include prokaryotic cells and eukaryotic cells. The prokaryotic cell is preferably a bacterial cell. Suitable bacterial cells include, but are not limited to, Escherichia coli, Corynebacterium and Pseudomonas fluorescens. Any E. coli cell with a DE3 lysogen, for example C41 (DE3), BL21 (DE3), JM109 (DE3), B834 (DE3), TUNER, Origami and Origami B, can express a vector comprising the T7 promoter.

Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf9, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other preferred mammalian cells include, but are not limited to, PC12, HEK293, HEK293A, HEK293T, CHO, BHK-21, HeLa, ARPE-19, RAW264.7 and COS cells.

The host cell may be HEK293T.

If the cell is being administered to a subject, the cell is preferably derived from the subject or a subject of the same species. For instance, a human cell is typically administered to a human subject. The host cell is preferably autologous. In other words, the cell is preferably derived from the subject into which the cell will be administered. Alternatively, the host cell is preferably allogeneic. In other words, the cell is preferably derived from a patient that is immunologically compatible with the patient into which the cell will be administered.

The cell may be isolated, substantially isolated, purified or substantially purified. The cell is isolated or purified if it is completely free of any other components, such as culture medium or other cell types. The cell is substantially isolated if it is mixed with carriers or diluents, such as culture medium and others discussed above and below, which will not interfere with its intended use. Alternatively, the host cell of the invention may be present in a growth matrix or immobilized on a surface as discussed below.

### Pharmaceutical Compositions

The invention also provides a pharmaceutical composition comprising (a) a combinatorial peptide entity of the invention, a peptide of the invention, a peptide combination of the invention, a polynucleotide of the invention, a polynucleotide combination of the invention, a vector of the invention or a host cell of the invention and (b) a pharmaceutically acceptable carrier or diluent. The carrier or diluent may be any of those discussed above with reference to the vectors of the invention.

The carrier(s) or diluent(s) present in the pharmaceutical composition must be “acceptable” in the sense of being compatible with the other ingredients of the composition and not deleterious to the recipient thereof. Typically, carriers for injection, and the final formulation, are sterile and pyrogen free. The carrier or diluent may be water. A pharmaceutically acceptable carrier or diluent may comprise as one of its components thioglycerol or thioanisole.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Pharmaceutically acceptable excipients, vehicles and auxiliary substances are well known in the art.

The active agents are typically present at 0.1% to 50% by weight in the pharmaceutical composition, more preferably at 0.1% to 5% by weight. They may be present at less than 0.1% by weight in the pharmaceutical composition.

The pharmaceutically acceptable carrier or diluent is typically present at 50% to 99.9% by weight in the pharmaceutical composition, more preferably at 95% to 99.9% by weight. The pharmaceutically acceptable carrier or diluents may be present at more than 99.9% by weight in the pharmaceutical composition.

Pharmaceutical compositions include, but are not limited to pharmaceutically acceptable solutions, lyophilisates, suspensions, emulsions in oily or aqueous vehicles, pastes, and implantable sustained-release or biodegradable compositions. Such pharmaceutical compositions may further comprise one or more additional ingredients including, but not limited to, suspending, stabilizing, or dispersing agents. A lyophilisate may comprise one or more of trehalose, thioglycerol and thioanisole. In one embodiment of a pharmaceutical composition for parenteral administration, the active ingredient is provided in dry form (e.g., a lyophilisate, powder or granules) for reconstitution with a suitable vehicle (e.g., sterile pyrogen-free water) prior to parenteral administration of the reconstituted pharmaceutical composition.

The pharmaceutical composition may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable compositions may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parenterally-administrable pharmaceutical compositions which are useful include those which comprise the active ingredient in microcrystalline form, in a liposomal preparation, or as a component of a biodegradable polymer systems. Pharmaceutical compositions for sustained release or implantation may comprise pharmaceutically acceptable polymeric or hydrophobic materials such as an emulsion, an ion exchange resin, a sparingly soluble polymer, or a sparingly soluble salt.

For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical compositions. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral compositions include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release compositions or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Polynucleotides may be present in combination with cationic lipids, polymers or targeting systems.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectins, for example, lipofectamine and transfectam. The dosage of the polynucleotide or oligonucleotide to be administered can be altered.

Alternatively, the active agent may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The composition will depend upon factors such as the nature of the active agent and the method of delivery. The pharmaceutical composition may be administered in a variety of dosage forms. It may be administered orally (e.g. as tablets, troches, lozenges, aqueous or oily suspensions, dispersible powders or granules), topically, parenterally, subcutaneously, by inhalation, intravenously, intramuscularly, intralymphatically (such as to lymph nodes in the groin), intrasternally, transdermally, intradermally, epidermally, sublingually, intranasally, buccally or by infusion techniques. The administration may be intratonsillar. The administration may be as suppositories. The administration may be made by iontophoresis. Preferably, the administration is intradermal, epidermal or transdermal. The administration may be made by a patch, such as a microtine patch. Administration is discussed in more detail below.

A physician will be able to determine the required route and means of administration for each particular individual.

The pharmaceutical compositions of the invention are preferably provided sealed in a container. The pharmaceutical compositions are typically provided in unit dose form, for example single dose form. They may alternatively be provided in multi-dose form. Where the pharmaceutical composition is a pharmaceutically acceptable solution, the solution may be provided in an ampoule, sealed vial, syringe, cartridge, flexible bag or glass bottle. Where the pharmaceutical composition is a lyophilisate, it is preferably provided in a sealed vial.

The pharmaceutical compositions of the invention will comprise a suitable concentration of each agent to be effective without causing adverse reaction. Where the pharmaceutical composition is for example a lyophilisate, the relevant concentration will be that of each polypeptide following reconstitution. Typically, the concentration of each agent in the pharmaceutical composition when in solution will be in the range of 0.03 to 200 nmol/ml. The concentration of each agent may be more preferably in the range of 0.3 to 200 nmol/ml, 3 to 180 nmol/ml, 5 to 160 nmol/ml, 10 to 150 nmol/ml, 50 to 200 nmol/ml or 30 to 120 nmol/ml, for example about 100 nmol/ml. The pharmaceutical composition should have a purity of greater than 95% or 98% or a purity of at least 99%.

In an embodiment where the invention involves combines therapy, the other therapeutic agents or adjuvants may be administered separately, simultaneously or sequentially. They may be administered in the same or different pharmaceutical compositions. A pharmaceutical composition may therefore be prepared which comprises an agent of the invention and also one or more other therapeutic agents or adjuvants. A pharmaceutical composition of the invention may alternatively be used simultaneously, sequentially or separately with one or more other therapeutic compositions as part of a combined treatment.

The invention encompasses any pharmaceutically acceptable salt of a peptide described herein. Said pharmaceutically acceptable salts include, for example, mineral acid salts such as chlorides, hydrochlorides, hydrobromides, phosphates, sulfates, and the like; and the salts of organic acids such as acetates, propionates, malonates, benzoates, and the like; and salts of monocationic metal ions such as sodium and potassium and the like; and salts of bases such as ammonia. The salt may be a hydrochloride salt or an acetate salt.

Pharmaceutically acceptable salts of peptides can be prepared by any suitable technique. Typically, salification involves reaction of the peptide or a salt thereof with a suitable reagent, typically acid, to obtain the pharmaceutically acceptable salt selected.

For example, a hydrochloride salt of a peptide can be prepared by initially cleaving the peptide from the solid phase using trifluoroacetic acid. The peptide will thus initially be a trifluoroacetate salt. The trifluoroacetate salt can then be converted into a hydrochloride salt by any known technique, such as ion exchange on a suitable column using hydrochloric acid as an eluent.

### Methods of Producing Combinatorial Peptide Entities

Also disclosed herein is a method of producing a combinatorial peptide entity. The method comprises identifying the chemokines associated with a disease, identifying a combination of two or more chemokine binding peptides that bind to the chemokines associated with the disease; and producing a combinatorial peptide entity from said two or more peptides.

A chemokine is associated with a disease if the disease has a chemokine component. In other words, one or more symptoms of the disease may be treated or prevented by inhibiting one or more chemokines. Typically, the disease is an inflammatory disease, i.e. a disease having an inflammatory component. Chemokines are molecules that are known to drive inflammation and are typically overexpressed in inflammatory diseases. As discussed above, the chemokine system is highly redundant, and so any number of chemokines may be expressed in and associated with the pathophysiology of the diseases. The chemokines are preferably selected from those shown in any of Tables 3 E, 3F, 5A or 5B.

Particular determined chemokine binding activities of chemokine binding peptides described herein are provided in Table 5A. Widened or enhanced chemokine binding activity of variant peptides of HD2 are illustrated in Table 5B. Chemokine binding activity of chemokine binding peptides can be routinely determined by experimental means or by homology, as discussed in detail above. The skilled person can identify a combination of chemokine binding peptides that bind to the disease by any means. The combination may be identified computationally, for example, through the use of a simple algorithm that selects the combination of chemokine binding peptides that best provides coverage of the chemokines associated with a disease. The combination may be identified manually, be selecting peptides that binds to the chemokines associated with a disease that have not yet been covered by a different chemokine binding peptide in the combinatorial peptide entity. Two or more different peptides may be present in the combinatorial peptide entity. In some cases, three or more different peptides may be present in the combinatorial peptide entity. In some cases, four or more different peptides may be present in the combinatorial peptide entity. In some cases, two, three, four or more different peptides may be present in the combinatorial peptide entity. Typically, the greater the number of different peptides in the combinatorial peptide entity, the greater the coverage of the chemokines associated with a disease.

For example, a combinatorial peptide entity may bind to at least 50% of the chemokines associated with a disease, such as at least 60%, at least 70%, at least 80%, at least 90%, at least 95% or all or substantially all of the chemokines associated with a disease. A combinatorial peptide entity may bind at least 5 chemokines associated with a disease, such as at least 6, at least 7, at least 8, at least 9, at least 10, at least 15 or at least 20 chemokines associated with a disease, provided that the number does not exceed the total number of chemokines associated with a disease. A combinatorial peptide entity may bind five, six, seven, eight, nine, ten, eleven, twelve, thirteen, fourteen, fifteen, sixteen, seventeen, eighteen, nineteen, twenty or more chemokines associated with a disease provided that the number does not exceed the total number of chemokines associated with a disease.

Typically, the chemokine binding peptides are selected such that they do not bind chemokines not associated with the disease, in order to reduce possible off target effects. For example, a chemokine binding peptide may binds 5 or fewer chemokines not associated with the disease, such as 4 or fewer, 3 or fewer, 2 or fewer or 1 chemokine not associated with the disease. A combinatorial peptide entity may bind 10 or fewer chemokines not associated with the disease such as 5 of fewer, 4 or fewer, 3 or fewer, 2 or fewer or 1 chemokine not associated with the disease. A combinatorial peptide entity may bind ten, nine, eight, seven, six, five, four, three, two or one chemokines not associated with the disease. A chemokine binding peptide or combinatorial peptide entity may only bind chemokines that are associated with the disease, and thus may not bind any other chemokines.

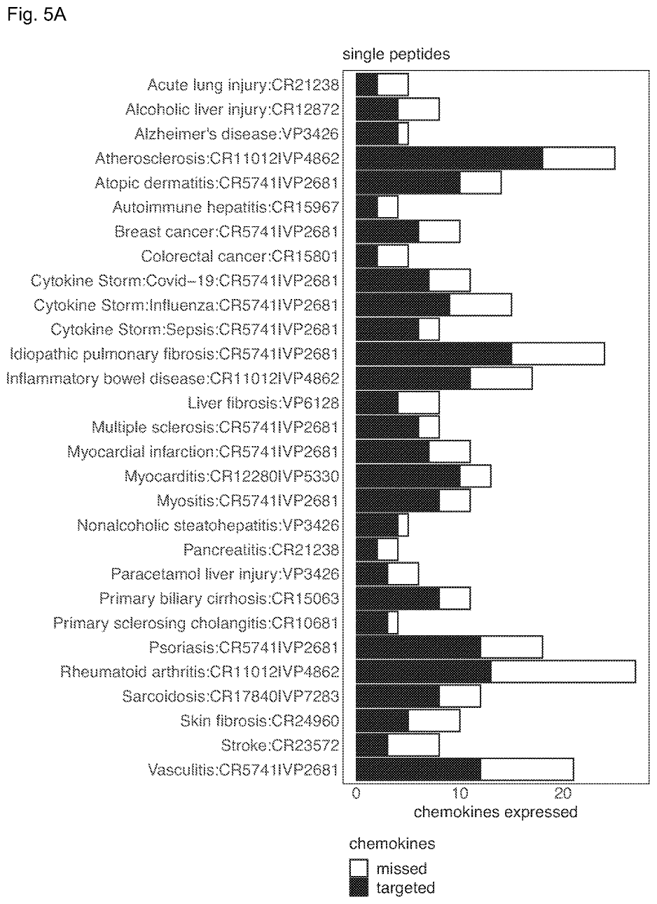

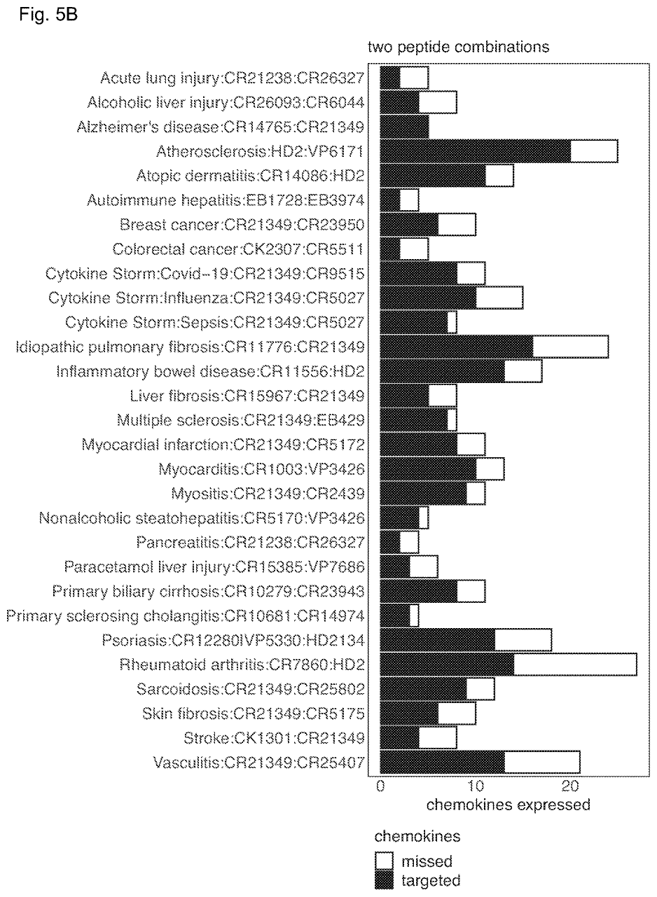

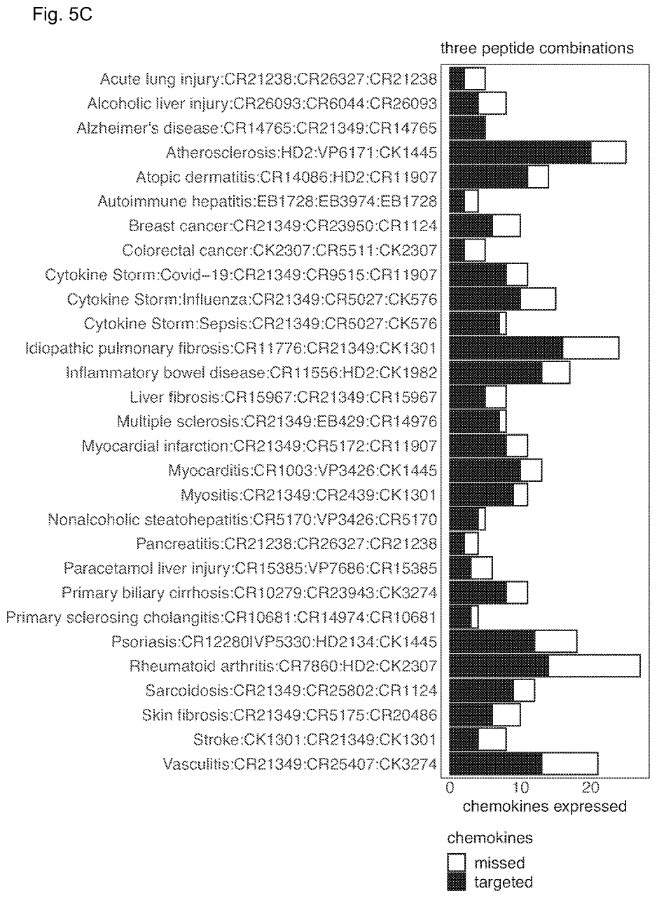

FIG. 5 provides examples of combinations of 1, 2 or 3 different chemokine binding peptides and the coverage of the number of chemokines associated with said disease. Such combinations of chemokine binding peptides may be suitable for use in a combinatorial peptide entity. Particular combinations of 2 or 3 different chemokine binding peptides shown in FIG. 5 include:
CR21349 and CR5175; or CR21349, CR5175 and CR20486; for treating skin fibrosis;
CR21238 and CR26327; or CR21238, CR26327 and CR21238; for treating acute lung injury;
CR21349 and CR9515; or CR21349, CR9515 and CR11907; for treating cytokine storm associated with Covid-19 infection.

Thus, a combinatorial peptide entity of the invention may comprise chemokine binding peptides having the amino acid sequences of CR21349 and CR5175, or variants thereof; chemokine binding peptides having the amino acid sequences of CR21349, CR5175 and CR20486, or variants thereof; chemokine binding peptides having the amino acid sequences of CR21238 and CR26327, or variants thereof; chemokine binding peptides having the amino acid sequences of CR21238, CR26327 and CR21238, or variants thereof; chemokine binding peptides having the amino acid sequences of CR21349 and CR9515, or variants thereof; or chemokine binding peptides having the amino acid sequences of CR21349, CR9515 and CR11907, or variants thereof. The chemokine binding peptides in the combinatorial peptide entity may be independently disposed. Other combinations are shown in FIG. 5 (see Tables 5A and 5B for corresponding SEQ ID NOs).

The combinatorial peptide entity may be any combinatorial peptide entity described herein, for example, as a peptibody, a branched peptide, a nanoparticle, a multicyclic peptide, a bacteria displaying the peptides, or a bacteriophage displaying the peptides.

The method may be for producing a combinatorial peptide entity suitable for binding the chemokines associated with any number of diseases, such as 2, 3, 4, 5, 6, 7, 8, 9, 10, 11, 12 or 13 diseases. The diseases may have a similar profile of chemokines associated with each disease. The combinatorial peptide entity may bind to additional chemokines that are expressed in and associated with the pathophysiology of additional diseases.

Typically, the chemokines associated with the disease have increased expression when compared to a non-disease state. Increased expression may be determined by any means known in the art. For example, increased expression may be measured at the RNA level using qRT-PCR. Increase expression may be measured at the protein level, such as by mass spectrometry or immunodetection techniques. The increased expression of a chemokine may be localised to a site of inflammation, for example an organ such as the skin (or a part thereof), the liver, serum or the lungs, or a body part, such as joints.

Typically, a chemokine is associated with a disease when its increased expression leads to inflammation. Inflammation may be determined by any means known, such as visually (such as by redness on the skin), by the increased presence of inflammatory cells (such as Tₕ1 cells, macrophages and dendritic cells), or by the presence of pro-inflammatory cytokines (such as TNF-α, IL-1 and IL-6 and the like).

The chemokines may be selected from any of those in Tables 3 E, 3F, 5A or 5B. The one or more diseases may be as identified in Table 4 or FIG. 5. The chemokines may be selected from any of those in Tables 3 E, 3F, 5A, 5B, 6, 7 or 8. The one or more diseases may be as identified in Table 4 or FIG. 5. Exemplary lists of chemokines expressed in various inflammatory diseases are shown in Table 4. The disease(s) may be selected from one or more of myocarditis, myocardial infarction, myocardial ischemia, atherosclerosis, vasculitis, stroke, multiple sclerosis, Alzheimer's disease, autoimmune hepatitis, myositis, primary biliary cirrhosis, primary sclerosing cholangitis, liver fibrosis, non-alcoholic steatohepatitis, paracetamol liver injury, alcohol liver injury, idiopathic pulmonary fibrosis, COVID-19, Covid-19 cytokine storm, sepsis, sepsis cytokine storm, acute lung injury, cardiac allograft vasculopathy, sarcoidosis, influenza, influenza cytokine storm, inflammatory bowel disease, pancreatitis, rheumatoid arthritis, psoriasis, skin fibrosis, kidney fibrosis, atopic dermatitis, acute respiratory distress syndrome, breast cancer and colorectal cancer, optionally wherein the disease is acute respiratory distress syndrome.

A combinatorial peptide entity may be used to bind to and inhibit multiple chemokines associated with a disease, such as five or more, eight or more or ten or more chemokines. A combinatorial peptide entity may be used to bind to and inhibit multiple chemokines associated with a disease, such as five, six, seven, eight, nine, ten or more chemokines. The multiple chemokines may comprise both CC and CXC chemokines. The multiple chemokines may comprise CC, CX3C and CXC chemokines, or XC, CC, CX3C and CXC chemokines. A combinatorial peptide entity binding both a CC chemokine and a CXC chemokine may bind chemokines associated with inter alia myocarditis, myocardial infarction, myositis, atherosclerosis, vasculitis, stroke, multiple sclerosis, Alzheimer's disease, autoimmune hepatitis, primary biliary cirrhosis, primary sclerosing cholangitis, liver fibrosis, non-alcoholic steatohepatitis, paracetamol liver injury, alcohol liver injury, idiopathic pulmonary fibrosis, acute lung injury, Covid-19 cytokine storm, influenza cytokine storm, sepsis cytokine storm, sarcoidosis, influenza, inflammatory bowel disease, pancreatitis, rheumatoid arthritis, psoriasis, skin fibrosis, breast cancer and colorectal cancer, which all comprise expression of both CC and CXC chemokines, as shown in Table 4. A combinatorial peptide entity may bind all or substantially all chemokines associated with any particular disease as shown in FIG. 5 or Table 4.

### Therapeutic Methods

Disclosed herein in a method of treating a disease associated with aberrant chemokine expression in a subject, comprising administering the pharmaceutical composition discussed above. Also disclosed is a pharmaceutical composition, a combinatorial peptide entity or a chemokine binding peptide provided herein for use in a method of treating a disease associated with aberrant chemokine expression in a subject. Also disclosed is the use of a combinatorial peptide entity or a chemokine binding peptide provided herein in the manufacture of a medicament for the therapeutic treatment of a disease associated with aberrant chemokine expression. Also disclosed is a use of a pharmaceutical composition, a combinatorial peptide entity or a chemokine binding peptide provided herein for the treatment of a disease associated with aberrant chemokine expression.

The diseases associated with aberrant chemokine expression may be an inflammatory disease. The disease associated with aberrant chemokine expression may be a disease with an inflammatory component. The inflammatory component is as a result of chemokine expression associated with the disease.

The disease associated with aberrant chemokine expression may be selected from any one of myocarditis, myocardial infarction, myocardial ischemia, atherosclerosis, vasculitis, stroke, multiple sclerosis, Alzheimer's disease, autoimmune hepatitis, myositis, primary biliary cirrhosis, primary sclerosing cholangitis, liver fibrosis, non-alcoholic steatohepatitis, paracetamol liver injury, alcohol liver injury, idiopathic pulmonary fibrosis, COVID-19, Covid-19 cytokine storm, sepsis, sepsis cytokine storm, acute lung injury, cardiac allograft vasculopathy, sarcoidosis, influenza, influenza cytokine storm, inflammatory bowel disease, pancreatitis, rheumatoid arthritis, psoriasis, skin fibrosis, kidney fibrosis, atopic dermatitis, acute respiratory distress syndrome, breast cancer and colorectal cancer, optionally wherein the disease is acute respiratory distress syndrome.

Also disclosed is a method of inhibiting the signalling of one or more chemokines in a subject, the method comprising administering to the subject a combinatorial peptide entity or a chemokine binding peptide, a combination, a polynucleotide, a vector or a host cell provided herein. The invention also provides a combinatorial peptide entity, a chemokine binding peptide, a combination, a polynucleotide, a vector or a host cell provided herein for use in a method of inhibiting the signalling of one or more chemokines in a subject. The invention also provides use of a combinatorial peptide entity, a chemokine binding peptide, a combination, a polynucleotide, a vector or a host cell provided herein in the manufacture of a medicament for use in inhibiting the signalling of one or more chemokines in a subject.

The aberrant chemokine expression is aberrant expression of a chemokine, or combination of chemokines, listed in any one of Tables 3 E, 3F, 5A or 5B.

The disease may be a disease listed in Table 4 or FIG. 5. The disease may be a disease listed in Table 4 and the chemokine(s) associated with the disease may be the chemokine(s) listed in the corresponding rows of Table 4.

The methods or uses may comprise inhibiting any number of chemokines, such as 1, 2, 3, 4, 5, 6, 7, 8, 9, 10, 11, 12, 13 or 14 chemokines. FIG. 5 provides particular combinations of chemokine-binding peptides to include in a combinatorial peptide entity for use in inhibiting particular chemokines associated with specified diseases.

As discussed above, the skilled person can design combinations chemokine binding peptides to include in a combinatorial peptide entity to inhibit specific combinations of chemokines.

The methods and uses may comprise treating or preventing any number of diseases associated with one or more chemokines, such as 1, 2, 3, 4, 5, 6, 7, 8, 9, 10, 11, 12 or 13 diseases. When treating or preventing any specific disease shown, a combinatorial peptide entity, a chemokine binding peptide, a combination, a polynucleotide, a vector or a host cell provided herein representing amino acid sequence(s) from chemokine binding peptides shown to bind chemokines associated with that disease is preferably used. Chemokine-binding properties of each of SEQ ID NOs 1-472, exemplary chemokine binding peptides, are shown in Tables 5A and 5B.

Any subject may be treated. The subject is typically human. However, the subject can be another animal or mammal, such as a research animal, such as a rat, a mouse, a rabbit or a guinea pig, a commercially farmed animal, such as a horse, a cow, a sheep or a pig, or a pet, such as a cat, a dog or a hamster.

The subject may be asymptomatic. A prophylactically effective amount of the combinatorial peptide entity, chemokine binding peptide, combination, polynucleotide, vector or host cell is administered to such a subject. A prophylactically effective amount is an amount which prevents the onset of one or more, preferably all of, symptoms of the one or more diseases.

Alternatively, the subject may be in need thereof. That is, the subject may exhibit one or more symptoms of the one or more diseases. A therapeutically effective amount of the combinatorial peptide entity, chemokine binding peptide, combination, polynucleotide, vector or host cell is administered to such a subject. A therapeutically effective amount is an amount which is effective to ameliorate one or more of, the symptoms of the one or more diseases. The therapeutically effective amount may be an amount which is effective to ameliorate inflammation associated with the one or more diseases (i.e. the symptom if inflammation).

The combinatorial peptide entity, chemokine binding peptide, combination, polynucleotide, vector or host cell may be administered to the subject in any appropriate way. The combinatorial peptide entity, chemokine binding peptide, combination, polynucleotide, vector or host cell may be administered in a variety of dosage forms. Thus, it can be administered orally, for example as tablets, troches, lozenges, aqueous or oily suspensions, dispersible powders or granules. It may also be administered by enteral or parenteral routes such as via buccal, anal, pulmonary, intravenous, intra-arterial, intramuscular, intraperitoneal, intraarticular, topical or other appropriate administration routes. A physician will be able to determine the required route of administration for each particular subject.

The combinatorial peptide entity, chemokine binding peptide, combination, polynucleotide, vector or host cell may be in any of the forms discussed above with reference to the pharmaceutical composition of the invention.

Methods for gene delivery are known in the art. The nucleic acid molecule or a modified nucleic acid molecule can be introduced directly into the recipient subject, such as by standard intramuscular or intradermal or intravenous or intra coronary artery or intramyocardial injection; transdermal particle delivery; inhalation; topically, or by oral, intranasal or mucosal modes of administration. The molecule alternatively can be introduced ex vivo into cells that have been removed from a subject. For example, a polynucleotide, expression cassette or vector of the invention may be introduced into APCs of an individual ex vivo. Cells containing the nucleic acid molecule of interest are re-introduced into the subject such that an immune response can be mounted against the peptide encoded by the nucleic acid molecule. The nucleic acid molecules used in such immunization are generally referred to herein as “nucleic acid vaccines.”

The dose may be determined according to various parameters, especially according to the substance used; the age, weight and condition of the subject to be treated; the route of administration; and the required regimen. Again, a physician will be able to determine the required route of administration and dosage for any particular subject. A typical daily dose may be from about 0.1 to 50 mg per kg of body weight, such as 5 mg per kg of body weight, according to the activity of the specific inhibitor, the age, weight and conditions of the subject to be treated and the frequency and route of administration. The dose may be provided as a single dose or may be provided as multiple doses, for example taken at regular intervals, for example 2, 3 or 4 doses administered hourly. Dosage levels of inhibitors are from 5 mg to 2 g.

The pharmaceutical composition, combinatorial peptide entity, chemokine binding peptide, combination, polynucleotide, vector or host cell may be administered at a dose of 0.1 to 1000 nmol, such as 1 to 100 nmol per kg of body weight.

Typically, polynucleotide or oligonucleotide inhibitors are administered in the range of 1 μg to 1 mg, preferably to 1 μg to 10 μg nucleic acid for particle mediated delivery and 10 μg to 1 mg for other routes.

The combinatorial peptide entity, chemokine binding peptide, combination, polynucleotide, vector or host cell may be administered in combination with another therapy

The pharmaceutical composition, combinatorial peptide entity, chemokine binding peptide, combination, polynucleotide, vector or host cell may be used in combination with one or more other therapies intended to treat the same subject. By a combination is meant that the therapies may be administered simultaneously, in a combined or separate form, to the subject. The therapies may be administered separately or sequentially to a subject as part of the same therapeutic regimen. For example, the polypeptide, the combination, the polynucleotide, the vector or the host cell be used in combination with another therapy intended to treat the one or more disease. The other therapy may be a general therapy aimed at treating or improving the condition of the subject. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with the pharmaceutical composition, combinatorial peptide entity, chemokine binding peptide, combination, polynucleotide, vector or host cell. The other therapy may be a specific treatment directed at the one or more diseases. Such treatments are known in the art. For instance in the treatment of rheumatoid arthritis this may include anti-TNFα or other biologics targeting other cytokines (e.g. IL7, IL17) or their receptors (e.g. IL1-R, IL-6R), that are in clinical use or development. In the treatment of inflammatory bowel disease, biologics such as vedolizumab may be used. For atherosclerosis simvastatin or other statins may be used.

### In Vitro Methods

Also provided is a method of inhibiting the signalling of one or more chemokines in an in vitro culture, the method comprising contacting the culture with a combinatorial peptide entity, a chemokine binding peptide, a combination, a polynucleotide, a vector or a host cell.

The method may comprise inhibiting any number of chemokines, such as 1, 2, 3, 4, 5, 6, 7, 8, 9, 10, 11, 12, 13 or 14 chemokines. The chemokines may be selected from any of those in Tables 3 E, 3F, 5A or 5B. The chemokines may be selected from any of those in Tables 3 E, 3F, 5A, 5B, 6, 7 or 8. When inhibiting the one or more chemokines in a particular box in Table 5A or 5B, a chemokine binding peptide in the same row, or a combinatorial peptide entity comprising a chemokine binding peptide in the same row is preferably used. For instance, when inhibiting one or more of CCL1, CCL11, CCL15, CCL17, CCL18, CCL20, CCL22, CCL3, CCL4, CCL5 and CCL8, the chemokine binding peptide HD2 (SEQ ID NO: 396) may be used. When inhibiting one or more of CCL1, CCL11, CCL17, CCL19, CCL20, CCL22, CCL25, CCL28, CXL10, CXL11, SDF1, CXL14 and IL8, the chemokine binding peptide VP6130 (SEQ ID NO: 429) may be used. When inhibiting one or more of CCL1, CCL11, CCL15, CCL17, CCL18, CCL20, CCL22, CCL3, CCL4, CCL5 and CCL8, and one or more of CCL1, CCL11, CCL17, CCL19, CCL20, CCL22, CCL25, CCL28, CXL10, CXL11, SDF1, CXL14 and IL8, a combinatorial peptide entity comprising chemokine binding peptides VP6130 and HD2 (SEQ ID NOs: 429 and 396, respectively) may be used.

The in vitro culture is preferable a culture of cells capable of undergoing chemotaxis. The in vitro culture is preferably a chemotactic assay. The culture may be present in a culture flask or the wells of a flat plate, such as a standard 96 or 384 well plate. Such plates are commercially available Fisher scientific, VWR suppliers, Nunc, Starstedt or Falcon. Conditions for culturing cells are known in the art.

The combinatorial peptide entity, a chemokine binding peptide, a combination, a polynucleotide, a vector or a host cell may be administered in any of the forms discussed above.

### Diagnostic Methods

Also provided is a method of detecting one or more chemokines in a tissue, comprising contacting the tissue with a detectably-labelled combinatorial peptide entity or a detectably labelled chemokine binding peptide provided herein, and detecting the binding of the combinatorial peptide entity or chemokine binding peptide to one or more chemokines in the tissue. The tissue may be in vitro or in vivo. Also provided is a detectably-labelled combinatorial peptide entity or a detectably labelled chemokine binding peptide for use in a method of detecting one or more chemokines in a tissue. The invention also provides use of a detectably-labelled combinatorial peptide entity or a detectably labelled chemokine binding peptide in the manufacture of medicament for detecting one or more chemokines in a tissue.

Any method of detecting binding may be used. The method may be positron emission tomography (PET) or magnetic resonance imaging (MRI).

The tissue may be any tissue. The tissue is preferably in a subject. The subject may be any those discussed above. The combinatorial peptide entity or chemokine binding peptide may be administered to the subject in any of the forms discussed above. The tissue may be an inflamed tissue.

Any of the combinatorial peptide entities or chemokine binding peptides discussed above may be used. Suitable detectable labels are also discussed above. The label may be a tracer that is suitable for positron emission tomography (PET), such as fluorodeoxyglucose (¹⁸F). The label is preferably a tracer suitable for magnetic resonance imaging (MRI), such as fluorine (¹⁹F).

The method may comprise detecting any number of chemokines, such as 1, 2, 3, 4, 5, 6, 7, 8, 9, 10, 11, 12, 13 or 14 chemokines. The chemokines may be selected from any of those in Tables 4, 5A and 5B. When detecting the one or more chemokines in a particular row in Tables 5A or 5B, a chemokine binding peptide in the same row, or a combinatorial peptide entity comprising a chemokine binding peptide in the same row is preferably used. For instance, when inhibiting one or more of CCL1, CCL11, CCL15, CCL17, CCL18, CCL20, CCL22, CCL3, CCL4, CCL5 and CCL8, the chemokine binding peptide HD2 (SEQ ID NO: 396) may be used. When inhibiting one or more of CCL1, CCL11, CCL17, CCL19, CCL20, CCL22, CCL25, CCL28, CXL10, CXL11, SDF1, CXL14 and IL8, the chemokine binding peptide VP6130 (SEQ ID NO: 429) may be used. When inhibiting one or more of CCL1, CCL11, CCL15, CCL17, CCL18, CCL20, CCL22, CCL3, CCL4, CCL5 and CCL8, and one or more of CCL1, CCL11, CCL17, CCL19, CCL20, CCL22, CCL25, CCL28, CXL10, CXL11, SDF1, CXL14 and IL8, a combinatorial peptide entity comprising chemokine binding peptides VP6130 and HD2 (SEQ ID NOs: 429 and 396, respectively) may be used.

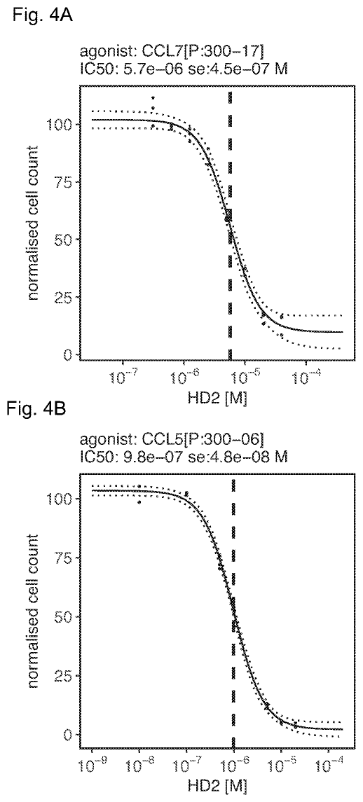

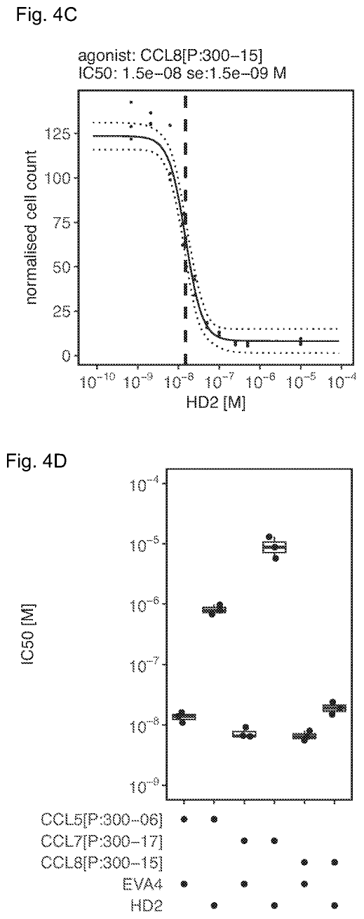

Particular selections of combinations of chemokine binding peptides, for instance, comprised within a single type of combinatorial peptide entity, may be used for diagnosis or prognosis of particular diseases according to the same criteria discussed above in relation to medical uses. Thus, a combinatorial binding peptide which is capable of binding both a CC chemokine and a CXC chemokine may be used to diagnose or prognose any of myocarditis, myocardial infarction, myositis, atherosclerosis, vasculitis, stroke, multiple sclerosis, Alzheimer's disease, autoimmune hepatitis, primary biliary cirrhosis, primary sclerosing cholangitis, liver fibrosis, non-alcoholic steatohepatitis, paracetamol liver injury, alcoholic liver injury, idiopathic pulmonary fibrosis, acute lung injury, Covid-19 cytokine storm, influenza cytokine storm, sepsis cytokine storm, sarcoidosis, influenza, inflammatory bowel disease, pancreatitis, rheumatoid arthritis, psoriasis, skin fibrosis, breast cancer and colorectal cancer, which all comprise expression of both CC and CXC chemokines, as shown in FIG. 4.

The skilled person can provide combinatorial peptide entities having appropriate combinations of chemokine-binding activities from chemokine binding peptides comprised within the combinatorial peptide entity to diagnose or prognose specific diseases or combinations of diseases.

### Antibodies

The invention also provides an antibody or a fragment thereof which specifically binds a combinatorial peptide entity described herein, or a peptide described herein. The antibody or fragment thereof preferably specifically binds to a combinatorial peptide entity comprising a peptide having an amino acid sequence shown in any one of SEQ ID NOs: 1 to 472, or binds to a peptide having an amino acid sequence shown in any one of SEQ ID NOs: 1 to 472. The antibody or fragment thereof may specifically bind to a combinatorial peptide entity comprising a peptide having an amino acid sequence shown in any one of SEQ ID NOs: 1 to 543, or binds to a peptide having an amino acid sequence shown in any one of SEQ ID NOs: 1 to 543.

An antibody “specifically binds” to a polypeptide when it binds with preferential or high affinity to that polypeptide but does not substantially bind, does not bind or binds with only low affinity to other polypeptides. For instance, an antibody “specifically binds” to SEQ ID NO: 1 or a variant thereof when it binds with preferential or high affinity to SEQ ID NO: 1 or a variant thereof but does not substantially bind, does not bind or binds with only low affinity to other polypeptides.

An antibody binds with preferential or high affinity if it binds with a Kd of 1×10−7 M or less, more preferably 5×10−8 M or less, more preferably 1×10−8 M or less or more preferably 5×10−9 M or less. An antibody binds with low affinity if it binds with a Kd of 1×10⁻⁶ M or more, more preferably 1×10−5 M or more, more preferably 1×10−4 M or more, more preferably 1×10−3 M or more, even more preferably 1×10−2 M or more. A variety of protocols for competitive binding or immunoradiometric assays to determine the specific binding capability of compounds, such as antibodies or antibody constructs and oligonucleotides are well known in the art.

The antibody may be, for example, a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)₂ or Fv fragment. Furthermore, the antibodies and fragment thereof may be chimeric antibodies, CDR-grafted antibodies or humanised antibodies.

Antibodies of the invention can be produced by any suitable method. Means for preparing and characterising antibodies are well known in the art. For example, an antibody may be produced by raising an antibody in a host animal against the peptide, hereinafter the “immunogen”, typically at least 10 or at least 15 amino acids long).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells.

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Methods of Producing Chemokine-Binding Peptides

The invention also provides a method of identifying a chemokine binding peptide. The method comprises constructing a phage-display library encoding a bacteriophage coat protein fused to 10-mer to 20-mer peptides of a chemokine-binding protein at single amino acid resolution to thereby produce a phage-display library comprising overlapping peptide sequences. The term “single amino acid resolution” refers to the differences in the sequence of the peptides presented by the phage display library, e.g. the different X-mer peptides present in the library differ by at least one amino acid at the N-terminus and/or the C-terminus. In contrast, two amino acid resolution would mean that that different X-mer peptides in the library differ by at least two amino acids at the N-terminus and/or the C-terminus of the peptide. The method further comprises contacting the phage-display library with one or more chemokines to thereby produce an enriched library comprising peptides capable of binding the one or more chemokines. The method further comprises sequencing the enriched library to thereby identify a chemokine-binding peptide.

The phage-display library may encode a bacteriophage coat protein fused to 12-mer to 19-mer peptides, 14-mer to 18-mer peptides, 15-mer to 17-mer peptides or 16-mer peptides. The bacteriophage coat protein may be bacteriophage coat protein p8. The phage display library may be constructed following the steps described in the Examples.

The invention also provides a method of enhancing and/or expanding chemokine binding activity of a chemokine-binding peptide. The method comprises substituting one or more codons encoding a chemokine-binding peptide in a phage-display system with the sequence NNK, to thereby produce a mutant phage display library encoding mutated chemokine-binding peptides. The method further comprises contacting the mutated phage-display library with one or more chemokines to thereby produce an enriched library comprising peptides capable of binding the one or more chemokines. The method further comprises sequencing the enriched library to thereby identify one or more mutated chemokine-binding peptide with enhanced or expanded chemokine binding activity.

The method may further comprise constructing a combinatorial mutant phage display library comprising combinations of the substitutions in the one of more mutated chemokine-binding peptides identified in the earlier step, contacting the combinatorial mutant phage display library with one or more chemokines to thereby produce an enriched combinatorial mutant library comprising peptides capable of binding the one or more chemokines; and sequencing the enriched combinatorial mutant library to thereby identify combinatorially mutated chemokine-binding peptides with further enhanced or expanded chemokine binding activity.

The method may comprise screening all possible combinations of single amino acid mutations. Accordingly, a method of enhancing and/or expanding chemokine binding activity of a chemokine-binding peptide may comprise substituting each codon encoding a chemokine-binding peptide in a phage-display system with the sequence NNK, to thereby produce a mutant phage display library encoding all possible combinations of the chemokine-binding peptide with a single amino acid substitution. The method further comprises contacting the mutated phage-display library with one or more chemokines to thereby produce an enriched library comprising peptides capable of binding the one or more chemokines. The method further comprises sequencing the enriched library to thereby identify one or more mutated chemokine-binding peptides with enhanced or expanded chemokine binding activity, wherein each mutated chemokine-binding peptide comprises a single amino acid substitution. The method may further comprise screening all possible combinations of the single amino acid substitutions identified as having enhanced or expanded chemokine binding activity. Accordingly, the method may further comprise creating a combinatorial mutant phage library with all possible combinations of the single amino acid substitutions identified as having enhanced or expanded chemokine binding activity; contacting the combinatorial mutant phage display library with one or more chemokines to thereby produce an enriched library comprising peptides capable of binding the one or more chemokines; and sequencing the enriched combinatorial library to thereby identify combinatorially mutated chemokine-binding peptides with further enhanced or expanded chemokine binding activity.

The chemokine-binding peptide may be fewer than 25 amino acids in length, such as fewer than 20 amino acids in length. The chemokine-binding peptide may be 10-20 amino acids in length, such as 12-19, 14-18, 15-17 or about 16 amino acids in length.

Each amino acid in the chemokine binding peptide is encoded by a codon. The method comprises substituting the codon for the nucleotide sequence NNK, to thereby replace the amino acid encoded by the library with a random amino acid. By repeating this process, a plurality of mutated chemokine binding peptides are produced with different amino acid substitutions. The method may comprise producing a mutant phage display library encoding mutated chemokine-binding peptides each comprising a single substitution relative to the starting chemokine-binding peptide. The method may comprise producing a mutant phage display library encoding mutated chemokine-binding peptides each comprising one or more, two or more, three or more, four or more, five or more, six or more, seven or more, eight or more, nine or more or ten or more substitutions relative to the starting chemokine-binding peptide. The method has the advantage that mutations can be screened in a high-throughput manner.

The methods comprise contacting the phage-display library with one or more chemokines to thereby produce an enriched library comprising peptides capable of binding the one or more chemokines. The method may comprise contacting the phage-display library with two or more chemokines, such as three or more, four or more, five or more or ten or more chemokines. The one or more chemokines may be one or more of the chemokines provided in Table 3 E. The method may comprise contacting the phage-display library with (i) one or more CC-class chemokines, (ii) one or more CXC-class chemokines, (iii) one or more XC-class chemokines, and/or (iv) a CX3C-class chemokine. The method may comprise contacting the phage-display library with (i) and (ii). The method may comprise contacting the phage-display library with (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv).

The one or more chemokines may be immobilised, for example, on a surface, such as a matrix, plate, gel or bead. For instance, the one or more chemokines may be biotinylated and immobilised on streptavidin. The step of contacting the phage-display library may further comprise washing to remove peptide that is not bound to the chemokine.

The methods comprise sequencing the enriched library. Sequence may be performed using next-generation sequencing or third-generation sequencing. The method may comprise mapping sequencing reads to the starting chemokine binding protein or to the starting chemokine-binding peptide. The methods may further comprise calculating peptide enrichment by determining the ratio of peptides in the enriched library to the peptides in the original library. The ratio may be expressed as log 2 E. A log 2 E of >5 may be used to identify a chemokine binding peptide and/or a mutant chemokine binding peptide.

In the method of enhancing and/or expanding chemokine binding activity of a chemokine-binding peptide, the methods may further comprise calculating mutant peptide enrichment by determining the change in log 2 E (Δlog 2 E) when compared to the log 2 E of starting chemokine binding peptide. Due to the random nature of the construction of the mutant phage-display library, the starting chemokine-binding peptide will be present in the library and allow for this analysis to take place. A Δlog 2 E of greater than 1 may be used to identify a mutated chemokine binding peptide with enhanced or expanded chemokine binding activity.

The log 2 E and/or Δlog 2 E values may be assessed for each chemokine tested. The log 2 E and/or Δlog 2 E values may be assessed as a mean over all chemokines tested and/or as a mean over all chemokines tested for each class of chemokines (CC, CXC, XC and/or CX3C).

Also provided are chemokine binding peptides identified by the methods described herein. The chemokine binding peptides bind to one or more chemokines. The chemokine binding peptides may bind to one or more CC-class chemokine, one or more CXC-class chemokines, one or more XC-class chemokines and/or a CX3C-class chemokine, such as one or more CC-class chemokines and/or one or more CXC-class chemokines.

### EXAMPLES

### Example 1. Identification of a Library of Chemokine-Binding Hexadecapeptides

Hexadecapeptides were identified using a phage-display next generation sequencing approach (McLaughlin, 2013). Libraries containing a total of 55662 overlapping peptides (from the tick chemokine binding proteins shown in Tables 3A and 3B, the viral chemokine binding proteins shown in Table 3C, the human chemokines shown in Table 3 E, and the human/viral chemokine receptors and other chemokine-binding proteins shown in Table 3D) were first constructed, and then screened against a panel of up to 42 human chemokines, as shown in Table 3F.

TABLE 3A
Class A Evasins

| Name/Uniprot ID | UniProt/GenBank Accession |
|---|---|
| AAM01 | A0A0C9S4J0 |
| ACA02 | JAC23884.1 |
| AMA01 | AEO36603.1 |
| ATR02 | A0A023G9N5 |
| E1180_AMBTT | A0A023G6B6 |
| E1181_AMBMU | G3MIX6 |
| E1182_AMBMU | G3MJ83 |
| E1183_AMBTT | A0A023G9N9 |
| E1243_AMBAM | A0A0C9S461 |
| EV467_RHIPC | L7M8Z8 |
| EV546_AMBCJ | A0A023FBW7 |
| EV672_RHIPC | L7MC74 |
| EV974_AMBCJ | A0A023FDY8 |
| EV983_AMBCJ | A0A023FFB5 |
| EV985_AMBPA | A0A023FT45 |
| EV991_AMBCJ | A0A023FFD0 |
| EVA1_RHISA | P0C8E7 |
| EVA4_RHISA | P0C8E9 |
| IHO01 | A0A223FZ07 |
| IRI01 | JAA70749.1 |
| RPU02 | JAA60771.1 |

TABLE 3B
Class B Evasins

| Name/Uniprot ID | UniProt/GenBank Accession |
|---|---|
| E1074_IXORI | A0A0K8RDH6 |
| E1077_IXORI | A0A090XE98 |
| E1078_IXORI | A0A0K8R726 |
| E1080_IXORI | A0A090XBL6 |
| E1086_IXORI | A0A0K8RK34 |
| E1090_IXORI | A0A090XBH9 |
| E1095_IXORI | A0A0K8RCE3 |
| E1096_IXORI | A0AOK8RKE4 |
| E1100_IXORI | A0A0K8R556 |
| E1104_IXORI | A0A090XA85 |
| E1124_IXORI | A0A0K8RG61 |
| E1126_AMBCJ | A0A023FF81 |
| E1127_IXORI | A0A0K8RJ67 |
| E1128_IXORI | A0A0K8R6B3 |
| E1132_IXORI | V5GZ08 |
| E1134_IXORI | A0A0K8RAI0 |
| E1142_AMBCJ | A0A023FBW4 |
| E1156_IXORI | V5HBW0 |
| E1162_IXORI | A0A0K8RDJ1 |
| E1166_IXORI | A0A0K8RE99 |
| E1168_IXORI | A0A0K8RCU3 |
| E1170_IXORI | A0A0K8RD51 |
| E1172_IXORI | V5I2G9 |
| E1174_IXORI | V5I3C5 |
| E1229_IXORI | A0A0K8R4R9 |
| EV458_IXORI | A0A0K8R7D4 |
| EV675_IXORI | A0A0K8R374 |
| EV942_IXORI | A0A0K8R512 |
| EVA3_RHISA | P0C8E8 |

TABLE 3C
Viral Chemokine Binding Proteins

| Name/Uniprot ID | UniProt/GenBank Accession |
|---|---|
| A0A0A1E5H7_9POXV | A0A0A1E5H7 |
| A41_VACCW | P24766 |
| C23L_VACCL | P19063 |
| C23L_VAR67 | P34016 |
| CRMB_VARV | P0DSV8 |
| E2CZQ0_9POXV | E2CZQ0 |
| E9M5R0_9GAMA | E9M5R0 |
| GG_EHV1B | P28967 |
| GN_BHV1C | Q77CE4 |
| I0AZ78_9POXV | I0AZ78 |
| O10625_9POXV | O10625 |
| O10647_9POXV | O10647 |
| O41925_MHV68 | O41925 |
| O57300_9POXV | O57300 |
| Q2F862_ORFV | Q2F862 |
| Q65588_9ALPH | Q65588 |
| Q6YM29_9POXV | Q6YM29 |
| Q7TDW8_9POXV | Q7TDW8 |
| Q85307_CWPXB | Q85307 |
| Q85308_CWPXB | Q85308 |
| Q8JL79_9POXV | Q8JL79 |
| Q8JLJ0_9POXV | Q8JLJ0 |
| Q8QMN0_CWPXB | Q8QMN0 |
| Q9JFT2_9POXV | Q9JFT2 |
| Q9YP87_COWPX | Q9YP87 |
| US28_HCMVM | F5HF62 |
| VT1_RFVKA | P25946 |
| X4YA71_HCMV | X4YA71 |

TABLE 3D
Human Chemokine/G-protein Coupled Receptors,
and Additional Chemokine Binding Proteins

| | UniProt |
|---|---|
| Uniprot ID | Accession |
| ACKR1_HUMAN | Q16570 |
| ACKR2_HUMAN | O00590 |
| ACKR3_HUMAN | P25106 |
| ACKR4_HUMAN | Q9NPB9 |
| BILF1_EBVB9 | P03208 |
| CCR1_HUMAN | P32246 |
| CCR10_HUMAN | P46092 |
| CCR2_HUMAN | P41597 |
| CCR3_HUMAN | P51677 |
| CCR4_HUMAN | P51679 |
| CCR5_HUMAN | P51681 |
| CCR6_HUMAN | P51684 |
| CCR7_HUMAN | P32248 |
| CCR8_HUMAN | P51685 |
| CCR9_HUMAN | P51686 |
| CCRL2_HUMAN | O00421 |
| CML1_HUMAN | Q99788 |
| CX3C1_HUMAN | P49238 |
| CXCR1_HUMAN | P25024 |
| CXCR2_HUMAN | P25025 |
| CXCR3_HUMAN | P49682 |
| CXCR4_HUMAN | P61073 |
| CXCR5_HUMAN | P32302 |
| CXCR6_HUMAN | O00574 |
| GPR17_HUMAN | Q13304 |
| GPR35_HUMAN | Q9HC97 |
| GPR75_HUMAN | O95800 |
| O41975_MHV68 | O41975 |
| TSG6_HUMAN | P98066 |
| UL33_HCMVA | P16849 |
| UL33_HCMVM | Q6SW98 |
| UL33_HHV6U | P52380 |
| UL33_HHV7J | P52381 |
| UL33_MUHVS | Q83207 |
| UL78_HCMVM | F5HET1 |
| US27_HCMVA | P09703 |
| US27_HCMVM | F5HDK1 |
| US28_HCMVA | P69332 |
| US28_HCMVM | F5HF62 |
| US28_HCMVT | P69333 |
| VG74_SHV21 | Q01035 |
| VGCR_HHV6Z | Q9QJ51 |
| VGPCR_HHV8P | Q98146 |
| VU51_HHV6U | P52382 |
| VU51_HHV6Z | P52542 |
| VU51_HHV7J | P52383 |
| XCR1_HUMAN | P46094 |

TABLE 3E
Chemokines

| | UniProt | | |
|---|---|---|---|
| UniProt ID | Accession | Chemokine Class | Synonyms |
| CC4L_HUMAN | Q8NHW4 | CC | CC4L, CCL4L1, |
| | | | CCL4L2, CCL4L, |
| | | | LAG1, SCYA4L1, |
| | | | SCYA4L2, AT744.2, |
| | | | LAG-1, MIP-1-beta, |
| | | | SCYA4L, SCYQ4L2 |
| CCL1_HUMAN | P22362 | CC | CCL1, SCYA1, I-309, |
| | | | P500, SISe, TCA3 |
| CCL11_HUMAN | P51671 | CC | CCL11, SCYA11 |
| CCL13_HUMAN | Q99616 | CC | CCL13, MCP4, NCC1, |
| | | | SCYA13, CKb10, |
| | | | MCP-4, NCC-1, |
| | | | SCYL1 |
| CCL14_HUMAN | Q16627 | CC | CCL14, NCC2, |
| | | | SCYA14, CC-1, CC-3, |
| | | | CKB1, HCC-1, HCC- |
| | | | 1(1-74), HCC-1/HCC- |
| | | | 3, HCC-3, MCIF, NCC- |
| | | | 2, SCYL2, SY14 |
| CCL15_HUMAN | Q16663 | CC | CCL15, MIP5, NCC3, |
| | | | SCYA15, HCC-2, |
| | | | HMRP-2B, LKN-1, |
| | | | LKN1, MIP-1 delta, |
| | | | MIP-1D, MIP-5, MRP- |
| | | | 2B, NCC-3, SCYL3, |
| | | | SY15, CCL15-CCL14, |
| | | | Mrp-2b, CCL15S |
| CCL16_HUMAN | O15467 | CC | CCL16, ILINCK, |
| | | | NCC4, SCYA16, |
| | | | CKb12, HCC-4, LCC- |
| | | | 1, LEC, LMC, Mtn-1, |
| | | | NCC-4, SCYL4 |
| CCL17_HUMAN | Q92583 | CC | CCL17, SCYA17, |
| | | | TARC, A-152E5.3, |
| | | | ABCD-2 |
| CCL18_HUMAN | P55774 | CC | CCL18, AMAC1, |
| | | | DCCK1, MIP4, PARC, |
| | | | SCYA18, AMAC-1, |
| | | | CKb7, DC-CK1, MIP-4 |
| CCL19_HUMAN | Q99731 | CC | CCL19, ELC, MIP3B, |
| | | | SCYA19, CKb11, MIP- |
| | | | 3b |
| CCL2_HUMAN | P13500 | CC | CCL2, MCP1, SCYA2, |
| | | | GDCF-2, HC11, |
| | | | HSMCR30, MCAF, |
| | | | MCP-1, SMC-CF |
| CCL20_HUMAN | P78556 | CC | CCL20, LARC, |
| | | | MIP3A, SCYA20, |
| | | | CKb4, Exodus, MIP-3- |
| | | | alpha, MIP-3a, ST38 |
| CCL21_HUMAN | O00585 | CC | CCL21, SCYA21, |
| | | | 6Ckine, CKb9, ECL, |
| | | | SLC, TCA4 |
| CCL22_HUMAN | O00626 | CC | CCL22, MDC, |
| | | | SCYA22, A-152E5.1, |
| | | | ABCD-1, DC/B-CK, |
| | | | STCP-1 |
| CCL23_HUMAN | P55773 | CC | CCL23, MIP3, MPIF1, |
| | | | SCYA23, CK-BETA-8, |
| | | | CKb8, Ckb-8, Ckb-8-1, |
| | | | MIP-3, MPIF-1, hmrp- |
| | | | 2a, CCL23S |
| CCL24_HUMAN | O00175 | CC | CCL24, MPIF2, |
| | | | SCYA24, Ckb-6, |
| | | | MPIF-2 |
| CCL25_HUMAN | O15444 | CC | CCL25, SCYA25, |
| | | | TECK, Ck beta-15, |
| | | | Ckb15 |
| CCL26_HUMAN | Q9Y258 | CC | CCL26, SCYA26, |
| | | | IMAC, MIP-4a, MIP- |
| | | | 4alpha, TSC-1 |
| CCL27_HUMAN | Q9Y4X3 | CC | CCL27, ILC, SCYA27, |
| | | | ALP, CTACK, CTAK, |
| | | | ESKINE, PESKY |
| CCL28_HUMAN | Q9NRJ3 | CC | CCL28, SCYA28, |
| | | | ENC1, ENC-1, |
| | | | KLHL35, KLHL37, |
| | | | NRPB, PIG10, |
| | | | TP53I10, CCK1, MEC |
| CCL3_HUMAN | P10147 | CC | CCL3, G0S19-1, |
| | | | MIP1A, SCYA3, |
| | | | ILD78ALPHA, MIP-1- |
| | | | alpha |
| CCL4_HUMAN | P13236 | CC | CCL4, LAG1, MIP1B, |
| | | | SCYA4, ACT2, |
| | | | AT744.1, G-26, HC21, |
| | | | LAG-1, MIP-1-beta, |
| | | | MIP1B1, SCYA2 |
| CCL5_HUMAN | P13501 | CC | CCL5, D17S136E, |
| | | | SCYA5, RANTES, |
| | | | SIS-delta, SISd, |
| | | | TCP228, eoCP |
| CCL7_HUMAN | P80098 | CC | CCL7, MCP3, SCYA6, |
| | | | SCYA7, FIC, MARC, |
| | | | MCP-3, NC28 |
| CCL8_HUMAN | P80075 | CC | CCL8, MCP2, |
| | | | SCYA10, SCYA8, |
| | | | HC14, MCP-2 |
| CL3L1_HUMAN | P16619 | CC | CL3L1, CCL3L1, |
| | | | CCL3L3, D17S1718, |
| | | | G0S19-2, SCYA3L1, |
| | | | 464.2, LD78, LD78- |
| | | | beta(1-70), |
| | | | LD78BETA, MIP1AP, |
| | | | SCYA3L |
| CXCL2_HUMAN | P19875 | CXC | CXCL2, GRO2, |
| | | | GROB, MIP2A, |
| | | | SCYB2, CINC-2a, |
| | | | GROb, MGSA-b, MIP- |
| | | | 2a, MIP2 |
| CXCL3_HUMAN | P19876 | CXC | CXCL3, GRO3, |
| | | | GROG, SCYB3, CINC- |
| | | | 2b, GROg, MIP-2b, |
| | | | MIP2B |
| CXCL5_HUMAN | P42830 | CXC | CXCL5, ENA78, |
| | | | SCYB5, ENA-78, |
| | | | CXCL5A, CXCL5B |
| CXCL6_HUMAN | P80162 | CXC | CXCL6, GCP2, |
| | | | SCYB6, CKA-3, GCP- |
| | | | 2 |
| CXCL7_HUMAN | P02775 | CXC | CXCL7, PPBP, |
| | | | CTAP3, SCYB7, |
| | | | TGB1, THBGB1, B- |
| | | | TG1, Beta-TG, CTAP- |
| | | | III, CTAPIII, LA-PF4, |
| | | | LDGF, MDGF, NAP-2, |
| | | | PBP, TC1, TC2, TGB, |
| | | | THBGB |
| CXCL9_HUMAN | Q07325 | CXC | CXCL9, CMK, MIG, |
| | | | SCYB9, Humig, crg-10 |
| CXL10_HUMAN | P02778 | CXC | CXL10, CXCL10, |
| | | | INP10, SCYB10, C7, |
| | | | IFI10, IP-10, crg-2, |
| | | | gIP-10, mob-1 |
| CXL11_HUMAN | O14625 | CXC | CXL11, CXCL11, |
| | | | ITAC, SCYB11, |
| | | | SCYB9B, H174, I- |
| | | | TAC, IP-9, IP9, b-R1 |
| CXL13_HUMAN | O43927 | CXC | CXL13, CXCL13, |
| | | | BCA1, BLC, SCYB13, |
| | | | ANGIE, ANGIE2, |
| | | | BCA-1, BLR1L |
| CXL14_HUMAN | O95715 | CXC | CXL14, CXCL14, |
| | | | MIP2G, NJAC, |
| | | | SCYB14, BMAC, |
| | | | BRAK, KEC, KS1, |
| | | | MIP-2g |
| CXL16_HUMAN | Q9H2A7 | CXC | CXL16, CXCL16, |
| | | | SCYB16, SRPSOX, |
| | | | CXCLG16, SR-PSOX |
| CXL17_HUMAN | Q6UXB2 | CXC | CXL17, CXCL17, |
| | | | VCC1, DMC, Dcip1, |
| | | | UNQ473, VCC-1 |
| GROA_HUMAN | P09341 | CXC | GROA, CXCL1, GRO, |
| | | | GRO1, MGSA, |
| | | | SCYB1, FSP, GROa, |
| | | | MGSA-a, NAP-3 |
| IL8_HUMAN | P10145 | CXC | IL8, CXCL8, GCP-1, |
| | | | GCP1, LECT, LUCT, |
| | | | LYNAP, MDNCF, |
| | | | MONAP, NAF, NAP-1, |
| | | | NAP1, SCYB8 |
| PF4V_HUMAN | P10720 | CXC | PF4V, PF4V1, |
| | | | CXCL4V1, SCYB4V1, |
| | | | CXCL4L1, PF4-ALT, |
| | | | PF4A |
| PLF4_HUMAN | P02776 | CXC | PLF4, PF4, CXCL4, |
| | | | SCYB4, PF-4 |
| SDF1_HUMAN | P48061 | CXC | SDF1, CXCL12, |
| | | | SDF1A, SDF1B, IRH, |
| | | | PBSF, SCYB12, TLSF, |
| | | | TPAR1, CXCL12A, |
| | | | CXCL12B |
| X3CL1_HUMAN | P78423 | CX3C | X3CL1, CX3CL1, |
| | | | FKN, NTT, SCYD1, |
| | | | ABCD-3, C3Xkine, |
| | | | CXC3, CXC3C, NTN, |
| | | | fractalkine, neurotactin |
| XCL1_HUMAN | P47992 | XC | XCL1, LTN, SCYC1, |
| | | | ATAC, LPTN, SCM-1, |
| | | | SCM-1a, SCM1, |
| | | | SCM1A |
| XCL2_HUMAN | Q9UBD3 | XC | XCL2, SCYC2, SCM- |
| | | | 1b, SCM1B |

TABLE 3F
Human Chemokine Panel Used In Phage-Display Screens. Chemokines are
referred to by the first part of the UniProt Entry Name. All libraries
were also screened with CO5 (complement C5a, as control).

| Phage-Display Library | Chemokines Used To Screen Library |
|---|---|
| Chemokines | CCL1, CCL11, CCL15, CCL17, CCL18, CCL19, |
| | CCL2, CCL20, CCL22, CCL25, CCL28, CCL3, |
| | CCL4, CCL5, CCL8, CXL10, CXL11, CXL13, |
| | CXL14, IL8, SDF1, X3CL1 |
| Receptors | CCL1, CCL11, CCL15, CCL17, CCL18, CCL19, |
| | CCL2, CCL20, CCL22, CCL25, CCL28, CCL3, |
| | CCL4, CCL5, CCL8, CXL10, CXL11, CXL13, |
| | CXL14, IL8, SDF1, X3CL1 |
| Tick Chemokine Binding | CCL1, CCL11, CCL15, CCL17, CCL18, CCL19, |
| Proteins - Class A Evasins | CCL2, CCL20, CCL22, CCL25, CCL28, CCL3, |
| | CCL4, CCL5, CCL8 |
| Tick Chemokine Binding | CXL10, CXL11, CXL13, CXL14, IL8, SDF1 |
| Proteins - Class B Evasins | |
| Viral Chemokine Binding | CCL1, CCL11, CCL15, CCL17, CCL18, CCL19, |
| Proteins | CCL2, CCL20, CCL22, CCL25, CCL28, CCL3, |
| | CCL4, CCL5, CCL8, CXL10, CXL11, CXL13, |
| | CXL14, IL8, SDF1, X3CL1 |

Peptides provided in this disclosure were selected such that they bind at least 3 or more chemokines with log 2 E>5 for each chemokine (log 2 E being a measure of binding affinity), and do not bind the control protein CO5 (complement C5a). A list of 451 peptide sequences with chemokine binding profiles as judged by the phage display outcome, is provided in Table 5A.

### Example 2. Testing of Individual Hexadecapeptide HD2 (SEQ ID NO: 396) and EB429 (SEQ ID NO: 392)

FIG. 2 shows inhibition of THP-1 monocyte and activated T-lymphocyte chemotaxis by the hexadecapeptides HD2 and EB429. HD2, EB429, a positive control (BK1.2 or P1834) and HD2SCR (scrambled, negative control) peptides (see US publication no. US-2020-0247855-A1), were tested at 10 μM. Y axis in each panel shows normalized count of migrated cells. HD2 is shown to inhibit cell migration induced by CCL5, CCL8, CCL7, CCL2 and CCL3 (FIG. 2A-FIG. 2E). EB429 is shown to inhibit cell migration induced by CXL9, CXL10 and CXL11 (FIG. 2F-FIG. 2H).

FIG. 3 shows alanine scanning mutagenesis of the HD2. Inhibition of human chemokine (CCL5, CCL7 or CCL8) induced THP-1 cell migration were screened in a transwell assay (Darlot, 2020).

FIG. 4 shows dose-response curves showing inhibition of human chemokine induced THP-1 cell migration by the hexadecapeptide HD2. FIG. 4D summaries the IC50 of the HD2 peptide next to the IC50 data achieved by the parent protein, EVA4_RHISA.

### Example 3. Identification of Peptide Combinations

Computer algorithms were devised to generate two-, three-, and four-peptide or more combinations of 525 peptides and predict their combinatorial chemokine binding profiles. Examples are provided where the predicted chemokine-binding profiles of 2— and 3-combinatorial peptide entities (CPEs, i.e. combinations of different peptides) are matched with disease chemokine profiles (based on literature), to identify CPEs that maximise binding to chemokines expressed in the disease, and minimise binding to chemokines not expressed in the disease (FIG. 5).

Administering chemokine binding protein/chemokine-derived hexadecapeptides of differing chemokine-binding specificities as cocktails to overcome redundancy is not desirable as each element in a cocktail must have therapeutic efficacy and safety independently established. Small peptides can however be chemically linked to create multivalent combinatorial peptide entities (CPEs) that combine the properties of the parental peptides and substantially enhance binding avidity. This approach will overcome the dual hurdles of reduced binding affinity and inability to overcome chemokine redundancy inherent in single hexadecapeptide agents, and furthermore will create novel non-natural molecular entities that can be patented.

### Example 4. Cell Migration Assay to Study Chemokine Inhibition

The ability of synthetic chemokine binding protein/chemokine-derived hexadecapeptides (with confirmed binding identified above) to inhibit chemokine function is determined using cell migration assays as previously described (Darlot, 2020) (Lee, 2019). Briefly, cell migration assays use THP1 cells (human monocyte) and primary human buffy coat white cells (consisting of neutrophil granulocytes, monocytes, and lymphocytes), and activated T-cells. THP1 cells and primary monocytes respond to CCL2, CCL3, CCL5, CCL8 and CXCL4, granulocytes to CXCL1 and CXCL8, activated T-cells to CXCL9/10/11. Assays are performed in 96-well Boyden chambers, and migrated cells counted and characterized using and ATTUNE Flow Cytometer as described. Triplicate assays are performed at the EC₈₀ dose of each chemokine at 10 μM peptide concentration. Peptides that inhibit chemotaxis significantly are identified using ANOVA followed by Dunnett's post-hoc test, and further characterized using dose-response assays to determine half-maximal inhibitory concentration (IC50) as described.

### Example 5. Synthesis and Screening of Combinatorial Peptide Entities

Chemokine binding protein/chemokine-derived hexadecapeptides shown to inhibit plaque chemokines are synthesized as tetra-branched hexadecapeptides to create-CPEs. CPEs are constructed from single hexadecapeptides (homo-tetramers) to determine if this enhances binding avidity to chemokines bound by the parental hexadecapeptide. CPEs are also constructed from two, three, or four hexadecapeptides (hetero-tetramers) to determine if the spectrum of chemokines bound combines the properties of the parental hexadecapeptides. Chemokine binding avidity (Kd) and half-maximal inhibitory concentration (IC50) are determined for each CPE using fluorescence polarization, and function using chemotaxis assays as described above.

### Example 6. Phage Display Results Using Saturation Mutagenesis

A phage-display library was constructed using the HD2 sequence, replacing each residue encoding codon with the degenerate sequence NNK. The 16 degenerate sequences (which also encoded the parental HD2 sequence) were pooled and cloned into display phage. Library screening was performed using the indicated chemokines displayed on streptavidin beads. Exemplar peptides were selected such that log 2 E for at least one chemokine was greater than 5, and log 2 E for binding to control (CO5, was less than zero). The X-axis of FIG. 7 shows individual residue changes by location in the peptide, and Y-axis shows the log 2 of fold enrichment (log 2 E) of the mutated sequence following chemokine affinity selection. The fold enrichment of parental HD2 is also indicated. The mutant peptide sequences are presented in Table 5B.

### Example 7. Library Construction and Screening of 21 Class A Evasins

A phage-display library was constructed where the major bacteriophage coat protein p8 was fused to hexadecapeptides derived from the mature sequences of 21 class A evasins (see Example 18 for further details). This approach resulted in multivalent phage display, and allowed identification of low affinity interactions. As unpaired cysteine residues were thought to compromise phage-display, they were mutated during library construction to alanine, a substitution that removes side-chains beyond the β-carbon without affecting conformational flexibility. The library was constructed so that it included peptides with Cys residues intact, and also had peptides with mutations of Cys to Ala, and the conservative substitution, Cys to Ser. Comparing counts in the wild-type Cys containing peptides, and those with Cys-Ser and Cys-Ala mutations obtained by next generation sequencing of the input library showed however that there was little or no impact on count numbers (data not shown). The library was selected with 25 biotinylated chemokines, individually attached to a streptavidin matrix. Following library selection with each chemokine, the enrichment (E) of each peptide was calculated in comparison to the input library, and expressed as log 2 E, as this metric is correlated with binding affinity.

### Example 8. Phage-Display Identifies Regions Overlapping with Known Chemokine Binding Sites

Mapping each peptide to the parental sequence (e.g. FIG. 1A-1D) by log 2 E obtained for each chemokine identified overlapping hexadecapeptides that clustered in regions of the parental sequences. This is most evident for instance for EVA4, EV672, EV974 and EV546. To identify regions of the parental protein that contribute to chemokine-binding hexadecapeptides, we calculated the contribution of each residue (rlog 2 E) as the sum of log 2 Es of peptides overlapping a residue. We defined regions of interest where rlog 2 E exceeded the upper boundary of the 95% confidence interval of median rlog 2 E. The regions of interest overlap the known chemokine-binding sites for EVA4, EV672, and EV974. Surprisingly, we found that hexadecapeptides from these regions, in addition to binding CC chemokines as expected, also bound CX3CL1 and the CXC chemokines (CXCL1, CXCL10, CXCL11, CXCL12B, CXCL13, CXCL5 and CXCL8), which are not known to bind the parental proteins.

### Example 9. Phage-Display Identifies Promiscuous Chemokine Binding Peptides

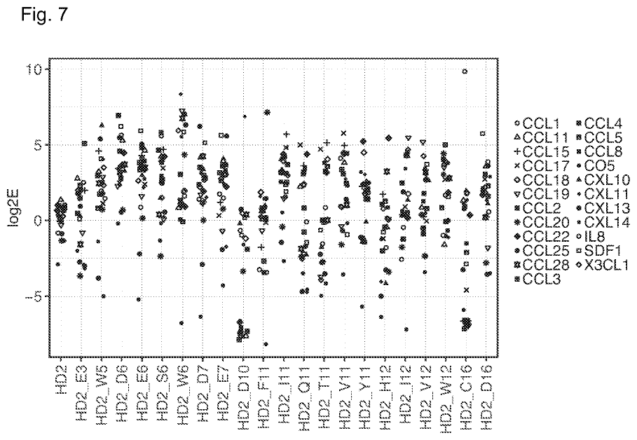

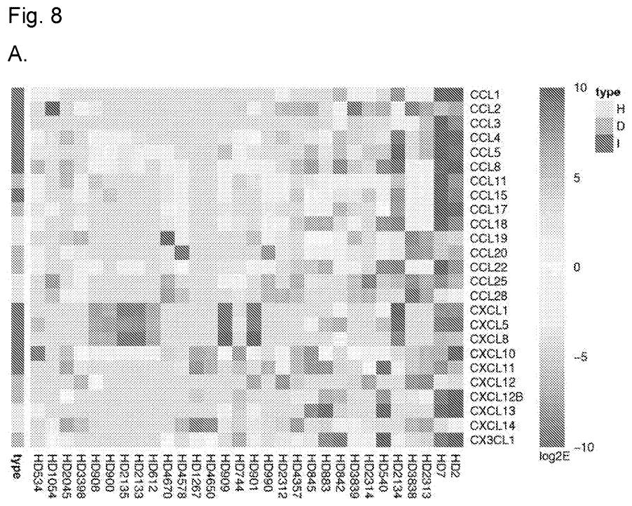

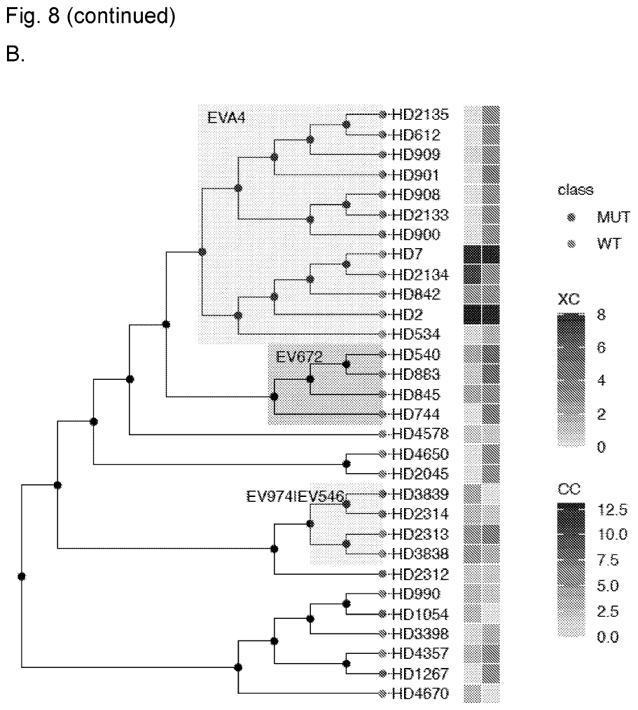

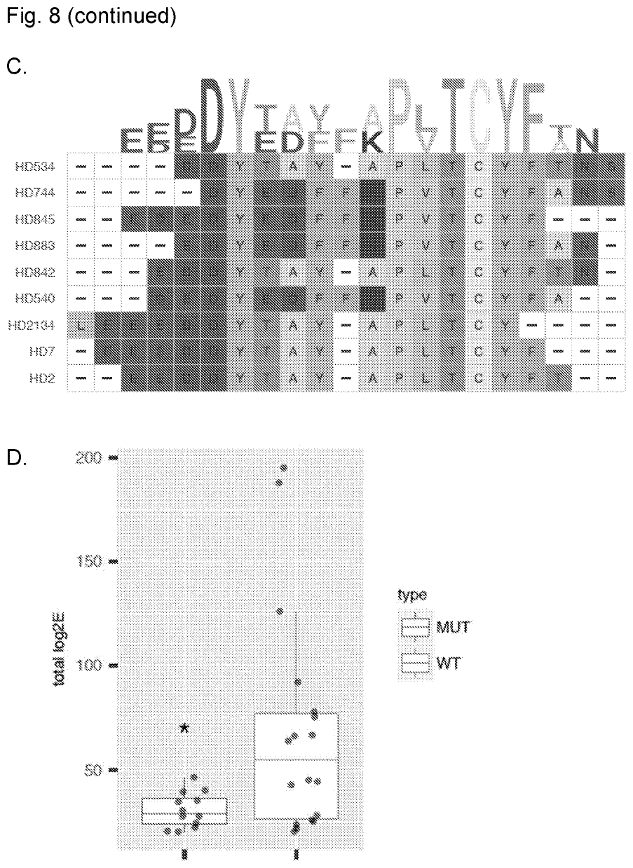

To characterize peptides that bound chemokines promiscuously, the peptides that bound at least three distinct chemokines with log 2 E>5 were selected. Of the 30 peptides identified as meeting these criteria (see Table 6), all of the 18 peptides that were identical to the wild-type sequences (i.e. no Cys→Ala or Cys→Ser substitutions) had one or more Cys residues. In each case (i.e., EVA4, EV991, EV974, EV672, E1243, E1180) where disulfide bond data was available in UniProt or from Alphafold, this Cys residue was disulfide bonded in the parental evasin sequence. Nine of 12 Cys-mutant peptides were mutant versions of wild-type peptides. The heatmap of individual log 2 E values indicates that certain peptides (e.g., HD2, HD7) are highly promiscuous, binding over 15 different chemokines including exemplars from CC, CXC and CX3C classes (FIG. 8A). The neighbor-joining cladogram (FIG. 8B) shows that EVA4, EV672, and the highly homologous evasins EV974 and EV546 contribute overlapping peptides.

### Example 10. Sequence Alignment Reveals a Conserved Motif Containing an Unpaired Cys Residue

The two largest groups of overlapping wild-type peptides are from EVA4 and EV672. Multiple sequence alignment of these peptides (FIG. 8D) identified two linear motifs with conserved residues: E(E/D)(E/D)DY and P(L/V)TCYF. The importance of having an unmutated Cys residue was examined by evaluating the total log 2 E for each peptide aggregated over all chemokines as a summary measure of binding affinity (FIG. 8D). This showed that wild-type peptides had significantly higher total log 2 E compared to mutant peptides i.e., where Cys had been mutated to either Ser or Ala. These results suggest that the unpaired Cys residue in the PL/VTCYF motif contributes to chemokine binding affinity.

### Example 11. Biolayer Interferometry Confirms Binding of HD2 to CC and CXC Chemokines

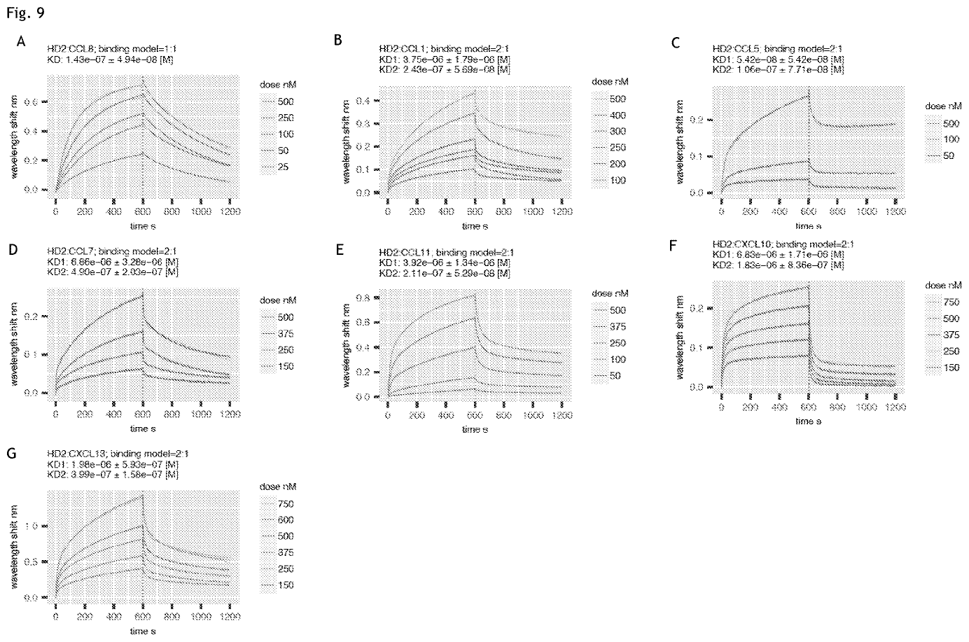

The ability of the exemplar peptide HD2 to bind a panel of chemokines (C5A, CCL1, CCL11, CCL15, CCL17, CCL18, CCL19, CCL2, CCL20, CCL22, CCL25, CCL28, CCL3, CCL4, CCL5, CCL8, CX3CL1, CXCL1, CXCL10, CXCL11, CXCL12, CXCL12B, CXCL13, CXCL14, CXCL5 and CXCL8) was examined using biolayer interferometry (BLI). HD2 and a scrambled control (HD2SCR) were generated as HIS:SUMO fusion proteins in E. coli. This approach resulted in soluble proteins that can be immobilized on to BLI sensors through the N-terminal HIS tag. The binding of HD2 to several CC and CXC-class chemokines (CCL1, CCL5, CCL7, CCL8, CCL11, CXCL10 and CXCL13) was characterized at different chemokine doses to evaluate binding affinity (K_{D}). Each chemokine showed dose-dependent binding (FIG. 9). 1:1, 2:1 and 1:2 binding models were examined and the model giving the best fit to the raw data was selected. In each case, a 2:1 binding model gave the best fit suggesting that two sites on immobilized HD2 were involved in the interaction. Estimated binding affinities ranged from 41 nM for CCL11 to 1.8 μM for CXCL10.

### Example 12. HD2 Inhibits Both CC- and CXC-Chemokines

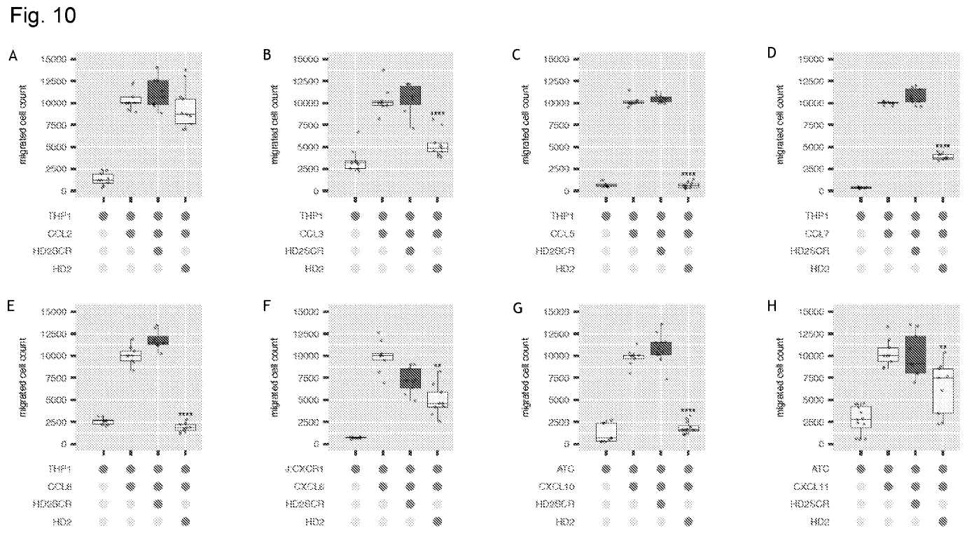

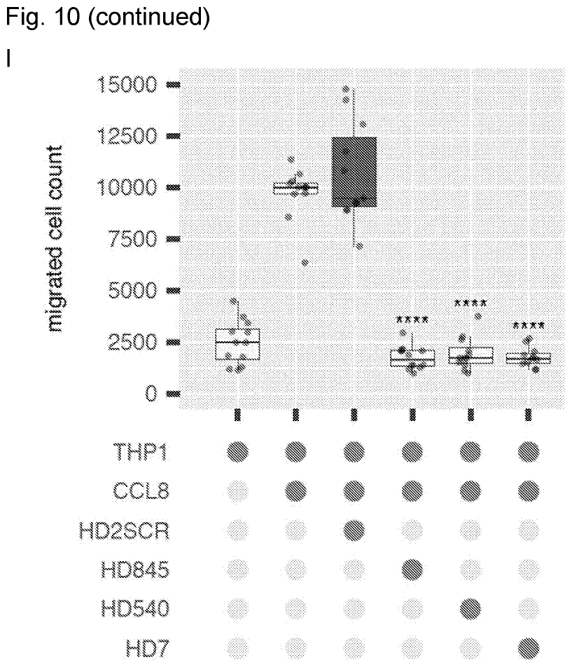

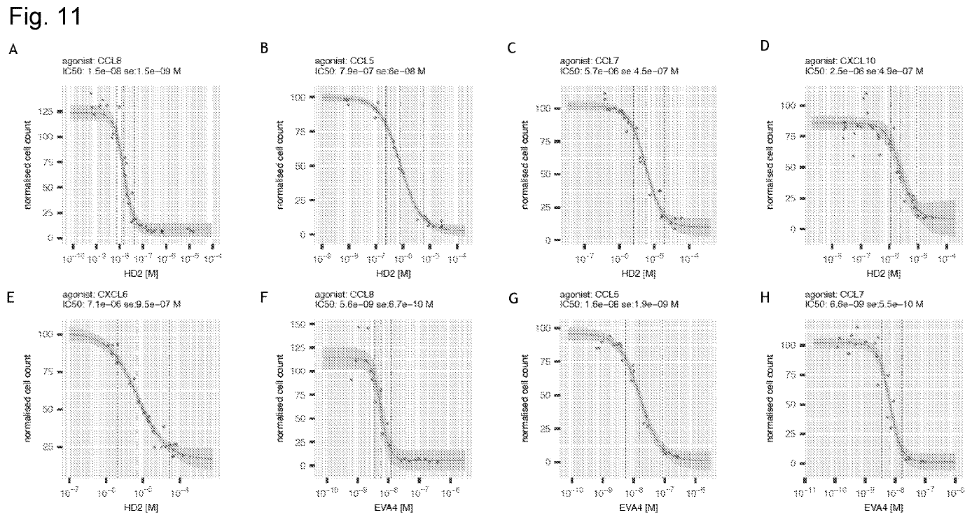

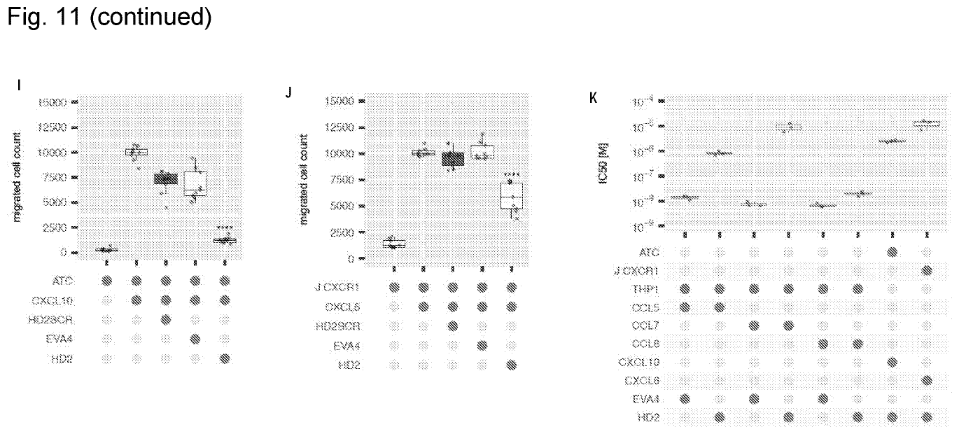

The ability of the exemplar peptide HD2 in inhibiting chemotaxis by CC and CXC chemokines was studied (FIG. 10). In comparison with a scrambled control HD2SCR, significant inhibition of cell migration induced by the CC chemokines CCL2, CCL3, CCL5, CCL7, CCL8, and CCL23 was observed. In addition, inhibition of the CXC chemokines CXCL10, CXCL11 and CXCL6 was observed. Where tested, inhibition of CCL8 by HD845 and HD540 was observed (FIG. 10). Dose-response experiments with HD2 against CCL8, CCL5, CCL7, CXCL10 and CXCL6 (FIG. 11). While EVA4 inhibited the CC chemokines (FIG. 11F-11H), it did not inhibit CXCL10 at a dose of 20 μM (FIG. 11H), and dose response curves could not be obtained. The IC50 of the HD2 peptide ranged from a median 1.9 E−8 for CCL8 to 1.4 E−5 molar (0.015 to 140 μM) for CXCL6 (FIG. 11K).

### Example 13. Alanine-Scanning Mutagenesis Identifies Contiguous Residues in HD2 Necessary for Binding

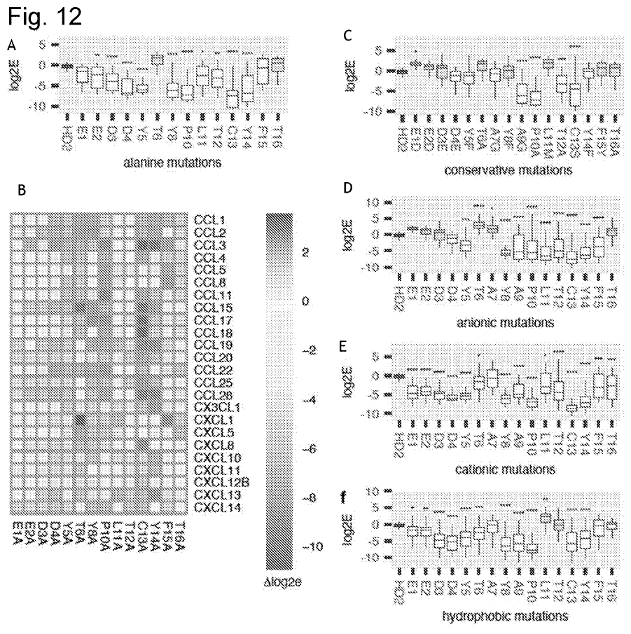

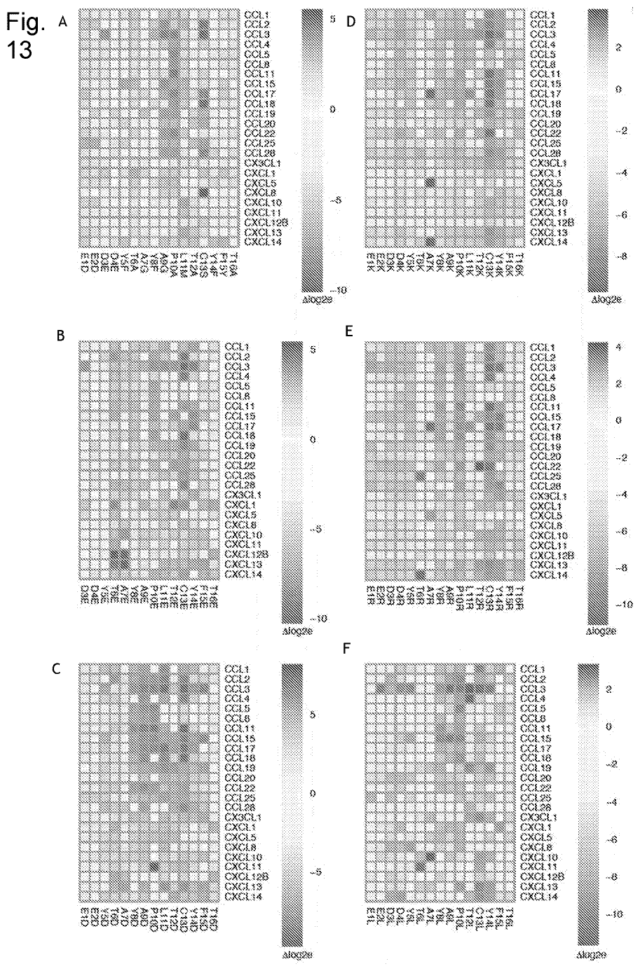

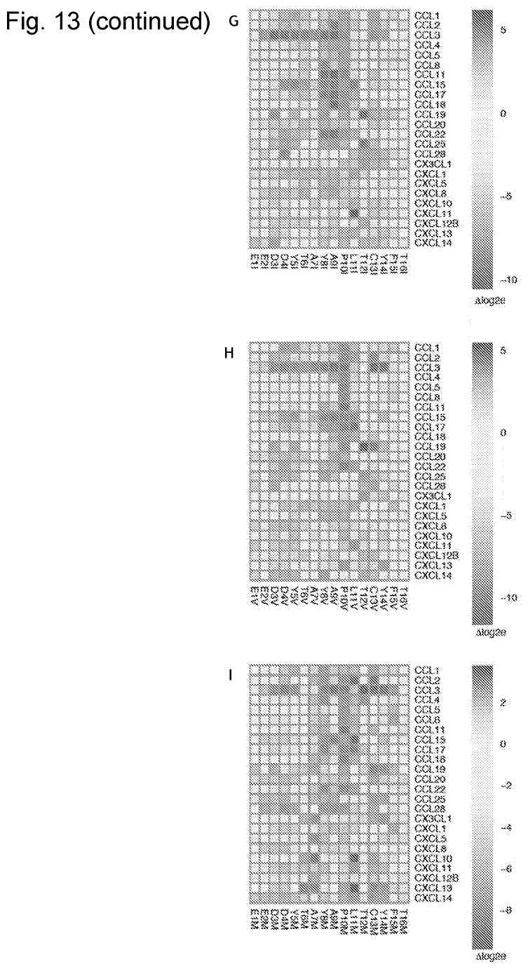

A molecular level understanding of binding mechanism is needed for development of peptides as therapeutics and is usually obtained from structural analyses. As HD2 binds many chemokines this becomes a challenging task, and we explored if we could elucidate this using phage display mutagenesis. The role of each residue in HD2 for binding was examined in phage-display. A library of HD2 mutations that had NNK substituted at each residue and performed phage-display selection against a panel of 24 biotinylated chemokines, in parallel, as described in Example 7. This strategy allowed us to evaluate the impact not only of Ala substitutions, but also conservative, hydrophilic and hydrophobic substitutions, and compare with binding of parental HD2 which is also included in the library. Ala substitution removes side-chains beyond the β-carbon and can be used to infer the role of side-chain functional groups without affecting conformational flexibility of the backbone. Analysis following selection with the chemokine panel showed that several mutations had large and significant impact on mean log 2 E when compared to parental HD2 (FIG. 12A). Contiguous regions of three or more residues that significantly reduced binding upon Ala substitution compared to parental HD2 were E2-Y5 and T12-Y14. These residues are within the two motifs identified previously. Other notable residues identified were Y8 and P10. We next judged the impact of Ala mutation by calculating the change in log 2 E (Δlog 2 E) between the mutant and parental HD2. A positive value of Δlog 2 E indicates increased binding, and a negative value decreased binding compared to parental HD2. A tile-plot of the data (FIG. 12B) showed that while certain chemokines were relatively tolerant to single point alanine mutagenesis (e.g., CCL8), others (e.g., CXCL10) were not. Analysis of conservative residue substitutions, identified from the Dayhoff PAM250 substitution matrix (FIG. 12C, 13A) indicated that these substitutions had no significant deleterious effect on Δlog 2 E excepting at A9G, P10A, T12A and C13S. As may be seen, the log 2 E for the parental HD2 was ~0 in these analyses indicating lack of enrichment. Examination of our data showed that there were several mutations that enhance binding to chemokines (e.g., anionic mutations at T6 and A7 or hydrophobic mutations at L11, see FIG. 12, and FIG. 13) providing a likely explanation of this result as they would compete with the parental HD2 peptide for binding.

### Example 14. Hydrophile-Scanning Mutagenesis Identifies an N-Terminal Anionic Patch in HD2

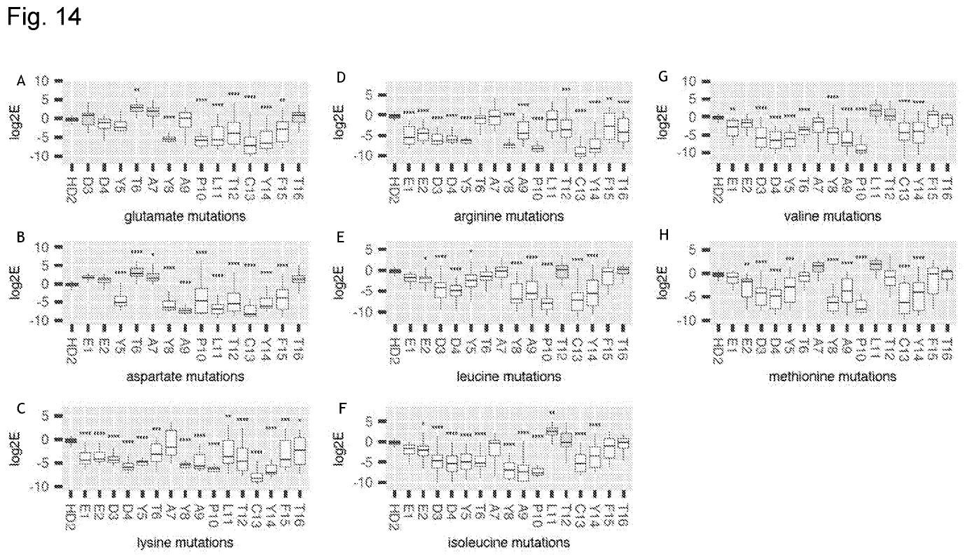

Ionic bonds are major interactions at protein interfaces, and occur between charged anionic and cationic residues. Hydrophile scanning uses systematic mutation to anionic (e.g. glutamic or aspartic) or cationic (e.g. lysine, arginine) residues and complements alanine-scanning mutagenesis. Analysis of these HD2 mutations present in the dataset generated above, shows that glutamic or aspartic acid (anionic) substitution at the N-terminus is not deleterious for binding, and in some cases e.g., at T6 improve binding, whereas they are significantly deleterious at the C-terminus (FIGS. 12D, 13B, 13C, 14A, 14B). In contrast, cationic (lysine or arginine) mutations are generally deleterious through the peptide (FIGS. 12E, 13D, 13E, 14C and 14D). Taken together these results indicate that an anionic N-terminal patch (EEDD) in HD2 mediates crucial interactions with target chemokines.

### Example 15. Hydrophobe-Scanning Mutagenesis Identifies a Role for C-Terminal Hydrophobicity in HD2

Protein interfaces are frequently characterized by hydrophobic interactions, leading to exclusion of these residues from the water exposed surface. Systematic mutation to the hydrophobic residues (i.e, valine, isoleucine, methionine and leucine) was tested to see if it would allow identification of HD2 residues that likely mediated hydrophobic interactions with chemokines. Analysis of these mutations from the phage-display dataset generated above, showed that certain residues at the C-terminus-L11, T12, F15 and T16 could be substituted with a hydrophobic residue without significant deleterious effect (FIGS. 12F, 13F-13I, 14E-14H).

### Example 16. Alanine-Scanning Mutagenesis Identifies Functionally Important Residues in HD2

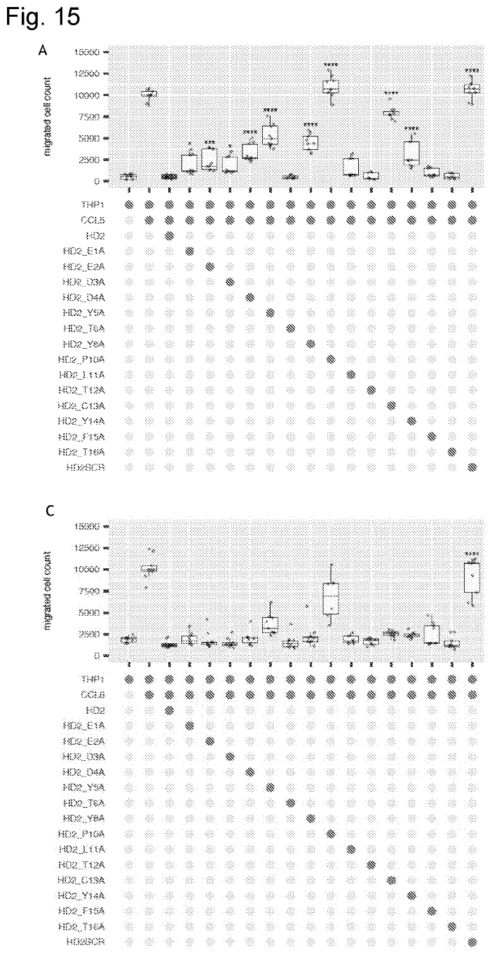

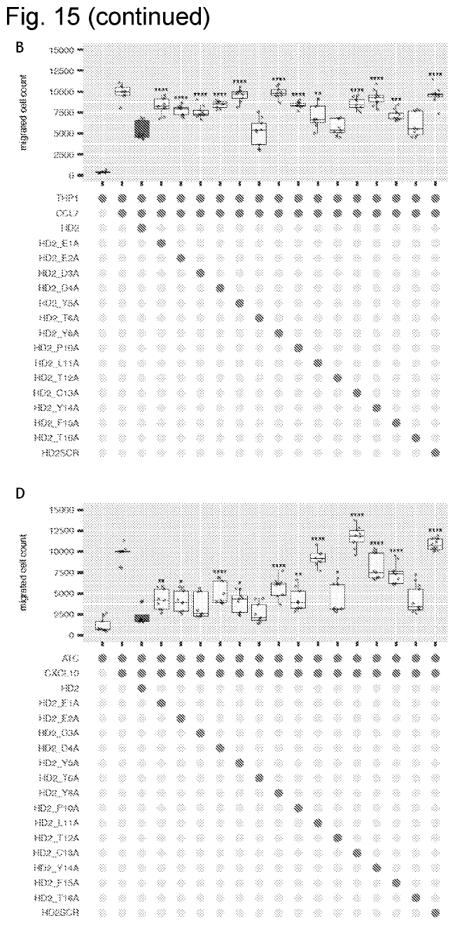

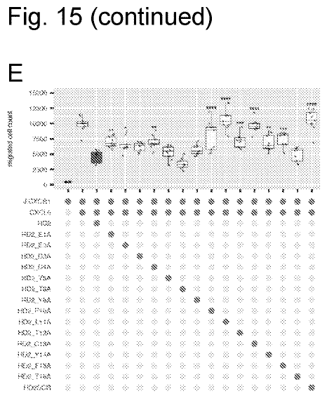

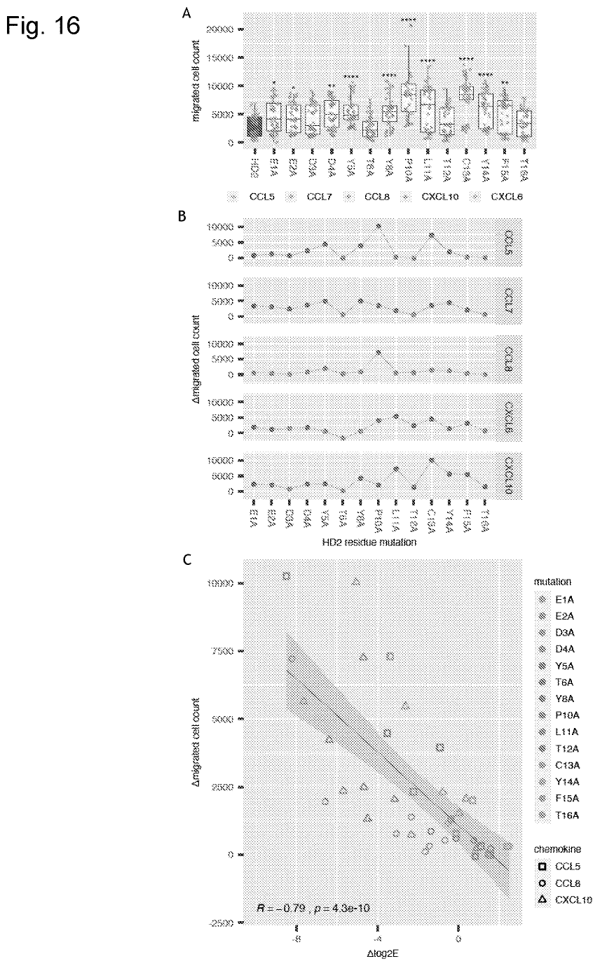

To determine if alanine substitutions impacted the ability of HD2 to inhibit chemokine function we performed chemotaxis assays with synthetic peptides. The effect of HD2 alanine mutants on migrated cell counts in chemotaxis assays was examined using chemokines CCL5, CCL7, CCL8, CXCL6 and CXCL10, and was compared to parental HD2 as control (FIG. 15). While several mutations significantly affected ability to inhibit chemotaxis (as evidenced by an increase in migrated cell count relative to parental HD2) by CCL5, CCL7, CCL8 and CXCL6 and CXCL10, we observed that only P10A significantly affected chemotaxis by CCL8. A meta-analysis of the alanine-mutant chemotaxis experiments was performed by pooling all datapoints to obtain an overview of residues critical for chemokine inhibition (FIG. 16A). This analysis revealed that key residues significantly impacting function when mutated were E1, E2, D4, Y5, Y8, P10, L11, and C13-Y15. This data is summarized by chemokine in FIG. 16B, where the effect of residue mutated to change in migrated cell count is shown. The correlation of chemotaxis inhibition with binding in phage-display was studied. The relationship between change in migrated cell count (Δmigrated) with change in log 2 E (Δlog 2 E) for each peptide:chemokine pair where such data was available (FIG. 16C). These two properties are highly correlated. Mutant peptide:chemokine pairs with lower Δlog 2 E (i.e., poor binding) have higher Δmigrated values (i.e., poor chemotaxis inhibition). Thus, poor binding in the phage-display experiment correlates with reduced inhibition of chemotaxis, indicating that the most likely explanation for loss of inhibition by a mutant is reduced binding affinity.

### Example 17. HD2 Binds CC and CXC Chemokines at Different Locations to Partially Occlude Receptor-Binding Sites

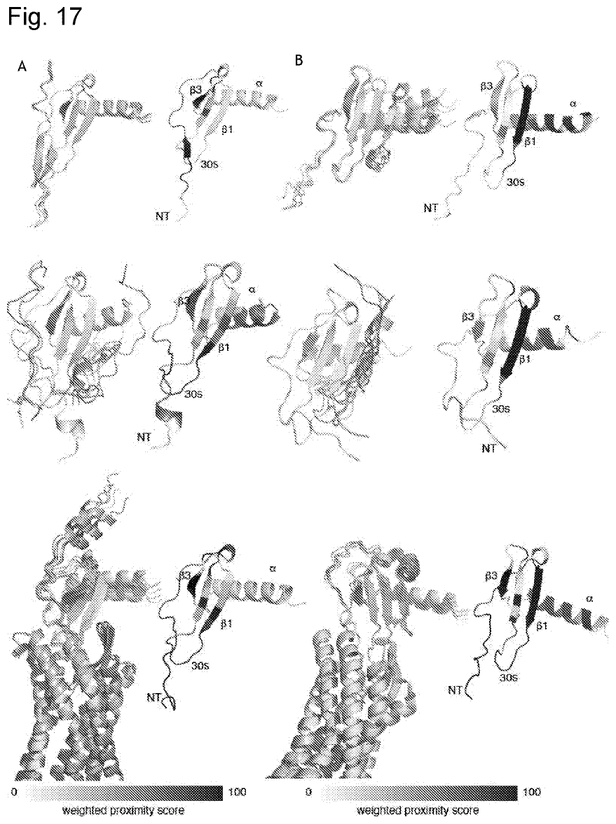

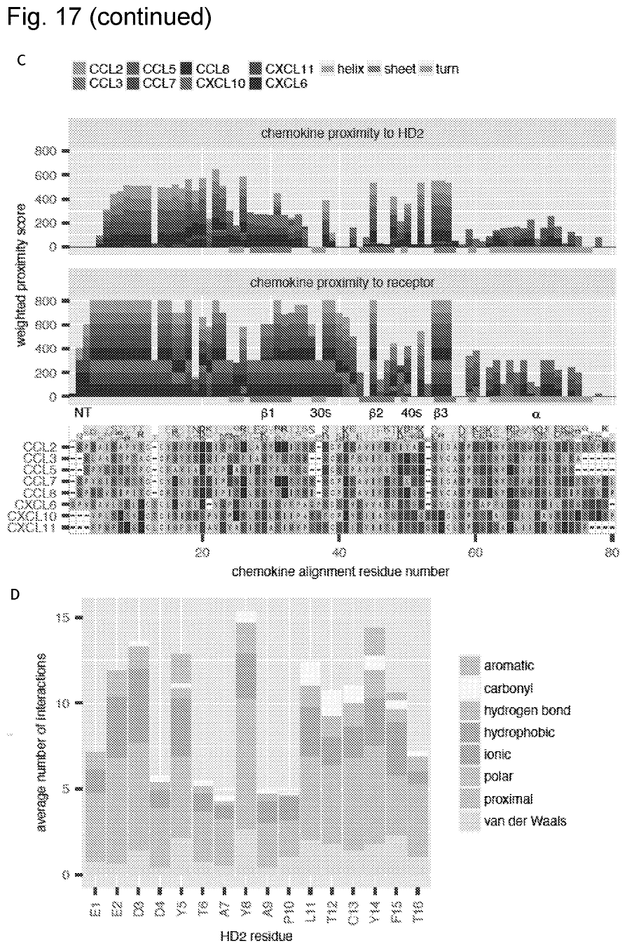

To understand how the peptide HD2 may bind and inhibit CC- and CXC-class chemokines we modelled the chemokine:HD2 complex using two different methods-AlphaFold2-Multimer (Weng, et al. J Chem Theory Comput 16, 3959-3969 (2020)) and AutoDock CrankPep (Zheng et al. Immunity 46, 1005-1017 e1005 (2017)) (FIG. 9). As models of these chemokines with their receptors and with EVA4 are not uniformly available, chemokine:receptor and chemokine: EVA4 complexes were modelled using AlphaFold2-Multimer (FIG. 17). For each model, five alternative docking poses were identified using AlphaFold2-Multimer and ten using AutoDock CrankPep. Rather than analyzing the single highest-ranked pose from each method, which is less likely to retrieve the native docking pose, data from the docking poses were aggregated into heat maps of weighted proximity scores (FIG. 17). The proximity scores were weighted either by confidence score (for AlphaFold2-Multimer) or by predicted free energy of binding (AutoDock CrankPep). Notably, the distal N-terminus of the cognate receptor is predicted to bind to the β1-strand of CXC-chemokines by wrapping around the chemokine, whereas this is not the case with the interaction of CC chemokines with their cognate receptors. AlphaFold2-Multimer docking suggested that HD2 is in proximity to the N-terminus, N-loop, and residues within the 30s and 40s loops of CCL2, CCL3, CCL5, CCL7 and CCL8, but in proximity to the β1-strand and α-helix of CXCL6, CXCL10 and CXCL11 (FIG. 17). The AlphaFold2-Multimer models of EVA4 with chemokines known to bind or be inhibited by EVA4 suggested that it is in proximity to residues in the N-terminus and the N-loop, and residues within the 30s and 40s loops of CCL2, CCL3, CCL5, CCL7 and CCL8. The HD2 segment within the chemokine: EVA4 models occupies a similar position to that observed in the corresponding chemokine:HD2 models, and the chemokine residues in proximity to EVA4 in these models are similar to those in proximity to HD2. Analysis using AutoDock CrankPep indicated overall concordance with the AlphaFold2-Multimer results, except CXCL11 and CXCL6 where the peptide is in proximity to the N-terminus. Weighted proximity heatmaps of chemokine:receptor complexes obtained using AlphaFold2-Multimer suggested that CCR1, CCR2, and CCR5 are in proximity to the N-terminus, N-loop, B1 and B3-strands and residues within the 30s and 40s loops, while CXCR3 and CXCR1 are also in proximity to residues in the α-helix respectively (FIGS. 17A-17C). The overlap between peptide-proximal and receptor-proximal regions (summarized in FIG. 17C) suggests that the peptide likely functions by partially occluding the receptor-proximal regions, interfering with binding. Analysis of the average number of chemokine bonds from the AlphaFold2-Multimer models for each HD2 residue indicated that the largest numbers of interactions, on average, are formed by the sequences E1:Y5 and L11:F15, i.e., the sequences EEDDY and LTCYF (FIG. 9E), with the unpaired Cys residue itself contributing many interactions.

A surprising and important result of our study was that several peptides—unlike the parental class A evasin proteins from which they were derived—bind and inhibit both CC and CXC class chemokines. The peptide HD2 is from the same region of EVA4 as an octadecapeptide synthesised based on NMR analysis of the EVA4: CCL5 interface (Denisov, S. S. (2020)). Unlike HD2, the octadecapeptide has a Cys-Ala mutation, and was reported to inhibit a single chemokine, CCL5. As shown, the example peptide HD2 also shares sequence homology with several EV672-derived peptides including HD845. None of the previously described class A evasin-derived peptides were shown to have anti-chemokine activity against CXC-class chemokines. Potential explanations for the lack of CXC-binding capacity of the parental evasin is that CXC-chemokine interacting residues on the peptide may be unavailable in the parental protein. For instance, the unpaired Cys residue in the peptide is invariably disulfide bonded in the parental evasin. As shown herein, mutation of Cys to Ala or Ser resulting in loss of binding activity in phage display, and loss of functional activity in chemotaxis assays, which suggests that the unpaired Cys residue in these peptides is important. Cys residues are known to have non-covalent interactions, and it is possible that these enhance chemokine binding and inhibition.

Following the discovery of the peptides by phage-display, it was demonstrated that the exemplar peptide HD2 binds multiple CC and CXC-chemokines and also inhibits multiple CC and CXC-chemokines in chemotaxis assays. These results suggest that the broad-spectrum chemokine-inhibiting activities of HD2 could be applied for therapeutic development. Sequence conservation across peptides derived from EVA4 and EV672 that bound both CC and CXC chemokines indicated the presence of conserved residues in two motifs, E(E/D)(E/D)DY at the N-terminus; and (L/V) TCYF at the C-terminus. The two motifs are biochemically distinctive, with the former characterized by an anionic patch and the latter by hydrophobic, aromatic and cysteine residues.

### Example 18. Methods for Examples 7 to 17

### Phage Display Library Design

Wild-type class A evasin nucleotide sequences (excluding the signal peptide encoding sequence) were first codon-optimized for E. coli expression using GeneDesigner, using default settings (i.e. codon bias threshold 0.1, and removing splicing, RNA destabilizing, prokaryotic ribosome binding site, Shine-Dalgarno sequences, optimizing the 5′ structure, and removing repeats). Codon optimization was also repeated after mutating each Cys residue to Ala and Ser. 81-mer oligonucleotides were designed such that they encoded hexadecapeptides overlapping by a single residue, and had the sequence 5′-GCAGCCTCTTCATCTGGC, and GGTGGAGGATCCGGA-3′ at respective ends to enable amplification and cloning. A total of 4741 distinct peptides (including peptides where Cys was mutated to Ala or Ser) were designed as oligonucleotides and synthesized as a pool (Genscript, 12K chip).

### Phage Display Library Construction

The plasmid prSTOP4 (kind gift from Dr Sachdev Sidhu, University of Toronto) was modified to have an NsiI restriction site and was amplified using primers:
pRSTOP4Nsi_fwd: 5′-GGAGGCGCCGAGGGTGAC and
pRSTOP4Nsi_rev: 5′-ATAGGCATTTGTAGCAATAGAAAAAACGAACATAGATGCAAG.

Oligonucleotide pools were amplified using primers: oligo_fwd: 5′-ctattgctacaaatgcctatgcagcctcttcatctggc and oligo_rev2: 5′-tcgtcaccctcggcgcctcctcoggatcctccacc. Oligonucleotide PCR products were cloned into plasmid prSTOP4Nsi using NEB Builder HiFi Assembly following instructions of the manufacturer. The vector pool was used to transform electrocompetent Invitrogen ElectroMAX™ DH5α-E™ cells. Plasmid DNA was harvested by MaxiPrep (GeneJet Plasmid Maxiprep Kit) and was transformed into SS320 phage display electrocompetent cells (Lucigen), following the manufacturer's protocol, resuspended in 950 μL recovery media for each electroporation, transferred to 50 mL Falcon tubes, 100 μL 1011 CFU/mL M13KO7 helper phage (NEB) added and shaken at 37° C., 220 RPM for 1 h. 1 mL recovery culture was added to 2YT medium containing +50 ug/mL carbenicillin and 25 ug/mL kanamycin, and grown overnight at 37C, 220 rpm. Phage was precipitated from the culture supernatant by adding 22.5 mL of supernatant to 4.5 mL 20% PEG 8000/2.5 M NaCl, incubating on ice for 30 min, and centrifuging at 10,000 RPM, 4° C. for 10 min, and resuspending phage in 1 ml of PBT, followed by centrifugation at 13000 RPM, 4° C., for 10 min. Glycerol (10% final (v/v) was added to supernatant, which was aliquoted and stored at −80C. For mutant libraries, 81-mer mutant oligonucleotides replacing NNK at each of the 16 peptide encoding residues were designed with the sequence arms described above. Oligonucleotides were individually amplified and cloned into plasmid prSTOP4Nsi. Screening of this library was performed as described above.

### Phage Display Library Screening

Phage display screening was performed using a protocol known in the art with certain modifications. Experiments were performed in 96-well plates. Briefly, biotinylated chemokines (lug, Almac or ProteinFoundry, Supplementary information Table 2) were immobilized on 5 μl streptavidin-coated magnetic beads (Dynabeads™ M-280 Streptavidin, Invitrogen). Chemokine-bound beads were blocked for two hours in blocking buffer (PBS (phosphate-buffered saline)+0.2% BSA (bovine serum albumin), and phage library (100 μl) allowed to bind for two hours at a concentration of 10¹⁰ CFU/mL. Beads were washed 15 times with PT buffer (PBS+0.05% TWEEN) to remove unbound phage and transferred to a fresh plate. Beads were incubated with 100 μL of phage-resistant Omnimax E. coli (Invitrogen) at optical density (OD₆₀₀) 0.6-0.8 and shaken at 37° C. for 30 min, following which 10 μL M13K07 helper phage (NEB, final concentration 10¹⁰ cfu/mL) was added and shaken at 37° C. for 45 min. Cells were transferred to 1 mL of 2YT medium supplemented with 150 μg/mL carbenicillin and 75 μg/mL kanamycin in a 96-deep well block with V-bottom and grown overnight shaking at 37° C., 200 rpm. Plates were either centrifuged at 2000 g (4° C. for 30 min) or transferred to screw cap tubes and pelleted by centrifugation at 4000 g and 4° C. for 15 min. 540 μL of supernatant was transferred to a new 96-deep well plate, 60 μL 10×PBT added and stored at 4° C. until further use in subsequent rounds. Phage display was carried out for three rounds.

### Next Generation Sequencing

Inserts from the input library and from the final phage population following selection were amplified by PCR using primers: 5′-ACACTCTTTCCCTACACGACGCTCTTCCGATCTCTAGCGCTATGCCTATGCAGCCTC0 TTCA and 5′-GACTGGAGTTCAGACGTGTGCTCTTCCGATCTCGTCTGCGATGACAACAACCATCG CCCA, (Life Technologies). The PCR products were cleaned up using Monarch PCR and DNA Cleanup Kit and subsequently sequenced at Azenta/GeneWiz using the AmpliconEZ protocol.

### Next Generation Sequence Analysis

Paired end read Fastq files were joined by the read id. Sequences containing both forward and reverse demultiplexing regions (5′-CTAGCGCT and 5′-CGCAGACG) and both constant regions (5′-ATGCCTATGCAGCCTCTTCATCTGGC and 5′-GGTGGAGGATCCGGAGGAGGCGCCGAGGGTGACGATCCCGCAAAAGCGGCCTTTA ACTCCCTGCAAGCCTCAGCGACCGAATATATCGGTTATGCGTGGGCGATGGTTGTT GTCAT were analysed. Insert sequences from each read were identified and reads with non-identical insert sequences or ambiguous sequences were excluded, as were inserts that were incorrect in size (i.e., not 48 nucleotides). Insert sequences were translated to peptide. Only inserts present in the designed library were counted and frequency determined. Library cloning efficiency was calculated as the ratio of distinct cloned peptide inserts to the number of inserts in the designed library and was 98%. Hexadecapeptide enrichment (E) following selection was calculated as ratio of output peptide frequency to input peptide frequency and expressed as log 2 E. Where proportion of count in input library was not available it was replaced with the lowest input proportion in the experiment rather than replacing it with 0. This is needed to avoid a value of E that is infinity. For mutagenesis experiments, where proportion of count in output was not available it was replaced with the lowest output proportion in the experiment rather than zero, which would result in a-Infinity log 2 E. R-packages used for analysis of NGS datasets were ShortRead_1.56.1 and Biostrings_2.66.0.

### Peptide Mapping to Parental Protein Sequences

Enriched peptides were mapped to parental proteins using custom R-scripts. Parental protein sequences were obtained from UniProt using RCurl_1.98-1.5. Briefly, collated enrichment data were filtered to include only those peptide-chemokine combinations where there was enrichment (i.e. log 2 E>0), and wild-type and mutant peptides were mapped to the originating wild-type protein. Residue log 2 E was calculated as the sum of log 2 Es of peptides overlapping a residue. The rolling median of residue log 2 E was calculated using the function rollmedian function in R-package zoo_1.8-12, with k=7 and fill=NA. Confidence interval of the median was calculated using the cimed function in R-package asbio_1.9-2.

### Annotation of Parental Proteins

PDB files for evasin complexes with chemokines 7S4N and 7SO0 (for EV974) and 3FPU (for EVA1) were retrieved and chemokine-binding site residues were identified using the bio3d binding.site function with the default cutoff of 5 Å. AlphaFold models were downloaded from https://alphafold.ebi.ac.uk/download and structural features extracted using bio3d.

### Peptide Sequence Analysis

Tile-plots were constructed using pheatmap_1.0.12. Neighbour-joining trees were constructed by aligning peptide sequences using “ClustalW” with the Gonnet substitution matrix using package msa_1.30.1, and aligned sequences were used to construct a matrix of pairwise distances, and neighbour-joining trees were constructed with 100 bootstrap replicates and midpoint-rooting using packages ape_5.7-1, phytools_1.5-1, and ggtree_3.2.1. Peptide logos were constructed using ggmsa_1.4.0 and ggseqlogo_0.1, and colored using the Taylor coloring scheme.

### Peptides

Peptides for all cell migration experiments were obtained from GenScript at >95% purity and were synthesized using Fmoc solid-phase synthesis to give peptides with a C-terminal amide. LC-MS data provided by the supplier show that HD2 peptide is at the expected molecular weight i.e., it is monomeric (Supplementary FIG. 10). Peptide sequences are provided in the data supplement Table 2. The HD2SCR sequence TLETDTFYECPDAYAY was designed by generating 50 random shuffles using the function “stri_rand_shuffle” (R-package stringi_1.7.6) and then selecting the peptide with the maximal “osa” string distance to wild-type using R-package stringdist_0.9.8.

### Protein Expression and Purification

HIS:SUMO: peptide plasmids were transformed into BL21 (DE3) cells (NEB) and grown in 5 mL LB+Kanamycin media overnight at 37° C., 200 rpm. 1% of this primary culture was inoculated into secondary culture and was grown for approximately 2 hours at 37° C., 200 rpm. At OD600 of 0.4, the culture was induced with 1 mM IPTG and grown for 4 hours at 37° C., with shaking at 200 rpm. The induced cells were harvested at 4000 rpm and the media was discarded. The pellet from a 200 mL culture was resuspended in 30 mL of Binding Buffer (PBS, 5 M NaCl, pH 7-7.2) containing 1 mM PMSF. The suspension was sonicated for 45 mins in ice using 0.7 sec on −0.3 sec off cycle at 40% power. The lysate was centrifuged at 10,000 rpm for 20 mins at 4° C. and the supernatant collected. 5 L of DNase I was added to the supernatant and incubated for 15 mins in ice. Supernatant was filtered and passed over IMAC Sepharose 6 Fast Flow column resin (pre-treated with 0.2 M NiCl₂ and equilibrated with binding buffer (PBS, 5 M NaCl pH 7-7.2)). The resin was incubated with the lysate supernatant for 1 hour at 4° C. at shaking conditions. 30 mL wash buffer (PBS, 500 mM NaCl, 20 mM Imidazole, pH 7-7.2) was applied to the resin. The peptide was then eluted in 6 mL Elution buffer (PBS, 500 mM NaCl, 500 mM Imidazole, pH 7-7.2), and buffer exchange in PBS was performed using Amicon Ultra Centrifugal Units. EVA4 was expressed in HEK293F cells and purified using nickel-charged IMAC Sepharose 6 Fast Flow resin (GE Healthcare) followed by size exclusion chromatography using methods known in the art.

### Biolayer Interferometry (BLI)

The binding of His-SUMO tagged peptide fusions to various chemokines (Supplementary information Table 3) was investigated using Ni-NTA biosensors (ForteBio). All BLI data were obtained at 25° C. using a ForteBio-Sartorius Octet RED 96e machine or a ForteBio-Sartorius Octet RED 384 machine. The biosensors were preincubated overnight at room temperature in BLI buffer (PBS, 500 mM NaCl, 0.01% BSA+0.002% Tween). 1 mg/ml His-SUMO tagged fusions were immobilised on the Ni-NTA biosensors using the BLI buffer. The HIS:SUMO fusion loaded biosensors were then incubated in the BLI buffer to allow signal stabilization. To study the association with the analyte (chemokines), the biosensors were then dipped in chemokine solutions of various concentrations (1 M for screening assay, a range of 10 nM-500 nM for kinetic assay) made in the BLI buffer for 300s or more. This was followed by the dissociation step, where the biosensors were incubated in BLI buffer for 300 s or longer. For chemokine cross-binding screens wavelength shift was normalised by subtracting the signal from buffer control, and normalising the highest Rmax (i.e., with CCL8 and HD2) obtained in the experiment to 1. Kinetic data was analysed using ForteBio Data Analysis HT 11.1 using 1:1, 1:2 and 2:1 binding models. We used fits where full R² (i.e., how well the fit and experimental data correlate)>0.95, full X² (i.e., measure of error between experimental data and fitted line) <3, and K_{D} standard error was less than one order of magnitude with respect to K_{D} (as recommended by the manufacturer). We calculated the mean and standard error of the K_{D} values obtained with at least three different doses for the different binding models. Data was visualised using R package ggplot2_3.4.2.

### Cell Migration Assays

The J:CXCR1 cell line was generated by transfecting Jurkat cells by electroporation with PvuI-linearized plasmid D1398 and selecting with blasticidin (Sigma-Aldrich, Cat #203350, 5 g/mL). CXCR1 expression was confirmed by labelling cells with anti-CXCR1 antibody and quantifying expression using flow-cytometry (data not shown). J:CXCR1 cells were cultured in RPMI-1640 media (Gibco), supplemented with 10% FBS and 5 mM L-Glutamine with blasticidin. For J:CXCR1 migration assays, 300,000 cells/well were added to the top chamber of a 3 μm Transwell insert (Corning) in 50 μL of migration media (RPMI-1640, 0.5% FBS, 4 mM L-Glutamine, 0.05% DMSO). The bottom chamber contained 150 μL of chemokine, with or without peptide. Cells were migrated at 37° C. in 5% CO₂ for 4 hours. The plate was incubated on a shaking platform for 10 minutes, 800 rpm, at 37° C., and 150 μL from the bottom of the plate transferred into a U-bottom plate containing 50 μL of migration media. Cell counts were determined by Attune NxT Flow Cytometer Plate Reader (ThermoFisher), based on cell size parameters FSC-H/SSC-H. Experiments were performed in 3 technical and 3 biological replicates. THP-1 migration assays were carried out using methods known in the art. Activated T-cell assays were carried out using methods known in the art, with the following modifications in the isolation process. Peripheral blood cells were obtained from leucocyte cones (NHS Blood Transfusion Services), following which, T-cells were isolated using two rounds of human CD8+ T-cell isolation kit (480011, BioLegend). At day 10 after activation, T-cells were frozen at 20 E6 cells/ml in TexMACS medium supplemented with 10% DMSO (D2650, Sigma) in liquid nitrogen. Prior to use, activated T-cells were recovered for 24 hours in TexMACs medium at 0.3 E6 cells/ml and incubated at 37° C. in 5% CO2. Chemokine sources are described in Supplementary information Table 3. Statistical significances between control experimental groups was evaluated using Dunnett's test. IC50 experiments were performed at the EC₈₀ dose of chemokine. EC₈₀ was calculated by fitting a chemokine dose-response curve with 3 parameters (fixing the top to 100%). IC₅₀ was calculated by fitting an inhibitor response curve with 4 parameters. All IC50 reported had p-value <0.05.

Modelling of Chemokine Complexes with Peptides, Receptors and Evasins

Models were generated using AlphaFold2-Multimer at the COSMIC² Science Gateway using default parameters and a full database search using UniProt mature protein sequences. The five highest confidence models were identified in each case from the confidence score and carried forward for heat map generation. As experimental structures often contain missing sidechain atoms, direct use of these structures will interfere with the atom-wise scoring function of AutoDock CrankPep (ADCP), and, also, as certain chemokines e.g., CXCL6, lack PDB structures, to maintain consistency, we first generated chemokine structures using AlphaFold for input to ADCP. The highest ranking structure for each chemokine was reduced with reduce (https://github.com/tlabduke/reduce), then the functions prepare_receptor, agfr, and adcp were called from ADFRsuite 1.0 (https://ccsb.scripps.edu/adcp/downloads/). Parameters were default for prepare_receptor and agfr, while adcp was run with N=300, n=48000000 and nc=0.8 for all runs. For ADCP the ten highest ranked poses (based on automatically calculated lowest free energy) were carried forward for heat map generation. Binding site heatmaps were generated by identifying residues within 5 Å distance of the two chains using bio3d for each docking pose. Weighted proximity scores were calculated as follows: A per-residue-score equal to the confidence score (AlphaFold) or calculated free energy (ADCP) was ascribed for each pose within a model, this was aggregated over the different poses in the model, and then normalized to a maximum score of 100 to allow comparison between models. Packages used were Python 2.7 and 3.9.2 and Biopython 1.79. Open-Source PyMOL (https://pymolwiki.org/index.php/MAC_Install) was used for scripting and PyMOL 2.5.2 (https://pymol.org/2/) for visualization of models. ADCP was run at the Oxford University BioMedical Research Computing Cluster (BMRCC).

### Example 19. NNK Mutagenesis Library Construction and Screening

The exemplar peptide HD2, has been found to have the surprising ability to bind 21 chemokines from CC and CXC/X3C (referred to as “XC”) classes in phage display, and to inhibit cell migration in response to CCL2, CCL3, CCL5, CCL7, CCL8, CCL23, CXCL10, CXCL11 and CXCL6. To identify mutations that improved breadth and affinity of binding, a library of HD2 mutations was generated that had NNK substituted at each residue and phage-display selection was performed against a panel of 24 biotinylated chemokines, in parallel. Following library selection with each chemokine, the enrichment (E) of each peptide was calculated in comparison to the input library, and expressed the enrichment as log 2 E, as this metric is correlated with binding affinity. This strategy allowed us to evaluate the impact of Alanine, conservative, hydrophilic and hydrophobic substitutions, and helped define the HD2 pharmacophore.

### Example 20. Identification of Mutations that Improve Chemokine Binding

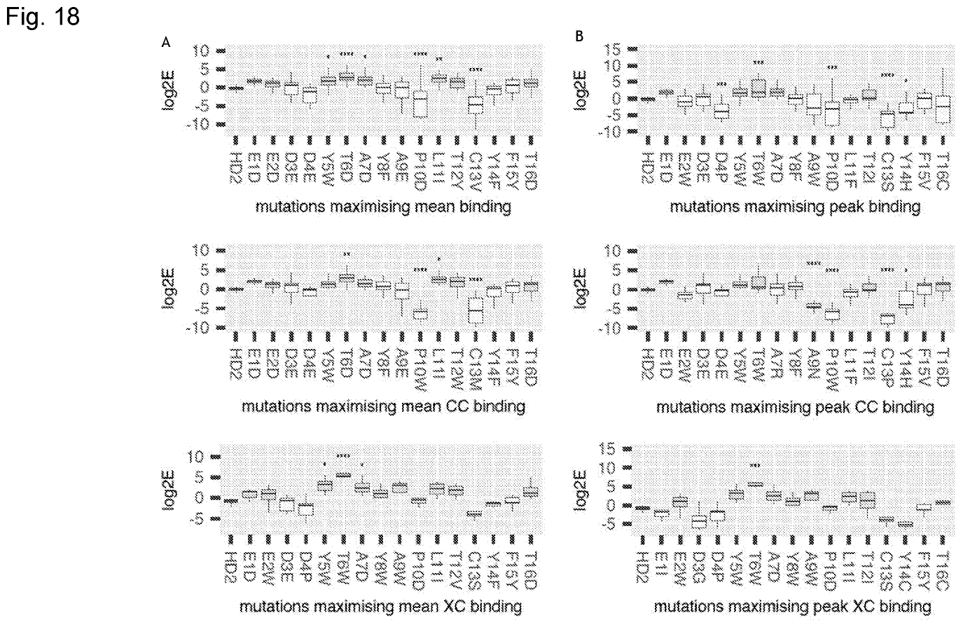

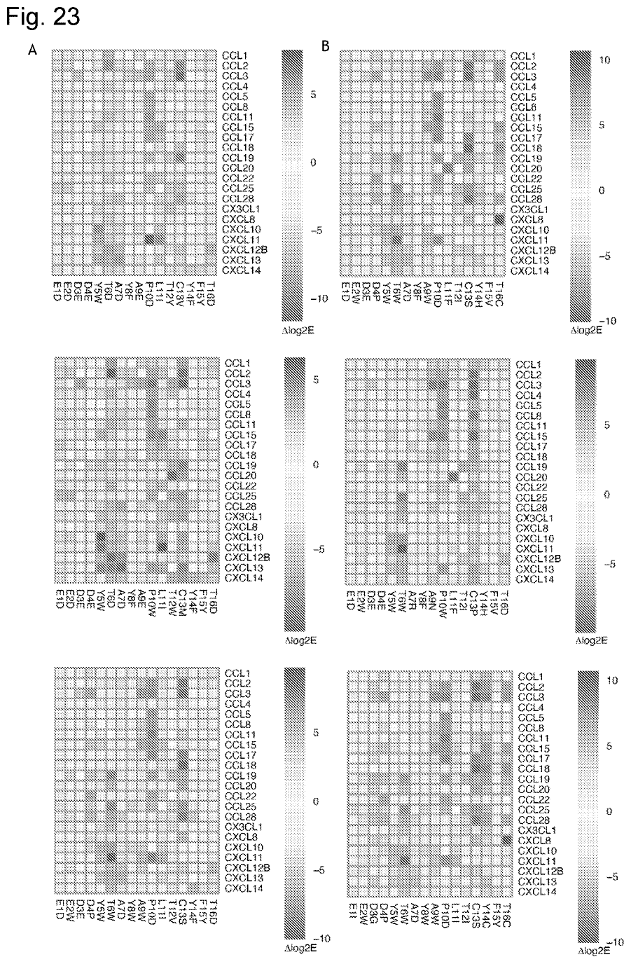

A two-pronged strategy was used to identify individual peptides with mutations that improve chemokine binding over the parental peptide HD2. To identify mutations that improve overall chemokine binding affinity, the best performing mutations were selected at each residue by their enhancement of mean log 2 E (FIG. 2a, Supplementary FIG. 1a). To add in mutations that improve chemokine binding diversity, the best performing mutations at each residue were identified by their enhancement of peak log 2 E (FIG. 18B, FIG. 23B). For mutations selected by the first strategy, Y5W, T6D, A7D, and L11I showed significant improvement when considered over all chemokines (FIG. 18A, top panel), T6D and L11I when considered over CC chemokines (FIG. 18A, middle panel), and Y5W, T6W and A7W when considered over XC chemokines (FIG. 18A, bottom panel). Several other mutations (e.g., E1D, E2D) showed enhancement over the parental that was not statistically significant. For mutations selected by the second strategy, several mutations—e.g., T16C, T12I, P10D—were identified that had the highest peak log 2 E at that residue. Notably, the log 2 E for the parental HD2 was ~0 in these analyses, indicating lack of enrichment, the most likely explanation of this result is that mutant peptides with enhanced binding out-compete the parental HD2 peptide.

### Example 21. Effect of Single Mutations on Chemotaxis

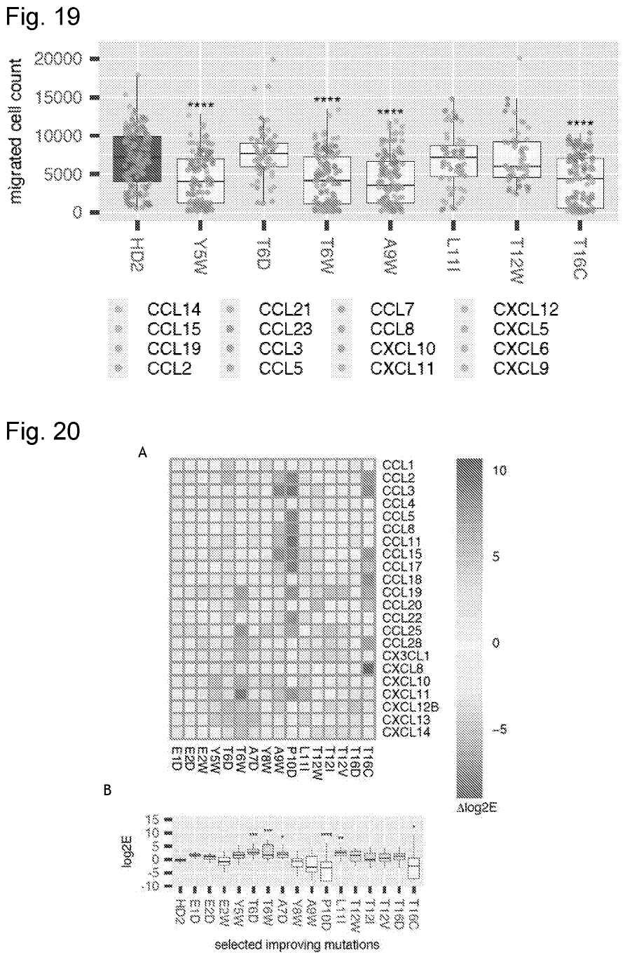

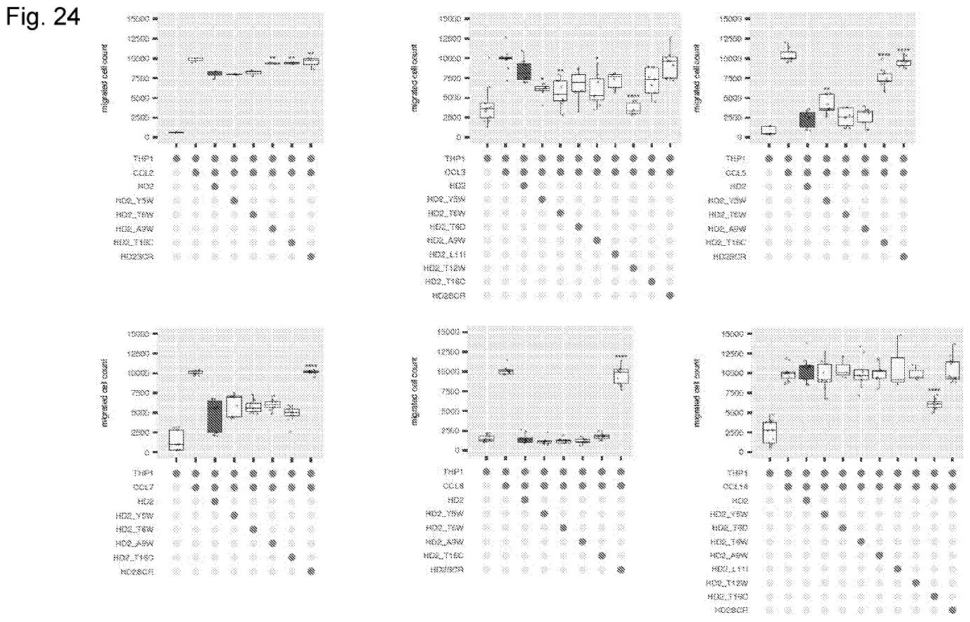

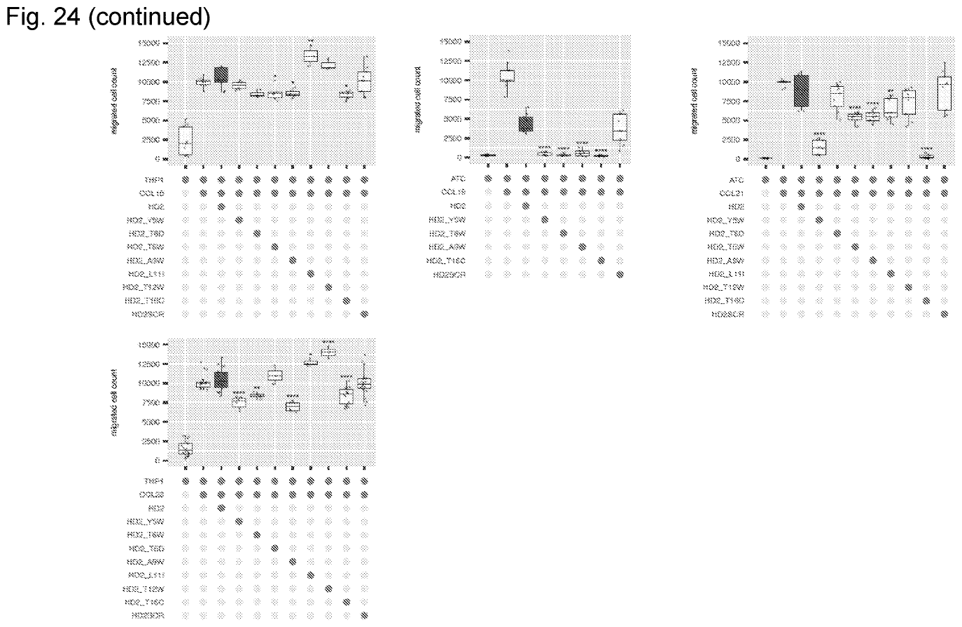

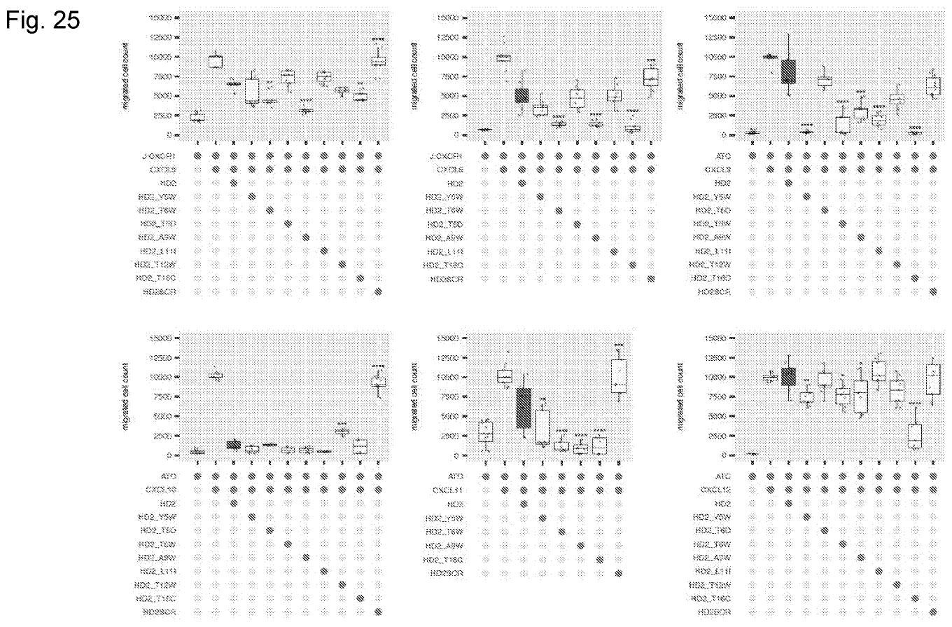

The effect of certain selected HD2 mutations identified above on chemotaxis was studied using nine CC and six CXC chemokines (FIGS. 23 and 24) and was compared with the parental HD2 peptide in each case. A significant reduction in chemotaxis was observed on chemotaxis induced by CCL3 (T12W), with T16C on CCL14 (T16C), CCL15 (T6W, A9W, T16C), CCL21 (Y5W, T6W, A9W, L11I, T16C), CXCL5 (T6W, A9W, T16C), CXCL6 (T6W, A9W, T16C), CXCL9 (Y5W, T6W, A9W, L11I, T16C), CXCL11 (Y5W, T6W, A9W, T16C), and CXCL12 (Y5W, A9W, T16C). No further improvement of inhibitory capacity was observed with any mutant on CCL8 or CXCL10. The mutation T6D did not improve chemotaxis in any assay. A meta-analysis of these results is shown in FIG. 19, and to summarize, the most effective individual mutations with significant improvement over HD2 are Y5W, T6W, A9W, and T16C.

### Example 22. Combinatorial Mutagenesis Library Construction and Screening

The results in Examples 19-21 indicated that it is possible using phage-display saturation mutagenesis to improve the chemokine-binding properties of a peptide identified from an evasin protein that has already evolved chemokine-binding function by natural selection in ticks. Combinations of different mutations identified by saturation mutagenesis selection were then studied to see if they could result in additive or cooperative effects, allowing further evolution of peptide function in a desired direction. Traditionally, the selection of two or more mutations in combination is guided by knowledge of the structure of the protein-peptide interface. This approach works well when designing agents against a single target where the interface structure has been experimentally characterised but is challenging when designing agents active against multiple targets, where the interface structures have not been experimentally characterised. To systematically identify mutation combinations that work well against multiple structurally related targets we explored the possibility of using phage-display to select combinations of mutations that bind CC or XC classes of chemokines. Using a strategy based on Δlog 2 E (where Δlog 2 E is the change in log 2 E compared to parental HD2, see Example 27 for the algorithm applied), 16 “improving” mutations at 11 different residues were identified. A tile-plot of the Δlog 2 E data for each of these mutations is shown in FIG. 20A. A library encoding all possible (i.e., 3585) combinations of these “improving” mutations (FIG. 20B) was constructed. The library contained in addition the parental HD2, and the 16 single mutations. Phage-display selection of the combinatorial library against a panel of 25 biotinylated chemokines was performed (FIG. 20B).

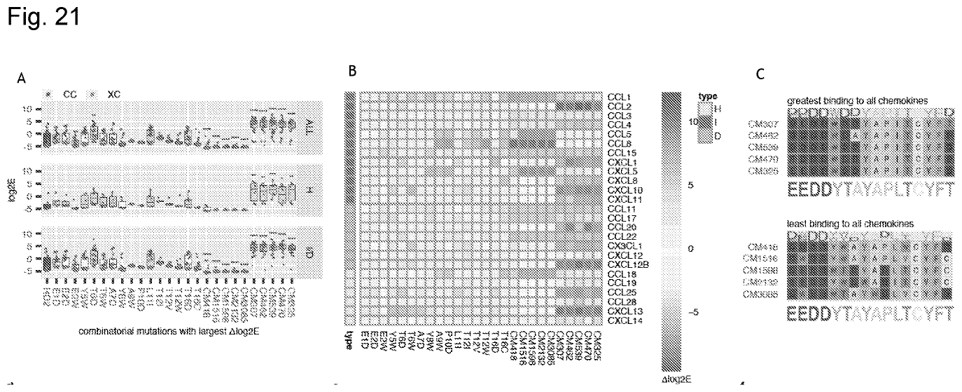

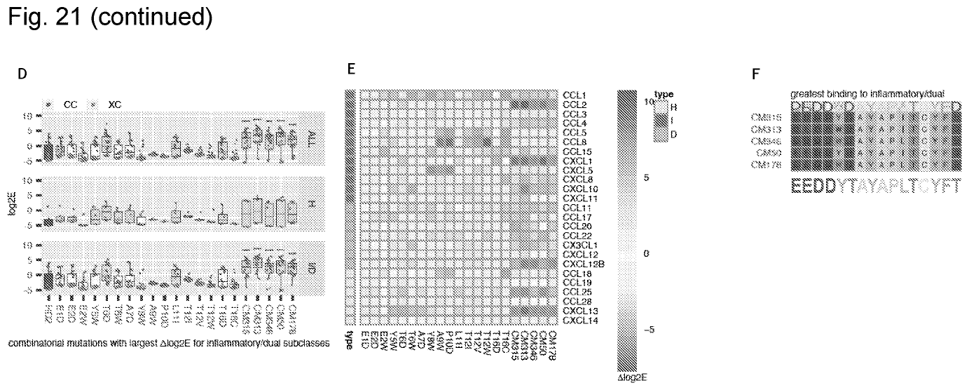

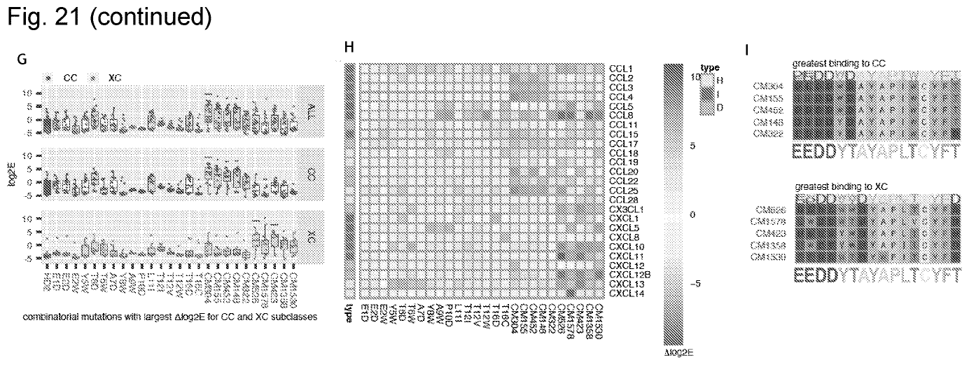

Δlog 2 E to the parental HD2 peptide was calculated for each single and combinatorial mutation and the five mutants with the highest and the five with the lowest median Δlog 2 E were identified. The log 2 E values for these 10 combinatorial mutants and for the single mutations and parental HD2 are shown in FIG. 21A, and a tile plot of Δlog 2 E values in FIG. 21B. The combinatorial mutants with highest median Δlog 2 E (CM307 to CM325) all have significantly greater log 2 E in comparison to the parental HD2, whereas combinatorial mutants with lowest median Δlog 2 E (CM418 to CM3085) have significantly lower log 2 E in comparison to the parental HD2. Notably, the combinatorial mutants with highest median Δlog 2 E (CM307 to CM325) have higher median log 2 E compared to any of the single mutants. These results indicate that certain combinations of “improving” single mutations may be surprisingly deleterious, and that phage display is an efficient way of identifying the best “improving” mutation combinations.

### Example 23. Sequence Analysis of Combinatorial Mutations Affecting Chemokine Binding

The worst and the best performing combinatorial mutation peptide sequences were aligned (FIG. 21C). No similarity between the worst performing sequences was identified except at residues D3, D4, and C13-F15, which had not been mutated in the combinatorial library. In contrast the best performing five peptides were very similar, with uniform replacement at Y5W, T6D, L11I and variable replacements at E1D, E2D, A7D, and T16D, suggesting that these mutations can act cooperatively, despite some of them (E1D, E2D, T16D) only having small and non-significant enhancement in individual experiments (FIG. 18). The mutation T16C was not present in the best five peptides but was frequently present in the worst five peptides suggesting that it does not cooperate effectively, at least in the context of phage-display. The numbers of mutations in the best performing five peptides range from 5 substitutions (CM307) to 7 substitutions (CM539). These mutations have significantly enhanced binding to both homeostatic and inflammatory/dual chemokines (FIG. 5a).

### Example 24. Identification of Combinatorial Mutants Selectively Affecting Binding to Homeostatic and Inflammatory/Dual Function Chemokines

Therapeutic development may require the specific targeting of inflammatory/dual functions and minimize targeting of homeostatic functions. It was explored whether combinatorial mutants that have such properties may be identified from the dataset. Certain combinatorial mutations (CM315-CM178, FIG. 21D-21F) were found have small and non-significant effect on homeostatic chemokine binding yet retain significant effect binding to inflammatory/dual function chemokines. These combinatorial mutants are distinguished from the series CM307-CM425 (see above) by the retention of the wild-type residue A at position 7 and suggest that a single residue change at this position could be important in affecting binding to homeostatic chemokines. A limitation of this analysis is that there are only 6 purely homeostatic chemokines that were analysed.

### Example 25. Identification of Combinatorial Mutants Selectively Affecting Chemokine Binding

As CC and XC chemokines have distinctive quaternary structures, we next examined the data to identify mutation combinations that are relatively selective for CC-chemokine and for XC-chemokine binding. The five best combinations in each category were identified. Log2 E values for these 10 combinatorial mutants and for the single mutations and parental HD2 are shown in FIG. 21G, and a tile plot of Δlog 2 E values in FIG. 21H. Four of five combinatorial mutants with highest median Δlog 2 E for CC-chemokines (CM304 to CM322) have significantly greater log 2 E in comparison to the parental HD2 (FIG. 21G, middle panel), and are not significantly enhanced for XC chemokines (FIG. 21G, bottom panel). Four of five combinatorial mutants with highest median Δlog 2 E for XC-chemokines (CM526 to CM1530) have significantly greater log 2 E in comparison to the parental HD2 (FIG. 21G, bottom panel), and are not significantly enhanced for CC chemokines (FIG. 21G, middle panel). This distinction in CC versus XC binding is clearly visible also in the tile-plot (FIG. 21H). These results indicate that the combinatorial mutagenesis strategy can identify mutation combinations that directionally enhance CC- or XC-chemokine binding. We next aligned the CC and XC-selective combinatorial mutation peptide sequences (FIG. 1I). The CC-binding sequences are characterised by constant substitutions of T6D, L11I and T12W, and variable substitutions EID, E2D, and Y5W. The XC-binding sequences are characterised by constant substitutions A7D, L11I and T16D, and the variable substitutions E1D, E2D, E2W, Y5W, T6W, and T12V. Thus, it appears feasible to identify combinatorial mutations that distinguish CC and XC-class chemokines.

### Example 26. Functional Analysis of Combinatorial Mutations

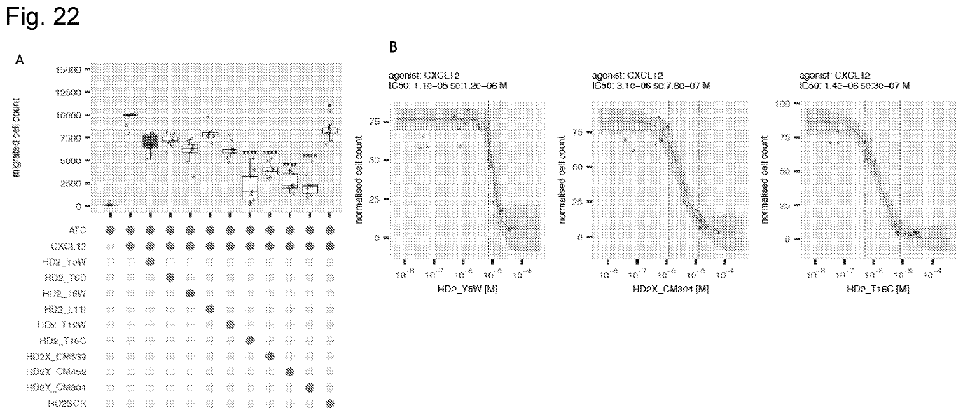

Whether the combinatorial mutations would enhance biological activity was then determined. The effect of combinatorial mutations CM304, CM452 and CM539 on the biological activity of CXCL12 was examined, which showed enhanced binding in phage display (FIG. 21B, 21H). Activity of these mutants was compared against the single mutant HD2_Y5W as negative control as this mutation was present in all three combinatorial mutations (FIG. 22). All three mutations displayed significantly enhanced activity in inhibiting migration of activated T cells in comparison to HD2_Y5W. This also confirmed the results observed previously with HD2_T16C being identified as a potent inhibitor. This was explored further using dose-response experiments (FIG. 22B) which showed that both CM304 and HD2_T16C had improved potency from 11 mM for HD2_Y5W to 3.1 mM for CM304 and 1.4 mM for HD2_T16C. These experiments show that the COSMOS method can not only identify improved chemokine binders but also that these binders have improved potency. The dose-response experiments showed that the single mutation T16C has unexpectedly potent activity that is not predicted by change in log 2 E compared to the parental HD2 in phage-display.

### Example 27. Methods for Examples 19 to 26

### R Package Versions

Bioinformatic and statistical analyses were performed using R version 4.2.2, tidyverse_2.0.0, DescTools_0.99.48, ggupset_0.3.0, ggplot2_3.4.1, pheatmap_1.0.12, seqinr_4.2-23, ggmsa_1.4.0, and ggseqlogo_0.1.

### Saturation Mutagenesis Phage Display Library Design, Construction and Screening

81-mer mutant oligonucleotides replacing NNK at each of the 16 peptide encoding residues were designed with the sequence arms 5′-GCAGCCTCTTCATCTGGC, and GGTGGAGGATCCGGA-3′ at respective ends to enable amplification and cloning. Oligonucleotides were individually amplified and cloned into plasmid prSTOP4Nsi (kind gift from Dr Sachdev Sidhu, University of Toronto) and phage display screening performed using biotinylated chemokines attached to streptavidin matrix as described previously. Inserts from the input library and from the final phage population following each chemokine selection were amplified by PCR and sequenced at Azenta/Gene Wiz using the AmpliconEZ protocol. Sequences were analyzed as described previously. Hexadecapeptide enrichment (E) following selection was calculated as ratio of output peptide frequency to input peptide frequency and expressed as log 2 E. Where proportion of count in output was not available it was replaced with the lowest output proportion in the experiment rather than zero, which would result in a-Infinity log 2 E. Δlog 2 E was calculated as the difference between log 2 E of peptide and that of parental HD2. Peptides that showed binding to control (C5a, log 2 E>0) were excluded from downstream analysis. R-packages used for design and analysis were R version 4.1.2, tidyverse_1.3.1, strex_1.4.2, ggplot2_3.3.5, ShortRead_1.5.2 and Biostrings_2.62.0.

### Combinatorial Mutagenesis Library Design and Screening

As CC and XC binding peptides appeared to have a different improving mutation spectrum we selected mutations using an algorithm that optimizes CC and XC improving mutations separately. Briefly, we identified at each peptide position the mutation giving highest mean Δlog 2 E and the highest peak Δlog 2 E. We took the following steps to limit the numbers of selected mutations. We set a threshold 5 for peak Δlog 2 E, and a threshold of 0.55 for mean Δlog 2 E, and then selected the top 10 mutations (arranged by mean Δlog 2 E and peak Δlog 2 E) for CC and top 12 mutations for XC chemokines. This resulted in a total of 16 mutations at 11 residues. We generated all possible combinations of the 10 CC-enhancing, and 12 XC-enhancing mutations using the R function “utils::combn”, pooled the output, added the parental and scrambled parental sequence, and reverse translated the peptides using R package reversetranslate_1.0.0, with ecoli_tbl and model=“gc_biased” to generate 3585 oligonucleotide sequences. The oligonucleotides were synthesized as a pool (Genscript, 12K chip) and were cloned into plasmid prSTOP4 as described previously. The library quality was assessed by NGS AmpliconEZ (50,000 reads), and 3584 of 3585 inserts were identified. The library was screened using the phage-display protocol described above.

### Biolayer Interferometry (BLI)

The binding of His-SUMO tagged peptide fusions to chemokines (Supplementary information Table 3) was investigated using Ni-NTA biosensors (ForteBio) as described previously.

### Cell Migration Assays

Migration assays using THP1, activated T-cell (ATC) and J:CXCR1 (Jurkat cells stably transfected with CXCR1 receptor) were performed as described previously. IC50 experiments were performed at the EC₈₀ dose of chemokine. EC₈₀ was calculated by fitting a chemokine dose-response curve with 3 parameters (fixing the top to 100%). IC50 was calculated by fitting an inhibitor response curve with 4 parameters. R-packages used were ggupset_0.3.0, DescTools_0.99.44, and drc_3.0-1.

### Statistical Information

We performed statistical analyses using the R-base package stats and DescTools_0.99.48. We used one-way ANOVA followed by Dunnett's post-hoc multiple comparison procedure for comparing several treatments with a control, with 95% family-wise confidence level. Tukey-style box-whisker plots were constructed using the ggplot function geom_boxplot, which displays median, lower and upper hinges (25th and 75th percentiles), and whiskers from hinge to 1.5* interquartile range.

### Example 28—Peptibody Comprising HD2

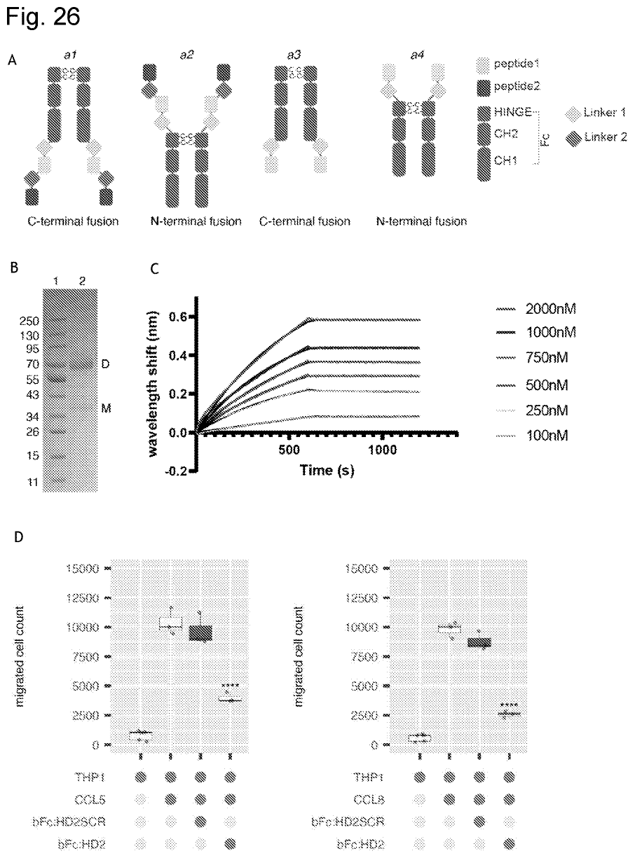

The concepts of peptibody design and production are well established and are shown in FIG. 26 (see also Shimamoto et al., MAbs, 2012. 4 (5): p. 586-91; Cavaco et al., Biopolymers, 2017). Briefly, biologically active peptides (peptide 1 and peptide 2 in diagram) may be converted to peptibodies by genetically fusing them to either the C-terminus or the N-terminus of the Fc fragment of immunoglobulins such as IgG1 or IgG4, using one or more linkers. The Fc fragment may contain mutations that improve pharmacokinetics or alter glycosylation. Dimeric species are created by disulfide (indicated as C—C) bonds between the hinge region of the monomers. Peptide 1 and peptide 2 may be identical peptides with identical chemokine binding properties. Alternatively, peptide 1 and peptide 2 may be non-identical peptides with differing chemokine-binding properties allowing tailoring of specificity to the disease chemokine-expression pattern. Peptibodies may be produced in a variety of expression systems e.g. mammalian, bacterial or baculoviral. Peptibodies were constructed using the design shown in Figure al (IgG1Fc:linker1:peptide1:linker2:peptide2), where peptide 1 and peptide 2 positions were replaced by HD2 to generate bFc:HD2, and by HD2SCR to generate bFc:HD2SCR. HD2SCR is a scrambled version of HD2 and serves as negative control. Linker1 is 5 glycine residues, linker2 is eight glycine residues. The constructs were cloned into a pET backbone vector, expressed in E. coli BL21DE3 and purified using Protein-A-sepharose affinity matrix and size-fractionation. SDS-PAGE of bFc:HD2 peptibody (FIG. 26B, lane 2) showing dimeric (D) and monomeric (M) species observed under non-reducing conditions. Molecular weight marker (FIG. 26B, lane 1) sizes are shown in KDa. A biolayer interferometry dose-response sensorgram showing CCL8 (immobilised to protein-A biosensor) binding to indicated doses of bFc:HD2 is shown in FIG. 26C. FIG. 26D shows the effect of bFc:HD2 peptibody on THP1 cell migration induced by indicated human chemokines.

The data demonstrate that a peptibody may be used as a scaffold for a combinatorial peptide entity comprising chemokine-binding peptides that are capable of binding to chemokine and inhibiting chemotaxis induced by various chemokines.

### Example 29—Mutagenesis of HD845

NNK mutagenesis library construction and screening was performed for the HD845 peptide using the methods described in Example 19. To identify mutations that improved breadth and affinity of binding, a library of HD2 mutations was generated that had NNK substituted at each residue and phage-display selection was performed against a panel of 25 biotinylated chemokines (15 CC-class chemokines, 10 CXC-class chemokines). Following library selection with each chemokine, the enrichment (E) of each peptide was calculated in comparison to the input library, and expressed the enrichment as log 2 E, as this metric is correlated with binding affinity.

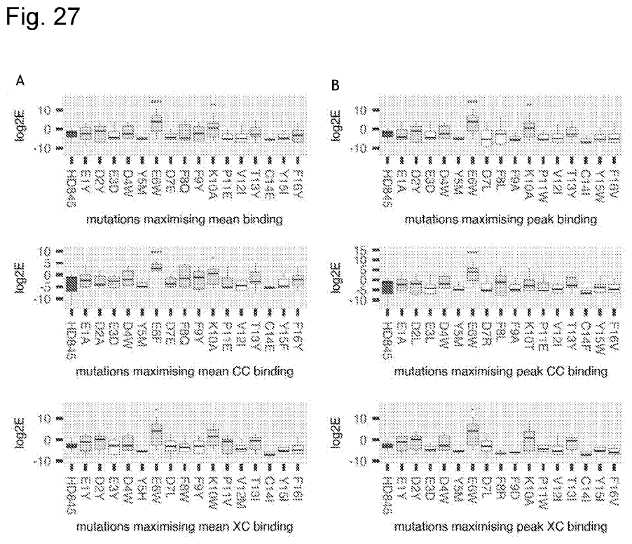

A two-pronged strategy was used to identify individual peptides with mutations that improve chemokine binding over the parental peptide HD845. To identify mutations that improve overall chemokine binding affinity, the best performing mutations were selected at each residue by their enhancement of mean log 2 E (FIG. 27A). To add in mutations that improve chemokine binding diversity, the best performing mutations at each residue were identified by their enhancement of peak log 2 E (FIG. 27B). For mutations selected by the first strategy, E6W and K10A showed significant improvement when considered over all chemokines (FIG. 27A, top panel), E6F and K10A when considered over CC chemokines (FIG. 27A, middle panel), and E6W when considered over XC chemokines (FIG. 27A, bottom panel). Several other mutations (e.g., E1Y, DIY, D4W) showed enhancement over the parental that was not statistically significant. For mutations selected by the second strategy, several mutations—e.g., E6W and K10A—were once again identified and had the highest peak log 2 E at that residue.

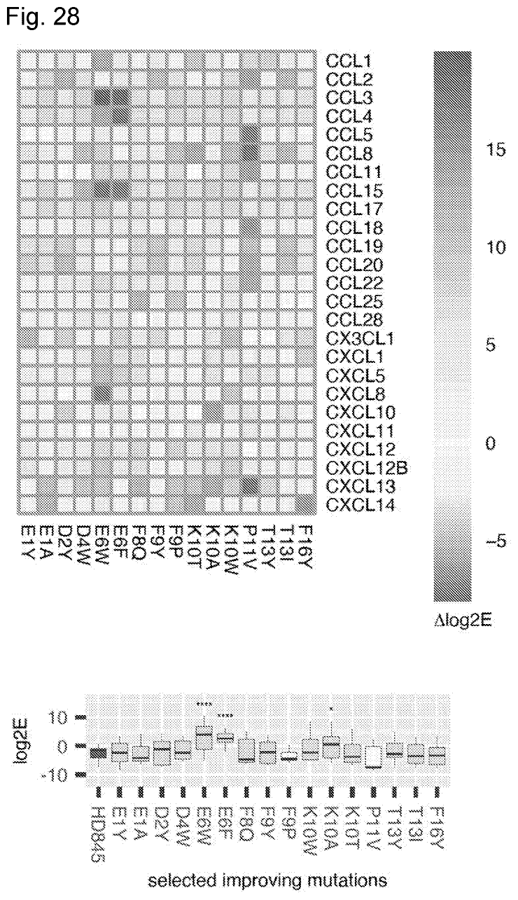

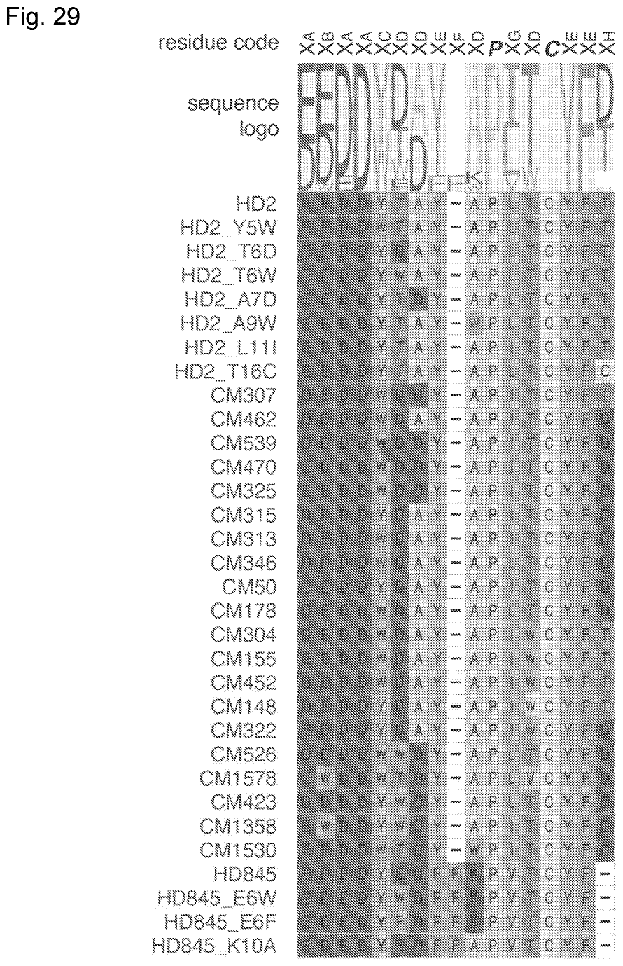

FIG. 28 shows the impact of selected improving mutations over all chemokines studied.

### Tables

TABLE 4
Exemplary Chemokines Expressed In Disease, (Bhattacharya,
2020) (Blanco-Melo, 2020) (Gao, 2013) (Mosevoll, 2018)

| Disease | Chemokines |
|---|---|
| Acute lung injury | CCL2, CCL7, CXCL5, GROA, IL8 |
| Alcoholic liver injury | CCL2, CCL20, CXCL5, CXCL6, CXL10, GROA, IL8, PLF4 |
| Alzheimer's disease | CCL2, CCL3, CCL5, CXL10, IL8 |
| Atherosclerosis | CCL1, CCL11, CCL13, CCL15, CCL17, CCL18, CCL19, |
| | CCL2, CCL20, CCL21, CCL22, CCL23, CCL25, CCL3, CCL4, |
| | CCL5, CCL8, CXCL9, CXL10, CXL11, CXL13, CXL16, IL8, |
| | SDF1, X3CL1 |
| Atopic dermatitis | CCL1, CCL11, CCL13, CCL17, CCL18, CCL2, CCL20, |
| | CCL22, CCL26, CCL27, CCL3, CCL4, CCL5, X3CL1 |
| Autoimmune hepatitis | CCL26, CXCL9, CXL10, SDF1 |
| Breast cancer | CCL18, CCL2, CCL20, CCL4, CCL5, CXCL3, CXCL5, |
| | CXCL6, GROA, IL8 |
| Colorectal cancer | CCL16, CCL20, CCL7, CXCL7, SDF1 |
| Cytokine Storm: Covid-19 | CCL11, CCL18, CCL19, CCL2, CCL3, CCL4, CCL7, CCL8, |
| | CXL10, CXL16, CXL17 |
| Cytokine Storm: Influenza | CCL16, CCL17, CCL18, CCL2, CCL20, CCL26, CCL3, CCL4, |
| | CCL5, CCL7, CXCL2, CXCL9, CXL10, CXL11, IL8 |
| Cytokine Storm: Sepsis | CCL2, CCL20, CCL3, CCL4, CXL10, CXL11, GROA, IL8 |
| Idiopathic pulmonary fibrosis | CCL11, CCL13, CCL17, CCL18, CCL19, CCL2, CCL20, |
| | CCL22, CCL3, CCL4, CCL5, CCL7, CCL8, CXCL2, CXCL3, |
| | CXCL5, CXCL6, CXCL9, CXL10, CXL13, CXL14, GROA, |
| | IL8, SDF1 |
| Inflammatory bowel disease | CCL11, CCL14, CCL15, CCL2, CCL20, CCL25, CCL3, CCL4, |
| | CCL5, CCL7, CCL8, CXCL2, CXL10, GROA, IL8, SDF1, |
| | X3CL1 |
| Liver fibrosis | CCL1, CCL2, CCL3, CCL5, CXL10, CXL16, GROA, PLF4 |
| Multiple sclerosis | CCL2, CCL3, CCL4, CCL5, CXCL9, CXL10, CXL13, SDF1 |
| Myocardial infarction | CCL11, CCL13, CCL2, CCL21, CCL3, CCL4, CCL5, CXL10, |
| | CXL16, IL8, SDF1 |
| Myocarditis | CCL13, CCL17, CCL18, CCL19, CCL2, CCL20, CCL5, |
| | CXCL9, CXL10, CXL16, IL8, SDF1, X3CL1 |
| Myositis | CCL11, CCL19, CCL2, CCL20, CCL21, CCL3, CCL4, CXCL9, |
| | CXL10, CXL13, IL8 |
| Nonalcoholic steatohepatitis | CCL2, CCL5, CXCL9, CXL10, IL8 |
| Pancreatitis | CCL2, CXCL5, CXCL9, IL8 |
| Paracetamol liver injury | CCL2, CCL5, CXCL2, CXL16, GROA, IL8 |
| Primary biliary cirrhosis | CCL2, CCL20, CCL26, CCL28, CXCL9, CXL10, CXL13, |
| | CXL16, IL8, SDF1, X3CL1 |
| Primary sclerosing cholangitis | CCL25, CCL28, CXL16, SDF1 |
| Psoriasis | CCL18, CCL19, CCL2, CCL20, CCL22, CCL4, CCL5, CCL7, |
| | CXCL2, CXCL5, CXCL9, CXL10, CXL11, CXL16, GROA, |
| | IL8, SDF1, X3CL1 |
| Rheumatoid arthritis | CCL13, CCL14, CCL15, CCL16, CCL17, CCL18, CCL19, |
| | CCL2, CCL20, CCL21, CCL28, CCL3, CCL5, CCL7, CCL8, |
| | CXCL5, CXCL6, CXCL7, CXCL9, CXL10, CXL13, GROA, |
| | IL8, PLF4, SDF1, XCL1, NA |
| Sarcoidosis | CCL15, CCL16, CCL18, CCL19, CCL2, CCL20, CCL24, |
| | CCL3, CCL5, CXCL9, CXL10, IL8 |
| Skin fibrosis | CCL17, CCL2, CCL22, CCL27, CCL3, CCL5, CCL7, CXL16, |
| | GROA, IL8 |
| Stroke | CCL2, CCL20, CCL21, CCL3, CCL7, CXCL5, GROA, IL8 |
| Vasculitis | CCL13, CCL17, CCL18, CCL19, CCL2, CCL20, CCL21, |
| | CCL3, CCL4, CCL5, CXCL2, CXCL6, CXCL9, CXL10, |
| | CXL11, CXL13, CXL16, GROA, IL8, X3CL1, XCL1 |

TABLE 5A
Phage-Display Screen Peptides Identified Using Chemokines Displayed on
Streptavidin Beads; log2E greater than 5. Chemokines are referred to by the first part of the
UniProt Entry Name. Peptide source indicates the UniProt entry name of the originating
protein, nature (WT = wild-type, CA = Cys to Ala mutation, CS = Cys to Serine mutation), and
residue location. Peptides binding to complement C5a (control) were excluded.

| SEQ | | | | |
|---|---|---|---|---|
| ID | Peptide | Peptide | | Chemokines |
| NO. | ID | Sequence | Peptide Source | Bound |
| 1 | CK1301 | FIADYFETSS | CL3L1_HUMAN: WT: 47: 62 | CCL20, CXL10, |
| | | QCSKPS | | CXL14, IL8 |
| 2 | CK1408 | FSRCCFSFA | CCL1_HUMAN: WT: 30: 45 | CCL1, CCL2, |
| | | EQEIPLR | | CCL8, CXL14 |
| 3 | CK1445 | FVVDYYETS | CC4L_HUMAN: WT: 47: 62, | CCL19, CCL2, |
| | | SLCSQPA | CCL4_HUMAN: WT: 47: 62 | X3CL1, SDF1, |
| | | | | IL8 |
| 4 | CK1982 | IFKTIVAKEI | CCL2_HUMAN: WT: 65: 80 | CCL1, CCL22, |
| | | CADPKQ | | CCL5, X3CL1, |
| | | | | CXL10, CXL13 |
| 5 | CK2307 | IVRNFHYLLI | CCL19_HUMAN: CS: 42: 57 | CCL19, CCL20, |
| | | KDGSRV | | CCL22 |
| 6 | CK3115 | LLESYFETN | CCL23_HUMAN: WT: 67: 82 | CCL19, CCL22, |
| | | SECSKPG | | X3CL1, CXL14 |
| 7 | CK3274 | LRCVCLQTT | CXCL5_HUMAN: WT: 47: 62 | X3CL1, CXL10, |
| | | QGVHPKM | | CXL11 |
| 8 | CK3750 | PEWVNTPST | CCL16_HUMAN: WT: 28: 43 | CCL18, CCL19, |
| | | CCLKYYE | | CCL22, X3CL1 |
| 9 | CK4643 | RITNIQCPKE | CCL8_HUMAN: WT: 53: 68 | CCL1, CCL19, |
| | | AVIFKT | | CCL20 |
| 10 | CK576 | CADPSESWV | CC4L_HUMAN: WT: 74: 89, | CCL17, CCL5, |
| | | QEYVYDL | CCL4_HUMAN: WT: 74: 89 | CXL10 |
| 11 | CK6066 | VCADPSESW | CC4L_HUMAN: WT: 73: 88, | CCL19, CCL28, |
| | | VQEYVYD | CCL4_HUMAN: WT: 73: 88 | X3CL1, IL8 |
| 12 | CR10008 | ITLFASDFVL | VGCR_HHV6Z: CA: 74: 89, | CCL1, CCL22, |
| | | MWAVFF | UL33_HHV6U: CA: 70: 85 | CXL10, CXL11 |
| 13 | CR1003 | ALAFIYVLM | VG74_SHV21: CA: 41: 56 | CCL17, CCL18, |
| | | FLANAIG | | CCL19, CCL20, |
| | | | | CCL28, X3CL1, |
| | | | | CXL10, SDF1, |
| | | | | IL8 |
| 14 | CR1004 | ALAFIYVLM | VG74_SHV21: WT: 41: 56 | CCL17, CCL18, |
| | | FLCNAIG | | CCL19, CCL20, |
| | | | | CXL10, CXL14 |
| 15 | CR1005 | ALAFIYVLM | VG74_SHV21: CS: 41: 56 | CCL19, CCL2, |
| | | FLSNAIG | | CCL20, CCL28, |
| | | | | CXL14 |
| 16 | CR10175 | IVTIIITFFLS | CML1_HUMAN: CS: 262: 277 | CCL1, CCL17, |
| | | WSPYH | | CCL18, CCL20, |
| | | | | CCL22, CCL25, |
| | | | | CCL28, X3CL1, |
| | | | | CXL10, CXL11, |
| | | | | CXL14 |
| 17 | CR10275 | IYLLNLAISD | CCR5_HUMAN: WT: 67: 82 | CCL17, CCL18, |
| | | LFFLLT | | CCL25, CXL10, |
| | | | | CXL13 |
| 18 | CR10279 | IYLLNLAISN | ACKR2_HUMAN: WT: 83: 98 | CCL28, SDF1, |
| | | LLFLVT | | CXL13, IL8 |
| 19 | CR10681 | KIIFSYVVVF | ACKR3_HUMAN: CA: 252: 267 | CCL25, CCL28, |
| | | LVAWLP | | SDF1 |
| 20 | CR1078\| | ALFSIFWWI | US28_HCMVA: CA: 145: 160, | CCL19, CCL20, |
| | VP396 | FAVIIAI | US28_HCMVT: CA: 145: 160, | CCL22, CCL25, |
| | | | | CXL14, CCL1, |
| | | | | CCL17, CCL5, |
| | | | | X3CL1, CXL10, |
| | | | | CXL11, SDF1, |
| | | | US28_HCMVM: CA: 145: 160 | CXL13, IL8 |
| 21 | CR10825 | KLIFAIVVA | XCR1_HUMAN: WT: 225: 240 | CCL17, CCL28, |
| | | YFLSWGP | | CXL14 |
| 22 | CR11007\| | KQAALFSIF | US28_HCMVA: CA: 142: 157, | CCL1, CCL18, |
| | VP4859 | WWIFAVI | US28_HCMVT: CA: 142: 157, | CCL19, CCL22, |
| | | | US28_HCMVM: CA: 142: 157 | CCL28, CXL10, |
| | | | | CXL13, CXL14, |
| | | | | IL8, CCL11, |
| | | | | CCL17, CCL2, |
| | | | | CCL20, CCL25, |
| | | | | CCL5 |
| 23 | CR11008\| | KQACLFSIF | US28_HCMVA: WT: 142: 157, | SDF1, CCL1, |
| | VP4860 | WWIFAVI | US28_HCMVT: WT: 142: 157, | CCL11, CCL15, |
| | | | US28_HCMVM: WT: 142: 157 | CCL17, CCL19, |
| | | | | CCL20, CCL22, |
| | | | | CCL5, X3CL1, |
| | | | | CXL10, CXL11, |
| | | | | CXL13, IL8 |
| 24 | CR11012\| | KQASLFSIF | US28_HCMVA: CS: 142: 157, | CCL1, CCL15, |
| | VP4862 | WWIFAVI | US28_HCMVT: CS: 142: 157, | CCL17, CCL18, |
| | | | US28_HCMVM: CS: 142: 157 | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8, |
| | | | | CCL11, CCL5 |
| 25 | CR1124 | ALIQMLEICI | ACKR4_HUMAN: WT: 198: 213 | CCL18, CCL19, |
| | | GFVVPF | | CCL20 |
| 26 | CR11295 | KVLWALQY | GPR75_HUMAN: CA: 386: 401 | CCL17, CCL18, |
| | | | | CCL2, CXL10, |
| | | IGLGFFAA | | SDF1, CXL14 |
| 27 | CR11478 | LAFIYVLMF | VG74_SHV21: CA: 42: 57 | CCL1, CCL20, |
| | | LANAIGN | | CXL11, SDF1, |
| | | | | CXL13, CXL14 |
| 28 | CR11556 | LAKLEAFFL | VG74_SHV21: CA: 108: 123 | CCL18, CCL2, |
| | | NLSIYWS | | CCL25, CCL28, |
| | | | | CCL8, X3CL1, |
| | | | | CXL10, CXL11, |
| | | | | SDF1, CXL13, |
| | | | | IL8 |
| 29 | CR11604 | LALSISLLAE | VGPCR_HHV8P: CA: 94: 109 | CCL17, CCL18, |
| | | VLMFLF | | CCL20, CCL25, |
| | | | | CXL10, CXL13, |
| | | | | CXL14, IL8 |
| 30 | CR11719 | LATFKILIAI | UL33_MUHVS: CA: 202: 217 | CCL1, CCL18, |
| | | VWGVMP | | CCL20, CCL22, |
| | | | | CCL25, CCL28, |
| | | | | CXL10, SDF1 |
| 31 | CR11720 | LATFKILICI | UL33_MUHVS: WT: 202: 217 | CCL17, CCL18, |
| | | VWGVMP | | CCL19, CCL20, |
| | | | | CCL25, CXL10, |
| | | | | CXL14 |
| 32 | CR11776 | LAVKIVFSFI | UL33_HHV6U: WT: 190: 205 | CCL17, CCL18, |
| | | WGVLPT | | CCL19, CCL20, |
| | | | | CCL22, CCL25, |
| | | | | CCL8, X3CL1, |
| | | | | CXL10, CXL11, |
| | | | | SDF1, CXL13, |
| | | | | CXL14, IL8 |
| 33 | CR11907 | LDFILALVG | GPR17_HUMAN: WT: 68: 83 | CCL11, CCL18, |
| | | NTLALWL | | CCL2 |
| 34 | CR12037 | LENMLFASF | GPR17_HUMAN: WT: 57: 72 | CCL22, CXL10, |
| | | YLLDFIL | | CXL11, SDF1, |
| | | | | CXL13, IL8 |
| 35 | CR12151\| | LFGSIGNFL | US28_HCMVA: WT: 45: 60, | CCL11, CCL15, |
| | VP5320 | VIFTITW | US28_HCMVT: WT: 45: 60, | CCL17, CCL19, |
| | | | US28_HCMVM: WT: 45: 60 | CCL20, CCL22, |
| | | | | CCL8, X3CL1, |
| | | | | CXL10, CXL11, |
| | | | | SDF1, CXL14, |
| | | | | IL8 |
| 36 | CR12163 | LFIGIKIVYA | UL33_HHV7J: CA: 205: 220 | CCL18, CCL19, |
| | | FLWGIF | | CCL2, CCL25, |
| | | | | CCL28, X3CL1, |
| | | | | CXL10, SDF1, |
| | | | | CXL13, CXL14 |
| 37 | CR12164 | LFIGIKIVYC | UL33_HHV7J: WT: 205: 220 | CCL1, CCL11, |
| | | FLWGIF | | CCL17, CCL18, |
| | | | | CCL19, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 38 | CR12238 | LFMFVLSVL | O41975_MHV68: CA: 54: 69 | CCL1, CCL11, |
| | | GNLTVVA | | CCL17, CCL18, |
| | | | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 39 | CR12239 | LFMFVLSVL | O41975_MHV68: WT: 54: 69 | CCL1, CCL11, |
| | | GNLTVVC | | CCL15, CCL17, |
| | | | | CCL18, CCL19, |
| | | | | CCL2, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 40 | CR12240 | LFMFVLSVL | O41975_MHV68: CS: 54: 69 | CCL17, CCL18, |
| | | GNLTVVS | | CCL19, CCL25, |
| | | | | CCL28, CXL10, |
| | | | | CXL11, SDF1 |
| 41 | CR12277 | LFSFNRFFC | VGCR_HHV6Z: WT: 97: 112, | CCL18, CCL25, |
| | | QLVYFIY | UL33_HHV6U: WT: 93: 108 | X3CL1, CXL10 |
| 42 | CR12280\| | LFSIFWWIF | US28_HCMVA: WT: 146: 161, | CCL19, CCL20, |
| | VP5330 | AVIIAIP | US28_HCMVT: WT: 146: 161, | CCL22, X3CL1, |
| | | | | CXL13, CCL1, |
| | | | US28_HCMVM: WT: 146: 161 | CCL11, CCL15, |
| | | | | CCL17, CCL18, |
| | | | | CCL2, CCL5, |
| | | | | CXL10, CXL11, |
| | | | | SDF1, IL8 |
| 43 | CR12308 | LFTLYVSTL | O41975_MHV68: WT: 119: 134 | CCL20, CCL22, |
| | | DFCIVFI | | CCL28, CXL10, |
| | | | | SDF1 |
| 44 | CR12330 | LFVMLFSYG | CXCR1_HUMAN: CS: 215: 230 | CCL17, CCL18, |
| | | FTLRTLF | | CCL20, CCL22, |
| | | | | CCL28, CXL14, |
| | | | | IL8 |
| 45 | CR12432 | LGIILFSYVE | XCR1_HUMAN: CS: 198: 213 | CCL17, CCL18, |
| | | ILRTLF | | CXL10, CXL11, |
| | | | | IL8 |
| 46 | CR12439 | LGIMLFAYS | CCR4_HUMAN: CA: 215: 230 | CCL17, CCL18, |
| | | MIIRTLQ | | CCL20, CCL25 |
| 47 | CR12569 | LGVWLMAI | US27_HCMVA: CA: 35: 50 | CCL2, X3CL1, |
| | | VGTFLNVL | | CXL10, CXL11 |
| 48 | CR1264 | ALSISLLAEV | VGPCR_HHV8P: CA: 95: 110 | CCL1, CCL11, |
| | | LMFLFP | | CCL17, CCL18, |
| | | | | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 49 | CR12869 | LIWVISLLVS | CXCR6_HUMAN: WT: 154: 169 | CCL17, CCL18, |
| | | LPQIIY | | X3CL1, CXL14, |
| | | | | IL8 |
| 50 | CR12872 | LIYLFLLLW | TSG6_HUMAN: WT: 4: 19 | CCL1, CCL18, |
| | | EDTQGWG | | CCL19, CCL2, |
| | | | | CCL20, CCL25, |
| | | | | CXL10, SDF1, |
| | | | | IL8 |
| 51 | CR13108 | LLFFVFSFPY | VGPCR_HHV8P: CS: 258: 273 | CCL17, CCL18, |
| | | HVLNLL | | CXL10, CXL11, |
| | | | | CXL14, IL8 |
| 52 | CR13203 | LLIMLFSYG | CXCR2_HUMAN: CS: 224: 239 | CCL18, CCL22, |
| | | FTLRTLF | | CCL28, X3CL1, |
| | | | | SDF1 |
| 53 | CR1325 | ALYSLLFLL | CXCR3_HUMAN: WT: 58: 73 | CCL17, CCL19, |
| | | GLLGNGA | | CCL2, CCL20, |
| | | | | CCL22, CXL11, |
| | | | | CXL14 |
| 54 | CR13324 | LLMLGLELL | CCR6_HUMAN: WT: 212: 227 | CCL1, CCL11, |
| | | FGFFIPL | | CCL17, CCL18, |
| | | | | CCL2, CCL20, |
| | | | | CCL22, CCL28, |
| | | | | CXL10, CXL11, |
| | | | | SDF1, CXL14 |
| 55 | CR13391 | LLQALYSAV | VGPCR_HHV8P: WT: 301: 316 | CCL1, CCL18, |
| | | VPLIYSC | | CXL14 |
| 56 | CR13458 | LLSVLFTLY | O41975_MHV68: CS: 115: 130 | CCL18, CCL2, |
| | | VSTLDFS | | CCL20, CXL10, |
| | | | | CXL11, SDF1, |
| | | | | CXL13, CXL14, |
| | | | | IL8 |
| 57 | CR13507 | LLTWLAGLI | UL33_HCMVA: WT: 151: 166 | CCL1, CCL11, |
| | | FSVPAAV | | CCL17, CCL18, |
| | | | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL14, IL8 |
| 58 | CR13605 | LMFMIFAYT | CCR6_HUMAN: CA: 227: 242 | CCL17, CCL18, |
| | | FIVKTLV | | CCL20, CCL22, |
| | | | | CXL10 |
| 59 | CR13634 | LMLVFSFVS | O41975_MHV68: CS: 84: 99 | CCL19, CCL28, |
| | | MLASLAH | | CXL13, CXL14 |
| 60 | CR13861 | LPFWGISVA | ACKR2_HUMAN: WT: 99: 114 | CCL1, CCL11, |
| | | WHWVFGS | | CCL15, CCL17, |
| | | | | CCL18, CCL19, |
| | | | | CCL2, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 61 | CR13966 | LPTMVFSFF | UL33_HHV6U: WT: 203: 218 | CCL20, CCL25, |
| | | YVIFCKA | | SDF1, CXL14 |
| 62 | CR13967 | LPTMVFSFF | UL33_HHV6U: CS: 203: 218 | CCL1, CCL17, |
| | | YVIFSKA | | CCL18, CCL19, |
| | | | | CCL20, CCL28, |
| | | | | CXL10 |
| 63 | CR14086 | LQTVITFAL | US27_HCMVM: CA: 270: 285 | CCL15, CCL2, |
| | | YVGQFLA | | CCL28, X3CL1 |
| 64 | CR14316 | LSISLLAEVL | VGPCR_HHV8P: WT: 96: 111 | CCL17, CCL18, |
| | | MFLFPN | | CCL19, CCL2, |
| | | | | CCL20, CCL22, |
| | | | | CCL25, X3CL1, |
| | | | | CXL14 |
| 65 | CR14365 | LSLSISLLAE | VGPCR_HHV8P: CS: 94: 109 | CCL11, CCL19, |
| | | VLMFLF | | CCL28, SDF1, |
| | | | | CXL13, IL8 |
| 66 | CR14515 | LSWKVLLTL | UL33_HCMVM: WT: 202: 217 | CCL1, CCL15, |
| | | VWGAAPV | | CCL17, CCL18, |
| | | | | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 67 | CR14548 | LTGFLFYLN | GPR17_HUMAN: WT: 134: 149 | CCL18, CCL19, |
| | | MYASIYF | | CCL22, CCL28, |
| | | | | CXL10, CXL11, |
| | | | | SDF1, CXL13, |
| | | | | IL8 |
| 68 | CR14585 | LTLKVILGFF | CCR9_HUMAN: WT: 215: 230 | CCL17, CCL18, |
| | | LPFVVM | | CCL19, CCL20, |
| | | | | CXL14 |
| 69 | CR1475 | ANVTFTGLI | BILF1_EBVB9: CA: 225: 240 | CCL2, CCL22, |
| | | AAFSWFM | | CXL11, CXL13 |
| 70 | CR14752 | LVFIFGFVG | CCR2_HUMAN: WT: 51: 66 | CCL17, CCL25, |
| | | NMLVVLI | | CXL10 |
| 71 | CR14753\| | LVFIIFWLPY | US28_HCMVA: WT: 235: 250, | CCL1, CCL15, |
| | VP5936 | HLTLFV | US28_HCMVT: WT: 235: 250, | X3CL1, CXL10, |
| | | | US28_HCMVM: WT: 235: 250 | CXL11, CXL13, |
| | | | | IL8 |
| 72 | CR14765 | LVFLLSLVG | XCR1_HUMAN: WT: 40: 55 | CCL2, CCL28, |
| | | NSLVLWV | | CXL10, IL8 |
| 73 | CR14780 | LVFVIGLVG | CCR1_HUMAN: WT: 43: 58 | CCL17, CCL18, |
| | | NILVVLV | | CCL19, CCL20, |
| | | | | CCL22, CCL25, |
| | | | | CCL28, X3CL1, |
| | | | | CXL10, CXL14 |
| 74 | CR14849 | LVLGLLLNS | GPR35_HUMAN: WT: 31: 46 | CCL17, CCL19, |
| | | LALWVFC | | CCL20, CCL28, |
| | | | | SDF1 |
| 75 | CR14850 | LVLGLLLNS | GPR35_HUMAN: CS: 31: 46 | CCL22, CCL28, |
| | | LALWVFS | | CXL10, CXL11, |
| | | | | SDF1, CXL14 |
| 76 | CR14974 | LVTLPFWGI | ACKR2_HUMAN: WT: 96: 111 | CCL18, CCL25, |
| | | SVAWHWV | | CCL28 |
| 77 | CR14976 | LVTMAVWV | XCR1_HUMAN: WT: 148: 163 | CCL2, SDF1, |
| | | ASILSSIL | | CXL13 |
| 78 | CR15063 | LWVVVFQF | CXCR4_HUMAN: WT: 194: 209 | CCL17, CCL18, |
| | | QHIMVGLI | | CCL19, CCL2, |
| | | | | CCL20, CCL22, |
| | | | | CCL28, CCL8, |
| | | | | X3CL1, CXL10, |
| | | | | CXL11, SDF1, |
| | | | | CXL13, CXL14, |
| | | | | IL8 |
| 79 | CR15077 | LYAHLAAA | GPR17_HUMAN: CA: 174: 189 | CCL18, CCL19, |
| | | FLWVVVAV | | CCL20, CCL22, |
| | | | | CCL25, CCL28, |
| | | | | CXL10, CXL11, |
| | | | | SDF1, IL8 |
| 80 | CR15079 | LYAHLASAF | GPR17_HUMAN: CS: 174: 189 | CCL17, CCL18, |
| | | LWVVVAV | | CCL20, CCL22, |
| | | | | CCL28, X3CL1, |
| | | | | CXL10, CXL11, |
| | | | | SDF1, CXL13, |
| | | | | CXL14, IL8 |
| 81 | CR15106\| | LYGVVFLFG | US28_HCMVA: WT: 39: 54, | CCL15, CXL10, |
| | VP5994 | SIGNFLV | US28_HCMVT: WT: 39: 54, | CXL11, IL8 |
| | | | US28_HCMVM: WT: 39: 54 | |
| 82 | CR15153 | LYSLLFLLG | CXCR3_HUMAN: WT: 59: 74 | CCL18, CCL22, |
| | | LLGNGAV | | CCL25 |
| 83 | CR15385 | MFLLNLAIA | CCR9_HUMAN: WT: 85: 100 | X3CL1, CXL14, |
| | | DLLFLVT | | L8 |
| 84 | CR15502 | MIFTWNLVL | UL78_HCMVM: WT: 75: 90 | CCL11, CCL17, |
| | | SQFFSIL | | CCL18, CCL19, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 85 | CR15569 | MLFASFYLL | GPR17_HUMAN: WT: 60: 75 | CCL1, CCL11, |
| | | DFILALV | | CCL17, CCL18, |
| | | | | CCL19, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 86 | CR15670 | MMTWFYAF | UL33_HCMVM: WT: 219: 234 | CCL18, CCL2, |
| | | FYSTVQRA | | CCL20, CCL25, |
| | | | | CCL28, CCL8, |
| | | | | X3CL1, CXL13, |
| | | | | IL8 |
| 87 | CR15801 | MSFFSGMLL | CCR7_HUMAN: WT: 138: 153 | CCL18, CCL20, |
| | | LLCISID | | SDF1 |
| 88 | CR15915 | MVFSFFYVI | UL33_HHV6U: CA: 206: 221 | CCL1, CCL18, |
| | | FAKALHD | | CCL19, CCL20 |
| 89 | CR15917 | MVFSFFYVI | UL33_HHV6U: CS: 206: 221 | CCL19, X3CL1, |
| | | FSKALHD | | SDF1, CXL13 |
| 90 | CR15930 | MVIWVLAFF | CML1_HUMAN: WT: 161: 176 | CCL28, CXL10, |
| | | LSSPSLV | | CXL14 |
| 91 | CR15967 | MVWANLLV | GPR35_HUMAN: CS: 218: 233 | CCL1, CXL10, |
| | | FVVSFLPL | | SDF1 |
| 92 | CR16988 | NVTFTGLIA | BILF1_EBVB9: CA: 226: 241 | CCL17, CCL18, |
| | | AFSWFML | | CCL20, CCL22, |
| | | | | CCL25, CCL28, |
| | | | | CXL10, SDF1, |
| | | | | CXL14 |
| 93 | CR17126 | PCGIITSVLA | CCRL2_HUMAN: WT: 146: 161 | CCL1, CCL20, |
| | | WVTAIL | | CCL22, CCL28, |
| | | | | X3CL1, CXL10, |
| | | | | CXL11, SDF1, |
| | | | | CXL14, IL8 |
| 94 | CR17298 | PGPWMNIL | ACKR1_HUMAN: WT: 239: 254 | CCL1, CCL17, |
| | | WAWFIFWW | | CCL18, CCL19, |
| | | | | CCL20, CCL28, |
| | | | | CXL10, CXL14 |
| 95 | CR17781 | PSWIISFSVW | ACKR4_HUMAN: CS: 153: 168 | CCL17, CCL18, |
| | | MAAILL | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 96 | CR1782\| | ASLFSIFWW | US28_HCMVA: CS: 144: 159, | CCL17, CCL18, |
| | VP667 | IFAVIIA | US28_HCMVT: CS: 144: 159, | CCL19, CCL20, |
| | | | US28_HCMVM: CS: 144: 159 | CCL22, CCL28, |
| | | | | X3CL1, CXL10, |
| | | | | CXL11, SDF1, |
| | | | | CXL14, IL8, |
| | | | | CCL1, CCL11, |
| | | | | CCL2, CCL5, |
| | | | | CXL13 |
| 97 | CR17840\| | PVKQASLFSI | US28_HCMVA: CS: 140: 155, | CCL19, CCL1, |
| | VP7283 | FWWIFA | US28_HCMVT: CS: 140: 155, | CCL11, CCL15, |
| | | | US28_HCMVM: CS: 140: 155 | CCL17, CCL18, |
| | | | | CCL2, CCL20, |
| | | | | CCL22, CCL25, |
| | | | | CCL28, CCL5, |
| | | | | CXL10, SDF1, |
| | | | | IL8 |
| 98 | CR17892\| | PVTLFLYGV | US28_HCMVA: WT: 34: 49, | CCL15, CCL2, |
| | VP7313 | VFLFGSI | US28_HCMVT: WT: 34: 49, | CXL10, CXL11, |
| | | | US28_HCMVM: WT: 34: 49 | CXL13, CXL14, |
| | | | | IL8 |
| 99 | CR17907 | PYHWGWVL | XCR1_HUMAN: CA: 90: 105 | CCL17, CCL18, |
| | | GDFLAKLL | | CCL25 |
| 100 | CR17948\| | QAALFSIFW | US28_HCMVA: CA: 143: 158, | CCL1, CCL11, |
| | VP7339 | WIFAVII | US28_HCMVT: CA: 143: 158, | CCL15, CCL17, |
| | | | US28_HCMVM: CA: 143: 158 | CCL18, CCL19, |
| | | | | CCL2, CCL20, |
| | | | | CCL22, CCL5, |
| | | | | X3CL1, CXL10, |
| | | | | CXL11, SDF1, |
| | | | | CXL13, IL8 |
| 101 | CR17951\| | QACLFSIFW | US28_HCMVA: WT: 143: 158, | CCL1, CCL17, |
| | VP7341 | WIFAVII | US28_HCMVT: WT: 143: 158, | CCL18, CCL19, |
| | | | US28_HCMVM: WT: 143: 158 | CCL20, CCL22, |
| | | | | CCL28, X3CL1, |
| | | | | CXL10, SDF1, |
| | | | | CCL11, CCL15, |
| | | | | CCL2, CCL5, |
| | | | | CXL11, CXL13, |
| | | | | CXL14, IL8 |
| 102 | CR18009\| | QASLFSIFW | US28_HCMVA: CS: 143: 158, | CCL1, CCL11, |
| | VP7359 | WIFAVII | US28_HCMVT: CS: 143: 158, | CCL15, CCL17, |
| | | | US28_HCMVM: CS: 143: 158 | CCL18, CCL19, |
| | | | | CCL2, CCL20, |
| | | | | CCL22, CCL5, |
| | | | | X3CL1, CXL10, |
| | | | | CXL11, SDF1, |
| | | | | CXL13, CXL14, |
| | | | | IL8 |
| 103 | CR1802 | ASMILESLF | O41975_MHV68: CA: 297: 312 | CCL17, CCL18, |
| | | AALVTLI | | CCL2, CCL20, |
| | | | | CCL22, CCL25, |
| | | | | CCL28, CXL10 |
| 104 | CR18189 | QHNLFFLLS | XCR1_HUMAN: WT: 189: 204 | CCL17, CCL18, |
| | | LGIILFC | | CCL22, CCL25, |
| | | | | X3CL1, CXL14 |
| 105 | CR18714 | QYLKAFLA | ACKR2_HUMAN: WT: 321: 336 | CCL18, CCL28, |
| | | AVLGWHLA | | CXL13, CXL14 |
| 106 | CR18933 | RFGVWLMSI | US27_HCMVM: CS: 36: 51 | CCL28, CXL10, |
| | | VGTFLNM | | IL8 |
| 107 | CR18955 | RFRQYLKAF | ACKR2_HUMAN: WT: 318: 333 | CCL17, CCL18, |
| | | LAAVLGW | | CXL14 |
| 108 | CR19192 | RLGVWLMSI | US27_HCMVA: CS: 34: 49 | CCL1, CCL17, |
| | | VGTFLNV | | CCL2, CCL25, |
| | | | | X3CL1, CXL11 |
| 109 | CR1923 | ATFKILISIV | UL33_MUHVS: CS: 203: 218 | CCL18, CCL28, |
| | | WGVMPV | | X3CL1, CXL10, |
| | | | | CXL11, SDF1, |
| | | | | CXL13, IL8 |
| 110 | CR19398\| | RPVKQACLF | US28_HCMVA: WT: 139: 154, | CCL19, CCL1, |
| | VP7889 | SIFWWIF | US28_HCMVT: WT: 139: 154, | CCL11, CCL17, |
| | | | US28_HCMVM: WT: 139: 154 | CCL28, CCL5, |
| | | | | X3CL1, CXL10, |
| | | | | CXL13 |
| 111 | CR19399\| | RPVKQASLF | US28_HCMVA: CS: 139: 154, | CCL1, CCL11, |
| | VP7890 | SIFWWIF | US28_HCMVT: CS: 139: 154, | CCL17, CCL19, |
| | | | US28_HCMVM: CS: 139: 154 | CCL2, CCL20, |
| | | | | CCL22, CCL25, |
| | | | | CCL28, CCL5, |
| | | | | X3CL1, CXL14 |
| 112 | CR19824 | RVTLTALAV | CXCR3_HUMAN: CA: 167: 182 | CCL2, CCL22, |
| | | WGLALLF | | CCL25, CXL10, |
| | | | | CXL11, SDF1, |
| | | | | CXL14, IL8 |
| 113 | CR19826 | RVTLTSLAV | CXCR3_HUMAN: CS: 167: 182 | CCL17, CCL2, |
| | | WGLSLLF | | X3CL1, CXL14 |
| 114 | CR20068 | SAVLTLKVI | CCR9_HUMAN: WT: 212: 227 | CCL17, CCL18, |
| | | LGFFLPF | | CCL20, CCL25, |
| | | | | CCL28, CXL11 |
| 115 | CR20273 | SESALAFIY | VG74_SHV21: WT: 38: 53 | CCL18, CCL19, |
| | | VLMFLCN | | CCL20, CXL14 |
| 116 | CR2028 | ATSWVVIF | US27_HCMVM: CS: 149: 164 | CCL17, CCL18, |
| | | WILAALMG | | CCL19, CCL20, |
| | | | | CCL28, SDF1, |
| | | | | CXL13, CXL14 |
| 117 | CR20429 | SFYISLFAGV | US27_HCMVA: CS: 109: 124, | CCL22, CCL25, |
| | | SFLINL | US27_HCMVM: CS: 111: 126 | CXL10, CXL11 |
| 118 | CR20433 | SFYVAMFAS | US28_HCMVA: CS: 110: 125, | CCL1, X3CL1, |
| | VP8386 | LSFITEI | US28_HCMVT: CS: 110: 125, | CXL10, CXL11, |
| | | | US28_HCMVM: CS: 110: 125 | IL8 |
| 119 | CR20481 | SGIFFIILLTI | CCR5_HUMAN: WT: 114: 129 | CCL1, CCL19, |
| | | DRYLA | | CCL20, CCL22, |
| | | | | CCL25, CCL28, |
| | | | | X3CL1, CXL10, |
| | | | | CXL11, SDF1 |
| 120 | CR20486 | SGIITSVLA | CCRL2_HUMAN: CS: 147: 162 | CCL11, CCL2, |
| | | WVTAILA | | CCL8, IL8 |
| 121 | CR2051 | ATYMLLVL | VG74_SHV21: CA: 284: 299 | CCL20, CCL22, |
| | | YYAMLPLM | | CCL28, X3CL1, |
| | | | | CXL10, SDF1, |
| | | | | CXL14, IL8 |
| 122 | CR20557 | SGYDMWLM | VU51_HHV6U: WT: 165: 180 | CCL1, CCL15, |
| | | LVYSVMIP | | CCL25 |
| 123 | CR20641\| | SIFWWIFAVI | US28_HCMVA: WT: 148: 163, | CCL15, CCL25, |
| | VP8515 | IAIPHF | US28_HCMVT: WT: 148: 163, | X3CL1, CXL13, |
| | | | US28_HCMVM: WT: 148: 163 | CCL1, CCL11, |
| | | | | CCL17, CCL18, |
| | | | | CCL19, CCL2, |
| | | | | CCL20, CCL22, |
| | | | | CCL28, CCL5, |
| | | | | CCL8, CXL10, |
| | | | | CXL11, SDF1, |
| | | | | CXL14, IL8 |
| 124 | CR20660 | SIILMFLSDF | VU51_HHV7J: CS: 79: 94 | CCL25, CXL13, |
| | | SFIFSS | | IL8 |
| 125 | CR20762 | SIYLLNLAIS | CCR1_HUMAN: WT: 70: 85 | CCL22, CCL28, |
| | | DLLFLF | | CXL10, CXL11, |
| | | | | SDF1, CXL13, |
| | | | | CXL14, IL8 |
| 126 | CR20939 | SLCLSISLLA | VGPCR_HHV8P: WT: 93: 108 | CCL1, CCL18, |
| | | EVLMFL | | CCL19, X3CL1 |
| 127 | CR20965 | SLFAALVTLI | O41975_MHV68: CA: 303: 318 | CCL11, CCL17, |
| | | TSLFGS | | CCL18, CCL19, |
| | | | | CCL2, CCL20, |
| | | | | CCL28, X3CL1, |
| | | | | CXL10, CXL11, |
| | | | | CXL14, IL8 |
| 128 | CR20967 | SLFCALVTLI | O41975_MHV68: WT: 303: 318 | CCL1, CCL11, |
| | | TSLFGS | | CCL15, CCL17, |
| | | | | CCL18, CCL19, |
| | | | | CCL2, CCL20, |
| | | | | CCL22, CCL25, |
| | | | | CCL28, CCL5, |
| | | | | CCL8, X3CL1, |
| | | | | CXL10, CXL11, |
| | | | | SDF1, CXL13, |
| | | | | CXL14, IL8 |
| 129 | CR20981 | SLFSALVTLI | O41975_MHV68: CS: 303: 318 | CCL1, CCL11, |
| | | TSLFGS | | CCL17, CCL18, |
| | | | | CCL2, CCL25, |
| | | | | CCL8, X3CL1, |
| | | | | CXL10, IL8 |
| 130 | CR20982\| | SLFSIFWWIF | US28_HCMVA: CS: 145: 160, | X3CL1, CXL10, |
| | VP8673 | AVIIAI | US28_HCMVT: CS: 145: 160, | CXL11, CXL13, |
| | | | US28_HCMVM: CS: 145: 160 | IL8 |
| 131 | CR21134 | SLSISLLAEV | VGPCR_HHV8P: CS: 95: 110 | CCL17, CCL18, |
| | | LMFLFP | | CCL2, CCL25, |
| | | | | CCL28, X3CL1, |
| | | | | CXL10, CXL11, |
| | | | | SDF1, CXL14, |
| | | | | IL8 |
| 132 | CR21168 | SLVFIFGFVG | CCR5_HUMAN: WT: 38: 53 | CCL1, CCL22, |
| | | NMLVIL | | CCL28, CXL10, |
| | | | | CXL13, CXL14 |
| 133 | CR21236 | SMILESLFA | O41975_MHV68: CA: 298: 313 | CCL22, CXL10, |
| | | ALVTLIT | | CXL11, SDF1, |
| | | | | CXL13, IL8 |
| 134 | CR21238 | SMILESLFSA | O41975_MHV68: CS: 298: 313 | CCL2, CXL11, |
| | | LVTLIT | | IL8 |
| 135 | CR21284 | SMVIWVLAF | CML1_HUMAN: CS: 160: 175 | CCL1, CCL18, |
| | | FLSSPSL | | CCL20 |
| 136 | CR2129 | AVFYALLFV | CCR8_HUMAN: CA: 39: 54 | CCL15, CCL18, |
| | | FSLLGNS | | CCL19, CCL20, |
| | | | | CCL25, CCL28, |
| | | | | SDF1 |
| 137 | CR21349 | SNVTFTGLIS | BILF1_EBVB9: CS: 225: 240 | CCL11, CCL2, |
| | | AFSWFM | | CCL3, CCL4, |
| | | | | CCL5, CCL8 |
| 138 | CR2158 | AVKIVFSFI | UL33_HHV6U: WT: 191: 206 | CCL18, CCL20, |
| | | WGVLPTM | | SDF1, CXL14 |
| 139 | CR22218 | SVSLVNLLTI | UL78_HCMVM: WT: 49: 64 | CCL19, X3CL1, |
| | | IGCLWV | | CXL13 |
| 140 | CR22271 | SWIISFSVW | ACKR4_HUMAN: CS: 154: 169 | CCL1, CCL17, |
| | | MAAILLS | | CXL10 |
| 141 | CR22272 | SWKVLLTL | UL33_HCMVM: WT: 203: 218 | CCL17, CCL18, |
| | | VWGAAPVI | | CCL20, CCL22, |
| | | | | CCL25, CCL28, |
| | | | | X3CL1, CXL10, |
| | | | | CXL11, SDF1, |
| | | | | CXL14, IL8 |
| 142 | CR22363 | SYRSTYIILL | UL33_HCMVM: WT: 143: 158 | CCL18, X3CL1, |
| | | LTWFAG | | CXL10 |
| 143 | CR22509\| | TASFYVAMF | US28_HCMVA: CS: 108: 123, | CCL1, CCL15, |
| | VP9421 | ASLSFIT | US28_HCMVT: CS: 108: 123, | X3CL1, CXL10, |
| | | | US28_HCMVM: CS: 108: 123 | CXL11, SDF1, |
| | | | | CXL13, IL8 |
| 144 | CR22631\| | TEFDYDEDA | US28_HCMVA: WT: 12: 27, | CCL11, CCL17, |
| | VP9535 | TPCVFTD | US28_HCMVT: WT: 12: 27, | CCL18, CCL19, |
| | | | US28_HCMVM: WT: 12: 27 | CCL20 |
| 145 | CR22658 | TENFEYDDL | CX3C1_HUMAN: WT: 9: 24 | CCL17, CCL18, |
| | | AEACYIG | | X3CL1 |
| 146 | CR22736 | TFGVVTSVI | CCR2_HUMAN: WT: 155: 170 | CCL1, CCL11, |
| | | TWLVAVF | | CCL17, CCL18, |
| | | | | CCL2, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 147 | CR22737 | TFGVVTSVI | CCR5_HUMAN: WT: 143: 158 | CCL1, CCL11, |
| | | TWVVAVF | | CCL15, CCL17, |
| | | | | CCL18, CCL19, |
| | | | | CCL2, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 148 | CR22747 | TFKILICIVW | UL33_MUHVS: WT: 204: 219 | CCL1, CCL17, |
| | | GVMPVV | | CCL18, CCL20, |
| | | | | CCL22, CCL28, |
| | | | | X3CL1, CXL10, |
| | | | | SDF1, CXL13, |
| | | | | CXL14 |
| 149 | CR22748 | TFKILISIVW | UL33_MUHVS: CS: 204: 219 | CCL17, CCL18, |
| | | GVMPVV | | CCL19, CCL20, |
| | | | | CCL22, CCL28, |
| | | | | X3CL1, CXL10, |
| | | | | CXL11, SDF1, |
| | | | | CXL14, IL8 |
| 150 | CR2278 | AVWGLSLLF | CXCR3_HUMAN: CS: 174: 189 | CCL18, CCL2, |
| | | ALPDFIF | | CCL22, CCL28, |
| | | | | CXL10, CXL11, |
| | | | | SDF1, CXL13, |
| | | | | CXL14, IL8 |
| 151 | CR22786 | TFTGLIAAFS | BILF1_EBVB9: CA: 228: 243 | CCL11, CCL17, |
| | | WFMLSL | | CCL18, CCL2, |
| | | | | CCL20, CCL22, |
| | | | | CCL28, X3CL1, |
| | | | | SDF1, IL8 |
| 152 | CR229\| | ACFYVAMF | US28_HCMVA: WT: 109: 124, | CXL10, CXL11, |
| | VP96 | ASLCFITE | US28_HCMVT: WT: 109: 124, | IL8 |
| | | | US28_HCMVM: WT: 109: 124 | |
| 153 | CR2290 | AWFLPIMYS | CCR7_HUMAN: WT: 58: 73 | CCL1, CCL17, |
| | | IICFVGL | | CCL18, CCL19, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 154 | CR2291 | AWFLPIMYS | CCR7_HUMAN: CS: 58: 73 | CCL1, CCL11, |
| | | IISFVGL | | IL8 |
| 155 | CR23002 | TIWLVGFLL | CXCR5_HUMAN: WT: 172: 187 | CCL17, CCL18, |
| | | ALPEILF | | CCL19, CCL20, |
| | | | | CCL25, CCL28, |
| | | | | X3CL1, SDF1, |
| | | | | CXL13, CXL14, |
| | | | | IL8 |
| 156 | CR2301 | AWLPYYIGI | CXCR4_HUMAN: CA: 251: 266 | CCL17, CCL19, |
| | | SIDSFIL | | CCL20 |
| 157 | CR23111 | TLFASDFVL | VGCR_HHV6Z: CA: 75: 90, | CCL15, CCL17, |
| | | MWAVFFM | UL33_HHV6U: CA: 71: 86 | CCL20, CCL25, |
| | | | | CXL14 |
| 158 | CR23112 | TLFASDFVL | VGCR_HHV6Z: WT: 75: 90, | CCL17, CCL19, |
| | | MWCVFFM | UL33_HHV6U: WT: 71: 86 | CCL20, CCL22, |
| | | | | CCL8, X3CL1 |
| 159 | CR23240 | TLPFWGISV | ACKR2_HUMAN: WT: 98: 113 | CCL17, CCL18, |
| | | AWHWVFG | | CCL20, X3CL1, |
| | | | | CXL11 |
| 160 | CR23379 | TNFDFMILN | GPR75_HUMAN: CA: 77: 92 | CCL17, CCL18, |
| | | LSFADLF | | X3CL1 |
| 161 | CR2339 | AYDLFIGIKI | UL33_HHV7J: CA: 202: 217 | CCL1, CCL11, |
| | | VYAFLW | | CCL15, CCL17, |
| | | | | CCL18, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 162 | CR2340 | AYDLFIGIKI | UL33_HHV7J: WT: 202: 217 | CCL15, CCL17, |
| | | VYCFLW | | CCL18, CCL19, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 163 | CR2341 | AYDLFIGIKI | UL33_HHV7J: CS: 202: 217 | CCL1, CCL11, |
| | | VYSFLW | | CCL15, CCL17, |
| | | | | CCL18, CCL19, |
| | | | | CCL2, CCL20, |
| | | | | CCL22, CCL25, |
| | | | | CCL28, CCL5, |
| | | | | CCL8, X3CL1, |
| | | | | CXL10, CXL11, |
| | | | | SDF1, CXL13, |
| | | | | CXL14, IL8 |
| 164 | CR23443 | TNWKLLAEI | VU51_HHV7J: WT: 7: 22 | CCL17, CCL18, |
| | | YEYLFFF | | X3CL1, CXL10, |
| | | | | CXL13 |
| 165 | CR23572 | TRFGVWLM | US27_HCMVM: CS: 35: 50 | CCL17, CCL18, |
| | | SIVGTFLN | | CCL2, CCL20, |
| | | | | CCL22, CCL25, |
| | | | | X3CL1, IL8 |
| 166 | CR23587 | TRLGVWLM | US27_HCMVA: CA: 33: 48 | CCL18, CCL20, |
| | | AIVGTFLN | | CCL22 |
| 167 | CR23773 | TSVITWLVA | CCR2_HUMAN: WT: 160: 175 | CCL1, CCL18, |
| | | VFASVPG | | CCL2, CCL20, |
| | | | | CCL8 |
| 168 | CR23809\| | TTEFDYDED | US28_HCMVA: WT: 11: 26, | CCL11, CCL15, |
| | VP10212 | ATPCVFT | US28_HCMVT: WT: 11: 26, | CCL17, CCL18, |
| | | | US28_HCMVM: WT: 11: 26 | CCL19, CCL20, |
| | | | | CCL25, CCL5, |
| | | | | X3CL1, CXL14 |
| 169 | CR23880 | TTVILILAFF | CXCR4_HUMAN: CS: 240: 255 | CCL1, CCL18, |
| | | ASWLPY | | CCL20, CCL25, |
| | | | | CCL28, SDF1, |
| | | | | CXL14 |
| 170 | CR23915 | TVFLSIFYSV | CX3C1_HUMAN: WT: 31: 46 | CCL17, CCL18, |
| | | IFAIGL | | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 171 | CR23943 | TVITFALYV | US27_HCMVM: CA: 272: 287 | CCL2, CCL20, |
| | | GQFLAYV | | CCL22, X3CL1, |
| | | | | CXL10, CXL11, |
| | | | | SDF1, CXL13 |
| 172 | CR23945 | TVITFSLYV | US27_HCMVM: CS: 272: 287 | CCL1, CCL15, |
| | | GQFLAYV | | CCL20, CCL22, |
| | | | | CCL28, CXL10, |
| | | | | CXL11, SDF1, |
| | | | | CXL13 |
| 173 | CR23947 | TVKLIFAIVV | XCR1_HUMAN: WT: 223: 238 | CCL17, CCL20, |
| | | AYFLSW | | CCL22, CCL28, |
| | | | | SDF1, CXL14 |
| 174 | CR23949 | TVLATFKILI | UL33_MUHVS: CA: 200: 215 | CCL1, CCL11, |
| | | AIVWGV | | CCL17, CCL18, |
| | | | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 175 | CR23950 | TVLATFKILI | UL33_MUHVS: WT: 200: 215 | CCL18, CCL19, |
| | | CIVWGV | | CCL20, CCL25, |
| | | | | SDF1, IL8 |
| 176 | CR23951 | TVLATFKILI | UL33_MUHVS: CS: 200: 215 | CCL11, CCL17, |
| | | SIVWGV | | CCL18, CCL19, |
| | | | | CCL2, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 177 | CR23957 | TVLLSWKV | UL33_HCMVM: WT: 199: 214 | CCL1, CCL17, |
| | | LLTLVWGA | | CCL18, CCL19, |
| | | | | CCL2, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 178 | CR23958 | TVLLSWKV | UL33_HCMVA: WT: 199: 214 | CCL1, CCL18, |
| | | LLTMVWGA | | CCL20, CCL25, |
| | | | | CCL28, CXL10, |
| | | | | CXL11, SDF1 |
| 179 | CR240\| | ACLFSIFWW | US28_HCMVA: WT: 144: 159, | CCL28, SDF1, |
| | VP103 | IFAVIIA | US28_HCMVT: WT: 144: 159, | CCL1, CCL15, |
| | | | US28_HCMVM: WT: 144: 159 | CCL17, CCL20, |
| | | | | CCL5, X3CL1, |
| | | | | CXL10, CXL11, |
| | | | | CXL13, IL8 |
| 180 | CR24063 | TWNLVLSQF | UL78_HCMVM: WT: 78: 93 | CCL1, CCL22, |
| | | FSILATM | | CXL11, CXL14 |
| 181 | CR24107 | TYIILLLTWF | UL33_HCMVM: WT: 147: 162 | CCL22, CCL25, |
| | | AGLIFS | | CXL11 |
| 182 | CR24347 | VAYSLIFLL | CXCR5_HUMAN: WT: 56: 71 | CCL18, CCL19, |
| | | GVIGNVL | | CCL20, CXL11, |
| | | | | CXL14 |
| 183 | CR24367 | VCILVWLLA | ACKR3_HUMAN: WT: 164: 179 | CCL11, CCL17, |
| | | FCVSLPD | | CCL18, CCL19, |
| | | | | CCL20, CCL22, |
| | | | | CCL25, CCL28, |
| | | | | CXL10, CXL11, |
| | | | | SDF1, CXL13, |
| | | | | CXL14, IL8 |
| 184 | CR2439 | CALVTLITSL | O41975_MHV68: WT: 306: 321 | CCL17, CCL18, |
| | | FGSIFK | | CCL19, CCL20, |
| | | | | CXL10, SDF1, |
| | | | | CXL13, CXL14, |
| | | | | IL8 |
| 185 | CR24556 | VFGTVFLSIF | CX3C1_HUMAN: WT: 28: 43 | CCL1, CCL17, |
| | | | | CCL18, CCL19, |
| | | YSVIFA | | CCL2, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 186 | CR24590\| | VFLFGSIGNF | US28_HCMVA: WT: 43: 58, | CCL1, CCL5, |
| | VP10605 | LVIFTI | US28_HCMVT: WT: 43: 58, | X3CL1, CXL10, |
| | | | US28_HCMVM: WT: 43: 58 | CXL11, SDF1, |
| | | | | CXL13, IL8 |
| 187 | CR24593 | VFLLSLLGN | CXCR2_HUMAN: WT: 58: 73, | CCL22, CCL25, |
| | | SLVMLVI | CXCR1_HUMAN: WT: 49: 64 | CXL10, CXL11, |
| | | | | SDF1, CXL13, |
| | | | | IL8 |
| 188 | CR24594 | VFLLSLVGN | XCR1_HUMAN: WT: 41: 56 | CCL22, X3CL1, |
| | | SLVLWVL | | CXL11, CXL13, |
| | | | | IL8 |
| 189 | CR24612 | VFLSIFYSVI | CX3C1_HUMAN: WT: 32: 47 | CCL17, CCL18, |
| | | FAIGLV | | CCL22, CCL25, |
| | | | | CCL28, X3CL1, |
| | | | | CXL10, CXL11, |
| | | | | SDF1, CXL13, |
| | | | | CXL14 |
| 190 | CR24702 | VFYSLLFVF | CCR8_HUMAN: CS: 40: 55 | CCL17, CCL18, |
| | | SLLGNSL | | CCL19, CCL20, |
| | | | | CCL22, CCL25, |
| | | | | CCL5, X3CL1, |
| | | | | CXL11, CXL14, |
| | | | | IL8 |
| 191 | CR24707 | VGALGNSL | CCR9_HUMAN: WT: 61: 76 | CCL17, CCL18, |
| | | VILVYWYC | | CCL19, CCL20 |
| 192 | CR24900 | VIGFLVPLL | CCR7_HUMAN: WT: 229: 244 | CCL15, CCL17, |
| | | AMSFCYL | | CCL18 |
| 193 | CR24960 | VIMMTWFY | UL33_HCMVM: WT: 217: 232, | CCL1, CCL17, |
| | | AFFYSTVQ | UL33_HCMVA: WT: 217: 232 | CCL18, CCL2, |
| | | | | CCL22, CCL25, |
| | | | | CCL28, CCL5, |
| | | | | X3CL1, CXL11, |
| | | | | CXL13, IL8 |
| 194 | CR24989 | VITFCLYVG | US27_HCMVM: WT: 273: 288 | CCL17, CCL18, |
| | | QFLAYVR | | CCL19, CCL20, |
| | | | | CCL25, X3CL1, |
| | | | | CXL10, SDF1, |
| | | | | CXL14 |
| 195 | CR25001 | VITWLVAVF | CCR2_HUMAN: WT: 162: 177 | CCL1, CCL19, |
| | | ASVPGII | | CCL2, CXL11, |
| | | | | CXL14 |
| 196 | CR25058\| | VKQAALFSI | US28_HCMVA: CA: 141: 156, | CCL1, CCL11, |
| | VP10767 | FWWIFAV | US28_HCMVT: CA: 141: 156, | CCL17, CCL19, |
| | | | US28_HCMVM: CA: 141: 156 | CCL2, CCL20, |
| | | | | CCL22, CCL25, |
| | | | | CCL28, CCL5, |
| | | | | X3CL1, CXL10, |
| | | | | SDF1, CXL13, |
| | | | | CXL14, IL8 |
| 197 | CR25059\| | VKQACLFSI | US28_HCMVA: WT: 141: 156, | CCL1, CCL17, |
| | VP10768 | FWWIFAV | US28_HCMVT: WT: 141: 156, | X3CL1, CXL10, |
| | | | US28_HCMVM: WT: 141: 156 | CXL11, CXL13, |
| | | | | IL8 |
| 198 | CR25060\| | VKQASLFSIF | US28_HCMVA: CS: 141: 156, | CCL15, CCL18, |
| | VP10769 | WWIFAV | US28_HCMVT: CS: 141: 156, | CCL25, CCL1, |
| | | | US28_HCMVM: CS: 141: 156 | CCL11, CCL17, |
| | | | | CCL19, CCL2, |
| | | | | CCL20, CCL22, |
| | | | | CCL28, CCL5, |
| | | | | X3CL1, CXL10, |
| | | | | CXL11, SDF1, |
| | | | | CXL13, CXL14, |
| | | | | IL8 |
| 199 | CR25151 | VLGDFLCKL | XCR1_HUMAN: WT: 96: 111 | CCL18, CCL2, |
| | | LNMIFSI | | CCL28, X3CL1, |
| | | | | CXL10, CXL14, |
| | | | | IL8 |
| 200 | CR25165 | VLGLLLNSL | GPR35_HUMAN: CA: 32: 47 | CCL19, CCL2, |
| | | ALWVFAA | | CCL20, CCL28 |
| 201 | CR25167 | VLGLLLNSL | GPR35_HUMAN: CS: 32: 47 | CCL11, CCL17, |
| | | ALWVFSS | | CCL18, CXL11, |
| | | | | CXL14 |
| 202 | CR2524 | CFLWGIFPT | UL33_HHV7J: WT: 214: 229 | CCL22, CCL25, |
| | | VIFSYFY | | CXL13, CXL14, |
| | | | | IL8 |
| 203 | CR25244 | VLLSWKVL | UL33_HCMVA: WT: 200: 215 | CCL11, CCL17, |
| | | LTMVWGAA | | CCL18, CCL19, |
| | | | | CCL2, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 204 | CR25357 | VLTLKVILG | CCR9_HUMAN: WT: 214: 229 | CCL1, CCL17, |
| | | FFLPFVV | | CCL18, CCL19, |
| | | | | CCL28, CXL10, |
| | | | | CXL11, SDF1, |
| | | | | CXL13, CXL14 |
| 205 | CR25375 | VLVIGSLVA | GPR35_HUMAN: WT: 142: 157 | CCL18, CCL19, |
| | | RWLLGIQ | | CCL20 |
| 206 | CR25407 | VLYFIADEV | UL33_HCMVM: WT: 189: 204 | CCL15, CCL17, |
| | | YTVLLSW | | CCL18, CCL19, |
| | | | | CCL20, CCL25, |
| | | | | X3CL1, CXL10, |
| | | | | CXL11, CXL13, |
| | | | | IL8 |
| 207 | CR2553 | CGIITSVLA | CCRL2_HUMAN: WT: 147: 162 | CCL11, CCL2, |
| | | WVTAILA | | CCL20, CCL22, |
| | | | | CCL28, CCL8, |
| | | | | X3CL1, CXL10, |
| | | | | SDF1, CXL14, |
| | | | | IL8 |
| 208 | CR25802 | VSILVWLLA | ACKR3_HUMAN: CS: 164: 179 | CCL15, CCL18, |
| | | FSVSLPD | | CCL19, CCL20, |
| | | | | CCL28, CXL10, |
| | | | | IL8 |
| 209 | CR2586 | CILVWLLAF | ACKR3_HUMAN: WT: 165: 180 | CCL20, CCL28, |
| | | CVSLPDT | | CXL10, CXL11, |
| | | | | CXL14 |
| 210 | CR25994\| | VTLFLYGVV | US28_HCMVA: WT: 35: 50, | CCL19, CCL1, |
| | VP11105 | FLFGSIG | US28_HCMVT: WT: 35: 50, | CCL17, CCL20, |
| | | | US28_HCMVM: WT: 35: 50 | CCL22, CCL28, |
| | | | | CCL5, X3CL1, |
| | | | | CXL10, CXL11, |
| | | | | CXL13, CXL14, |
| | | | | IL8 |
| 211 | CR26001 | VTLTSLAV | CXCR3_HUMAN: CS: 168: 183 | CCL11, CCL2, |
| | | WGLSLLFA | | CCL28, SDF1 |
| 212 | CR26036 | VTSLLIWVIS | CXCR6_HUMAN: WT: 150: 165 | CCL28, X3CL1, |
| | | LLVSLP | | SDF1, CXL14, |
| | | | | IL8 |
| 213 | CR26041 | VTSVITWLV | CCR2_HUMAN: WT: 159: 174 | CCL2, X3CL1, |
| | | AVFASVP | | CXL11, IL8 |
| 214 | CR26093 | VVCILVWLL | ACKR3_HUMAN: WT: 163: 178 | CCL20, X3CL1, |
| | | AFCVSLP | | CXL10, IL8 |
| 215 | CR26115\| | VVFLFGSIG | US28_HCMVA: WT: 42: 57, | CCL1, CCL17, |
| | VP11147 | NFLVIFT | US28_HCMVT: WT: 42: 57, | CCL18, CCL19, |
| | | | US28_HCMVM: WT: 42: 57 | CCL20, CCL22, |
| | | | | CCL5, X3CL1, |
| | | | | CXL10, CXL11, |
| | | | | SDF1, CXL14, |
| | | | | IL8 |
| 216 | CR26155 | VVLIFLLSW | CXCR2_HUMAN: CS: 256: 271, | CCL15, CCL28, |
| | | LPYNLVL | CXCR1_HUMAN: CS: 247: 262 | CXL10, CXL13 |
| 217 | CR26231 | VVTSVITWL | CCR2_HUMAN: WT: 158: 173 | CCL11, CCL17, |
| | | VAVFASV | | CCL18, CCL2, |
| | | | | CCL20, CCL25, |
| | | | | CCL8, CXL10, |
| | | | | CXL11, CXL14, |
| | | | | IL8 |
| 218 | CR26232 | VVTSVITWV | CCR5_HUMAN: WT: 146: 161 | CCL17, CCL18, |
| | | VAVFASL | | CCL28, CCL8, |
| | | | | X3CL1, CXL10, |
| | | | | SDF1, CXL14 |
| 219 | CR26306 | VWLMAIVG | US27_HCMVA: CA: 37: 52 | CCL22, CXL11, |
| | | TFLNVLVI | | CXL13 |
| 220 | CR26308 | VWLMCIVG | US27_HCMVA: WT: 37: 52 | CCL18, CCL20, |
| | | TFLNVLVI | | CCL22, CCL28, |
| | | | | CXL10, CXL11, |
| | | | | SDF1 |
| 221 | CR26309 | VWLMSIVG | US27_HCMVM: CS: 39: 54 | CCL18, CXL10, |
| | | TFLNMLVI | | CXL11, CXL13, |
| | | | | CXL14 |
| 222 | CR26310 | VWLMSIVG | US27_HCMVA: CS: 37: 52 | CCL17, CCL18, |
| | | TFLNVLVI | | CCL2, CXL10, |
| | | | | CXL11, SDF1, |
| | | | | CXL14, IL8 |
| 223 | CR26327 | VWVASILSSI | XCR1_HUMAN: WT: 153: 168 | CCL2, CXL11, |
| | | LDTIFH | | IL8 |
| 224 | CR26399 | VYTVLLSW | UL33_HCMVM: WT: 197: 212 | CCL1, CCL17, |
| | | KVLLTLVW | | CCL18, CCL19, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 225 | CR26500 | WFLPIMYSII | CCR7_HUMAN: WT: 59: 74 | CCL28, SDF1, |
| | | CFVGLL | | CXL14 |
| 226 | CR26513 | WGAAPVIM | UL33_HCMVM: WT: 212: 227, | CCL11, CCL17, |
| | | MTWFYAFF | UL33_HCMVA: WT: 212: 227 | CCL18, CCL19, |
| | | | | CCL20, CCL22, |
| | | | | CCL25, CCL28, |
| | | | | X3CL1, CXL10, |
| | | | | CXL11, CXL13, |
| | | | | IL8 |
| 227 | CR26521 | WGLALLFA | CXCR3_HUMAN: CA: 176: 191 | CCL15, CXL13, |
| | | LPDFIFLS | | IL8 |
| 228 | CR26536 | WGWVLGDF | XCR1_HUMAN: WT: 93: 108 | CCL1, CCL11, |
| | | LCKLLNMI | | CCL17, CCL18, |
| | | | | CCL20, CCL22, |
| | | | | CCL28, CCL5, |
| | | | | X3CL1, CXL10, |
| | | | | SDF1, IL8 |
| 229 | CR26547 | WHWVFGSF | ACKR2_HUMAN: CA: 108: 123 | CCL1, X3CL1, |
| | | LAKMVSTL | | CXL14 |
| 230 | CR26551\| | WIFAVIIAIP | US28_HCMVA: WT: 152: 167, | CCL1, CCL5, |
| | VP11283 | HFMVVT | US28_HCMVT: WT: 152: 167, | X3CL1, CXL11, |
| | | | US28_HCMVM: WT: 152: 167 | SDF1, CXL13, |
| | | | | IL8 |
| 231 | CR26592 | WKLLAEIYE | VU51_HHV7J: WT: 9: 24 | CCL19, CCL20, |
| | | YLFFFSF | | CCL22, CXL14 |
| 232 | CR26630 | WLMCIVGTF | US27_HCMVA: WT: 38: 53 | CCL1, CCL17, |
| | | LNVLVIT | | CCL18, CCL19, |
| | | | | CCL20, CCL22, |
| | | | | CCL28, CCL8, |
| | | | | X3CL1, CXL10, |
| | | | | CXL11, SDF1, |
| | | | | CXL14, IL8 |
| 233 | CR26633 | WLMSIVGTF | US27_HCMVM: CS: 40: 55 | CCL11, CCL17, |
| | | LNMLVIT | | CCL18, CCL19, |
| | | | | CCL20, CCL22, |
| | | | | CCL25, CCL28, |
| | | | | X3CL1, CXL10 |
| 234 | CR26655 | WMNILWAW | ACKR1_HUMAN: WT: 242: 257 | CCL17, CCL18, |
| | | FIFWWPHG | | CCL19, CCL20, |
| | | | | CCL28, CXL10, |
| | | | | SDF1, IL8 |
| 235 | CR26667 | WNNFHTIM | CCR2_HUMAN: WT: 198: 213 | CCL17, CCL18, |
| | | RNILGLVL | | CXL14 |
| 236 | CR26721 | WSLVFMAIE | VU51_HHV6Z: CA: 96: 111 | CCL17, CCL18, |
| | | RINYFAY | | CXL10 |
| 237 | CR26762 | WVASILSSIL | XCR1_HUMAN: WT: 154: 169 | CCL17, CCL18, |
| | | DTIFHK | | X3CL1, CXL10, |
| | | | | CXL11, IL8 |
| 238 | CR26799 | WVFIFSSIQL | VU51_HHV6Z: CA: 19: 34 | CCL18, CCL19, |
| | | ATVVFL | | CCL20, CCL22, |
| | | | | CCL28, X3CL1, |
| | | | | CXL10, CXL11, |
| | | | | IL8 |
| 239 | CR26801 | WVFIFSSIQL | VU51_HHV6Z: WT: 19: 34 | CCL1, CCL17, |
| | | CTVVFL | | CCL18, CCL19, |
| | | | | CCL20, CCL28, |
| | | | | X3CL1, SDF1 |
| 240 | CR26817 | WVLGDFLC | XCR1_HUMAN: WT: 95: 110 | CCL1, CCL17, |
| | | KLLNMIFS | | CCL18, CCL20, |
| | | | | CCL28, X3CL1, |
| | | | | CXL10, CXL11, |
| | | | | CXL13, CXL14, |
| | | | | IL8 |
| 241 | CR26831 | WVLVIGSLV | GPR35_HUMAN: WT: 141: 156 | CCL17, CCL2, |
| | | ARWLLGI | | X3CL1, CXL10 |
| 242 | CR26850 | WVVVFQFQ | CXCR4_HUMAN: WT: 195: 210 | CCL1, CCL19, |
| | | HIMVGLIL | | CCL20, CCL28, |
| | | | | SDF1 |
| 243 | CR26852\| | WWIFAVIIAI | US28_HCMVA: WT: 151: 166, | CCL1, CCL17, |
| | VP11322 | PHFMVV | US28_HCMVT: WT: 151: 166, | CCL8, X3CL1, |
| | | | US28_HCMVM: WT: 151: 166 | CXL10, IL8 |
| 244 | CR27049 | YDLFIGIKIV | UL33_HHV7J: CA: 203: 218 | CCL1, CCL18, |
| | | YAFLWG | | CCL22, CCL25, |
| | | | | IL8 |
| 245 | CR27050 | YDLFIGIKIV | UL33_HHV7J: WT: 203: 218 | CCL1, CCL17, |
| | | YCFLWG | | CCL19, CCL20, |
| | | | | CCL22, X3CL1, |
| | | | | CXL10, CXL11, |
| | | | | SDF1, CXL13, |
| | | | | CXL14, IL8 |
| 246 | CR27051 | YDLFIGIKIV | UL33_HHV7J: CS: 203: 218 | CCL18, CCL2, |
| | | YSFLWG | | CCL20, CCL25, |
| | | | | CCL28, CXL10, |
| | | | | CXL11, CXL14 |
| 247 | CR27055 | YDMWLML | VU51_HHV6U: WT: 167: 182 | CCL1, CCL11, |
| | | VYSVMIPV | | CCL17, CCL18, |
| | | M | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 248 | CR27265 | YHWGWVL | XCR1_HUMAN: WT: 91: 106 | CCL1, CCL11, |
| | | GDFLCKLLN | | CCL15, CCL17, |
| | | | | CCL18, CCL19, |
| | | | | CCL2, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 249 | CR27428 | YLKAFLAA | ACKR2_HUMAN: WT: 322: 337 | CCL17, CCL18, |
| | | VLGWHLAP | | CCL2, CCL20, |
| | | | | X3CL1, CXL10, |
| | | | | CXL11, SDF1, |
| | | | | CXL14 |
| 250 | CR27433 | YLLDFILAL | GPR17_HUMAN: WT: 66: 81 | CCL18, CCL19, |
| | | VGNTLAL | | CCL20, CCL25, |
| | | | | CCL28, CXL10, |
| | | | | CXL11 |
| 251 | CR27448 | YLLNLAISD | CCR4_HUMAN: WT: 77: 92 | CCL1, CCL2, |
| | | LLFVFSL | | CXL11, IL8 |
| 252 | CR27514 | YLYVYLDIF | VGPCR_HHV8P: CS: 126: 141 | CCL1, CCL19, |
| | | SVVSVSL | | SDF1 |
| 253 | CR27677 | YSAFLSGMT | BILF1_EBVB9: WT: 32: 47 | CCL17, CCL18, |
| | | SLLLVLL | | CCL20, CCL25, |
| | | | | CXL10, CXL14 |
| 254 | CR27944\| | YVAMFASLS | US28_HCMVA: CS: 112: 127, | X3CL1, CXL10, |
| | VP11737 | FITEIAL | US28_HCMVT: CS: 112: 127, | CXL11 |
| | | | US28_HCMVM: CS: 112: 127 | |
| 255 | CR28001 | YVSFISTTIL | O41975_MHV68: CS: 249: 264 | CCL1, CCL15, |
| | | FLIFSV | | CCL17, CCL19, |
| | | | | CCL20, CCL22, |
| | | | | CCL25, CCL28, |
| | | | | X3CL1, CXL10, |
| | | | | CXL11, CXL14 |
| 256 | CR28005 | YVSTLDFAI | O41975_MHV68: CA: 123: 138 | CCL17, CCL18, |
| | | VFILMII | | CCL19, CCL20, |
| | | | | CCL22, CCL28, |
| | | | | X3CL1, CXL10, |
| | | | | CXL11, SDF1, |
| | | | | CXL13 |
| 257 | CR3107 | DFAAILLYID | VU51_HHV6Z: CA: 78: 93 | CCL18, X3CL1, |
| | | DFSAYF | | CXL13 |
| 258 | CR3135 | DFLCKLLN | XCR1_HUMAN: WT: 99: 114 | CCL17, CCL22, |
| | | MIFSISLY | | CCL25, CCL28, |
| | | | | CXL11, IL8 |
| 259 | CR3146 | DFLSKLLNM | XCR1_HUMAN: CS: 99: 114 | CCL17, CCL18, |
| | | IFSISLY | | CCL19, CCL2, |
| | | | | CCL20, CXL14 |
| 260 | CR3161 | DFSAILLYID | VU51_HHV6Z: CS: 78: 93 | CCL17, CCL18, |
| | | DFSAYF | | X3CL1, CXL10, |
| | | | | SDF1, CXL13, |
| | | | | CXL14 |
| 261 | CR3302 | DLFIGIKIVY | UL33_HHV7J: CS: 204: 219 | CCL1, CCL17, |
| | | SFLWGI | | CCL18, CCL20, |
| | | | | CCL22, CCL28, |
| | | | | CXL10, CXL11, |
| | | | | CXL13, IL8 |
| 262 | CR3373 | DLTNWKLL | VU51_HHV7J: WT: 5: 20 | CCL2, CCL22, |
| | | AEIYEYLF | | CCL28, CXL10, |
| | | | | CXL11, SDF1, |
| | | | | CXL13, CXL14, |
| | | | | IL8 |
| 263 | CR362 | AEIYEYLFFF | VU51_HHV7J: CA: 13: 28 | CCL22, CXL11, |
| | | SFFFLA | | CXL13 |
| 264 | CR3716 | DVYLLNLAI | CCR4_HUMAN: WT: 75: 90 | CCL11, CCL2, |
| | | SDLLFVF | | CCL8, CXL11 |
| 265 | CR3800 | DYSIILMFLS | VU51_HHV7J: CS: 77: 92 | CCL1, CCL11, |
| | | DFSFIF | | CCL15, CCL18, |
| | | | | CCL28, X3CL1, |
| | | | | CXL10, CXL11, |
| | | | | SDF1, CXL13, |
| | | | | IL8 |
| 266 | CR3983\| | EFDYDEDAT | US28_HCMVA: WT: 13: 28, | CCL11, CCL15, |
| | VP2025 | PCVFTDV | US28_HCMVT: WT: 13: 28, | CCL17, CCL18, |
| | | | US28_HCMVM: WT: 13: 28 | CCL19, CCL20, |
| | | | | CCL25, X3CL1, |
| | | | | CXL14 |
| 267 | CR4135 | EIYEYLFFFS | VU51_HHV7J: CA: 14: 29 | CCL20, CCL25, |
| | | FFFLAL | | CXL11, CXL13 |
| 268 | CR4205 | ELFLAVKIV | UL33_HHV6U: WT: 187: 202 | CCL11, CCL17, |
| | | FSFIWGV | | CCL18, CCL19, |
| | | | | CCL2, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 269 | CR4463 | ESLFAALVT | O41975_MHV68: CA: 302: 317 | CCL1, CCL17, |
| | | LITSLFG | | CCL18, CCL2, |
| | | | | X3CL1, CXL10, |
| | | | | CXL11, IL8 |
| 270 | CR4464 | ESLFCALVT | O41975_MHV68: WT: 302: 317 | CCL1, CCL11, |
| | | LITSLFG | | CCL17, CCL18, |
| | | | | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 271 | CR4465 | ESLFSALVT | O41975_MHV68: CS: 302: 317 | CCL18, CCL8, |
| | | LITSLFG | | CXL10 |
| 272 | CR4712 | FAIVVAYFL | XCR1_HUMAN: WT: 228: 243 | CCL18, CCL19, |
| | | SWGPYNF | | CCL20, CCL22, |
| | | | | CCL25, SDF1 |
| 273 | CR4753 | FAQLVYFIY | VGCR_HHV6Z: CA: 104: 119, | CCL2, CXL13, |
| | | HAVASYS | UL33_HHV6U: CA: 100: 115 | IL8 |
| 274 | CR4754 | FARAIDIIYS | ACKR4_HUMAN: CA: 261: 276 | CCL18, CCL28, |
| | | LITSAN | | X3CL1 |
| 275 | CR4756 | FASDFVLM | VGCR_HHV6Z: CA: 77: 92, | CCL17, X3CL1, |
| | | WAVFFMTV | UL33_HHV6U: CA: 73: 88 | CXL10 |
| 276 | CR4760 | FASFYLLDFI | GPR17_HUMAN: WT: 62: 77 | CCL22, CXL10, |
| | | LALVGN | | CXL11, SDF1, |
| | | | | CXL13, IL8 |
| 277 | CR4802 | FCALVTLITS | O41975_MHV68: WT: 305: 320 | CCL11, CCL17, |
| | | LFGSIF | | CCL18, CCL19, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 278 | CR4845 | FDFMILNLS | GPR75_HUMAN: WT: 79: 94 | X3CL1, CXL13, |
| | | FCDLFIC | | IL8 |
| 279 | CR4860\| | FDYDEDATP | US28_HCMVA: WT: 14: 29, | CCL17, CCL18, |
| | VP2555 | CVFTDVL | US28_HCMVT: WT: 14: 29, | CCL19, CCL20 |
| | | | US28_HCMVM: WT: 14: 29 | |
| 280 | CR498 | AFLWGIFPT | UL33_HHV7J: CA: 214: 229 | CCL17, CCL18, |
| | | VIFSYFY | | CCL19, CCL20, |
| | | | | CCL25, CXL13 |
| 281 | CR5021 | FFSGMLLLL | CCR7_HUMAN: CS: 140: 155 | CCL18, CCL19, |
| | | SISIDRY | | CCL20, CCL28 |
| 282 | CR5027 | FFSQLVYFIY | VGCR_HHV6Z: CS: 103: 118, | CCL17, CCL18, |
| | | HAVSSY | UL33_HHV6U: CS: 99: 114 | CCL20, CXL10, |
| | | | | CXL11, SDF1, |
| | | | | IL8 |
| 283 | CR5055 | FFYYLYVYL | VGPCR_HHV8P: CA: 123: 138 | CCL18, CCL2, |
| | | DIFSVVA | | CCL22, CXL10, |
| | | | | SDF1 |
| 284 | CR5056 | FFYYLYVYL | VGPCR_HHV8P: WT: 123: 138 | CCL1, CCL11, |
| | | DIFSVVC | | CCL17, CCL18, |
| | | | | CCL19, CCL20, |
| | | | | CCL22, CCL25, |
| | | | | CCL28, CCL8, |
| | | | | X3CL1, CXL10, |
| | | | | CXL11, SDF1, |
| | | | | CXL13, CXL14, |
| | | | | IL8 |
| 285 | CR5057 | FFYYLYVYL | VGPCR_HHV8P: CS: 123: 138 | CCL1, CCL11, |
| | | DIFSVVS | | CCL15, CCL17, |
| | | | | CCL18, CCL19, |
| | | | | CCL2, CCL20, |
| | | | | CCL22, CCL25, |
| | | | | CCL8, X3CL1, |
| | | | | CXL10, CXL11, |
| | | | | SDF1, CXL14, |
| | | | | IL8 |
| 286 | CR5077 | FGFFIPLMF | CCR6_HUMAN: CS: 221: 236 | CCL18, X3CL1, |
| | | MIFSYTF | | CXL14 |
| 287 | CR5103 | FGLLGNILV | CCR6_HUMAN: WT: 59: 74 | CCL19, CCL20, |
| | | VITFAFY | | CCL22, CCL28, |
| | | | | CXL10, SDF1, |
| | | | | CXL14 |
| 288 | CR5170 | FGVITSIVT | CCR3_HUMAN: WT: 149: 164 | X3CL1, CXL10, |
| | | WGLAVLA | | IL8 |
| 289 | CR5171 | FGVVTSVIT | CCR2_HUMAN: WT: 156: 171 | CCL11, CCL17, |
| | | WLVAVFA | | CCL18, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 290 | CR5172 | FGVVTSVIT | CCR5_HUMAN: WT: 144: 159 | CCL1, CCL18, |
| | | WVVAVFA | | CXL10, SDF1, |
| | | | | IL8 |
| 291 | CR5175 | FGVWLMSIV | US27_HCMVM: CS: 37: 52 | CCL17, CCL22, |
| | | GTFLNML | | IL8 |
| 292 | CR5236 | FIFVIGMIAN | ACKR3_HUMAN: WT: 53: 68 | CCL19, CCL28, |
| | | SVVVWV | | CXL11, SDF1, |
| | | | | CXL13, IL8 |
| 293 | CR5242 | FIGFFGSIFFI | CX3C1_HUMAN: WT: 110: 125 | CCL17, CCL18, |
| | | TVISI | | CCL20, X3CL1, |
| | | | | CXL13 |
| 294 | CR5243 | FIGFFSGIFFI | CCR5_HUMAN: WT: 109: 124 | CCL17, CCL18, |
| | | ILLTI | | CCL19, CCL20, |
| | | | | CCL22, CXL11, |
| | | | | SDF1, CXL14 |
| 295 | CR5245 | FIGIKIVYCF | UL33_HHV7J: WT: 206: 221 | CCL1, CCL15, |
| | | LWGIFP | | CCL17, CCL18, |
| | | | | CCL19, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 296 | CR5246 | FIGIKIVYSF | UL33_HHV7J: CS: 206: 221 | CCL20, CCL22, |
| | | LWGIFP | | X3CL1, CXL10, |
| | | | | CXL11 |
| 297 | CR5262 | FILALVGNT | GPR17_HUMAN: WT: 70: 85 | CCL22, CCL28, |
| | | LALWLFI | | SDF1, IL8 |
| 298 | CR5277 | FILYQFELFG | GPR75_HUMAN: WT: 352: 367 | CCL17, CCL22, |
| | | FTLIFF | | CCL25, CCL28, |
| | | | | CXL10, CXL11, |
| | | | | SDF1 |
| 299 | CR5349 | FIYVLMFLC | VG74_SHV21: WT: 44: 59 | CCL19, CCL20, |
| | | NAIGNSL | | CXL10 |
| 300 | CR5416 | FLAFLRSGY | VU51_HHV6U: WT: 159: 174 | CCL1, CCL11, |
| | | DMWLMLV | | CCL17, CCL18, |
| | | | | CCL2, CCL20, |
| | | | | CXL11, CXL13, |
| | | | | IL8 |
| 301 | CR5474\| | FLFGSIGNFL | US28_HCMVA: WT: 44: 59, | CCL1, CCL11, |
| | VP2659 | VIFTIT | US28_HCMVT: WT: 44: 59, | CCL15, CCL20, |
| | | | US28_HCMVM: WT: 44: 59 | CCL5, X3CL1, |
| | | | | CXL10, CXL11, |
| | | | | IL8 |
| 302 | CR5475 | FLFHLAVAD | CXCR5_HUMAN: WT: 89: 104 | CCL20, CCL22, |
| | | LLLVFIL | | CCL28, CXL10, |
| | | | | CXL11, SDF1, |
| | | | | CXL14 |
| 303 | CR5511 | FLGILGNGL | CML1_HUMAN: WT: 52: 67 | CCL20, CCL22, |
| | | VIIIATF | | SDF1 |
| 304 | CR5518 | FLGLMASV | BILF1_EBVB9: CA: 105: 120 | CCL2, CXL10, |
| | | WTHLGMAL | | CXL11 |
| 305 | CR5519 | FLGLMCSV | BILF1_EBVB9: WT: 105: 120 | CCL1, CCL18, |
| | | WTHLGMAL | | CCL2, CCL22, |
| | | | | CCL28, X3CL1, |
| | | | | CXL10, CXL11, |
| | | | | SDF1, CXL13, |
| | | | | CXL14, IL8 |
| 306 | CR5598 | FLLSLGIILFS | XCR1_HUMAN: CS: 194: 209 | CCL18, CCL19, |
| | | YVEIL | | CXL10, CXL11, |
| | | | | SDF1, CXL14, |
| | | | | IL8 |
| 307 | CR5669 | FLSIFYSVIF | CX3C1_HUMAN: WT: 33: 48 | CCL17, CCL18, |
| | | AIGLVG | | CCL19, CCL20, |
| | | | | CCL28, X3CL1, |
| | | | | CXL10, CXL14 |
| 308 | CR5737 | FLVVVYSIV | CML1_HUMAN: CS: 42: 57 | CCL17, CCL18, |
| | | SFLGILG | | CCL19, CCL2, |
| | | | | CCL20, CCL22, |
| | | | | CCL25, CCL28, |
| | | | | X3CL1, CXL10, |
| | | | | CXL13, CXL14 |
| 309 | CR5738 | FLWGIFPTVI | UL33_HHV7J: WT: 215: 230 | CCL18, CCL22, |
| | | FSYFYV | | CCL8, X3CL1, |
| | | | | CXL10, CXL11, |
| | | | | IL8 |
| 310 | CR5741\| | FLYGVVFLF | US28_HCMVA: WT: 38: 53, | CCL1, CCL20, |
| | VP2681 | GSIGNFL | US28_HCMVT: WT: 38: 53, | CCL22, CCL28, |
| | | | US28_HCMVM: WT: 38: 53 | X3CL1, CXL10, |
| | | | | CXL11, IL8, |
| | | | | CCL11, CCL17, |
| | | | | CCL18, CCL19, |
| | | | | CCL2, CCL25, |
| | | | | CCL4, CCL5, |
| | | | | CCL8, SDF1, |
| | | | | CXL13, CXL14 |
| 311 | CR5767 | FMFVLSVLG | O41975_MHV68: CA: 55: 70 | CCL1, CCL15, |
| | | NLTVVAV | | CCL17, CCL18, |
| | | | | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 312 | CR5768 | FMFVLSVLG | O41975_MHV68: WT: 55: 70 | CCL19, CCL22, |
| | | NLTVVCV | | CCL25 |
| 313 | CR5775 | FMIFAYTFIV | CCR6_HUMAN: CA: 229: 244 | CCL17, CCL18, |
| | | KTLVQA | | CCL28, X3CL1, |
| | | | | CXL11, SDF1, |
| | | | | CXL13, CXL14, |
| | | | | IL8 |
| 314 | CR5776 | FMIFCYTFIV | CCR6_HUMAN: WT: 229: 244 | CCL1, CCL17, |
| | | KTLVQA | | CCL19, CCL20, |
| | | | | SDF1, CXL13 |
| 315 | CR6041 | FSAILLYIDD | VU51_HHV6Z: CS: 79: 94 | CCL17, CCL18, |
| | | FSAYFS | | X3CL1, IL8 |
| 316 | CR6044 | FSALVTLITS | O41975_MHV68: CS: 305: 320 | CCL2, X3CL1, |
| | | LFGSIF | | CXL10, IL8 |
| 317 | CR6068 | FSFFYVIFSK | UL33_HHV6U: CS: 208: 223 | CCL1, CCL18, |
| | | ALHDVT | | CCL22, CCL28, |
| | | | | SDF1 |
| 318 | CR6097\| | FSIFWWIFA | US28_HCMVA: WT: 147: 162, | CCL20, CCL28, |
| | VP2744 | VIIAIPH | US28_HCMVT: WT: 147: 162, | CXL10, SDF1, |
| | | | US28_HCMVM: WT: 147: 162 | IL8, CCL1, |
| | | | | CCL15, CCL22, |
| | | | | CCL5, X3CL1, |
| | | | | CXL11, CXL13 |
| 319 | CR6098 | FSIIAVFYFL | ACKR3_HUMAN: WT: 225: 240 | CCL17, CCL20, |
| | | LARAIS | | CCL22, CXL10, |
| | | | | CXL11, CXL13, |
| | | | | CXL14, IL8 |
| 320 | CR6163 | FSRAIDIIYS | ACKR4_HUMAN: CS: 261: 276 | CCL17, CCL18, |
| | | LITSSN | | CCL2, CCL22, |
| | | | | CCL28, X3CL1, |
| | | | | CXL14 |
| 321 | CR6212 | FSTFSGMLV | VU51_HHV7J: WT: 48: 63 | CXL11, CXL13, |
| | | YILLLPV | | IL8 |
| 322 | CR6235 | FSYVVVFLV | ACKR3_HUMAN: CS: 255: 270 | CCL1, CCL15, |
| | | SWLPYHV | | CCL17, CCL18, |
| | | | | CCL19, CCL2, |
| | | | | CCL20, CCL22, |
| | | | | CCL25, CCL28, |
| | | | | CCL8, X3CL1, |
| | | | | CXL10, CXL11, |
| | | | | SDF1, CXL13, |
| | | | | CXL14, IL8 |
| 323 | CR6269 | FTGMPPADE | CXCR1_HUMAN: WT: 17: 32 | CCL11, CCL17, |
| | | DYSPCML | | CCL18, CCL19, |
| | | | | CCL20 |
| 324 | CR6324 | FTWNLVLSQ | UL78_HCMVM: WT: 77: 92 | CCL11, CCL28, |
| | | FFSILAT | | X3CL1, CXL11, |
| | | | | CXL13 |
| 325 | CR6489 | FWAVDAVA | CXCR4_HUMAN: WT: 93: 108 | CCL17, CCL18, |
| | | NWYFGNFL | | CCL22, CCL28, |
| | | | | CXL10, CXL14, |
| | | | | IL8 |
| 326 | CR6515 | FWTPYNIVL | CCR4_HUMAN: WT: 254: 269 | CCL20, CCL22, |
| | | FLETLVE | | CXL10, CXL13, |
| | | | | IL8 |
| 327 | CR6520\| | FWWIFAVII | US28_HCMVA: WT: 150: 165, | CCL1, X3CL1, |
| | VP2866 | AIPHFMV | US28_HCMVT: WT: 150: 165, | CXL10, CXL11 |
| | | | US28_HCMVM: WT: 150: 165 | |
| 328 | CR6558 | FYISLFAGVS | US27_HCMVA: CS: 110: 125, | CCL17, CCL18, |
| | | FLINLS | US27_HCMVM: CS: 112: 127 | CCL19, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 329 | CR6604\| | FYVAMFASL | US28_HCMVA: WT: 111: 126, | CCL15, X3CL1, |
| | VP2872 | CFITEIA | US28_HCMVT: WT: 111: 126, | CXL10, CXL11, |
| | | | US28_HCMVM: WT: 111: 126 | SDF1, CXL13, |
| | | | | IL8 |
| 330 | CR6605\| | FYVAMFASL | US28_HCMVA: CS: 111: 126, | CCL1, CCL5, |
| | VP2873 | SFITEIA | US28_HCMVT: CS: 111: 126, | CXL10, CXL11, |
| | | | US28_HCMVM: CS: 111: 126 | IL8 |
| 331 | CR6611 | FYVITLFAS | VGCR_HHV6Z: WT: 71: 86, | CCL22, CCL25, |
| | | DFVLMWC | UL33_HHV6U: WT: 67: 82 | X3CL1, CXL13 |
| 332 | CR6623 | FYYIGFYSS | CCR8_HUMAN: WT: 112: 127 | CCL1, CCL17, |
| | | MFFITLM | | CCL18, CCL19, |
| | | | | CCL20, CCL22, |
| | | | | CCL28, X3CL1, |
| | | | | CXL10, CXL11, |
| | | | | SDF1, CXL14 |
| 333 | CR6624 | FYYLYVYL | VGPCR_HHV8P: CA: 124: 139 | CCL15, CCL17, |
| | | DIFSVVAV | | CCL18, CCL19, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 334 | CR6746 | GDFLAKLLN | XCR1_HUMAN: CA: 98: 113 | CCL20, CCL25, |
| | | MIFSISL | | CCL28 |
| 335 | CR6836 | GFFIPLMFMI | CCR6_HUMAN: CS: 222: 237 | CCL22, CXL14, |
| | | FSYTFI | | IL8 |
| 336 | CR7000 | GIFFIILLTID | CCR5_HUMAN: WT: 115: 130 | CCL17, X3CL1, |
| | | RYLAV | | CXL10 |
| 337 | CR7024 | GIITSVLAW | CCRL2_HUMAN: WT: 148: 163 | CCL2, CCL22, |
| | | VTAILAT | | CCL28, CXL10, |
| | | | | CXL11, SDF1, |
| | | | | CXL13, IL8 |
| 338 | CR7344 | GMGPGPWM | ACKR1_HUMAN: WT: 236: 251 | CCL1, CCL11, |
| | | NILWAWFI | | CCL17, CCL18, |
| | | | | CCL19, CCL2, |
| | | | | CCL20, CCL22, |
| | | | | CCL25, CCL8, |
| | | | | X3CL1, CXL10, |
| | | | | CXL11, SDF1, |
| | | | | CXL14, IL8 |
| 339 | CR7367 | GMPPADED | CXCR1_HUMAN: WT: 19: 34 | CCL11, CCL18, |
| | | YSPCMLET | | CCL19, CCL20 |
| 340 | CR7477 | GPGPWMNIL | ACKR1_HUMAN: WT: 238: 253 | CCL17, CCL18, |
| | | WAWFIFW | | CCL19, CCL20, |
| | | | | CCL25, CXL13, |
| | | | | IL8 |
| 341 | CR7642 | GSMLLSVLF | O41975_MHV68: CS: 112: 127 | CCL25, CXL10, |
| | | TLYVSTL | | CXL11, CXL13 |
| 342 | CR7666 | GSVSLVNLL | UL78_HCMVM: WT: 48: 63 | CCL17, CCL20, |
| | | TIIGCLW | | CCL25 |
| 343 | CR7733 | GTVFLSIFYS | CX3C1_HUMAN: WT: 30: 45 | CCL1, CCL11, |
| | | VIFAIG | | CCL17, CCL18, |
| | | | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 344 | CR7814 | GVVTSVITW | CCR2_HUMAN: WT: 157: 172 | CCL11, CCL2, |
| | | LVAVFAS | | CCL22, CCL8, |
| | | | | IL8 |
| 345 | CR7815 | GVVTSVITW | CCR5_HUMAN: WT: 145: 160 | CCL1, CCL15, |
| | | VVAVFAS | | CCL2, CCL20, |
| | | | | CCL22, CCL25, |
| | | | | CCL28, CCL8, |
| | | | | CXL10, CXL13 |
| 346 | CR7818 | GVWLMAIV | US27_HCMVM: CA: 38: 53 | CCL1, CCL18, |
| | | GTFLNMLV | | CXL11, SDF1, |
| | | | | CXL14 |
| 347 | CR7819 | GVWLMAIV | US27_HCMVA: CA: 36: 51 | CCL17, CCL18, |
| | | GTFLNVLV | | CCL2, CCL20, |
| | | | | CCL22, CXL11, |
| | | | | SDF1, CXL13, |
| | | | | IL8 |
| 348 | CR7823 | GVWLMSIV | US27_HCMVA: CS: 36: 51 | CCL11, CCL17, |
| | | GTFLNVLV | | CCL18, CCL2, |
| | | | | CCL8, CXL10, |
| | | | | CXL11, CXL14, |
| | | | | IL8 |
| 349 | CR7854 | GWVFIFSSIQ | VU51_HHV6Z: CA: 18: 33 | CCL1, CCL15, |
| | | LATVVF | | CCL17, CCL19, |
| | | | | CCL20, CCL22, |
| | | | | CCL28, X3CL1, |
| | | | | CXL10, CXL11, |
| | | | | SDF1, CXL13, |
| | | | | IL8 |
| 350 | CR7856 | GWVFIFSSIQ | VU51_HHV6Z: WT: 18: 33 | CCL1, CCL19, |
| | | LCTVVF | | CCL20, CCL25, |
| | | | | CXL10, SDF1, |
| | | | | IL8 |
| 351 | CR7860 | GWVLGDFL | XCR1_HUMAN: WT: 94: 109 | CCL1, CCL17, |
| | | CKLLNMIF | | CCL18, CCL19, |
| | | | | CCL2, CCL20, |
| | | | | CCL22, CCL28, |
| | | | | CCL8, CXL10, |
| | | | | SDF1, CXL13, |
| | | | | IL8 |
| 352 | CR8126 | HIGYFGGIFF | CCR2_HUMAN: WT: 121: 136 | CCL18, CCL22, |
| | | IILLTI | | CCL25, CXL11, |
| | | | | IL8 |
| 353 | CR8251 | HNKTLSFISL | UL33_HHV7J: WT: 244: 259 | CCL2, X3CL1, |
| | | LILSFL | | IL8 |
| 354 | CR8339 | HRTVKLIFAI | XCR1_HUMAN: WT: 221: 236 | CCL28, X3CL1, |
| | | VVAYFL | | CXL11, SDF1, |
| | | | | CXL14 |
| 355 | CR8427 | HTLRMTIFC | CCR3_HUMAN: WT: 199: 214 | CCL28, CXL10, |
| | | LVLPLLV | | CXL11, SDF1, |
| | | | | IL8 |
| 356 | CR8437 | HTVLLSWK | UL33_HCMVA: WT: 198: 213 | CCL1, CCL18, |
| | | VLLTMVWG | | CCL19, CCL20, |
| | | | | CXL13 |
| 357 | CR8496 | HWGWVLG | XCR1_HUMAN: WT: 92: 107 | CCL11, CCL17, |
| | | DFLCKLLN | | CCL18, CCL19, |
| | | M | | CCL20, CCL22, |
| | | | | CCL25, CCL28, |
| | | | | X3CL1, CXL10, |
| | | | | CXL11, SDF1, |
| | | | | CXL13, CXL14, |
| | | | | IL8 |
| 358 | CR8546 | IAAFSWFML | BILF1_EBVB9: CA: 233: 248 | CCL22, CCL28, |
| | | SLPLLFL | | CXL14 |
| 359 | CR8655 | IAVFGLLGNI | CCR6_HUMAN: CA: 56: 71 | CCL17, CCL22, |
| | | LVVITF | | CCL25, IL8 |
| 360 | CR8675 | IAYSLIAVFG | CCR6_HUMAN: CA: 51: 66 | CCL17, CCL22, |
| | | LLGNIL | | CCL25, CCL28, |
| | | | | CXL11 |
| 361 | CR8677 | IAYSLISVFG | CCR6_HUMAN: CS: 51: 66 | CCL2, CXL10, |
| | | LLGNIL | | CXL13 |
| 362 | CR8784 | IFAIVVAYFL | XCR1_HUMAN: WT: 227: 242 | CCL1, CCL15, |
| | | SWGPYN | | CCL17, CCL18, |
| | | | | CCL2, CCL20, |
| | | | | CCL22, CCL25, |
| | | | | CCL28, X3CL1, |
| | | | | CXL10, CXL11, |
| | | | | SDF1, CXL14, |
| | | | | IL8 |
| 363 | CR8812 | IFFIILLTIDR | CCR3_HUMAN: WT: 121: 136, | CCL18, CCL19, |
| | | YLAIV | CCR1_HUMAN: WT: 121: 136, | CCL22, CXL14 |
| | | | CCR2_HUMAN: WT: 128: 143 | |
| 364 | CR8813 | IFFIILLTIDR | CCR5_HUMAN: WT: 116: 131 | CCL17, CCL18, |
| | | YLAVV | | CCL20, CCL25, |
| | | | | CCL28, CXL10, |
| | | | | CXL11, CXL13, |
| | | | | CXL14, IL8 |
| 365 | CR8966\| | IFWWIFAVII | US28_HCMVA: WT: 149: 164, | CCL22, X3CL1, |
| | VP3949 | AIPHFM | US28_HCMVT: WT: 149: 164, | IL8 |
| | | | US28_HCMVM: WT: 149: 164 | |
| 366 | CR9087 | IIALVVWGL | CCR6_HUMAN: CA: 166: 181 | CCL1, CCL17, |
| | | SVIISSS | | CCL18, CCL19, |
| | | | | CCL2, CCL20, |
| | | | | CCL22, CCL25, |
| | | | | CCL28, X3CL1, |
| | | | | CXL10, CXL11, |
| | | | | SDF1, CXL13, |
| | | | | CXL14, IL8 |
| 367 | CR9111 | IIFSYVVVFL | ACKR3_HUMAN: CA: 253: 268 | CCL20, CCL22, |
| | | VAWLPY | | CCL28, CXL11, |
| | | | | CXL13 |
| 368 | CR9113 | IIFSYVVVFL | ACKR3_HUMAN: CS: 253: 268 | CCL17, CCL20, |
| | | VSWLPY | | CCL22, CCL28, |
| | | | | X3CL1, CXL10, |
| | | | | CXL11, SDF1, |
| | | | | CXL13, CXL14, |
| | | | | IL8 |
| 369 | CR9141 | IIIWALAILA | CCR1_HUMAN: WT: 155: 170 | CCL20, CCL28, |
| | | SMPGLY | | CXL11, SDF1 |
| 370 | CR9192 | IISLVVWGL | CCR6_HUMAN: CS: 166: 181 | CCL18, CXL10, |
| | | SVIISSS | | CXL13, CXL14, |
| | | | | IL8 |
| 371 | CR9218 | IITSVLAWV | CCRL2_HUMAN: WT: 149: 164 | CCL17, CCL18, |
| | | TAILATL | | CXL11, CXL14, |
| | | | | IL8 |
| 372 | CR9282 | ILAFFAAWL | CXCR4_HUMAN: CA: 245: 260 | CCL11, CCL18, |
| | | PYYIGIS | | X3CL1, CXL10, |
| | | | | CXL11, CXL13 |
| 373 | CR9284 | ILAFFASWL | CXCR4_HUMAN: CS: 245: 260 | CCL17, CCL18, |
| | | PYYIGIS | | CCL25 |
| 374 | CR9291 | ILALVGNTL | GPR17_HUMAN: WT: 71: 86 | CCL2, CCL22, |
| | | ALWLFIR | | CCL25, IL8 |
| 375 | CR9432 | ILMFLSDFSF | VU51_HHV7J: WT: 81: 96 | CCL1, CXL10, |
| | | IFSSWA | | CXL11, SDF1, |
| | | | | CXL14, IL8 |
| 376 | CR9515 | ILYQFELFGF | GPR75_HUMAN: WT: 353: 368 | CCL18, CCL19, |
| | | TLIFFK | | CXL10, CXL14 |
| 377 | CR9587 | IMYSIIAFVG | CCR7_HUMAN: CA: 63: 78 | CCL17, CCL18, |
| | | LLGNGL | | CCL20, CXL14 |
| 378 | CR97\| | AALFSIFWW | US28_HCMVA: CA: 144: 159, | CCL1, CCL17, |
| | VP40 | IFAVIIA | US28_HCMVT: CA: 144: 159, | CCL18, CCL20, |
| | | | US28_HCMVM: CA: 144: 159 | CCL25, CXL10, |
| | | | | SDF1, CXL13, |
| | | | | CXL14, IL8, |
| | | | | CCL11, CCL22, |
| | | | | CCL5, X3CL1, |
| | | | | CXL11 |
| 379 | CR9887 | ISLFAGVSFL | US27_HCMVA: CS: 112: 127, | CCL17, CCL18, |
| | | INLSMD | US27_HCMVM: CS: 114: 129 | CCL25 |
| 380 | CR9889 | ISLLAEVLM | VGPCR_HHV8P: WT: 98: 113 | CCL17, CCL18, |
| | | FLFPNII | | CCL20, CCL25, |
| | | | | CCL28, CXL13 |
| 381 | CR9948 | ISVAWHWV | ACKR2_HUMAN: CS: 104: 119 | CCL17, CCL18, |
| | | FGSFLSKM | | CCL20, CCL22, |
| | | | | CCL25, X3CL1, |
| | | | | CXL11, CXL14, |
| | | | | IL8 |
| 382 | EB1522 | GNSTEGRCI | EV675_IXORI: WT: 69: 84 | CXL11, SDF1, |
| | | ELIGDFD | | CXL14 |
| 383 | EB1728 | HEDGKKVG | E1132_IXORI: WT: 64: 79 | CXL10, SDF1, |
| | | LCLSTEYT | | CXL14 |
| 384 | EB2047 | KSDEYELFT | E1168_IXORI: WT: 33: 48 | CXL10, CXL11, |
| | | VEYCGTN | | SDF1, CXL14 |
| 385 | EB2053 | KSDVYELFT | E1166_IXORI: WT: 33: 48 | CXL10, CXL11, |
| | | VEYCGTN | | CXL13 |
| 386 | EB2359 | NDKKVGLC | E1168_IXORI: WT: 73: 88 | CXL10, CXL11, |
| | | LSTEYTDF | | SDF1, CXL14 |
| 387 | EB2905 | SACSGDCVC | E1096_IXORI: WT: 59: 74 | CXL10, CXL11, |
| | | VHRDNEP | | SDF1 |
| 388 | EB3085 | SGLCLSTTYI | E1127_IXORI: WT: 73: 88 | CXL10, CXL11, |
| | | DFSEYG | | SDF1, CXL14 |
| 389 | EB3094 | SGNCTCYHE | E1132_IXORI: WT: 57: 72 | CXL10, CXL11, |
| | | DGKKVGL | | SDF1 |
| 390 | EB3974 | VGLCLSTEY | E1168_IXORI: WT: 77: 92, | CXL10, SDF1, |
| | | TDFSEYP | E1166_IXORI: WT: 77: 92 | CXL14 |
| 391 | EB3975 | VGLCLSTEY | E1132_IXORI: WT: 70: 85 | CXL10, CXL11, |
| | | TDFTKFP | | SDF1, CXL14 |
| 392 | EB429 | CVEITYFGD | E1096_IXORI: WT: 78: 93 | CXL10, CXL11, |
| | | FGDPSQD | | SDF1, CXL13 |
| 393 | EB630 | DKKVGLCLS | E1168_IXORI: WT: 74: 89 | CXL10, SDF1, |
| | | TEYTDFS | | CXL14 |
| 394 | EB936 | EKSDEYELF | E1168_IXORI: WT: 32: 47 | CXL10, CXL11, |
| | | TVEYCGT | | SDF1, CXL13 |
| 395 | EB963 | ENDKKVGL | E1168_IXORI: WT: 72: 87 | CXL10, CXL11, |
| | | CLSTEYTD | | SDF1 |
| 396 | HD2 | EEDDYTAY | EVA4_RHISA: WT: 38: 53 | CCL1, CCL11, |
| | | APLTCYFT | | CCL15, CCL17, |
| | | | | CCL18, CCL20 |
| | | | | CCL22, CCL3, |
| | | | | CCL4, CCL5, |
| | | | | CCL8 |
| 397 | HD2134 | LEEEDDYTA | EVA4_RHISA: WT: 36: 51 | CCL1, CCL11, |
| | | YAPLTCY | | CCL15, CCL17, |
| | | | | CCL18, CCL22, |
| | | | | CCL4, CCL5, |
| | | | | CCL8 |
| 398 | HD2313 | LTDGTACY | EV974_AMBCJ: WT: 66: 81, | CCL19, CCL20, |
| | | VVERKVWD | EV546_AMBCJ: WT: 66: 81 | CCL28 |
| 399 | HD3838 | TDGTACYV | EV974_AMBCJ: WT: 67: 82, | CCL19, CCL2, |
| | | VERKVWDR | EV546_AMBCJ: WT: 67: 82 | CCL20, CCL28 |
| 400 | HD540 | DEDYEDFFK | EV672_RHIPC: WT: 39: 54 | CCL18, CCL22, |
| | | PVTCYFA | | CCL5, CCL8 |
| 401 | HD7 | EEEDDYTAY | EVA4_RHISA: WT: 37: 52 | CCL1, CCL11, |
| | | APLTCYF | | CCL15, CCL17, |
| | | | | CCL18, CCL20, |
| | | | | CCL22, CCL3, |
| | | | | CCL4, CCL5, |
| | | | | CCL8 |
| 402 | HD842 | EDDYTAYA | EVA4_RHISA: WT: 39: 54 | CCL1, CCL17, |
| | | PLTCYFTN | | CCL4, CCL5, |
| | | | | CCL8 |
| 403 | HD845 | EDEDYEDFF | EV672_RHIPC: WT: 38: 53 | CCL18, CCL22, |
| | | KPVTCYF | | CCL5, CCL8 |
| 404 | VP10385 | TYLLFALSL | A0A0A1E5H7_9POXV: WT: 4: 19 | CCL1, CCL11, |
| | | VLSAALS | | CCL17, CCL19, |
| | | | | CCL2, CCL20, |
| | | | | CCL22, CCL25, |
| | | | | CCL28, CCL5, |
| | | | | X3CL1, CXL10, |
| | | | | CXL11, SDF1, |
| | | | | CXL13, CXL14, |
| | | | | IL8 |
| 405 | VP10866 | VNASIAWFF | GG_EHVIB: WT: 82: 97 | X3CL1, CXL10, |
| | | DFGACRM | | CXL11 |
| 406 | VP11546 | YLLFALSLV | A0A0A1E5H7_9POXV: WT: 5: 20 | CCL1, CCL11, |
| | | LSAALSA | | CCL15, CCL17, |
| | | | | CCL18, CCL19, |
| | | | | CCL2, CCL20, |
| | | | | CCL22, CCL25, |
| | | | | CCL28, CCL5, |
| | | | | X3CL1, CXL10, |
| | | | | CXL11, SDF1, |
| | | | | CXL13, CXL14, |
| | | | | IL8 |
| 407 | VP11558 | YLYILFLSAI | CRMB_VARV: CA: 6: 21 | CCL1, CXL10, |
| | | IINGRD | | CXL11, IL8 |
| 408 | VP11559 | YLYILFLSCII | CRMB_VARV: WT: 6: 21 | CXL10, CXL11, |
| | | INGRD | | CXL13, IL8 |
| 409 | VP11668 | YSLVFVILM | A41_VACCW: CA: 2: 17 | CCL1, X3CL1, |
| | | AIPFSFQ | | CXL10, CXL11, |
| | | | | IL8 |
| 410 | VP11669 | YSLVFVILM | A41_VACCW: WT: 2: 17 | X3CL1, CXL10, |
| | | CIPFSFQ | | CXL11, IL8 |
| 411 | VP11670 | YSLVFVILM | A41_VACCW: CS: 2: 17 | X3CL1, CXL10, |
| | | | | CXL11, SDF1, |
| | | SIPFSFQ | | CXL13, IL8 |
| 412 | VP1485 | DLAGLDSLT | O41925_MHV68: WT: 307: 322 | CCL20, X3CL1, |
| | | SPVYVMA | | SDF1 |
| 413 | VP1531 | DMAYYRGD | Q8QMN0_CWPXB: WT: 145: 160 | X3CL1, CXL11, |
| | | CLEVVYVT | | SDF1, CXL13 |
| 414 | VP1624 | DSHMPGRA | Q85308_CWPXB: CA: 330: 345 | CCL11, CCL19, |
| | | DTHKLITN | | CCL20, SDF1, |
| | | | | IL8 |
| 415 | VP291 | AIILLVYVY | VT1_RFVKA: CA: 5: 20 | X3CL1, CXL10, |
| | | ATFATKG | | CXL11, CXL13, |
| | | | | IL8 |
| 416 | VP3426 | GSMMLSLLI | E9M5R0_9GAMA: CS: 9: 24 | CCL2, CCL5, |
| | | AGMSLML | | X3CL1, CXL10, |
| | | | | IL8 |
| 417 | VP4048 | IILLVYVYA | VT1_RFVKA: WT: 6: 21 | CCL1, CCL5, |
| | | TFATKGI | | CCL8, X3CL1, |
| | | | | CXL10, CXL11, |
| | | | | CXL13, IL8 |
| 418 | VP4071 | IKAESKGSE | GG_EHVIB: CS: 56: 71 | CCL17, CCL20, |
| | | LILLDPP | | CCL22 |
| 419 | VP4226 | INTILIFASLF | Q8JLJ0_9POXV: WT: 6: 21, | CCL11, CCL17, |
| | | VASFA | I0AZ78_9POXV: WT: 6: 21 | CCL19, CCL20, |
| | | | | CCL22, CCL28, |
| | | | | CXL10 |
| 420 | VP4486 | IYGLIASLIF | Q8QMN0_CWPXB: CS: 3: 18 | CCL1, CCL17, |
| | | VTSSTA | | CCL19, CCL2, |
| | | | | CCL20, CCL22, |
| | | | | CCL25, CCL28, |
| | | | | CXL13, IL8 |
| 421 | VP485 | AMMLSLLIA | E9M5R0_9GAMA: CA: 10: 25 | CCL11, CCL25, |
| | | GMSLMLG | | CCL28, CCL5, |
| | | | | CXL11, CXL14, |
| | | | | IL8 |
| 422 | VP5438 | LIKAAILLLA | O41925_MHV68: CA: 9: 24 | CCL17, CCL20, |
| | | GGLAES | | CCL25, CCL28, |
| | | | | CCL5, CXL10 |
| 423 | VP5520 | LLFALSLVL | A0A0A1E5H7_9POXV: WT: 6: 21 | CCL1, CCL11, |
| | | SAALSAP | | CCL17, CCL18, |
| | | | | CCL19, CCL2, |
| | | | | CCL20, CCL22, |
| | | | | CCL25, CCL28, |
| | | | | X3CL1, CXL10, |
| | | | | CXL11, SDF1 |
| 424 | VP5543 | LLLALLGAF | Q2F862_ORFV: WT: 5: 20 | CCL11, CCL17, |
| | | TNAAPLL | | CCL19, CCL2, |
| | | | | CCL20, CCL22, |
| | | | | CCL25, CCL28, |
| | | | | X3CL1, CXL14 |
| 425 | VP5770 | LSIILLVYVY | VT1_RFVKA: CS: 4: 19 | X3CL1, CXL10, |
| | | ATFATK | | CXL11 |
| 426 | VP5782 | LSLLIAGMS | E9M5R0_9GAMA: CA: 13: 28 | CCL11, CCL28, |
| | | LMLGHAG | | CCL8, CXL10, |
| | | | | CXL11, SDF1 |
| 427 | VP6009 | LYLYILFLSS | CRMB_VARV: CS: 5: 20 | CCL1, CCL19, |
| | | IIINGR | | CCL20, CCL22, |
| | | | | CCL5, CXL10, |
| | | | | CXL13 |
| 428 | VP6128 | MIYGLIAALI | Q8QMN0_CWPXB: CA: 2: 17 | CCL1, CCL17, |
| | | FVTSST | | CCL19, CCL2, |
| | | | | CCL20, CCL22, |
| | | | | CCL25, CCL28, |
| | | | | CCL5, CCL8, |
| | | | | X3CL1, CXL10, |
| | | | | CXL11, IL8 |
| 429 | VP6130 | MIYGLIASLI | Q8QMN0_CWPXB: CS: 2: 17 | CCL1, CCL11, |
| | | FVTSST | | CCL17, CCL19, |
| | | | | CCL20, CCL22, |
| | | | | CCL25, CCL28, |
| | | | | CXL10, CXL11, |
| | | | | SDF1, CXL14, |
| | | | | IL8 |
| 430 | VP6153 | MKSVLYLYI | CRMB_VARV: CA: 1: 16 | CCL1, CCL17, |
| | | LFLSAII | | CCL20, CCL22, |
| | | | | CCL28, X3CL1, |
| | | | | CXL10, CXL11, |
| | | | | SDF1, CXL14, |
| | | | | IL8 |
| 431 | VP6154 | MKSVLYLYI | CRMB_VARV: WT: 1: 16 | CCL1, X3CL1, |
| | | LFLSCII | | CXL10, CXL11, |
| | | | | IL8 |
| 432 | VP6155 | MKSVLYLYI | CRMB_VARV: CS: 1: 16 | CCL11, CCL17, |
| | | LFLSSII | | CCL19, CCL2, |
| | | | | CCL22, CCL28, |
| | | | | X3CL1, CXL10, |
| | | | | SDF1 |
| 433 | VP6156 | MKSYILLLL | Q85308_CWPXB: CA: 1: 16 | CCL11, CCL18, |
| | | LSAIIII | | CCL19, CCL20, |
| | | | | CCL22, CCL28, |
| | | | | CXL13 |
| 434 | VP6169 | MLSLLIAGM | E9M5R0_9GAMA: CA: 12: 27 | CCL1, CCL11, |
| | | SLMLGHA | | CCL17, CCL18, |
| | | | | CCL19, CCL2, |
| | | | | CCL20, CCL22. |
| | | | | CCL25, CCL28, |
| | | | | CCL5, CXL10, |
| | | | | CXL11, SDF1, |
| | | | | CXL13, CXL14, |
| | | | | IL8 |
| 435 | VP6171 | MLSLLIAGM | E9M5R0_9GAMA: CS: 12: 27 | CCL1, CCL11, |
| | | SLMLGHS | | CCL17, CCL18, |
| | | | | CCL19, CCL2, |
| | | | | CCL20, CCL22, |
| | | | | CCL25, CCL28, |
| | | | | CCL5, X3CL1, |
| | | | | CXL10, CXL11, |
| | | | | SDF1, CXL13, |
| | | | | IL8 |
| 436 | VP6175 | MMIYGLIAA | Q8QMN0_CWPXB: CA: 1: 16 | CCL11, CCL15, |
| | | LIFVTSS | | CCL17, CCL18, |
| | | | | CCL19, CCL20, |
| | | | | CCL22, CCL25, |
| | | | | CCL28, CCL5, |
| | | | | CXL10, SDF1 |
| 437 | VP6177 | MMIYGLIAS | Q8QMN0_CWPXB: CS: 1: 16 | CCL1, CCL11, |
| | | LIFVTSS | | CCL17, CCL18, |
| | | | | CCL19, CCL2, |
| | | | | CCL20, CCL22, |
| | | | | CCL25, CCL28, |
| | | | | X3CL1, CXL10, |
| | | | | CXL11, SDF1, |
| | | | | CXL14, IL8 |
| 438 | VP6179 | MMLSLLIAG | E9M5R0_9GAMA: WT: 11: 26 | CCL1, CXL11, |
| | | MSLMLGH | | CXL14 |
| 439 | VP6281 | MTPSYILLV | O57300_9POXV: WT: 4: 19, | X3CL1, CXL11, |
| | | YMFVVVS | Q7TDW8_9POXV: WT: 4: 19 | CXL13 |
| 440 | VP6289 | MVAVALYA | GN_BHVIC: CA: 67: 82 | CCL1, CCL11, |
| | | YGLAFRLM | | CCL15, CCL17, |
| | | | | CCL19, CCL2, |
| | | | | CCL20, CCL22, |
| | | | | CCL25, CCL28, |
| | | | | CCL5, CXL10, |
| | | | | SDF1, IL8 |
| 441 | VP6306 | MYSLVFVIL | A41_VACCW: CA: 1: 16 | CCL8, CXL11, |
| | | MAIPFSF | | IL8 |
| 442 | VP6307 | MYSLVFVIL | A41_VACCW: WT: 1: 16 | X3CL1, CXL11, |
| | | MCIPFSF | | CXL13, IL8 |
| 443 | VP7686 | RDTNPVSM | O10625_9POXV: WT: 145: 160 | CCL2, CCL5, |
| | | NRTEALAL | | CXL14 |
| 444 | VP7819 | RLEELYLTV | E2CZQ0_9POXV: WT: 242: 257 | CCL1, CCL11, |
| | | ASMFERL | | CCL17, CCL19, |
| | | | | CCL2, CCL22, |
| | | | | CCL28, CXL10 |
| 445 | VP826 | AVLLLALLG | Q2F862_ORFV: WT: 3: 18 | CCL11, CCL15, |
| | | AFTNAAP | | CCL17, CCL19, |
| | | | | CCL20, CCL28, |
| | | | | CCL5, CCL8, |
| | | | | SDF1 |
| 446 | VP8767 | SMMLSLLIA | E9M5R0_9GAMA: CS: 10: 25 | CCL1, CCL11, |
| | | GMSLMLG | | CCL17, CCL19, |
| | | | | CCL2, CCL20, |
| | | | | CCL22, CCL28, |
| | | | | CXL10, CXL11 |
| 447 | VP9245 | STYLLFALS | A0A0A1E5H7_9POXV: WT: 3: 18 | CCL1, CCL11, |
| | | LVLSAAL | | CCL17, CCL18, |
| | | | | CCL19, CCL2, |
| | | | | CCL20, CCL22, |
| | | | | CCL25, CCL28, |
| | | | | CCL5, CCL8, |
| | | | | X3CL1, CXL10, |
| | | | | CXL11, SDF1, |
| | | | | CXL13, CXL14, |
| | | | | IL8 |
| 448 | VP9298 | SVLYLYILFL | CRMB_VARV: CS: 3: 18 | CCL1, CXL10, |
| | | SSIIIN | | IL8 |
| 449 | VP9371 | SYILLLLLSS | Q85308_CWPXB: CS: 3: 18 | CXL10, CXL11, |
| | | IIIINS | | IL8 |
| 450 | VP9710 | TILIFASLFV | Q8JLJ0_9POXV: WT: 8: 23, | CCL1, X3CL1, |
| | | ASFAND | I0AZ78_9POXV: WT: 8: 23 | CXL10, CXL11, |
| | | | | IL8 |
| 451 | VP988 | CIILLVYVY | VT1_RFVKA: WT: 5: 20 | CCL1, CCL8, |
| | | ATFATKG | | X3CL1, CXL10, |
| | | | | CXL11 |

TABLE 5B
Phage-Display Screen Peptides Identified from Saturation Mutagenesis of HD2 Using
Chemokines Displayed on Streptavidin Beads. Chemokines are referred to by the first
part of the UniProt Entry Name.

| SEQ ID | Peptide | Peptide | Chemokines | |
|---|---|---|---|---|
| NO. | ID | Sequence | Bound log2E > 5 | Chemokines Bound log2E 1-5 |
| 452 | HD2_E3 | EEEDYT | CCL3 | CCL1, CCL11, CCL15, CCL17, |
| | | AYAPLT | | CCL18, CCL22, CCL4, CCL5, |
| | | CYFT | | CCL8, X3CL1 |
| 453 | HD2_W5 | EEDDW | CXL10, CXL13 | CCL1, CCL11, CCL15, CCL17, |
| | | TAYAPL | | CCL18, CCL19, CCL2, CCL22, |
| | | TCYFT | | CCL28, CCL3, CCL4, X3CL1, |
| | | | | CXL11, SDF1, IL8 |
| 454 | HD2_D6 | EEDDYD | CCL1, CCL11, | CCL15, CCL17, CCL18, CCL19, |
| | | AYAPLT | CCL2, SDF1, | CCL22, CCL28, CCL3, CCL4, |
| | | CYFT | CXL13 | CCL5, CCL8, X3CL1, CXL10, |
| | | | | CXL11, CXL14, IL8 |
| 455 | HD2_E6 | EEDDYE | SDF1, CXL13 | CCL1, CCL11, CCL15, CCL17, |
| | | AYAPLT | | CCL18, CCL19, CCL2, CCL22, |
| | | CYFT | | CCL28, CCL3, CCL4, CCL5, CCL8, |
| | | | | X3CL1, CXL10, CXL11, CXL14 |
| 456 | HD2_S6 | EEDDYS | CCL1, CCL3 | CCL11, CCL15, CCL17, CCL18, |
| | | AYAPLT | | CCL2, CCL22, CCL4, CCL5, CCL8, |
| | | CYFT | | X3CL1, CXL10, SDF1, CXL13, IL8 |
| 457 | HD2_W6 | EEDDY | CCL19, CCL25, | CCL1, CCL17, CCL18, CCL2, |
| | | WAYAP | CCL28, X3CL1, | CCL20, CCL3, CCL4, IL8 |
| | | LTCYFT | CXL10, CXL11, | |
| | | | SDF1, CXL13, | |
| | | | CXL14 | |
| 458 | HD2_D7 | EEDDYT | SDF1, CXL13 | CCL1, CCL11, CCL15, CCL17, |
| | | DYAPLT | | CCL18, CCL19, CCL2, CCL22, |
| | | CYFT | | CCL28, CCL3, CCL4, CCL5, CCL8, |
| | | | | X3CL1, CXL10, CXL11, CXL14, |
| | | | | IL8 |
| 459 | HD2_E7 | EEDDYT | SDF1, CXL13 | CCL1, CCL11, CCL15, CCL18, |
| | | EYAPLT | | CCL2, CCL22, CCL28, CCL3, |
| | | CYFT | | CCL4, CCL5, CCL8, X3CL1, |
| | | | | CXL10, CXL14, IL8 |
| 460 | HD2_D1C | EEDDYT | CXL11 | |
| | | AYADLT | | |
| | | CYFT | | |
| 461 | HD2_F11 | EEDDYT | CCL20 | CCL18, CXL11, CXL13, CXL14 |
| | | AYAPFT | | |
| | | CYFT | | |
| 462 | HD2_I11 | EEDDYT | CCL15 | CCL1, CCL11, CCL17, CCL18, |
| | | AYAPIT | | CCL19, CCL2, CCL22, CCL28, |
| | | CYFT | | CCL3, CCL4, CCL5, CCL8, X3CL1, |
| | | | | CXL10, CXL11, SDF1, CXL13, IL8 |
| 463 | HD2_Q11 | EEDDYT | CCL17 | CCL1, CCL11, CCL15, CCL18, |
| | | AYAPQT | | CCL22, CCL4, CCL5, CCL8 |
| | | CYFT | | |
| 464 | HD2_T11 | EEDDYT | CCL15 | CCL11, CCL17, CCL18, CCL22, |
| | | AYAPTT | | CCL4, CCL5, CCL8 |
| | | CYFT | | |
| 465 | HD2_V11 | EEDDYT | CCL17 | CCL1, CCL11, CCL15, CCL18, |
| | | AYAPVT | | CCL2, CCL22, CCL28, CCL3, |
| | | CYFT | | CCL4, CCL5, CCL8, CXL10, |
| | | | | CXL11, CXL13 |
| 466 | HD2_Y11 | EEDDYT | CCL22 | CCL1, CCL11, CCL15, CCL17, |
| | | AYAPYT | | CCL18, CCL19, CCL2, CCL20, |
| | | CYFT | | CCL3, CCL4, CCL5, CCL8, X3CL1, |
| | | | | CXL11, CXL13, CXL14 |
| 467 | HD2_H12 | EEDDYT | CCL22 | CCL15, CCL28, CCL4, CCL5 |
| | | AYAPLH | | |
| | | CYFT | | |
| 468 | HD2_112 | EEDDYT | CCL19 | CCL18, CCL25, CCL28, CCL4, |
| | | AYAPLI | | X3CL1, CXL10, SDF1, CXL13, |
| | | CYFT | | CXL14 |
| 469 | HD2_V12 | EEDDYT | CCL19 | CCL18, CCL25, CCL28, CCL4, |
| | | AYAPLV | | CCL8, X3CL1, CXL10, SDF1, |
| | | CYFT | | CXL13, CXL14 |
| 470 | HD2_W12 | EEDDYT | CCL3 | CCL1, CCL15, CCL17, CCL18, |
| | | AYAPL | | CCL19, CCL20, CCL22, CCL25, |
| | | WCYFT | | CCL28, CCL4, X3CL1, SDF1, |
| | | | | CXL14 |
| 471 | HD2_C16 | EEDDYT | IL8 | CCL1, X3CL1, CXL10, CXL11, |
| | | AYAPLT | | CXL14 |
| | | CYFC | | |
| 472 | HD2_D16 | EEDDYT | SDF1 | CCL1, CCL11, CCL17, CCL18, |
| | | AYAPLT | | CCL2, CCL22, CCL3, CCL4, CCL5, |
| | | CYFD | | CCL8, X3CL1, CXL10, CXL11, |
| | | | | CXL13, CXL14 |

TABLE 6
Peptide Sequences and Originating Proteins

| SEQ ID | Peptide | | |
|---|---|---|---|
| NO. | ID | Peptide Sequence | UniProtID: GenBank Accesion |
| 473 | HD990 | ELNCWHDMPPPVSTDT | AMA01: AEO36603.1 |
| 474 | HD2045 | KVANRMQGNVTYTCPV | ATR02: A0A023G9N5 |
| 475 | HD4578 | YDYTEGCPFVVLGNGT | E1180 AMBTT: A0A023G6B6 |
| 476 | HD4357 | VLKLLGCHYFCNGTLC | E1243 AMBAM: A0A0C9S461 |
| 477 | HD540 | DEDYEDFFKPVTCYFA | EV672 RHIPC: L7MC74 |
| 478 | HD744 | DYEDFFKPVTCYFANS | EV672_RHIPC: L7MC74 |
| 479 | HD845 | EDEDYEDFFKPVTCYF | EV672 RHIPC: L7MC74 |
| 480 | HD883 | EDYEDFFKPVTCYFAN | EV672 RHIPC: L7MC74 |
| 481 | HD1054 | ERKVWDRMTPMLWYES | EV974 AMBCJ: A0A023FDY8 |
| 482 | HD2312 | LTDGTAAYVVERKVWD | EV974 AMBCJ: A0A023FDY8, |
| | | | EV546_AMBCJ: A0A023FBW7 |
| 483 | HD2313 | LTDGTACYVVERKVWD | EV974 AMBCJ: A0A023FDY8, |
| | | | EV546 AMBCJ: A0A023FBW7 |
| 484 | HD2314 | LTDGTASYVVERKVWD | EV974 AMBCJ: A0A023FDY8, |
| | | | EV546 AMBCJ: A0A023FBW7 |
| 485 | HD3838 | TDGTACYVVERKVWDR | EV974_AMBCJ: A0A023FDY8, |
| | | | EV546_AMBCJ: A0A023FBW7 |
| 486 | HD3839 | TDGTASYVVERKVWDR | EV974 AMBCJ: A0A023FDY8, |
| | | | EV546 AMBCJ: A0A023FBW7 |
| 487 | HD4670 | YPCYNLTEHQAKNLTT | EV991_AMBCJ: A0A023FFD0 |
| 488 | HD2 | EEDDYTAYAPLTCYFT | EVA4 RHISA: P0C8E9 |
| 489 | HD2133 | LEEEDDYTAYAPLTAY | EVA4 RHISA: P0C8E9 |
| 490 | HD2134 | LEEEDDYTAYAPLTCY | EVA4 RHISA: P0C8E9 |
| 491 | HD2135 | LEEEDDYTAYAPLTSY | EVA4 RHISA: P0C8E9 |
| 492 | HD534 | DDYTAYAPLTCYFTNS | EVA4 RHISA: P0C8E9 |
| 493 | HD612 | DLEEEDDYTAYAPLTS | EVA4 RHISA: P0C8E9 |
| 494 | HD7 | EEEDDYTAYAPLTCYF | EVA4 RHISA: P0C8E9 |
| 495 | HD842 | EDDYTAYAPLTCYFTN | EVA4 RHISA: P0C8E9 |
| 496 | HD900 | EEDDYTAYAPLTAYFT | EVA4 RHISA: P0C8E9 |
| 497 | HD901 | EEDDYTAYAPLTSYFT | EVA4 RHISA: P0C8E9 |
| 498 | HD908 | EEEDDYTAYAPLTAYF | EVA4 RHISA: P0C8E9 |
| 499 | HD909 | EEEDDYTAYAPLTSYF | EVA4 RHISA: P0C8E9 |
| 500 | HD1267 | GAPVVGIGGLDNKTWH | IHO01: A0A223FZ07 |
| 501 | HD3398 | SCVAKVDSLSKEEATV | IRI01: JAA70749.1 |
| 502 | HD4650 | YNIVITKNKTLVVNCT | RPU02: JAA60771.1 |

TABLE 7
Exemplary HD2 variants with improved chemokine-binding
and/or chemotaxis properties

| SEQ ID NO | HD2 mutation | Sequence |
|---|---|---|
| 503 | T6D | EEDDYDAYAPLTCYFT |
| 504 | T6E | EEDDYEAYAPLTCYFT |
| 505 | Y5W | EEDDWTAYAPLTCYFT |
| 506 | A7D | EEDDYTDYAPLTCYFT |
| 507 | L11I | EEDDYTAYAPITCYFT |
| 508 | T6W | EEDDYWAYAPLTCYFT |
| 509 | A7W | EEDDYTWYAPLTCYFT |
| 510 | A9W | EEDDYTAYWPLTCYFT |
| 511 | T12W | EEDDYTAYAPLWCYFT |
| 512 | T16C | EEDDYTAYAPLTCYFC |
| 513 | E1D | DEDDYTAYAPLTCYFT |
| 514 | E2D | EDDDYTAYAPLTCYFT |
| 515 | T16D | EEDDYTAYAPLTCYFD |
| 516 | Y5W; T6D | EEDDWDAYAPLTCYFT |
| 517 | Y5W; L11I | EEDDWTAYAPITCYFT |
| 518 | T6D; L11I | EEDDYDAYAPITCYFT |
| 519 | Y5W; T6D; L11I | EEDDWDAYAPITCYFT |
| 520 | CM307 | DEDDWDDYAPITCYFT |
| 521 | CM462 | DDDDWDAYAPITCYFD |
| 522 | CM539 | DDDDWDDYAPITCYFD |
| 523 | CM470 | EDDDWDDYAPITCYFD |
| 524 | CM325 | EEDDWDDYAPITCYFD |
| 525 | CM315 | DDDDYDAYAPITCYFD |
| 526 | CM313 | DEDDWDAYAPITCYFD |
| 527 | CM346 | DDDDWDAYAPLTCYFD |
| 528 | CM50 | EEDDYDAYAPITCYFD |
| 529 | CM178 | DEDDWDAYAPLTCYFD |
| 530 | CM304 | DEDDWDAYAPIWCYFT |
| 531 | CM155 | EEDDWDAYAPIWCYFT |
| 532 | CM452 | DDDDWDAYAPIWCYFT |
| 533 | CM148 | DEDDYDAYAPIWCYFT |
| 534 | CM322 | EEDDYDAYAPIWCYFD |
| 535 | CM526 | DDDDWWDYAPLTCYFD |
| 536 | CM1578 | EWDDWTDYAPLVCYFD |
| 537 | CM424 | DDDDYWDYAPLTCYFD |
| 538 | CM1358 | EWDDYWDYAPITCYFD |
| 539 | CM1530 | EEDDWTDYWPITCYFD |

TABLE 8
Exemplary HD845 variants with improved chemokine-binding
and/or chemotaxis properties

| SEQ ID NO | HD845 mutation | Sequence |
|---|---|---|
| 540 | E6W | EDEDYWDFFKPVTCYF |
| 541 | E6F | EDEDYFDFFKPVTCYF |
| 542 | K10A | EDEDYEDFFAPVTCYF |
| 543 | K10W | EDEDYEDFFWPVTCYF |

### REFERENCES

Abraham, M. e. (2017). Development of novel promiscuous anti-chemokine peptibodies for treating autoimmunity and inflammation. Frontiers in immunology, 1432.
Ahangarzadeh, S. e. (2019). Bicyclic peptides: types, synthesis and applications. Drug Discovery Today, 1311-1319.
Ahn, G. e. (2021). LYTACs that engage the asialoglycoprotein receptor for targeted protein degradation. Nature chemical biology, 937-946.
Asgari E, M. M. (2015). Continuous Distributed Representation of Biological Sequences for Deep Proteomics and Genomics. PLoS One, e0141287.
Banik, S. M. (2020). Lysosome-targeting chimaeras for degradation of extracellular proteins. Nature, 291-297.
Bhattacharya, S. a. (2020). Using evasins to target the chemokine network in inflammation. Advances in protein chemistry and structural biology, 1-38.
Blanco-Melo, D. e. (2020). Imbalanced host response to SARS-COV-2 drives development of COVID-19. Cell, 1036-1045.
Bonvin, P. C. (2016). vasins: therapeutic potential of a new family of chemokine-binding proteins from ticks. Frontiers in immunology, 208.
Brunetti, J. e. (2018). Branched Peptides as Bioactive Molecules for Drug Design. Peptide Science, e24089.
Buentzel, J. a. (2017). The Use of Glycosylation Tags as Reporters for Protein Entry into the Endoplasmic Reticulum in Yeast and Mammalian Cells. Peroxisomes, 221-232.
Darlot, B. E. A. (2020). Engineered anti-inflammatory peptides inspired by mapping an evasin-chemokine interaction. Journal of Biological Chemistry, 10926-10939.
Denisov, S. S. (2020). Structural characterization of anti-CCL5 activity of the tick salivary protein evasin-4. Journal of Biological Chemistry, 14367-14378.
Ernst, C. e. (2018). Switching Between Bicyclic and Linear Peptides—The Sulfhydryl-Specific Linker TPSMB Enables Reversible Cyclization of Peptides. Frontiers in chemistry, 484.
Gao, R. e. (2013). Cytokine and chemokine profiles in lung tissues from fatal cases of 2009 pandemic influenza A (H1N1): role of the host immune response in pathogenesis. The American journal of pathology, 1258-1268.
Hamley, I. W. (2014). PEG-peptide conjugates. Biomacromolecules, 1543-1559.
Hayes, e. a. (2021). Approaches for peptide and protein cyclisation. Organic & biomolecular chemistry, 3983-4001.
Heinis, C. e. (2009). Phage-encoded combinatorial chemical libraries based on bicyclic peptides. Nature chemical biology, 502-507.
Jeong, W. j. e. (2018). Peptide-Nanoparticle Conjugates: A Next Generation of Diagnostic and Therapeutic Platforms? Nano Convergence, 1-8.
Jumper J, E. R. P. (2021). Highly accurate protein structure prediction with AlphaFold. Nature, 583-589.
Lee, A. D. (2019). A knottin scaffold directs the CXC-chemokine-binding specificity of tick evasins. Journal of Biological Chemistry, 11199-11212.
Loktev, A. e. (2017). Multicyclic Peptides as Scaffolds for the Development of Tumor Targeting Agents. Current Medicinal Chemistry, 2141-2155.
McLaughlin, M. a. (2013). Engineering and analysis of peptide-recognition domain specificities by phage display and deep sequencing. Methods in enzymology, 327-349.
McNaughton, E. F. (2018). Novel anti-inflammatory peptides based on chemokine-glycosaminoglycan interactions reduce leukocyte migration and disease severity in a model of rheumatoid arthritis. The Journal of Immunology, 3201-3217.
Mézière, C. e. (1997). In vivo T helper cell response to retro-inverso peptidomimetics. The Journal of Immunology, 3230-3237.
Mosevoll, K. A. (2018). Inflammatory mediator profiles differ in sepsis patients with and without bacteremia. Frontiers in immunology, 691.
Sapra, R. e. (2019). Designer Peptide and Protein Dendrimers: A Cross-Sectional Analysis. Chemical reviews, 11391-11441.
Shimamoto, G. e. (2012). Peptibodies: A flexible alternative format to antibodies. MAbs. Škrlec, K. e. (2017). Evasin-displaying lactic acid bacteria bind different chemokines and neutralize CXCL 8 production in Caco-2 cells. Microbial biotechnology, 1732-1743.
Tan, H. e. (2018). Recent Advances in Half-Life Extension Strategies for Therapeutic Peptides and Proteins. Current Pharmaceutical Design, 4932-4946.
Tonikian, e. a. (2007). Identifying specificity profiles for peptide recognition modules from phage-displayed peptide libraries. Nature protocols, 1368-1386.
Yaron, J. R. (2020). Deriving immune modulating drugs from viruses-A new class of biologics. Journal of clinical medicine, 972.

### Further Aspects of the Invention

1. A combinatorial peptide entity comprising a plurality of independently disposed heterologous chemokine-binding peptides.
2. The combinatorial peptide entity of aspect 1, which has an altered chemokine binding profile when compared to any one of the chemokine-binding peptides individually.
3. The combinatorial peptide entity of aspect 1 or 2, wherein the combinatorial peptide entity comprises three or more chemokine-binding peptides, optionally wherein the combinatorial peptide entity comprises four or more chemokine-binding peptides.
4. The combinatorial peptide entity of any one of the preceding aspects, wherein the chemokine-binding peptides are fragments of viral chemokine-binding proteins, tick chemokine-binding proteins, mammalian chemokines, or variants thereof.
5. The combinatorial peptide entity of any one of the preceding aspects, wherein the combinatorial peptide entity is:
   (a) a peptibody;
   (b) a branched peptide;
   (c) a multicyclic peptide;
   (d) a nanoparticle;
   (e) a terminally linked peptide;
   (f) a side-chain linked peptide;
   (g) a PEG-linked peptide; or
   (h) a dendrimer.
6. The combinatorial peptide entity of any one of the preceding aspects, wherein the combinatorial peptide entity is chemically modified to enhance (a) bioactivity and/or (b) absorption, distribution, metabolism and/or excretion characteristics.
7. The combinatorial peptide entity of any one of the preceding aspects, wherein each chemokine-binding peptide is 50 or fewer amino acids in length, such as 40 or fewer, 30 or fewer, or 20 or fewer amino acids in length.
8. The combinatorial peptide entity of any one of the preceding aspects, wherein each chemokine-binding peptide is at least 10 amino acids in length, such as at least 15 amino acids in length.
9. The combinatorial peptide entity of any one of the preceding aspects, wherein each chemokine-binding peptide is 5 to 50 amino acids in length, such as 10 to 40, 10 to 30, 10 to 20, or 15 to 20 amino acids in length.
10. A chemokine-binding peptide comprising the amino acid sequence of any one of SEQ ID NOs: 1 to 472 or a variant thereof that retains the ability to bind to at least one chemokine.
11. The chemokine-binding peptide of aspect 10, which binds to at least three different chemokines.
12. The chemokine-binding peptide of aspect 10 or 11, wherein a variant comprises 1 to 6 amino acid modifications to a said amino acid sequence, optionally wherein the modification is an amino acid substitution, deletion or addition.
13. The combinatorial peptide entity of any one of aspects 1 to 9, which comprises one or more chemokine binding peptides according to any one of aspects 10 to 12.
14. A combinatorial peptide entity comprising a plurality of chemokine-binding peptides according to any one of aspects 10 to 12, optionally wherein the combinatorial peptide entity comprises:
   (i) two or more copies of the same chemokine-binding peptide or variants thereof and/or two or more different chemokine-binding peptides, optionally wherein the plurality of chemokine-binding peptides is linked in-series or independently disposed; and/or
   (ii) at least four copies of the same chemokine binding peptide or variants thereof and/or at least four different chemokine-binding peptides.
15. The combinatorial peptide entity of any one of aspects 1 to 9, 13 and 14, wherein the combinatorial peptide entity binds to CXC-class chemokines and CC-class chemokines.
16. A polynucleotide that encodes a chemokine-binding peptide according to any one of aspects 10 to 12, a vector that encodes the polynucleotide, or a host cell comprising the polynucleotide or the vector.
17. A pharmaceutical composition comprising a chemokine-binding peptide according to any one of aspects 10 to 12, a combinatorial peptide entity according to any one of aspects 1 to 9 and 13 to 15, or a vector, a polynucleotide or a host cell according to aspect 16; and (b) a pharmaceutically acceptable carrier or diluent.
18. A library comprising a plurality of chemokine-binding peptides according to any one of aspects 10 to 12.
19. A method of producing a combinatorial peptide entity, comprising:
   (a) identifying the chemokines associated with a disease;
   (b) identifying a combination of two or more chemokine binding peptides that bind to the chemokines associated with the disease; and
   (c) producing a combinatorial peptide entity from said two or more peptides.
20. The method of aspect 19, wherein:
   (a) the chemokines associated with the disease have increased expression when compared to a non-disease state;
   (b) the two or more chemokine binding peptides bind to at least 50% of the chemokines associated with the disease; and/or
   (c) the chemokines contribute to inflammation in the disease.
21. A method of treating a disease associated with aberrant chemokine expression in a subject, comprising administering a pharmaceutical composition according to aspect 17.
22. The method of any one of aspects 19 to 21, wherein the disease is:
   (a) an inflammatory disease; and/or
   (b) selected from any one of myocarditis, myocardial infarction, myocardial ischemia, atherosclerosis, vasculitis, stroke, multiple sclerosis, Alzheimer's disease, autoimmune hepatitis, myositis, primary biliary cirrhosis, primary sclerosing cholangitis, liver fibrosis, non-alcoholic steatohepatitis, paracetamol liver injury, alcohol liver injury, idiopathic pulmonary fibrosis, COVID-19, Covid-19 cytokine storm, sepsis, sepsis cytokine storm, acute lung injury, cardiac allograft vasculopathy, sarcoidosis, influenza, influenza cytokine storm, inflammatory bowel disease, pancreatitis, rheumatoid arthritis, psoriasis, skin fibrosis, kidney fibrosis, atopic dermatitis, acute respiratory distress syndrome, breast cancer and colorectal cancer, optionally wherein the disease is acute respiratory distress syndrome.
23. The method of aspect 21 or 22, wherein the aberrant chemokine expression is aberrant expression of a chemokine, or combination of chemokines, listed in Table 4, 5A or 5B.
24. A method of binding a plurality of chemokines, comprising contacting said chemokines with a combinatorial peptide entity according to any one of aspects 1 to 9 and 13 to 15, optionally wherein the method is an in vitro method or an in vivo method.
25. A method of detecting one or more chemokines in a tissue, comprising contacting the tissue with a detectably-labelled combinatorial peptide entity according to any one of aspects 1 to 9 and 13 to 15, or a detectably labelled chemokine binding peptide according to any one of aspects 10 to 12, and detecting the binding of the combinatorial peptide entity or the chemokine binding peptide to one or more chemokines in the tissue.

1-30. (canceled)

31. A composition comprising:
(i) a combinatorial peptide entity comprising a plurality of independently disposed heterologous chemokine-binding peptides;
(ii) a chemokine-binding peptide comprising:
(a) the amino acid sequence of SEQ ID NO: 396, or a variant thereof that retains the ability to bind to one or more CC-class chemokines and one or more CXC-class chemokines;
(b) the amino acid sequence of any one of SEQ ID NO: 396, 403 and 392, or a variant thereof that retains the ability to bind to one or more CC-class chemokines and one or more CXC-class chemokines; or
(c) the amino acid sequence of any one of SEQ ID NOs: 1 to 543, or a variant thereof that retains the ability to bind to at least one chemokine;
(iii) a chemokine-binding peptide capable of binding to one or more CC-class chemokines and one or more CXC- and/or XC-class chemokines, the chemokine binding peptide comprising an amino acid sequence of the formula:
X_{A}X_{B}X_{A}X_{A}X_{C}X_{D}X_{D}X_{E}X_{F}X_{D}PX_{G}X_{D}C X_{E}X_{E}X_{H}
wherein:
X_{A} is D or E
X_{B} is D, E or W
X_{C} is Y or W
X_{D} is any amino acid
X_{E} is an aromatic amino acid, preferably Y or F
X_{F} is an aromatic amino acid or absent, preferably Y or For absent
X_{G} is a hydrophobic amino acid, preferably V, L or I
X_{H} is T, C, D or absent;
(iv) a combinatorial peptide entity comprising a plurality of chemokine-binding peptides according to (ii) or (iii);
(v) a polynucleotide that encodes a chemokine-binding peptide according to (ii) or (iii), a vector that encodes the polynucleotide, or a host cell comprising the polynucleotide or the vector;
(vi) a pharmaceutical composition comprising
(a) a chemokine-binding peptide according to (ii) or (iii), a combinatorial peptide entity according to (i) or (iv), or a vector, a polynucleotide, or a host cell according to (v); and
(b) a pharmaceutically acceptable carrier or diluent; or
(vii) a library comprising a plurality of chemokine-binding peptides according to (ii) or (iii).

32. The composition of claim 31, wherein the combinatorial peptide entity of (i) has an altered chemokine binding profile when compared to any one of the chemokine-binding peptides individually.

33. The composition of claim 31, wherein the combinatorial peptide entity of (i) comprises three or more chemokine-binding peptides, optionally wherein the combinatorial peptide entity comprises four or more chemokine-binding peptides.

34. The composition of claim 31, wherein the plurality of independently disposed heterologous chemokine-binding peptides of (i) are fragments of viral chemokine-binding proteins, tick chemokine-binding proteins, mammalian chemokines, or variants thereof.

35. The composition of claim 31, wherein the combinatorial peptide entity of (i) is:
(a) a peptibody;
(b) a branched peptide;
(c) a multicyclic peptide;
(d) a nanoparticle;
(e) a terminally linked peptide;
(f) a side-chain linked peptide;
(g) a PEG-linked peptide; or
(h) a dendrimer.

36. The composition of claim 31, wherein the combinatorial peptide entity of (i) is chemically modified to enhance (a) bioactivity and/or (b) absorption, distribution, metabolism and/or excretion characteristics.

37. The composition of claim 31, wherein each of the plurality of independently disposed heterologous chemokine-binding peptides of (i):
(a) is 50 or fewer amino acids in length, such as 40 or fewer, 30 or fewer, or 20 or fewer amino acids in length; or
(b) at least 10 amino acids in length, such as at least 15 amino acids in length.

38. The composition of claim 31, wherein each of the plurality of independently disposed heterologous chemokine-binding peptides of (i) is 5 to 50 amino acids in length, such as 10 to 40, 10 to 30, 10 to 20, or 15 to 20 amino acids in length.

39. The composition of claim 31, wherein the chemokine-binding peptide of (ii) is a variant of SEQ ID NO: 396 and comprises:
(a) one or more of the following amino acid substitutions: E1D, E2D/W, D3 E, D4 E, Y5W, T6D/E/W, A7W/D, A9W, L11I, T12W and T16C/D, optionally which comprises one or more of Y5W, T6W, A9W and T16C; and/or
(b) the amino acid sequence of any one of SEQ ID NOs: 503-539.

40. The composition of claim 31, wherein the chemokine-binding peptide of (ii) binds to at least three different chemokines.

41. The composition of claim 31, wherein the chemokine-binding peptide of (ii) comprises a variant comprising 1 to 6 amino acid modifications to a said amino acid sequence, optionally wherein the modification is an amino acid substitution, deletion or addition.

42. The composition of claim 31, wherein the combinatorial peptide entity of (i) comprises one or more chemokine binding peptides according to (ii) or (iii).

43. The composition of claim 31, wherein the combinatorial peptide entity of (iv) comprises:
(a) two or more copies of the same chemokine-binding peptide or variants thereof and/or two or more different chemokine-binding peptides, optionally wherein the plurality of chemokine-binding peptides is linked in-series or independently disposed; and/or
(b) at least four copies of the same chemokine binding peptide or variants thereof and/or at least four different chemokine-binding peptides;

44. The composition of claim 31, wherein the combinatorial peptide entity of (i) or (iv) binds to CXC-class chemokines and CC-class chemokines.

45. A method comprising:
(i) producing a combinatorial peptide entity, comprising:
(a) identifying the chemokines associated with a disease;
(b) identifying a combination of two or more chemokine binding peptides that bind to the chemokines associated with the disease; and
(c) producing a combinatorial peptide entity from said two or more peptides;
(ii) treating a disease associated with aberrant chemokine expression in a subject, comprising administering a pharmaceutical composition comprising the composition of claim 31 and a pharmaceutically acceptable carrier;
(iii) binding a plurality of chemokines, comprising contacting said chemokines with a combinatorial peptide entity according to claim 31, optionally wherein the method is an in vitro method or an in vivo method; or
(iv) detecting one or more chemokines in a tissue,
comprising contacting the tissue with the combinatorial peptide entity or the chemokine-binding peptide of claim 31, wherein the combinatorial peptide entity and the chemokine-binding peptide are detectably-labeled, and detecting the binding of the combinatorial peptide entity or the chemokine binding peptide to one or more chemokines in the tissue.

46. The method of claim 45, wherein:
(a) the chemokines associated with the disease of (i) have increased expression when compared to a non-disease state;
(b) the two or more chemokine binding peptides of (i) bind to at least 50% of the chemokines associated with the disease; and/or
(c) the chemokines of (i) contribute to inflammation in the disease.

47. The method of claim 45, wherein the disease is:
(a) an inflammatory disease; and/or
(b) selected from any one of myocarditis, myocardial infarction, myocardial ischemia, atherosclerosis, vasculitis, stroke, multiple sclerosis, Alzheimer's disease, autoimmune hepatitis, myositis, primary biliary cirrhosis, primary sclerosing cholangitis, liver fibrosis, non-alcoholic steatohepatitis, paracetamol liver injury, alcohol liver injury, idiopathic pulmonary fibrosis, COVID-19, COVID-19 cytokine storm, sepsis, sepsis cytokine storm, acute lung injury, cardiac allograft vasculopathy, sarcoidosis, influenza, influenza cytokine storm, inflammatory bowel disease, pancreatitis, rheumatoid arthritis, psoriasis, skin fibrosis, kidney fibrosis, atopic dermatitis, acute respiratory distress syndrome, breast cancer and colorectal cancer, optionally wherein the disease is acute respiratory distress syndrome.

48. The method of claim 45, wherein the aberrant chemokine expression of (ii) is aberrant expression of a chemokine, or combination of chemokines as listed in Table 4, Table 5A or Table 5B.

49. A method comprising:
(i) identifying a chemokine-binding peptide, comprising:
(a) constructing a phage-display library encoding a bacteriophage coat protein fused to 10-mer to 20-mer peptides of a chemokine-binding protein at single amino acid resolution to thereby produce a phage-display library of overlapping peptides;
(b) contacting the phage-display library with one or more chemokines to thereby produce an enriched library comprising peptides capable of binding the one or more chemokines; and
(c) sequencing the enriched library to thereby identify a chemokine-binding peptide; or
(ii) enhancing and/or expanding chemokine binding activity of a chemokine-binding peptide comprising:
(a) substituting one or more codons encoding a chemokine-binding peptide in a phage-display system with the sequence NNK, to thereby produce a mutant phage display library encoding mutated chemokine-binding peptide;
(b) contacting the mutated phage-display library with one or more chemokines to thereby produce an enriched library comprising peptides capable of binding the one or more chemokines; and
(c) sequencing the enriched library to thereby identify one or more mutated chemokine-binding peptides with enhanced or expanded chemokine binding activity.

50. The method of claim 49, wherein the enhancing and/or expanding chemokine binding activity of a chemokine-binding peptide further comprises:
(d) constructing a combinatorial mutant phage display library comprising combinations of the substitutions in the one of more mutated chemokine-binding peptides identified in step (c);
(e) contacting the combinatorial mutant phage display library with one or more chemokines to thereby produce an enriched combinatorial mutant library comprising peptides capable of binding the one or more chemokines; and
(f) sequencing the enriched combinatorial mutant library to thereby identify combinatorially mutated chemokine-binding peptides with further enhanced or expanded chemokine binding activity.
